(19) United States
(12) Patent Application Publication
(10) Pub. No.: US 2024/0327544 A1
(43) Pub. Date: Oct. 3, 2024

(54) MULTISPECIFIC BINDING AGENTS AGAINST CD40 AND CD137 IN COMBINATION THERAPY FOR CANCER

(71) Applicants: BIONTECH SE, Mainz (DE); GENMAB A/S, Valby (DK)

(72) Inventors: Ugur SAHIN, Mainz (DE); Alexander MUIK, Mainz (DE); Sina FELLERMEIER-KOPF, Mainz (DE); Yali FU, Plainsboro, NJ (US); Homer ADAMS, III, Plainsboro, NJ (US); Gaurav BAJAJ, Plainsboro, NJ (US); Brandon HIGGS, Plainsboro, NJ (US); Mark FERESHTEH, Plainsboro, NJ (US); Vanessa SPIRES, Plainsboro, NJ (US); Jordan BLUM, Plainsboro, NJ (US); Patricia GARRIDO CASTRO, Utrecht (NL); Michelle NIEWOOD, Plainsboro, NJ (US); Friederike GIESEKE, Mainz (DE); Karsten BECKMANN, Mainz (DE); Claudia PAULMANN, Mainz (DE); Ivan KUZMANOV, Mainz (DE); Esther Cornelia Wilhelmina BREIJ, Utrecht (NL); Lars GUELEN, Utrecht (NL); Jost NEIJSSEN, Utrecht (NL); Bart-Jan DE KREUK, Utrecht (NL); Richard HIBBERT, Utrecht (NL); Janine SCHUURMAN, Utrecht (NL); Aran Frank LABRIJN, Utrecht (NL)

(21) Appl. No.: 18/579,086

(22) PCT Filed: Jul. 13, 2022
(86) PCT No.: PCT/EP2022/069639

§ 371 Date: Jan. 12, 2024

Related U.S. Application Data
(60) Provisional application No. 63/364,594, filed on May 12, 2022.
(60) Provisional application No. 63/221,340, filed on Jul. 13, 2021.

(51) Int. Cl. C07K 16/46; A61K 39/00

(52) U.S. Cl. CPC C07K 16/468; A61K 2039/505; C07K 2317/31; C07K 2317/33; C07K 2317/52; C07K 2317/565; C07K 2317/567; C07K 2317/71; C07K 2317/76

(57) ABSTRACT

The present invention relates to combination therapy using a binding agent that binds to human CD40 and to human CD137 in combination with a checkpoint inhibitor to reduce or prevent progression of a tumor or treating cancer.

### TECHNICAL FIELD

The present invention relates to combination therapy using a binding agent that binds to human CD40 and to human CD137 in combination with a checkpoint inhibitor to reduce or prevent progression of a tumor or treating cancer.

### BACKGROUND

CD40 is a member of the tumor necrosis factor (TNF) receptor (TNFR) family and is known as a co-stimulatory protein found on a diversity of cell types. CD40 is constitutively expressed by antigen-presenting cells (APCs), including dendritic cells (DCs), B cells and macrophages. It can also be expressed by endothelial cells, platelets, smooth muscle cells, fibroblasts and epithelial cells. Consistent with its widespread expression on normal cells, CD40 is also expressed on a wide range of tumor cells.

The presentation of peptide antigens in the context of MHC class II molecules to antigen-specific CD4⁺ T cells, together with co-stimulatory signals (from CD80 and/or CD86), results in the activation of CD4⁺ T cells and the up-regulation of the DC licensing factors CD40 ligand (CD40L) and lymphotoxin-α1β2 (LTα1β2). Expression of CD40L and LT LTα1β2 on activated antigen-specific CD4+ T cells induces signaling through CD40 and the LTβ receptor (LTβR), and this licenses DCs to induce CD8⁺ T-cell responses. CD40 signaling results in the production of interleukin-12 (IL-12) and the up-regulation of CD70, CD86, 4-1BB ligand (4-1BBL), OX40 ligand (OX40L) and GITR ligand (GITRL), whereas LTβR signaling leads to the production of type 1 interferons (IFNs). The signaling system that controls the activity of nuclear factor kappaB (NF-κB) is responsive to virtually all TNFR superfamily members. Pathogen-associated molecular patterns (PAMPs) and damage-associated molecular patterns (DAMPs) also contribute to these events. Priming of CD8⁺ T cells by MHC class I-restricted peptides results in the up-regulation of CD27, 4-1BB, OX40 and glucocorticoid-induced TNFR-related protein (GITR). Stimulation of these receptors on CD8⁺ T cells by their cognate TNF superfamily ligands, in combination with IL-12 and type 1 IFNs, results in robust CD8⁺ T cell activation, proliferation and effector function, as well as the formation and maintenance of CD8⁺ T cell memory. CD40 antibodies can exert different actions: CD40-expressing tumor cell kill by induction of antibody-dependent cell-mediated cytotoxicity (ADCC), complement-dependent cytotoxicity (CDC) or antibody-dependent cell-mediated phagocytosis (ADCP), induction of cell signaling to induce direct apoptosis or growth arrest, but also, independent of CD40 expression on the tumor cells, through licensing of APCs to stimulate an anti-cancer immune response. Antibodies binding to CD40 can trigger CD40 on APCs to prime effector cytotoxic T lymphocytes (CTLs) and induce release of IL-2 by these cells, and indirectly activate NK cells. Antibodies stimulating CD40 have been disclosed in the prior art, and include CP-870,893, a human IgG2 antibody (WO 03/040170); dacetuzumab, a humanized IgG1 antibody (WO 00/075348) and Chi Lob 7/4, a chimeric IgG1 antibody (US 2009/0074711). Furthermore, an antagonistic CD40 antibody has been disclosed, lucatumumab, a human IgG1 antibody (WO 02/028481).

CD137 (4-1BB) is also a member of the TNFR family. CD137 is a co-stimulatory molecule on CD8⁺ and CD4+ T cells, regulatory T cells (Tregs), Natural Killer T cells (NK(T) cells), B cells and neutrophils. On T cells, CD137 is not constitutively expressed, but induced upon T-cell receptor (TCR) activation (for example, on tumor infiltrating lymphocytes (TILs) (Gros et al., J. Clin Invest 2014; 124(5):2246-59)). Stimulation via its natural ligand 4-1BBL or agonist antibodies leads to signaling using TRAF-2 and TRAF-1 as adaptors. Early signaling by CD137 involves K-63 poly-ubiquitination reactions that ultimately result in activation of the nuclear factor (NF)-κB and mitogen-activated protein (MAP)-kinase pathways. Signaling leads to increased T cell co-stimulation, proliferation, cytokine production, maturation and prolonged CD8+ T-cell survival. Agonistic antibodies against CD137 have been shown to promote anti-tumor control by T cells in various pre-clinical models (Murillo et al., Clin Cancer Res 2008; 14(21):6895-906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (AU 2004279877) and utomilumab, a human IgG2 antibody (Fisher et al., 2012, Cancer Immunol. Immunother. 61: 1721-1733).

Westwood J A, et al., Leukemia Research 38 (2014), 948-954 discloses “Combination anti-CD137 and anti-CD40 antibody therapy in murine myc-driven hematological cancers”. WO 2018/011421 provides binding agents, such as bispecific antibodies, binding human CD40 and binding human CD137. Such bispecific antibodies crosslink CD40 on antigen presenting cells (APCs) with 4-1BB on activated T cells, and thereby induce conditional stimulation of and co-stimulatory activity in both cell types useful for the treatment of solid tumors.

PD-1, CTLA4, PD-L1, TIM-3, KIR or LAG-3 are inhibitory checkpoint molecules regulating the immune system and enabling self-tolerance. At the same time inhibitory checkpoint molecules are ideal targets for cancer immunotherapy.

In tumor-draining lymph nodes and within the tumor microenvironment, 4-1BB is expressed by a subset of CD4+ and CD8+ T cells that are characterized by the co-expression of multiple TCR-inducible molecules including high levels of programmed cell death 1 (PD-1) (Gros et al., J. Clin Invest 2014; 124(5):2246-59; Seifert et al., Cancers (Basel) 12; Simoni et al., Nature 557: 575-579). Upregulation of PD-1 on T cells can contribute to T-cell exhaustion and reduce T-cell activation upon binding to its ligand programmed cell death 1 ligand 1 (PD-L1) (Yu et al., Eur J Pharmacol 881: 173240). PD-L1 expression is often upregulated by tumor cells, particularly in inflamed tumors (Teng, et al., Cancer Res 75: 2139-2145). Thereby, the tumor cells provide an inhibitory signal to the activated T cells through which they can evade T-cell mediated cytotoxicity. Antibodies that block the PD-1/PD-L1 inhibitory axis can restore T-cell function (Boussiotis et al., N Engl J Med 375: 1767-1778; Chen et al., Nature 541: 321-330).

However, despite these advances in the art there is a considerable need for improved therapies to prevent progression of a tumor or treating cancer.

### SUMMARY

The present inventors have surprisingly found that a combination of(i) stimulation with a binding agent binding human CD40 and binding human CD137 and (ii) checkpoint inhibition (in particular inhibition of the PD-1/PD-L1 axis) amplifies the immune response.

Thus, in a first aspect, the present disclosure provides a binding agent for use in a method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137.

In a second aspect, the present disclosure provides a kit comprising (i) a binding agent comprising a first binding region binding to CD40 and a second binding region binding to CD137, (ii) a checkpoint inhibitor, and optionally (iii) one or more additional therapeutic agents.

In a third aspect, the present disclosure provides a kit of the second aspect for use in a method for reducing or preventing progression of a tumor or treating cancer in a subject.

In a fourth aspect, the present disclosure provides a method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137.

### BRIEF DESCRIPTION OF THE FIGURES

FIG. 1 shows a schematic representation of the anticipated mode of action of the CD40×4-1BB bispecific antibody. CD40 is expressed on antigen-presenting cells (APCs) as well as on tumor cells. 4-11BB (CD137) is expressed on activated T cells. DuoBody-CD40×4-1BB (GEN1042/BNT312) is a bispecific antibody that crosslinks CD40 on antigen presenting cells (APCs) with 4-1BB on activated T cells, thereby conditionally stimulating both cell types. Thereby CD40×4-1BB bispecific antibodies may enhance DC licensing, T-cell clonal expansion, cytokine production, T-cell survival and T-cell and NK-cell-mediated cytotoxicity.

FIG. 2 shows IFNγ production induced by bsIgG1-CD40×4-1BB in combination with pembrolizumab in a mixed lymphocyte reaction (MLR) of mature dendritic cells (mDCs) and purified CD8+ T-cells. Purified CD8+ T cells were co-cultured with allogeneic LPS-matured DCs in the presence of bsIgG1-CD40×4-1BB (0.001-30 μg/mL), pembrolizumab (0.1-30 μg/mL) either alone or in combination for 5 days. IFNγ secretion was analyzed by ELISA. Data shown are mean IFNγ±standard deviation (SD) of duplicate wells from one representative donor pair of five donor pairs included in three experiments. The horizontal lines in the graph show from top to bottom the IFNγ production for 10 μg/mL bsIgG1-CD40×4-1BB without pembrolizumab (interrupted line), for 10 μg/mL pembrolizumab without bsIgG1-CD40×4-1BB (interrupted, dotted line), and without treatment (simple thin line).

FIG. 3 shows IFNγ production induced by bsIgG1-CD40×4-1BB in combination with pembrolizumab in a mixed lymphocyte reaction (MLR) of mature dendritic cells (mDCs) and purified CD8+ T-cells. Purified CD8+ T cells were co-cultured with allogeneic LPS-matured DCs in the presence of bsIgG1-CD40×4-1BB (0.001-30 μg/mL), pembrolizumab (0.1-30 μg/mL) either alone or in combination for 5 days. IFNγ secretion was analyzed by ELISA. Data shown are mean IFNγ±standard deviation (SD) of duplicate wells in three experiments. Each individual graph represents one of five donor pairs.

FIG. 4 shows IFNγ production induced by DuoBody-CD40×4-1BB in combination with pembrolizumab in a mixed lymphocyte reaction (MLR) of mature dendritic cells (mDCs) and purified CD8+ T-cells. Purified CD8+ T cells were co-cultured with allogeneic LPS-matured DCs in the presence of DuoBody-CD40×4-1BB (0.001-30 μg/mL), pembrolizumab (0.1-100 μg/mL) either alone or in combination, or control antibodies bsIgG1-CD40×ctrl, bsIgG1-ctrl×4-1BB, IgG1-ctrl-FEAL (all 30 μg/mL) or IgG4 isotype control (100 μg/mL) for 5 days. IFNγ secretion was analyzed by ELISA. Data shown are mean IFNγ±standard deviation (SD) of duplicate wells of one donor pair (n=1).

FIG. 5 shows IFNγ production induced by DuoBody-CD40×4-1BB or bsIgG1-CD40×4-1BB either alone or in combination with pembrolizumab in a mixed lymphocyte reaction (MLR) of mature dendritic cells (mDCs) and purified CD8+ T-cells. Purified CD8+ T cells were co-cultured with allogeneic LPS-matured DCs in the presence of DuoBody-CD40×4-1BB (0.001-30 μg/mL), bsIgG1-CD40×4-1BB (0.001-30 μg/mL), either alone or in combination with pembrolizumab (1 μg/mL) for 5 days. An additional monotherapy control treatment of 30 μg/mL IgG1-ctrl-FEAL was evaluated. IFNγ secretion was analyzed by ELISA. Data shown are mean IFNγ±standard deviation (SD) of duplicate wells of one donor pair (n=1).

FIG. 6 shows IFNγ production induced by bsIgG1-CD40×4-1BB in combination with an in house derived nivolumab in a mixed lymphocyte reaction (MLR) of mature dendritic cells (mDCs) and purified CD8+ T-cells. Purified CD8+ T cells were co-cultured with allogeneic LPS-matured DCs in the presence of bsIgG1-CD40×4-1BB (0.001-10 μg/mL), a nivolumab (α-PD-1; 0.0005-5 μg/mL) either alone or in combination for 5 days. IFNγ secretion was analyzed by ELISA. Data shown are mean IFNγ±standard deviation (SD) of duplicate wells from one donor pair.

FIG. 7 shows IFNγ secretion induced by IgG1-PD1 in combination with DuoBody-CD40×4-1BB in an allogeneic mixed lymphocyte reaction (MLR) assay. Two unique donor pairs of allogeneic human mature dendritic cells (mDCs) and CD8+ T cells were co-cultured for 5 days in the presence of IgG1-PD1 (0.001-100 μg/mL), DuoBody-CD40×4-1BB (0.001-30 μg/mL), or the combination of IgG1-PD1 and DuoBody-CD40×4-1BB. IgG1-ctrl-FERR (100 μg/mL), bsIgG1-CD40×ctrl (30 μg/mL), bsIgG1-ctrl×4-1BB (30 μg/mL), and IgG1-ctrl-FEAL (30 μg/mL) were included as controls. IFNγ secretion was analyzed in the supernatant using an IFNγ-specific AlphaLISA immunoassay. Data shown are the mean IFNγ levels±standard error of the mean (SEM) of 2 unique allogeneic donor pairs treated with one representative concentration of 1 μg/mL IgG1-PD1.

FIG. 8 shows synergy analysis for IFNγ secretion induced by the combination of IgG1-PD1 and DuoBody-CD40×4-1BB in an allogeneic mixed lymphocyte reaction (MLR) assay. The synergistic effect of IgG1-PD1 and DuoBody-CD40×4-1BB treatment combinations in a dose-response matrix of 7×7 in the allogeneic MLR assay was determined using the Bliss and Highest Single Agent (HSA) synergy scoring models for donor pair 1 (A) and 2 (B). A score of ≥10 indicates synergy.

FIG. 9 shows enhancement of CD8+ T-cell proliferation by IgG1-PD1 in combination with DuoBody-CD40×4-1BB in an antigen-specific T-cell stimulation assay. Human CD8+ T cells were electroporated with RNA encoding a claudin 6 (CLDN6)-specific T-cell receptor (TCR) and RNA encoding programmed cell death protein 1 (PD-1) and labeled with carboxyfluorescein succinimidyl ester (CFSE). The T cells were then co-cultured with immature dendritic cells (iDCs) electroporated with CLDN6, in the presence of 0.8 μg/mL IgG1-PD1, pembrolizumab, or IgG1-ctrl-FERR, either alone or in combination with the indicated concentrations of DuoBody-CD40×4-1BB. CFSE dilution in T cells was analyzed by flow cytometry after 4 days and used to calculate the expansion index. Data from one representative donor out of four donors evaluated in two independent experiments are shown. Error bars represent standard deviation (SD) of duplicate wells. Dotted line indicates expansion index of CD8+ T cells co-cultured with mock-electroporated (i.e. not expressing CLDN6) iDCs.

FIG. 10 shows enhancement of cytokine secretion by IgG1-PD1 in combination with DuoBody-CD40×4-1BB after antigen-specific CD8+ T-cell stimulation. Human CD8+ T cells expressing a claudin 6 (CLDN6)-specific T-cell receptor (TCR) and programmed cell death protein 1 (PD-1) were co-cultured with CLDN6-expressing iDCs as in FIG. 9, in the presence of 0.8 μg/mL IgG1-PD1, pembrolizumab, or IgG1-ctrl-FERR, either alone or in combination with the indicated concentrations of DuoBody-CD40×4-1BB. Cytokine concentrations in culture supernatants were determined after 4 days. Data from one representative donor out of four donors evaluated in two independent experiments are shown. Error bars represent standard deviation (SD) of duplicate wells.

FIG. 11 shows binding of IgG1-PD1 to PD-1 of different species. CHO-S cells transiently transfected with PD-1 of different species were incubated with IgG1-PD1, pembrolizumab, or non-binding control antibodies IgG1-ctrl-FERR and IgG4-ctrl and binding analyzed using flow cytometry. Non-transfected CHO-S cells incubated with IgG1-PD1 were included as a negative control. A-B. Data shown are the geometric mean fluorescence intensities (gMFI)±SD of duplicate wells from one representative experiment out of four experiments. C-D. Data shown are the gMFI±SD of duplicate wells from one representative experiment out of two experiments. E. Data shown are the geometric mean fluorescence intensities (gMFI)±SD of duplicate wells from one representative experiment out of four experiments. Abbreviations: gMFI=geometric mean fluorescence intensity; PD-1=programmed cell death protein 1; PE=R-Phycoerythrin.

FIG. 12 shows competitive binding of IgG1-PD1 with PD-L1 and PD-L2 to human PD-1. CHO-S cells transiently transfected with human PD-1 were incubated with 1 μg/mL biotinylated recombinant human PD-L1 (A) or PD-L2 (B) in the presence of IgG1-PD1 or pembrolizumab. IgG1-ctrl-FERR was included as a negative control. Cells were stained with streptavidin-allophycocyanin, and the percentage of cells binding biotinylated PD-L1 or PD-L2 was determined by measuring the percentage of streptavidin-allophycocyanin⁺ cells using flow cytometry. The percentage of streptavidin-allophycocyanin-cells in the no antibody control and in a non-transfected sample are indicated with dashed lines. Data shown are from single replicates from one representative experiment out of three separate experiments. Abbreviations: Ab=antibody; CHO-S=Chinese hamster ovary, suspension; ctrl=control; FERR=L234F/L235E/G236R-K409R; PD-1=programmed cell death protein 1; PD-L1=programmed cell death 1 ligand 1; PD-L2=programmed cell death 1 ligand 2.

FIG. 13 shows functional inhibition of the PD-1/PD-L1 checkpoint by IgG1-PD1. Blockade of the PD-1/PD-L1 axis was tested using a cell-based bioluminescent PD-1/PD-L1 blockade reporter assay. Data shown are mean luminescence±SD of duplicate wells in one representative experiment out of five (pembrolizumab and IgG1-PD1), three (IgG1-ctrl-FERR) or two (nivolumab) experiments. Abbreviations: FERR=L234F/L235E/G236R-K409R; PD1=programmed cell death protein 1; PD-L1=programmed cell death 1 ligand 1; RLU=relative light units; SD=standard deviation.

FIG. 14 shows the enhancement of CD8⁺ T-cell proliferation by IgG1-PD1 in an antigen-specific T-cell proliferation assay. Human CD8⁺ T cells were electroporated with RNA encoding a CLDN6-specific TCR and RNA encoding PD-1 and labeled with CFSE. The T cells were then co-cultured with iDCs electroporated with CLDN6-encoding RNA, in the presence of IgG1-PD1, pembrolizumab, nivolumab, or IgG1-ctrl-FERR. CFSE dilution in T cells was analyzed by flow cytometry after 4 d and used to calculate the expansion index. Data from one representative donor (26268_B) out of four donors evaluated in three independent experiments are shown. Error bars represent SD of duplicate wells. Curves were fitted by 4-parameter logarithmic fit using GraphPad Prism. Abbreviations: CFSE=carboxyfluorescein succinimidyl ester; FERR=L234F/L235E/G236R-K409R; PD1=programmed cell death protein 1; SD=standard deviation.

FIG. 15 shows IgG1-PD1-induced IFNγ secretion in an allogeneic MLR assay. Three unique donor pairs of allogeneic human mDCs and CD8+ T cells were cocultured in the presence of IgG1-PD1 or pembrolizumab for 5 d. IgG1-ctrl-FERR and an IgG4 isotype control were included as negative controls. IFNγ secretion was analyzed in the supernatant using an IFNγ-specific immunoassay. Data shown are mean±standard error of the mean (SEM) concentration for three unique allogeneic donor pairs. Abbreviations: FERR=L234F/L235E/G236R-K409R; IFN=interferon; IgG=immunoglobulin G; mDC=mature dendritic cell; MLR=mixed lymphocyte reaction; SEM=standard error of the mean.

FIG. 16 shows IgG1-PD1-induced cytokine secretion in an allogeneic MLR assay. Three unique donor pairs of allogeneic human mDCs and CD8⁺ T cells were cocultured in the presence of 1 μg/mL IgG1-PD1 or pembrolizumab for 5 d. IgG1-ctrl-FERR was included as a negative control. Cytokine secretion was analyzed in the supernatant using Luminex. (A) Cytokine levels are represented as the average fold change over the cytokine levels measured in untreated cocultures. (B) Shown are the levels of cytokine production of three unique allogeneic donor pairs, with horizontal lines indicating the mean, upper, and lower limits. Abbreviations: FC=fold change; FERR=L234F/L235E/G236R-K409R; GM-CSF=granulocyte macrophage colony-stimulating factor; IgG=immunoglobulin G; IL=interleukin; MCP-1=monocyte chemoattractant protein 1; mDC=mature dendritic cell; MLR=mixed lymphocyte reaction; TNF=tumor necrosis factor.

FIG. 17 shows C1q binding to membrane-bound IgG1-PD1. Binding of C1q to IgG1-PD1 was analyzed using stimulated human CD8⁺ T cells. After incubation with IgG1-PD1, IgG1-ctrl-FERR, IgG1-ctrl, or positive control antibody IgG1-CD52-E430G (without inertness mutations and with a hexamerization-enhancing mutation), cells were incubated with human serum as a source of C1q. Binding of C1q was detected with a FITC-conjugated rabbit anti-C1q antibody. Data shown are the geometric mean fluorescence intensities (gMFI) t standard deviation (SD) from duplicate wells from one representative donor out of seven donors across three comparable experiments. Abbreviations: FITC=fluorescein isothiocyanate; gMFI=geometric mean fluorescence intensity; PE=R-phycoerythrocyanin.

FIG. 18 shows FcγR binding of IgG1-PD1. The binding of IgG1-PD1 to immobilized human recombinant FcγR constructs was analyzed by SPR in a qualified assay (n=1). FcγRIa (A), FcγRIIa-H131 (B), FcγRIIa-R131 (C), FcγRIIb (D), FcγRIIIa-F158 (E), and FcγRIIIa-V158 (F) binding of IgG1-PD1. The antibody IgG1-ctrl (without the FER inertness mutations) was included as a positive control for binding. Abbreviations: ctrl=control; FcγR=Fe gamma receptor; IgG=immunoglobulin G; PD-1=programmed cell death protein 1; RU=resonance units.

FIG. 19 shows FcγR binding of IgG1-PD1 and several other anti-PD-1 antibodies. The binding of IgG1-PD1, nivolumab, pembrolizumab, dostarlimab, and cemiplimab to immobilized human recombinant FcγR constructs was analyzed by SPR (n=3). FcγRIa (A), FcγRIIa-H131 (B), FcγRIIa-R131 (C), FcγRIIb (D), FcγRIIIa-F158 (E), and FcγRIIIa-V158 (F) binding of the test antibodies. The IgG1-ctrl and IgG4-ctrl antibodies were included as positive controls for FcγR binding of IgG1 and IgG4 molecules with wild-type Fe regions. Shown is the binding response±SD of three separate experiments. Abbreviations: ctrl=control; FcγR=Fe gamma receptor; IgG=immunoglobulin G; PD-1=programmed cell death protein 1; RU=resonance units.

FIG. 20 shows FcγRIa binding of IgG1-PD1 and several other anti-PD-1 antibodies. The binding of IgG1-PD1, nivolumab, pembrolizumab, dostarlimab, and cemiplimab to CHO-S cells transiently expressing human FcγRIa was analyzed by flow cytometry. IgG1-ctrl and IgG1-ctrl-FERR were included as a positive and negative control, respectively. Abbreviations: ctrl=control; FcγR=Fc gamma receptor; FERR=L234F/L235E/G236R-K409R; huIgG=human immunoglobulin G; PD-1=programmed cell death protein 1; PE=R-phycoerythrin.

FIG. 21 shows total human IgG in mouse plasma samples. Mice were injected intravenously with 1 or 10 mg/kg IgG1-PD1 at t=0 and serial plasma samples were taken at 10 min, 4 h, 1 d, 2 d, 8 d, 14 d, and 21 d after injection. Total huIgG in plasma samples was determined by ECLIA for each mouse. Data are represented as mean huIgG concentration±SD of three individual mice. Dashed lines indicate the plasma concentrations of wild-type (wt) huIgG predicted by a two-compartment model based on IgG clearance in humans (Bleeker et al., 2001, Blood. 98(10):3136-42). Dotted lines indicate the LLOQ and ULOQ. Abbreviations: huIgG=human IgG; IgG=immunoglobulin G; LLOQ=lower limit of quantitation; PD-1=programmed cell death protein 1; SD=standard deviation; ULOQ=upper limit of quantitation.

FIG. 22 shows antitumor activity of IgG1-PD1 in human PD-1 knock-in mice. The MC38 colon cancer syngeneic tumor model was established by SC implantation in hPD-1 K1 mice. Mice were administered 0.5, 2, or 10 mg/kg IgG1-PD1 or pembrolizumab or 10 mg/kg IgG1-ctrl-FERR 2QW×3 (9 mice per group). (A) Average tumor volume±SEM in each group, until the last time point the group was complete. (B) Tumor volumes of the different groups on the last day all groups were complete (Day 11). Data shown are the tumor volumes in individual mice in each treatment group, as well as mean tumor volume±SEM per treatment group. Mann-Whitney analysis was used to compare tumor volumes of the treatment groups to the IgG1-ctrl-FERR-treated group, with *p<0.05, **p<0.01, and ***p<0.001. C. Progression-free survival, defined as the percentage of mice with tumor volume smaller than 500 mm³, is shown as a Kaplan-Meier curve. Analysis excluded one mouse from the 2 mg/kg IgG1-PD1 group that was found dead due to undetermined cause on day 16, before the tumor volume had exceeded 500 mm³. Abbreviations: 2QW×3=twice per week for three weeks; ctrl=control; FERR=L234F/L235E/G236R/K409R mutations; IgG=immunoglobulin G; KI=knock-in; PD-1=programmed cell death protein 1; SC=subcutaneous; SEM=standard error of the mean.

FIG. 23 shows secretion of IFNγ (A), GM-CSF (B), TNFα (C), IL-2 (D) and IL-6 (E) induced by DuoBody-CD40×4-1BB in combination with atezolizumab, nivolumab or pembrolizumab in a mixed lymphocyte reaction (MLR) of mature dendritic cells (mDCs) and purified CD8+ T-cells. Purified CD8+ T cells were co-cultured with allogeneic LPS-matured DCs in the presence of DuoBody-CD40×4-1BB (0.001-30 μg/mL), atezolizumab (1 μg/mL), nivolumab (I μg/mL) or pembrolizumab (1 μg/mL) alone or DuoBody-CD40×4-1BB in combination with atezolizumab, nivolumab or pembrolizumab for 5 days. Co-cultures without treatment (No Tx), or treated with bsIgG1-CD40×ctrl (30 μg/mL), bsIgG1-ctrl×4-1BB (30 μg/mL) or IgG1-ctrl-FEAL (30 μg/mL) were included as controls. Secretion of IFNγ was analyzed by ELISA and secretion of GM-CSF, TNFα, IL-2 and IL-6 by Luminex. Data shown are mean+standard deviation (SD) of duplicate wells of one representative donor pair out of 4 pairs tested in one experiment.

FIG. 24. shows the effect of DuoBody-CD40×4-1BB in combination with anti-PD-(L)1 antibodies on T-cell proliferation in vitro. Human CD8+ T cells were electroporated with RNA encoding a CLDN6-specific TCR together with RNA encoding PD-1 and labeled with CFSE. The T cells were then co-cultured with iDCs electroporated with CLDN6-encoding RNA, in the presence or absence of DuoBody-CD40×4-1BB (0.2, 0.0067, or 0.0022 μg/mL) and the anti-PD-1 antibodies IgG1-PD1 (0.8 μg/mL), pembrolizumab (0.8 μg/mL), or nivolumab (1.6 μg/mL), the anti-PD-L1 antibody atezolizumab (0.4 μg/mL), or the negative control antibody IgG1-ctrl-FERR (0.8 μg/mL), for 4 days. CFSE dilution in T cells was analyzed by flow cytometry and used to calculate the expansion index. Data from one representative donor out of four donors tested are shown. Error bars indicate SD of duplicate wells. Dotted line represents expansion index of CD8+ T cells co-cultured with iDCs without antibody treatment. CFSE=Carboxyfluorescein succinimidyl ester; CLDN6=claudin-6; iDC=immature dendritic cell; PD-(L)1=programmed cell death protein (ligand) 1; SD=standard deviation; TCR=T-cell receptor.

FIG. 25 shows the effect of DuoBody-CD40×4-1BB in combination with anti-PD-(L)1 antibodies on cytokine secretion in vitro. Human CD8+ T cells expressing a CLDN6-specific TCR and PD-1 were co-cultured with CLDN6-expressing iDCs as in FIG. 24 in the presence of DuoBody-CD40×4-1BB (0.2, 0.0067, or 0.0022 μg/mL) and the anti-PD-1 antibodies IgG1-PD1 (0.8 μg/mL), pembrolizumab (0.8 μg/mL), or nivolumab (1.6 μg/mL), the anti-PD-L1 antibody atezolizumab (0.4 μg/mL), or the negative control antibody IgG1-ctrl-FERR (0.8 μg/mL), for 4 days. Cytokine concentrations were determined in the supernatants by multiplexed ECLIA. Data from one representative donor out of four donors tested are shown. Error bars indicate SD of duplicate wells. CLDN6=claudin-6; ECLIA=electrochemiluminescence immunoassay; GM-CSF=granulocyte/macrophage colony-stimulating factor; iDC=immature dendritic cell; IFN=interferon; IL=interleukin; PD-(L)1=programmed cell death protein (ligand) 1; SD=standard deviation; TCR=T-cell receptor; TNF=tumor necrosis factor.

FIG. 26 shows the effect of DuoBody-CD40×4-1BB in combination with anti-PD-(L)1 antibodies on T-cell proliferation in vitro. CellTrace Violet-labeled human PBMCs were stimulated with an anti-CD3 antibody (0.09 μg/mL) in the presence of DuoBody-CD40×4-1BB (0.2 μg/mL) and the anti-PD-1 antibodies pembrolizumab or nivolumab or the anti-PD-L1 antibody atezolizumab (all 0.05, 0.5, or 5 μg/mL), either alone or in combination, or the negative control antibody IgG1-ctrl-FEAL (0.2 μg/mL), for 4 days. CellTrace Violet dilution in CD8+ (Fig. A) and CD4+T (Fig. B) cells was analyzed by flow cytometry and used to calculate the expansion index. Data from one representative donor out of three donors tested are shown. Error bars indicate SD of triplicate wells. Dotted line represents expansion index of cells treated with IgG1-ctrl-FEAL. Dashed line represents expansion index of cells treated with single-agent DuoBody-CD40×4-1BB. PD-(L)1=programmed cell death protein (ligand) 1; PBMC=peripheral blood mononuclear cell; SD=standard deviation.

FIG. 27 shows characterization of the exhausted-like phenotype of CD3+ T cells after two rounds of CD3/CD28 stimulation. (A) Expression of LAG3 on in vitro exhausted CD3+ T cells was determined by flow cytometry. Data shown are the median fluorescence intensity corrected for background fluorescence (ΔMFI). (B) In vitro exhausted CD3+ T cells were co-cultured with allogeneic LPS-matured DCs without treatment or in the presence of 1 μg/mL pembrolizumab. Secretion of IFNγ was analyzed by AlphaLISA and secretion of IL-2 by MSD multiplex. Data shown are mean+standard deviation (SD) of duplicate wells of one representative donor pair out of two pairs tested in two experiments.

FIG. 28 shows secretion of IFNγ (A) and IL-2 (B) induced by DuoBody-CD40×4-1BB in combination with pembrolizumab in a mixed lymphocyte reaction (MLR) of mature dendritic cells (mDCs) and in vitro exhausted CD3+ T cells (Tex). Tex were co-cultured with allogeneic LPS-matured DCs in the presence of DuoBody-CD40×4-1BB (0.001-30 μg/mL) or pembrolizumab (1 hg/mL) alone or in combination for 5 days. Co-cultures without treatment (No Tx) or treated with bsIgG1-CD40×ctrl (30 μg/mL), bsIgG1-ctrl×4-1BB (30 μg/mL) or IgG1-ctrl-FEAL (30 μg/mL) were included as controls. Secretion of IFNγ was analyzed by AlphaLISA and secretion of IL-2 by MSD multiplex. Data shown are mean+standard deviation (SD) of duplicate wells of one representative donor pair out of two donor pairs tested in two experiments.

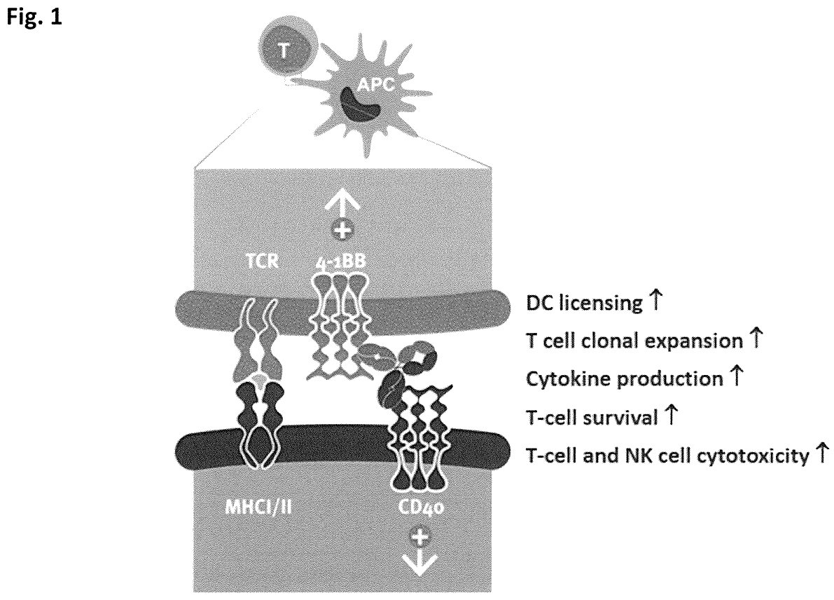

TABLE 1
Sequences

| Name | Type | Sequence | SEQ ID |
|---|---|---|---|
| CD40-001 | VH CDR1 | GYTFTEYI | 1 |
| antibody | | | |
| (mouse) | | | |
| | VH CDR2 | IIPNNGGT | 2 |
| | VH CDR3 | TRREVYGRNYYALDY | 3 |
| | VL CDR1 | QGINNY | 4 |
| | VL CDR2 | YTS | 5 |
| | VL CDR3 | QQYSNLPYT | 6 |
| | VH | EVQLQQSGPDLVKPGASVKISCKTSGYTFTEYIMHWV | 7 |
| | | KQSHGKSLEWIGGIIPNNGGTSYNQKFKDKATMTVDK | |
| | | SSSTGYMELRSLTSEDSAVYYCTRREVYGRNYYALDY | |
| | | WGQGTLVTVSS | |
| | VL | DIQMTQTTSSLSASLGDRVTITCSASQGINNYLNWYQ | 8 |
| | | QKPDGTVKLLIYYTSSLHSGVPSRFSGSGSGTDYSLT | |
| | | ISNLEPEDIATYYCQQYSNLPYTFGGGTKLEIK | |
| | VH | EVQLVQSGAEVKKPGASVKVSCKTSGYTFTEYIMHWV | 9 |
| | humanized | RQAPGQGLEWMGGIIPNNGGTSYNQKFQGRVTMTVDK | |
| | | STSTGYMELSSLRSEDTAVYYCTRREVYGRNYYALDY | |
| | | WGQGTLVTVSS | |
| | VL | DIQMTQSPSSLSASVGDRVTITCSASQGINNYLNWYQ | 10 |
| | humanized | QKPGKAVKLLIYYTSSLHSGVPSRFSGSGSGTDYTFT | |
| | | ISSLQPEDIATYYCQQYSNLPYTFGGGTKVEIK | |
| CD137 | VH CDR1 | GFSLNDYW | 11 |
| antibody | | | |
| clone 009 | | | |
| (rabbit) | | | |
| | VH CDR2 | IDVGGSL | 12 |
| | VH CDR3 | ARGGLTYGFDL | 13 |
| | VL CDR1 | EDISSY | 14 |
| | VL CDR2 | GAS | 15 |
| | VL CDR3 | HYYATISGLGVA | 16 |
| | VH | QSLEESGGRLVTPGTPLTLTCTVSGFSLNDYWMSWVR | 17 |
| | | QAPGKGLEWIGYIDVGGSLYYASWAKGRFTISRTSTT | |
| | | VDLKMTSLTTEDTATYFCARGGLTYGFDLWGPGTLVT | |
| | | VSS | |
| | VL | DIVMTQTPASVSEPVGGTVTINCQASEDISSYLAWYQ | 18 |
| | | QKPGQRPKRLIYGASDLASGVPSRFSASGSGTEYALT | |
| | | ISDLESADAATYYCHYYATISGLGVAFGGGTEVVVK | |
| | VH | EVQLVESGGGLVQPGRSLRLSCTASGFSLNDYWMSWV | 19 |
| | humanized | RQAPGKGLEWVGYIDVGGSLYYAASVKGRFTISRDDS | |
| | | KSIAYLQMNSLKTEDTAVYYCARGGLTYGFDLWGQGT | |
| | | LVTVSS | |
| | VL | DIVMTQSPSSLSASVGDRVTITCQASEDISSYLAWYQ | 20 |
| | humanized | QKPGKAPKRLIYGASDLASGVPSRFSASGSGTDYTFT | |
| | | ISSLQPEDIATYYCHYYATISGLGVAFGGGTKVEIK | |
| IgG1-Fc | | ASTKGPSVFPLAPSSKSTSGGTAALGCLVKDYFPEPV | 21 |
| | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPELLGGPSVFLFPPKPKDTLMISRTPEVTCVVVD | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSKLTVD | |
| | | KSRWQQGNVFSCSVMHEALHNHYTQKSLSLSPGK | |
| IgG1- | | ASTKGPSVFPLAPSSKSTSGGTAALGCLVKDYFPEPV | 22 |
| Fc_F405L | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPELLGGPSVFLFPPKPKDTLMISRTPEVTCVVVD | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFLLYSKLTVD | |
| | | KSRWQQGNVFSCSVMHEALHNHYTQKSLSLSPGK | |
| IgG1- | | ASTKGPSVFPLAPSSKSTSGGTAALGCLVKDYFPEPV | 23 |
| Fc_K409R | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPELLGGPSVFLFPPKPKDTLMISRTPEVTCVVVD | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSRLTVD | |
| | | KSRWQQGNVFSCSVMHEALHNHYTQKSLSLSPGK | |
| IgG1- | | ASTKGPSVFPLAPSSKSTSGGTAALGCLVKDYFPEPV | 24 |
| Fc_FEA | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPEFEGGPSVFLFPPKPKDTLMISRTPEVTCVVVA | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSKLTVD | |
| | | KSRWQQGNVFSCSVMHEALHNHYTQKSLSLSPGK | |
| IgG1-FEAL- | | ASTKGPSVFPLAPSSKSTSGGTAALGCLVKDYFPEPV | 25 |
| Fc | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPEFEGGPSVFLFPPKPKDTLMISRTPEVTCVVVA | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFLLYSKLTVD | |
| | | KSRWQQGNVFSCSVMHEALHNHYTQKSLSLSPGK | |
| IgG1-FEAR- | | ASTKGPSVFPLAPSSKSTSGGTAALGCLVKDYFPEPV | 26 |
| Fc | | TVSWNSGALTSGVHTFPAVLQSSGLYSLSSVVTVPSS | |
| | | SLGTQTYICNVNHKPSNTKVDKRVEPKSCDKTHTCPP | |
| | | CPAPEFEGGPSVFLFPPKPKDTLMISRTPEVTCVVVA | |
| | | VSHEDPEVKFNWYVDGVEVHNAKTKPREEQYNSTYRV | |
| | | VSVLTVLHQDWLNGKEYKCKVSNKALPAPIEKTISKA | |
| | | GKQPREPQVYTLPPSREEMTKNQVSLTCLVKGFYPSD | |
| | | IAVEWESNGQPENNYKTTPPVLDSDGSFFLYSRLTVD | |
| | | KSRWQQGNVFSCSVMHEALHNHYTQKSLSLSPGK | |
| Kappa-C | | RTVAAPSVFIFPPSDEQLKSGTASVVCLLNNFYPREA | 27 |
| | | KVQWKVDNALQSGNSQESVTEQDSKDSTYSLSSTLTL | |
| | | SKADYEKHKVYACEVTHQGLSSPVTKSFNRGEC | |
| Lambda-C | | GQPKAAPSVTLFPPSSEELQANKATLVCLISDFYPGA | 28 |
| | | VTVAWKADSSPVKAGVETTTPSKQSNNKYAASSYLSL | |
| | | TPEQWKSHRSYSCQVTHEGSTVEKTVAPTECS | |
| IgG1-Fc | | ASTKGPSVFPLAPSSKSTSGGTAALGCLVKDYFPEPV | 29 |
| without | | TVSWNSGALTSGVHTFPAVLQSSGLYSLSSVVTVPSS | |
| C-terminal | | SLGTQTYICNVNHKPSNTKVDKRVEPKSCDKTHTCPP | |
| Lys | | CPAPELLGGPSVFLFPPKPKDTLMISRTPEVTCVVVD | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSKLTVD | |
| | | KSRWQQGNVFSCSVMHEALHNHYTQKSLSLSPG | |
| IgG1- | | ASTKGPSVFPLAPSSKSTSGGTAALGCLVKDYFPEPV | 30 |
| Fc_F405L | | TVSWNSGALTSGVHTFPAVLQSSGLYSLSSVVTVPSS | |
| without | | SLGTQTYICNVNHKPSNTKVDKRVEPKSCDKTHTCPP | |
| C-terminal | | CPAPELLGGPSVFLFPPKPKDTLMISRTPEVTCVVVD | |
| Lys | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFLLYSKLTVD | |
| | | KSRWQQGNVFSCSVMHEALHNHYTQKSLSLSPG | |
| IgG1- | | ASTKGPSVFPLAPSSKSTSGGTAALGCLVKDYFPEPV | 31 |
| Fc_K409R | | TVSWNSGALTSGVHTFPAVLQSSGLYSLSSVVTVPSS | |
| without | | SLGTQTYICNVNHKPSNTKVDKRVEPKSCDKTHTCPP | |
| C-terminal | | CPAPELLGGPSVFLFPPKPKDTLMISRTPEVTCVVVD | |
| Lys | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSRLTVD | |
| | | KSRWQQGNVFSCSVMHEALHNHYTQKSLSLSPG | |
| IgG1- | | ASTKGPSVFPLAPSSKSTSGGTAALGCLVKDYFPEPV | 32 |
| Fc_FEA | | TVSWNSGALTSGVHTFPAVLQSSGLYSLSSVVTVPSS | |
| without | | SLGTQTYICNVNHKPSNTKVDKRVEPKSCDKTHTCPP | |
| C-terminal | | CPAPEFEGGPSVFLFPPKPKDTLMISRTPEVTCVVVA | |
| Lys | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSKLTVD | |
| | | KSRWQQGNVFSCSVMHEALHNHYTQKSLSLSPG | |
| IgG1- | | ASTKGPSVFPLAPSSKSTSGGTAALGCLVKDYFPEPV | 33 |
| FEAL-Fc | | TVSWNSGALTSGVHTFPAVLQSSGLYSLSSVVTVPSS | |
| without | | SLGTQTYICNVNHKPSNTKVDKRVEPKSCDKTHTCPP | |
| C-terminal | | CPAPEFEGGPSVFLFPPKPKDTLMISRTPEVTCVVVA | |
| Lys | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFLLYSKLTVD | |
| | | KSRWQQGNVFSCSVMHEALHNHYTQKSLSLSPG | |
| IgG1- | | ASTKGPSVFPLAPSSKSTSGGTAALGCLVKDYFPEPV | 34 |
| FEAR-Fc | | TVSWNSGALTSGVHTFPAVLQSSGLYSLSSVVTVPSS | |
| without | | SLGTQTYICNVNHKPSNTKVDKRVEPKSCDKTHTCPP | |
| C-terminal | | CPAPEFEGGPSVFLFPPKPKDTLMISRTPEVTCVVVA | |
| Lys | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSRLTVD | |
| | | KSRWQQGNVFSCSVMHEALHNHYTQKSLSLSPG | |
| Human CD40 | | MVRLPLQCVLWGCLLTAVHPEPPTACREKQYLINSQC | 35 |
| precursor | | CSLCQPGQKLVSDCTEFTETECLPCGESEFLDTWNRE | |
| | | THCHQHKYCDPNLGLRVQQKGTSETDTICTCEEGWHC | |
| | | TSEACESCVLHRSCSPGFGVKQIATGVSDTICEPCPV | |
| | | GFFSNVSSAFEKCHPWTSCETKDLVVQQAGTNKTDVV | |
| | | CGPQDRLRALVVIPIIFGILFAILLVLVFIKKVAKKP | |
| | | TNKAPHPKQEPQEINFPDDLPGSNTAAPVQETLHGCQ | |
| | | PVTQEDGKESRISVQERQ | |
| Human CD40 | | EPPTACREKQYLINSQCCSLCQPGQKLVSDCTEFTET | 36 |
| mature | | ECLPCGESEFLDTWNRETHCHQHKYCDPNLGLRVQQK | |
| protein | | GTSETDTICTCEEGWHCTSEACESCVLHRSCSPGFGV | |
| | | KQIATGVSDTICEPCPVGFFSNVSSAFEKCHPWTSCE | |
| | | TKDLVVQQAGTNKTDVVCGPQDRLRALVVIPIIFGIL | |
| | | FAILLVLVFIKKVAKKPTNKAPHPKQEPQEINFPDDL | |
| | | PGSNTAAPVQETLHGCQPVTQEDGKESRISVQERQ | |
| Human CD137 | | MGNSCYNIVATLLLVLNFERTRSLQDPCSNCPAGTFC | 37 |
| precursor | | DNNRNQICSPCPPNSFSSAGGQRTCDICRQCKGVFRT | |
| | | RKECSSTSNAECDCTPGFHCLGAGCSMCEQDCKQGQE | |
| | | LTKKGCKDCCFGTFNDQKRGICRPWTNCSLDGKSVLV | |
| | | NGTKERDVVCGPSPADLSPGASSVTPPAPAREPGHSP | |
| | | QIISFFLALTSTALLFLLFFLTLRFSVVKRGRKKLLY | |
| | | IFKQPFMRPVQTTQEEDGCSCRFPEEEEGGCEL | |
| Human CD137 | | LQDPCSNCPAGTFCDNNRNQICSPCPPNSFSSAGGQR | 38 |
| mature | | TCDICRQCKGVFRTRKECSSTSNAECDCTPGFHCLGA | |
| protein | | GCSMCEQDCKQGQELTKKGCKDCCFGTFNDQKRGICR | |
| | | PWTNCSLDGKSVLVNGTKERDVVCGPSPADLSPGASS | |
| | | VTPPAPAREPGHSPQIISFFLALTSTALLFLLFFLTL | |
| | | RFSVVKRGRKKLLYIFKQPFMRPVQTTQEEDGCSCRF | |
| | | PEEEEGGCEL | |
| Human PD-1 | | MQIPQAPWPVVWAVLQLGWRPGWFLDSPDRPWNPPTF | 39 |
| | | SPALLVVTEGDNATFTCSFSNTSESFVLNWYRMSPSN | |
| | | QTDKLAAFPEDRSQPGQDCRFRVTQLPNGRDFHMSVV | |
| | | RARRNDSGTYLCGAISLAPKAQIKESLRAELRVTERR | |
| | | AEVPTAHPSPSPRPAGQFQTLVVGVVGGLLGSLVLLV | |
| | | WVLAVICSRAARGTIGARRTGQPLKEDPSAVPVFSVD | |
| | | YGELDFQWREKTPEPPVPCVPEQTEYATIVFPSGMGT | |
| | | SSPARRGSADGPRSAQPLRPEDGHCSWPL | |
| Human PD-L1 | | MRIFAVFIFMTYWHLLNAFTVTVPKDLYVVEYGSNMT | 40 |
| | | IECKFPVEKQLDLAALIVYWEMEDKNIIQFVHGEEDL | |
| | | KVQHSSYRQRARLLKDQLSLGNAALQITDVKLQDAGV | |
| | | YRCMISYGGADYKRITVKVNAPYNKINQRILVVDPVT | |
| | | SEHELTCQAEGYPKAEVIWTSSDHQVLSGKTTTTNSK | |
| | | REEKLFNVTSTLRINTTTNEIFYCTFRRLDPEENHTA | |
| | | ELVIPELPLAHPPNERTHLVILGAILLCLGVALTFIF | |
| | | RLRKGRMMDVKKCGIQDTNSKKQSDTHLEET | |
| Macaque | | MRIFAVFIFTYWHLLNAFTVTVPKDLYVVEYGSNMTI | 41 |
| PD-L1 | | ECKFPVEKQLDLTSLIVYWEMEDKNIIQFVHGEEDLK | |
| | | VQHSNYRQRAQLLKDQLSLGNAALRITDVKLQDAGVY | |
| | | RCMISYGGADYKRITVKVNAPYNKINQRILVVDPVTS | |
| | | EHELTCQAEGYPKAEVIWTSSDHQVLSGKTTTTNSKR | |
| | | EEKLLNVTSTLRINTTANEIFYCIFRRLDPEENHTAE | |
| | | LVIPELPLALPPNERTHLVILGAIFLLLGVALTFIFY | |
| | | LRKGRMMDMKKCGIRVTNSKKQRDTQLEET | |
| CTLA-4 | | MACLGFQRHKAQLNLATRTWPCTLLFFLLFIPVFCKA | 42 |
| | | MHVAQPAVVLASSRGIASFVCEYASPGKATEVRVTVL | |
| | | RQADSQVTEVCAATYMMGNELTFLDDSICTGTSSGNQ | |
| | | VNLTIQGLRAMDTGLYICKVELMYPPPYYLGIGNGTQ | |
| | | IYVIDPEPCPDSDFLLWILAAVSSGLFFYSFLLTAVS | |
| | | LSKMLKKRSPLTTGVYVKMPPTEPECEKQFQPYFIPI | |
| | | N | |
| MAB-19-0618 | VH | QVQLVESGGGLVQPGTSLRLSCSVSGFSLYSYNMGWV | 43 |
| IgG1-PD1 | | RQAPGKGLEYIGIISGGTIGHYASWAKGRFTISRDTS | |
| | | KTTLYLQMNSLTTEDTATYFCARAFYDDYDYNVWGPG | |
| | | TLVTVSS | |
| MAB-19-0618 | VL | AIQLTQSPSSLSASVGGTVTITCQSSQSVYGNNQLSW | 44 |
| IgG1-PD1 | | YQQKPGQPPKLLIYQASKLETGVPSRFRGSGSGTQFT | |
| | | LTISSLQSEDFATYYCAGGYSSSSDTTFGGGTEVVVK | |
| MAB-19-0618 | VH CDR1 | GFSLYSYN | 45 |
| IgG1-PD1 | | | |
| MAB-19-0618 | VH CDR2 | IISGGTIG | 46 |
| IgG1-PD1 | | | |
| MAB-19-0618 | VH CDR3 | AFYDDYDYNV | 47 |
| IgG1-PD1 | | | |
| MAB-19-0618 | VL CDR1 | QSVYGNNQ | 48 |
| IgG1-PD1 | | | |
| MAB-19-0618 | VL CDR2 | QAS | 49 |
| IgG1-PD1 | | | |
| MAB-19-0618 | VL CDR3 | AGGYSSSSDTT | 50 |
| IgG1-PD1 | | | |
| IgG1-PD1- | VH CDR1 | GITFSNSG | 51 |
| MDX1106- | | | |
| FEAL | | | |
| | VH CDR2 | IWYDGSKR | 52 |
| | VH CDR3 | ATNDDY | 53 |
| | VL CDR1 | QSVSSY | 54 |
| | VL CDR2 | DAS | 55 |
| | VL CDR3 | QQSSNWPRT | 56 |
| | VH | QVQLVESGGGVVQPGRSLRLDCKASGITFSNSGMHWV | 57 |
| | | RQAPGKGLEWVAVIWYDGSKRYYADSVKGRFTISRDN | |
| | | SKNTLFLQMNSLRAEDTAVYYCATNDDYWGQGTLVTV | |
| | | SS | |
| | VL | EIVLTQSPATLSLSPGERATLSCRASQSVSSYLAWYQ | 58 |
| | | QKPGQAPRLLIYDASNRATGIPARFSGSGSGTDFTLT | |
| | | ISSLEPEDFAVYYCQQSSNWPRTFGQGTKVEIK | |
| IgG1-b12 | VH | QVQLVQSGAEVKKPGASVKVSCQASGYRFSNFVIHWV | 59 |
| | | RQAPGQRFEWMGWINPYNGNKEFSAKFQDRVTFTADT | |
| | | SANTAYMELRSLRSADTAVYYCARVGPYSWDDSPQDN | |
| | | YYMDVWGKGTTVIVSS | |
| IgG1-b12 | VL | EIVLTQSPGTLSLSPGERATFSCRSSHSIRSRRVAWY | 60 |
| | | QHKPGQAPRLVIHGVSNRASGISDRFSGSGSGTDFTL | |
| | | TITRVEPEDFALYYCQVYGASSYTFGQGTKLERK | |
| IgG1m(f)- | | ASTKGPSVFPLAPSSKSTSGGTAALGCLVKDYFPEPV | 61 |
| L234F- | | TVSWNSGALTSGVHTFPAVLQSSGLYSLSSVVTVPSS | |
| L235E- | | SLGTQTYICNVNHKPSNTKVDKRVEPKSCDKTHTCPP | |
| G236R | | CPAPEFERGPSVFLFPPKPKDTLMISRTPEVTCVVVD | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSKLTVD | |
| | | KSRWQQGNVFSCSVMHEALHNHYTQKSLSLSPG | |
| IgG1m(f)- | | ASTKGPSVFPLAPSSKSTSGGTAALGCLVKDYFPEPV | 62 |
| L234F- | | TVSWNSGALTSGVHTFPAVLQSSGLYSLSSVVTVPSS | |
| L235E- | | SLGTQTYICNVNHKPSNTKVDKRVEPKSCDKTHTCPP | |
| G236R- | | CPAPEFERGPSVFLFPPKPKDTLMISRTPEVTCVVVD | |
| K409R | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSRLTVD | |
| | | KSRWQQGNVFSCSVMHEALHNHYTQKSLSLSPG | |
| MAB-19-0618 | VH CDR1 | SYN | 63 |
| gG1-PD1 | | | |
| MAB-19-0618 | VH CDR1 | SYNMG | 64 |
| IgG1-PD1 | | | |
| MAB-19-0618 | VH CDR2 | ISGGTIG | 65 |
| IgG1-PD1 | | | |
| MAB-19-0618 | VH CDR2 | IISGGTIGHYASWAKG | 66 |
| IgG1-PD1 | | | |
| MAB-19-0618 | VH CDR3 | ARAFYDDYDYNV | 67 |
| IgG1-PD1 | | | |
| MAB-19-0618 | VL CDR1 | QSSQSVYGNNQLS | 68 |
| IgG1-PD1 | | | |
| MAB-19-0618 | VL CDR2 | QASKLET | 69 |
| IgG1-PD1 | | | |
| Heavy chain | | ASTKGPSVFPLAPSSKSTSGGTAALGCLVKDYFPEPV | 70 |
| human | | TVSWNSGALTSGVHTFPAVLQSSGLYSLSSVVTVPSS | |
| IgG1-LALA | | SLGTQTYICNVNHKPSNTKVDKKVEPKSCDKTHTCPP | |
| | | CPAPEAAGGPSVFLFPPKPKDTLMISRTPEVTCVVVD | |
| | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSKLTVD | |
| | | KSRWQQGNVFSCSVMHEALHNHYTQKSLSLSPG | |
| MAB-19-0202 | VH | QSVEESGGRLVTPGTPLTLTCTVSGFSLYSYNMGWVR | 71 |
| | | QAPGKGLEYIGIISGGTIGHYASWAKGRFTISKTSST | |
| | | TVDLKMTSLTTEDTATYFCARAFYDDYDYNVWGPGTL | |
| | | VTVSS | |
| MAB-19-0202 | VL | AAVLTQTPSPVSAAVGGTVTISCQSSQSVYGNNQLSW | 72 |
| | | YQQKPGQPPKLLIYQASKLETGVPSRFKGSGSGTQFT | |
| | | LTISDLESDDAATYYCAGGYSSSSDTTFGGGTEVVVK | |
| constant | HC | ASTKGPSVFPLAPSSKSTSGGTAALGCLVKDYFPEPV | 73 |
| region | | TVSWNSGALTSGVHTFPAVLQSSGLYSLSSVVTVPSS | |
| human HC | | SLGTQTYICNVNHKPSNTKVDKRVEPKSCDKTHTCPP | |
| IgG1m(f)- | | CPAPELLGGPSVFLFPPKPKDTLMISRTPEVTCVVVD | |
| E430G | | VSHEDPEVKFNWYVDGVEVHNAKTKPREEQYNSTYRV | |
| | | VSVLTVLHQDWLNGKEYKCKVSNKALPAPIEKTISKA | |
| | | KGQPREPQVYTLPPSREEMTKNQVSLTCLVKGFYPSD | |
| | | IAVEWESNGQPENNYKTTPPVLDSDGSFFLYSKLTVD | |
| | | KSRWQQGNVFSCSVMHGALHNHYTQKSLSLSPG | |
| VH CD52- | VH | QVQLQESGPGLVRPSQTLSLTCTVSGFTFTDFYMNWV | 74 |
| CAMPATH-1H | | RQPPGRGLEWIGFIRDKAKGYTTEYNPSVKGRVTMLV | |
| | | DTSKNQFSLRLSSVTAADTAVYYCAREGHTAAPFDYW | |
| | | GQGSLVTVSS | |
| VL CD52- | VL | DIQMTQSPSSLSASVGDRVTITCKASQNIDKYLNWYQ | 75 |
| CAMPATH-1H | | QKPGKAPKLLIYNTNNLQTGVPSRFSGSGSGTDFTFT | |
| | | ISSLQPEDIATYYCLQHISRPRTFGQGTKVEIK | |
| constant | VH | ASTKGPSVFPLAPCSRSTSESTAALGCLVKDYFPEPV | 76 |
| region | | TVSWNSGALTSGVHTFPAVLQSSGLYSLSSVVTVPSS | |
| human HC | | SLGTKTYTCNVDHKPSNTKVDKRVESKYGPPCPSCPA | |
| IgG4 | | PEFLGGPSVFLFPPKPKDTLMISRTPEVTCVVVDVSQ | |
| | | EDPEVQFNWYVDGVEVHNAKTKPREEQFNSTYRVVSV | |
| | | LTVLHQDWLNGKEYKCKVSNKGLPSSIEKTISKAKGQ | |
| | | PREPQVYTLPPSQEEMTKNQVSLTCLVKGFYPSDIAV | |
| | | EWESNGQPENNYKTTPPVLDSDGSFFLYSRLTVDKSR | |
| | | WQEGNVFSCSVMHEALHNHYTQKSLSLSLGK | |

| | | |
|---|---|---|
| SEQ ID NO: 63 | HCDR1 (MAB-19-0618) | intersection of Kabat and |
| | | IMGT |
| SEQ ID NO: 64 | HCDR1 (MAB-19-0618) | Kabat |
| SEQ ID NO: 45 | HCDR1 (MAB-19-0618) | IMGT |
| SEQ ID NO: 65 | HCDR2 (MAB-19-0618) | intersection of Kabat and |
| | | IMGT (=IMGT) |
| SEQ ID NO: 66 | HCDR2 (MAB-19-0618) | Kabat |
| SEQ ID NO: 47 | HCDR3 (MAB-19-0618) | intersection of Kabat and |
| | | IMGT (=Kabat) |
| SEQ ID NO: 67 | HCDR3 (MAB-19-0618) | IMGT |
| SEQ ID NO: 48 | LCDR1 (MAB-19-0618) | intersection of Kabat and |
| | | IMGT (=IMGT) |
| SEQ ID NO: 68 | LCDR1 (MAB-19-0618) | Kabat |
| SEQ ID NO: 49 | LCDR2 (MAB-19-0618) | intersection of Kabat and |
| | | IMGT (=IMGT) |
| SEQ ID NO: 69 | LCDR2 (MAB-19-0618) | Kabat |
| SEQ ID NO: 50 | LCDR3 (MAB-19-0618) | intersection = Kabat = |
| | | IMGT |

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in a preferred embodiment of the binding agent used herein the first heavy chain comprises or consists essentially of or consists of an amino acid sequence set forth in SEQ ID NO: 26 or 34 [IgG1-Fc_FEAR] and in another preferred embodiment of the binding agent used herein the second heavy chain comprises or consists essentially of or consists of an amino acid sequence set forth in SEQ ID NO: 25 or 33 [IgG1-Fc_FEAL], then in a further preferred embodiment of the binding agent used herein the first heavy chain comprises or consists essentially of or consists of an amino acid sequence set forth in SEQ ID NO: 26 or 34 [IgG1-Fc_FEAR] and the second heavy chain comprises or consists essentially of or consists of an amino acid sequence set forth in SEQ ID NO: 25 or 33 [IgG1-Fc_FEAL].

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional chemistry, biochemistry, cell biology, immunology, and recombinant DNA techniques which are explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Römpp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, ±4%, 3%, ±2%, ±1%, ±0.9%, ±0.8%, ±0.7%, 10.6%, ±0.5%, ±0.4%, 0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

The term “binding agent” in the context of the present disclosure refers to any agent capable of binding to desired antigens. In certain embodiments of the present disclosure, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

As used herein, “immune checkpoint” refers to regulators of the immune system, and, in particular, co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1 and/or PD-L2. In certain embodiments, the inhibitory signal is the interaction between CTLA-4 and CD80 or CD86 to displace CD28 binding. In certain embodiments the inhibitory signal is the interaction between LAG-3 and MHC class II molecules. In certain embodiments, the inhibitory signal is the interaction between TIM-3 and one or more of its ligands, such as galectin 9, PtdSer, HMGB1 and CEACAM1. In certain embodiments, the inhibitory signal is the interaction between one or several KIRs and their ligands. In certain embodiments, the inhibitory signal is the interaction between TIGIT and one or more of its ligands, PVR, PVRL2 and PVRL3. In certain embodiments, the inhibitory signal is the interaction between CD94/NKG2A and HLA-E. In certain embodiments, the inhibitory signal is the interaction between VISTA and its binding partner(s). In certain embodiments, the inhibitory signal is the interaction between one or more Siglecs and their ligands. In certain embodiments, the inhibitory signal is the interaction between GARP and one or more of its ligands. In certain embodiments, the inhibitory signal is the interaction between CD47 and SIRPα. In certain embodiments, the inhibitory signal is the interaction between PVRIG and PVRL2. In certain embodiments, the inhibitory signal is the interaction between CSF1R and CSF1. In certain embodiments, the inhibitory signal is the interaction between BTLA and HVEM. In certain embodiments, the inhibitory signal is part of the adenosinergic pathway, e.g., the interaction between A2AR and/or A2BR and adenosine, produced by CD39 and CD73. In certain embodiments, the inhibitory signal is the interaction between B7-H3 and its receptor and/or B7-H4 and its receptor. In certain embodiments, the inhibitory signal is mediated by IDO, CD20, NOX or TDO.

The terms “checkpoint inhibitor” (CPI) and “immune checkpoint (ICP) inhibitor” are used herein synonymously. The terms refer to molecules, such as binding agents, which totally or partially reduce, inhibit, interfere with or negatively modulate one or more checkpoint proteins or that totally or partially reduce, inhibit, interfere with or negatively modulate expression of one or more checkpoint proteins, like molecules, such as binding agents, which inhibit an immune checkpoint, in particular, which inhibit the inhibitory signal of an immune checkpoint. In one embodiment, the immune checkpoint inhibitor binds to one or more checkpoint proteins. In one embodiment, the immune checkpoint inhibitor binds to one or more molecules regulating checkpoint proteins. In one embodiment, the immune checkpoint inhibitor binds to precursors of one or more checkpoint proteins e.g., on DNA- or RNA-level. Any agent that functions as a checkpoint inhibitor according to the present disclosure can be used. The term “partially” as used herein means at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% in the level, e.g., in the level of inhibition of a checkpoint protein.

In one embodiment, the checkpoint inhibitor can be any compound, such as any binding agent, which inhibits the inhibitory signal of an immune checkpoint, wherein the inhibitory signal is selected from the group consisting of: the interaction between PD-1 and PD-L1 and/or PD-L2; the interaction between CTLA-4 and CD80 or CD86 to displace CD28 binding; the interaction between LAG-3 and MHC class II molecules; the interaction between TIM-3 and one or more of its ligands, such as galectin 9, PtdSer, HMGB1 and CEACAM1; the interaction between one or several KIRs and their ligands; the interaction between TIGIT and one or more of its ligands, PVR, PVRL2 and PVRL3; the interaction between CD94/NKG2A and HLA-E; the interaction between VISTA and its binding partner(s); the interaction between one or more Siglecs and their ligands; the interaction between GARP and one or more of its ligands; the interaction between CD47 and SIRPα; the interaction between PVRIG and PVRL2; the interaction between CSF1R and CSF1; the interaction between BTLA and HVEM; part of the adenosinergic pathway, e.g., the interaction between A2AR and/or A2BR and adenosine, produced by CD39 and CD73; the interaction between B7-H3 and its receptor and/or B7-H4 and its receptor; an inhibitory signal mediated by IDO, CD20, NOX or TDO. In one embodiment, the checkpoint inhibitor is at least one selected from the group consisting of PD-1 inhibitors, PD-L1 inhibitors, PD-L2 inhibitors, CTLA-4 inhibitors, TIM-3 inhibitors, KIR inhibitors, LAG-3 inhibitors, TIGIT inhibitors, VISTA inhibitors, and GARP inhibitors. In one embodiment, the checkpoint inhibitor may be a blocking antibody, such as a PD-1 blocking antibody, a CTLA4 blocking antibody, a PD-L1 blocking antibody, a PD-L2 blocking antibody, a TIM-3 blocking antibody, a KIR blocking antibody, a LAG-3 blocking antibody, a TIGIT blocking antibody, a VISTA blocking antibody, or a GARP blocking antibody. Examples of a PD-1 blocking antibody include pembrolizumab, nivolumab, cemiplimab, and spartalizumab. Examples of a CTLA4 blocking antibody include ipilimumab and tremelimumab. Examples of a PD-L1 blocking antibody include atezolizumab, durvalumab, and avelumab.

In one embodiment, the anti-PD-1 antibody or antigen-binding fragment thereof comprises a heavy chain variable region (VH) comprising the CDR1, CDR2, and CDR3 sequences of SEQ ID NO: 43, and a light chain variable region (VL) comprising the CDR1, CDR2, and CDR3 sequences of SEQ ID NO: 44.

In one embodiment, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO: 45;
(ii) a CDR-H2 comprising the amino acid sequence of SEQ ID NO: 46; and
(iii) a CDR-H3 comprising the amino acid sequence of SEQ ID NO: 47; and
   wherein the light chain variable region comprises:
(i) a CDR-L1 comprising the amino acid sequence of SEQ ID NO: 48;
(ii) a CDR-L2 comprising the amino acid sequence of SEQ ID NO: 49; and
(iii) a CDR-L3 comprising the amino acid sequence of SEQ ID NO: 50.

In one embodiment of the anti-PD-1 antibodies described herein, the heavy chain variable domain comprises the amino acid sequence of SEQ ID NO: 43 and the light chain variable domain comprises the amino acid sequence of SEQ ID NO: 44.

In one embodiment, the immune checkpoint inhibitor suitable for use in the methods disclosed herein, is an antagonist of inhibitory signals, e.g., an antibody which targets, for example, PD-1, PD-L1, CTLA-4, TIM-3, LAG-3, B7-H3, or B7-1-14. These ligands and receptors are reviewed in Pardoll, D., Nature. 12: 252-264, 2012. Further immune checkpoint proteins that can be targeted according the disclosure are described herein.

The term “immunoglobulin” relates to proteins of the immunoglobulin superfamily, preferably to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies.

The structure of immunoglobulins has been well characterized. See, e.g., Fundamental Immunology Ch. 7 (Paul, W., ed., 2^{nd} ed. Raven Press, N.Y. (1989)). Briefly, immunoglobulins generally comprise several chains, typically two identical heavy chains and two identical light chains which are linked via disulfide bonds. These chains are primarily composed of immunoglobulin domains or regions, such as the V_{L} or VL (variable light chain) domain/region, C_{L} or CL (constant light chain) domain/region, V_{H} or VII (variable heavy chain) domain/region, and the C_{H} or CH (constant heavy chain) domains/regions C_{H}1 (CH1), C_{H}2 (CH2), C_{H}3 (CH3), and C_{H}4 (CH4). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a VL and a CL. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc M P., Nucleic Acids Research 1999; 27:209-212 and Ehrenmann F., Kaas Q. and Lefranc M.-P. Nucleic Acids Res., 38, D301-307 (2010); see also internet http address www.imgt.org. Unless otherwise stated or contradicted by context, reference to amino acid positions in the constant regions in the present disclosure is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242).

There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present disclosure, amino acids may be classified based on structure and chemical characteristics. Thus, classes of amino acids may be reflected in one or both of the following tables:

TABLE 2
Main classification based on structure and general
chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Alternative Physical and Functional Classifications
of Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, |
| | P, and T |
| Flexible residues | Q, T, K, S, G, P, D, E, and R |

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants.

Amino acid substitution variants are characterized by at least one residue in the sequence being removed and another residue being inserted in its place. Substitution of one amino acid for another may be classified as a conservative or non-conservative substitution. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. In the context of the present disclosure, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues. Naturally occurring amino acids may also be generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

The term “amino acid corresponding to position . . . ” and similar expressions as used herein refer to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present disclosure.

The term “antibody” (Ab) in the context of the present disclosure refers to an immunoglobulin molecule, a fragment of an immunoglobulin molecule, or a derivative of either thereof, which has the ability to specifically bind to an antigen (in particular an epitope on an antigen) under typical physiological conditions, preferably with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3 (or CDR-H1, CDR-H2 and CDR-H3), the CDRs of a VL are termed LCDR1, LCDR2 and LCDR3 (or CDR-L1, CDR-L2 and CDR-L3). The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and components of the complement system such as C1q. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The variable regions of the heavy and light chains of the immunoglobulin molecule contain a binding domain that interacts with an antigen. The terms “binding region” and “antigen-binding region” are used herein interchangeably and refer to the region which interacts with the antigen and comprises both a VH region and a VL region. An antibody as used herein comprises not only monospecific antibodies, but also multispecific antibodies which comprise multiple, such as two or more, e.g., three or more, different antigen-binding regions.

As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VII, CL and CH1 domains, or a monovalent antibody as described in WO 2007/059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody; (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January; 5(1): 11-24); and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present disclosure, exhibiting different biological properties and utility. These and other useful antibody fragments in the context of the present disclosure, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

An antibody as generated can possess any isotype. As used herein, the term “isotype” refers to the immunoglobulin class (for instance IgG (such as IgG1, IgG2, IgG3, IgG4), IgD, IgA (such as IgA1, IgA2), IgE, IgM, or IgY) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody disclosed herein may be a sequence variant of a naturally-occurring IgG1 antibody, including variations in the constant regions.

IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The term “multispecific antibody” in the context of the present disclosure refers to an antibody having at least two different antigen-binding regions defined by different antibody sequences. In some embodiments, said different antigen-binding regions bind different epitopes on the same antigen. However, in preferred embodiments, said different antigen-binding regions bind different target antigens. In one embodiment, the multispecific antibody is a “bispecific antibody” or “bs”. A multispecific antibody, such as a bispecific antibody, can be of any format, including any of the bispecific or multispecific antibody formats described herein below.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

The term “human antibody”, as used herein, is intended to include antibodies having variable and framework regions derived from human germline immunoglobulin sequences and a human immunoglobulin constant domain. The human antibodies disclosed herein may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generically for different kinds of modifications of antibodies, and processes for antibody engineering are well-known for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody may be performed by other methods than those described herein. Chimeric monoclonal antibodies for therapeutic applications in humans are developed to reduce anticipated antibody immunogenicity of non-human antibodies, e.g. rodent antibodies. They may typically contain non-human (e.g. murine or rabbit) variable regions, which are specific for the antigen of interest, and human constant antibody heavy and light chain domains. The terms “variable region” or “variable domain” as used in the context of chimeric antibodies, refer to a region which comprises the CDRs and framework regions of both the heavy and light chains of an immunoglobulin, as described below.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO 92/22653 and EP 0 629 240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

As used herein, a protein which is “derived from” another protein, e.g., a parent protein, means that one or more amino acid sequences of the protein are identical or similar to one or more amino acid sequences in the other or parent protein. For example, in an antibody, binding arm, antigen-binding region, constant region, or the like which is derived from another or a parent antibody, binding arm, antigen-binding region, or constant region, one or more amino acid sequences are identical or similar to those of the other or parent antibody, binding arm, antigen-binding region, or constant region. Examples of such one or more amino acid sequences include, but are not limited to, those of the VH and VL CDRs and/or one or more or all of the framework regions, VH, VL, CL, hinge, or CH regions. For example, a humanized antibody can be described herein as “derived from” a non-human parent antibody, meaning that at least the VL and VH CDR sequences are identical or similar to the VH and VL CDR sequences of said non-human parent antibody. A chimeric antibody can be described herein as being “derived from” a non-human parent antibody, meaning that typically the VH and VL sequences may be identical or similar to those of the non-human parent antibody. Another example is a binding arm or an antigen-binding region which may be described herein as being “derived from” a particular parent antibody, meaning that said binding arm or antigen-binding region typically comprises identical or similar VII and/or VL CDRs, or VH and/or VL sequences to the binding arm or antigen-binding region of said parent antibody. As described elsewhere herein, however, amino acid modifications such as mutations can be made in the CDRs, constant regions or elsewhere in the antibody, binding arm, antigen-binding region or the like, to introduce desired characteristics. When used in the context of one or more sequences derived from a first or parent protein, a “similar” amino acid sequence preferably has a sequence identity of at least about 50%, such as at least about 60%, at least about 70%, at least about 80%, at least about 90%, at least about 95%, or at least about 97%, 98% or 99%.

Non-human antibodies can be generated in a number of different species, such as mouse, rabbit, chicken, guinea pig, llama and goat.

Monoclonal antibodies can be produced by a variety of techniques, including conventional monoclonal antibody methodology, e.g., the standard somatic cell hybridization technique of Kohler and Milstein, Nature 256: 495 (1975). Other techniques for producing monoclonal antibodies can be employed, e.g., viral or oncogenic transformation of B-lymphocytes or phage display techniques using libraries of antibody genes, and such methods are well known to a person skilled in the art.

Hybridoma production in such non-human species is a very well established procedure. Immunization protocols and techniques for isolation of splenocytes of immunized animals/non-human species for fusion are known in the art. Fusion partners (e.g., murine myeloma cells) and fusion procedures are also known.

When used herein, unless contradicted by context, the term “Fab-arm” or “arm” refers to one heavy chain-light chain pair and is used interchangeably with “half molecules” herein.

The term “binding arm comprising an antigen-binding region” means an antibody molecule or fragment that comprises an antigen-binding region. Thus, a binding arm can comprise, e.g., the six VH and VL CDR sequences, the VII and VL sequences, a Fab or Fab′ fragment, or a Fab-arm.

When used herein, unless contradicted by context, the term “Fe region” refers to an antibody region consisting of the two Fc sequences of the heavy chains of an immunoglobulin, wherein said Fe sequences comprise at least a hinge region, a CH2 domain, and a CH3 domain. In one embodiment, the term “Fe region”, as used herein, refers to a region comprising, in the direction from the N- to C-terminal end of the antibody, at least a hinge region, a CH2 region and a CH3 region. An Fe region of the antibody may mediate the binding of the immunoglobulin to host tissues or factors, including various cells of the immune system (such as effector cells) and components of the complement system.

In the context of the present disclosure, the term “induce Fe-mediated effector function to a lesser extent” used in relation to an antibody, including a multispecific antibody, means that the antibody induces Fe-mediated effector functions, such function in particular being selected from the list of IgG Fe receptor (FcgammaR, FcγR) binding, C1q binding, ADCC or CDC, to a lesser extent compared to a human IgG1 antibody comprising (i) the same CDR sequences, in particular comprising the same first and second antigen-binding regions, as said antibody and (ii) two heavy chains comprising human IgG1 hinge, CH2 and CH3 regions.

Fc-mediated effector function may be measured by binding to FcγRs, binding to C1q, or induction of Fe-mediated cross-linking via FcγRs.

The term “hinge region” as used herein refers to the hinge region of an immunoglobulin heavy chain. Thus, for example, the hinge region of a human IgG1 antibody corresponds to amino acids 216-230 according to the EU numbering as set forth in Kabat (Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example, the CH1 region of a human IgG1 antibody corresponds to amino acids 118-215 according to the EU numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example, the CH2 region of a human IgG1 antibody corresponds to amino acids 231-340 according to the EU numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus, for example, the CH3 region of a human IgG1 antibody corresponds to amino acids 341-447 according to the EU numbering as set forth in Kabat (ibid). However, the CH3 region may also be any of the other subtypes as described herein.

The term “monovalent antibody” means in the context of the present disclosure that an antibody molecule is capable of binding a single molecule of the antigen, and thus is not capable of antigen cross-linking.

A “CD40 antibody” or “anti-CD40 antibody” is an antibody as described above, which binds specifically to the antigen CD40.

A “CD137 antibody” or “anti-CD137 antibody” is an antibody as described above, which binds specifically to the antigen CD137.

A “CD40×CD137 antibody” or “anti-CD40×CD137 antibody” is a bispecific antibody, which comprises two different antigen-binding regions, one of which binds specifically to the antigen CD40 and one of which binds specifically to the antigen CD137.

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸ M or less, such as about 10⁻¹⁰ M or less, about 10⁻¹⁰ M or less, or about 10⁻¹¹ M or even less, when determined using Bio-Layer Interferometry (BLI) or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein) other than the predetermined antigen or a closely-related antigen. The amount with which the affinity is higher is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction.

Two antibodies have the “same specificity” if they bind to the same antigen and to the same epitope. Whether an antibody to be tested recognizes the same epitope as a certain antigen-binding antibody, i.e., the antibodies bind to the same epitope, may be tested by different methods well known to a person skilled in the art.

The competition between the antibodies can be detected by a cross-blocking assay. For example, a competitive ELISA assay may be used as a cross-blocking assay. E.g., target antigen may be coated on the wells of a microtiter plate and antigen-binding antibody and candidate competing test antibody may be added. The amount of the antigen-binding antibody bound to the antigen in the well indirectly correlates with the binding ability of the candidate competing test antibody that competes therewith for binding to the same epitope. Specifically, the larger the affinity of the candidate competing test antibody is for the same epitope, the smaller the amount of the antigen-binding antibody bound to the antigen-coated well. The amount of the antigen-binding antibody bound to the well can be measured by labeling the antibody with detectable or measurable labeling substances.

An antibody competing for binding to an antigen with another antibody, e.g., an antibody comprising heavy and light chain variable regions as described herein, or an antibody having the specificity for an antigen of another antibody, e.g., an antibody comprising heavy and light chain variable regions as described herein, may be an antibody comprising variants of said heavy and/or light chain variable regions as described herein, e.g. modifications in the CDRs and/or a certain degree of identity as described herein.

An “isolated multispecific antibody” as used herein is intended to refer to a multispecific antibody which is substantially free of other antibodies having different antigenic specificities (for instance an isolated bispecific antibody that specifically binds to CD40 and CD137 is substantially free of monospecific antibodies that specifically bind to CD40 or CD137).

The term “monoclonal antibody” as used herein refers to a preparation of antibody molecules of single molecular composition. A monoclonal antibody composition displays a single binding specificity and affinity for a particular epitope.

When used herein the term “heterodimeric interaction between the first and second CH3 regions” refers to the interaction between the first CH3 region and the second CH3 region in a first-CH3/second-CH3 heterodimeric antibody.

When used herein the term “homodimeric interactions of the first and second C1H3 regions” refers to the interaction between a first CH3 region and another first CH3 region in a first-CH3/first-CH3 homodimeric antibody and the interaction between a second CH3 region and another second CH3 region in a second-CH3/second-CH3 homodimeric antibody.

When used herein the term “homodimeric antibody” refers to an antibody comprising two first Fab-arms or half-molecules, wherein the amino acid sequence of said Fab-arms or half-molecules is the same.

When used herein the term “heterodimeric antibody” refers to an antibody comprising a first and a second Fab-arm or half-molecule, wherein the amino acid sequence of said first and second Fab-arms or half-molecules are different. In particular, the CH3 region, or the antigen-binding region, or the CH3 region and the antigen-binding region of said first and second Fab-arms/half-molecules are different.

The term “reducing conditions” or “reducing environment” refers to a condition or an environment in which a substrate, such as a cysteine residue in the hinge region of an antibody, is more likely to become reduced than oxidized.

The present disclosure also describes multispecific antibodies, such as bispecific antibodies, comprising functional variants of the VL regions, VH regions, or one or more CDRs of the bispecific antibodies of the examples. A functional variant of a VL, VH, or CDR used in the context of a bispecific antibody still allows each antigen-binding region of the bispecific antibody to retain at least a substantial proportion (at least about 50%, 60%, 70%, 80%, 90%, 95% or more) of the affinity and/or the specificity/selectivity of the parent bispecific antibody and in some cases such a bispecific antibody may be associated with greater affinity, selectivity and/or specificity than the parent bispecific antibody.

Such functional variants typically retain significant sequence identity to the parent bispecific antibody. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

In the context of the present disclosure, unless otherwise indicated, the following notations are used to describe a mutation: i) substitution of an amino acid in a given position is written as e.g. K409R which means a substitution of a lysine in position 409 of the protein with an arginine; and ii) for specific variants the specific three or one letter codes are used, including the codes Xaa and X to indicate any amino acid residue. Thus, the substitution of lysine with arginine in position 409 is designated as: K409R, and the substitution of lysine with any amino acid residue in position 409 is designated as K409X. In case of deletion of lysine in position 409 it is indicated by K409*.

Exemplary variants include those which differ from the VII and/or VL and/or CDRs of the parent sequences mainly by conservative substitutions; for example, 12, such as 11, 10, 9, 8, 7, 6, 5, 4, 3, 2 or 1 of the substitutions in the variant are conservative amino acid residue replacements.

In the context of the present disclosure, conservative substitutions may be defined by substitutions within the classes of amino acids as defined in tables 2 and 3.

The term “CD40” as used herein, refers to CD40, also referred to as tumor necrosis factor receptor superfamily member 5 (TNFRSF5), which is the receptor for the ligand TNFSF5/CD40L. CD40 is known to transduce TRAF6- and MAP3K8-mediated signals that activate ERK in macrophages and B cells, leading to induction of immunoglobulin secretion by the B cells. Other synonyms used for CD40 include, but are not limited to, B-cell surface antigen CD40, Bp50, CD40L receptor and CDw40. In one embodiment, CD40 is human CD40, having UniProt accession number P25942. The sequence of human CD40 is also shown in SEQ ID NO: 35. Amino acids 1-20 of SEQ ID NO: 35 correspond to the signal peptide of human CD40; while amino acids 21-193 of SEQ ID NO: 35 correspond to the extracellular domain of human CD40; and the remainder of the protein; i.e. from amino acids 194-215 and 216-277 of SEQ ID NO: 35 is transmembrane and cytoplasmic domain, respectively.

The term “CD137” as used herein, refers to CD137 (4-1BB), also referred to as tumor necrosis factor receptor superfamily member 9 (TNFRSF9), which is the receptor for the ligand TNFSF9/4-1BBL. CD137 (4-1BB) is believed to be involved in T-cell activation. Other synonyms for CD137 include, but are not limited to, 4-1BB ligand receptor, CDw137, T-cell antigen 4-1BB homolog and T-cell antigen ILA. In one embodiment, CD137 (4-1BB) is human CD137 (4-1BB), having UniProt accession number Q07011. The sequence of human CD137 is also shown in SEQ ID NO: 37. Amino acids 1-23 of SEQ ID NO: 37 correspond to the signal peptide of human CD137; while amino acids 24-186 of SEQ ID NO: 37 correspond to the extracellular domain of human CD137; and the remainder of the protein, i.e. from amino acids 187-213 and 214-255 of SEQ ID NO: 37 are transmembrane and cytoplasmic domain, respectively.

The “Programmed Death-1 (PD-1)” receptor refers to an immuno-inhibitory receptor belonging to the CD28 family. PD-1 (also known as CD279) is expressed predominantly on previously activated T cells in vivo, and binds to two ligands, PD-L1 (also known as B7-H1 or CD274) and PD-L2 (also known as B7-DC or CD273). The term “PD-1” as used herein includes human PD-1 (hPD-1), variants, isoforms, and species homologs of hPD-1, and analogs having at least one common epitope with hPD-1. The sequence of human PD-1 is also shown in SEQ ID NO: 39. “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, such as macaque (cynomolgus monkey), African elephant, wild boar and mouse PD-L1 (cf., e.g., Genbank accession no. NP_054862.1, XP 005581836, XP_003413533, XP_005665023 and NP_068693, respectively), and analogs having at least one common epitope with hPD-L1. The sequence of human PD-L1 is also shown in SEQ ID NO: 40, wherein amino acids 1-18 are predicted to be a signal peptide. The sequence of macaque (cynomolgus monkey) PD-L1 is also shown in SEQ ID NO: 41, wherein amino acids 1-18 are predicted to be a signal peptide. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 what results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

“Cytotoxic T Lymphocyte Associated Antigen-4 (CTLA-4)” (also known as CD152) is a T cell surface molecule and is a member of the immunoglobulin superfamily. This protein downregulates the immune system by binding to CD80 (B7-1) and CD86 (B7-2). The term “CTLA-4” as used herein includes human CTLA-4 (hCTLA-4), variants, isoforms, and species homologs of hCTLA-4, and analogs having at least one common epitope with hCTLA-4. CTLA-4 is a homolog of the stimulatory checkpoint protein CD28 with much higher binding affinity for CD80 and CD86. CTLA4 is expressed on the surface of activated T cells and its ligands are expressed on the surface of professional antigen-presenting cells. Binding of CTLA 4 to its ligands prevents the co-stimulatory signal of CD28 and produces an inhibitory signal. Thus, CTLA-4 downregulates T cell activation. The sequence of human CTLA-4 is also shown in SEQ ID NO: 42.

“T cell Immunoreceptor with Ig and ITIM domains” (TIGIT, also known as WUCAM or Vstm3) is an immune receptor on T cells and natural killer (NK) cells and binds to PVR (CD155) on DCs, macrophages etc., and PVRL2 (CD112; nectin-2) and PVRL3 (CD113; nectin-3) and regulates T cell-mediated immunity. The term “TIGIT” as used herein includes human TIGIT (hTIGIT), variants, isoforms, and species homologs of hTIGIT, and analogs having at least one common epitope with hTIGIT. The term “PVR” as used herein includes human PVR (hPVR), variants, isoforms, and species homologs of hPVR, and analogs having at least one common epitope with hPVR. The term “PVRL2” as used herein includes human PVRL2 (hPVRL2), variants, isoforms, and species homologs of hPVRL2, and analogs having at least one common epitope with hPVRL2. The term “PVRL3” as used herein includes human PVRL3 (hPVRL3), variants, isoforms, and species homologs of hPVRL3, and analogs having at least one common epitope with hPVRL3.

“B and T Lymphocyte Attenuator” (BTLA, also known as CD272) is a TNFR family member expressed in Th1 but not Th2 cells. BTLA expression is induced during activation of T cells and is in particular expressed on surfaces of CD8+ T cells. The term “BTLA” as used herein includes human BTLA (hBTLA), variants, isoforms, and species homologs of hBTLA, and analogs having at least one common epitope with hBTLA. BTLA expression is gradually downregulated during differentiation of human CD8+ T cells to effector cell phenotype. Tumor-specific human CD8+ T cells express high levels of BTLA. BTLA binds to “Herpesvirus entry mediator” (HVEM, also known as TNFRSF14 or CD270) and is involved in T cell inhibition. The term “HVEM” as used herein includes human HVEM (hHVEM), variants, isoforms, and species homologs of hHVEM, and analogs having at least one common epitope with hHVEM. BTLA-HVEM complexes negatively regulate T cell immune responses.

“Killer-cell Immunoglobulin-like Receptors” (KIRs) are receptors for MHC Class I molecules on NK T cells and NK cells that are involved in differentiation between healthy and diseased cells. KIRs bind to human leukocyte antigen (HLA) A, B and C, what suppresses normal immune cell activation. The term “KIRs” as used herein includes human KIRs (hKIRs), variants, isoforms, and species homologs of hKIRs, and analogs having at least one common epitope with a hKIR. The term “HLA” as used herein includes variants, isofonms, and species homologs of HLA, and analogs having at least one common epitope with a HLA. KIR as used herein in particular refers to KIR2DL1, KIR2DL2, and/or KIR2DL3.

“Lymphocyte Activation Gene-3 (LAG-3)” (also known as CD223) is an inhibitory receptor associated with inhibition of lymphocyte activity by binding to MHC class II molecules. This receptor enhances the function of Treg cells and inhibits CD8+ effector T cell function leading to immune response suppression. LAG-3 is expressed on activated T cells, NK cells, B cells and DCs. The term “LAG-3” as used herein includes human LAG-3 (hLAG-3), variants, isoforms, and species homologs of hLAG-3, and analogs having at least one common epitope.

“T Cell Membrane Protein-3 (TIM-3)” (also known as HAVcr-2) is an inhibitory receptor involved in the inhibition of lymphocyte activity by inhibition of Th1 cell responses. Its ligand is galectin 9 (GAL9), which is upregulated in various types of cancers. Other TIM-3 ligands include phosphatidyl serine (PtdSer), High Mobility Group Protein 1 (HMGB1) and Carcinoembryonic Antigen Related Cell Adhesion Molecule 1 (CEACAM1). The term “TIM-3” as used herein includes human TIM3 (hTIM-3), variants, isoforms, and species homologs of hTIM-3, and analogs having at least one common epitope. The term “GAL9” as used herein includes human GAL9 (hGAL9), variants, isoforms, and species homologs of hGAL9, and analogs having at least one common epitope. The term “PdtSer” as used herein includes variants and analogs having at least one common epitope. The term “HMGB1” as used herein includes human HMGB1 (hHMGB1), variants, isoforms, and species homologs of hHMGB1, and analogs having at least one common epitope. The term “CEACAM1” as used herein includes human CEACAM1 (hCEACAM1), variants, isoforms, and species homologs of hCEACAM1, and analogs having at least one common epitope.

“CD94/NKG2A” is an inhibitory receptor predominantly expressed on the surface of natural killer cells and of CD8+ T cells. The term “CD94/NKG2A” as used herein includes human CD94/NKG2A (hCD94/NKG2A), variants, isoforms, and species homologs of hCD94/NKG2A, and analogs having at least one common epitope. The CD94/NKG2A receptor is a heterodimer comprising CD94 and NKG2A. It suppresses NK cell activation and CD8+ T cell function, probably by binding to ligands such as HLA-E. CD94/NKG2A restricts cytokine release and cytotoxic response of natural killer cells (NK cells), natural killer T cells (NK-T cells) and T cells (a/p and 7/6). NKG2A is frequently expressed in tumor infiltrating cells and HLA-E is overexpressed in several cancers.

“Indoleamine 2,3-dioxygenase” (IDO) is a tryptophan catabolic enzyme with immune-inhibitory properties. The term “IDO” as used herein includes human IDO (hIDO), variants, isoforms, and species homologs of hIDO, and analogs having at least one common epitope. IDO is the rate limiting enzyme in tryptophan degradation catalyzing its conversion to kynurenine. Therefore, IDO is involved in depletion of essential amino acids. It is known to be involved in suppression of T and NK cells, generation and activation of Tregs and myeloid-derived suppressor cells, and promotion of tumor angiogenesis. IDO is overexpressed in many cancers and was shown to promote immune system escape of tumor cells and to facilitate chronic tumor progression when induced by local inflammation.

In the “adenosinergic pathway” or “adenosine signaling pathway” as used herein ATP is converted to adenosine by the ectonucleotidases CD39 and CD73 resulting in inhibitory signaling through adenosine binding by one or more of the inhibitory adenosine receptors “Adenosine A2A Receptor” (A2AR, also known as ADORA2A) and “Adenosine A2B Receptor” (A2BR, also known as ADORA2B). Adenosine is a nucleoside with immunosuppressive properties and is present in high concentrations in the tumor microenvironment restricting immune cell infiltration, cytotoxicity and cytokine production. Thus, adenosine signaling is a strategy of cancer cells to avoid host immune system clearance. Adenosine signaling through A2AR and A2BR is an important checkpoint in cancer therapy that is activated by high adenosine concentrations typically present in the tumor microenvironment. CD39, CD73, A2AR and A2BR are expressed by most immune cells, including T cells, invariant natural killer cells, B cells, platelets, mast cells and eosinophils. Adenosine signaling through A2AR and A2BR counteracts T cell receptor mediated activation of immune cells and results in increased numbers of Tregs and decreased activation of DCs and effector T cells. The term “CD39” as used herein includes human CD39 (hCD39), variants, isoforms, and species homologs of hCD39, and analogs having at least one common epitope. The term “CD73” as used herein includes human CD73 (hCD73), variants, isoforms, and species homologs of hCD73, and analogs having at least one common epitope. The term “A2AR” as used herein includes human A2AR (hA2AR), variants, isoforms, and species homologs of hA2AR, and analogs having at least one common epitope. The term “A2BR” as used herein includes human A2BR (hA2BR), variants, isoforms, and species homologs of hA2BR, and analogs having at least one common epitope.

“V-domain Ig suppressor of T cell activation” (VISTA, also known as C10orf54) bears homology to PD-L1 but displays a unique expression pattern restricted to the hematopoietic compartment. The term “VISTA” as used herein includes human VISTA (hVISTA), variants, isoforms, and species homologs of hVISTA, and analogs having at least one common epitope. VISTA induces T cell suppression and is expressed by leukocytes within tumors.

The “Sialic acid binding immunoglobulin type lectin” (Siglec) family members recognize sialic acids and are involved in distinction between “self” and “non-self”. The term “Siglecs” as used herein includes human Siglecs (hSiglecs), variants, isoforms, and species homologs of hSiglecs, and analogs having at least one common epitope with one or more hSiglecs. The human genome contains 14 Siglecs of which several are involved in immunosuppression, including, without limitation, Siglec-2, Siglec-3, Siglec-7 and Siglec-9. Siglec receptors bind glycans containing sialic acid, but differ in their recognition of the linkage regiochemistry and spatial distribution of sialic residues. The members of the family also have distinct expression patterns. A broad range of malignancies overexpress one or more Siglecs.

“CD20” is an antigen expressed on the surface of B and T cells. High expression of CD20 can be found in cancers, such as B cell lymphomas, hairy cell leukemia, B cell chronic lymphocytic leukemia, and melanoma cancer stem cells. The term “CD20” as used herein includes human CD20 (hCD20), variants, isoforms, and species homologs of hCD20, and analogs having at least one common epitope.

“Glycoprotein A repetitions predominant” (GARP) plays a role in immune tolerance and the ability of tumors to escape the patient's immune system. The term “GARP” as used herein includes human GARP (hGARP), variants, isoforms, and species homologs of hGARP, and analogs having at least one common epitope. GARP is expressed on lymphocytes including Tregs in peripheral blood and tumor infiltrating T cells at tumor sites. It probably binds to latent “transforming growth factor β” (TGF-β). Disruption of GARP signaling in Tregs cells results in decreased tolerance and inhibits migration of Tregs to the gut and increased proliferation of cytotoxic T cells.

“CD47” is a transmembrane protein that binds to the ligand “signal-regulatory protein alpha” (SIRPα). The term “CD47” as used herein includes human CD47 (hCD47), variants, isoforms, and species homologs of hCD47, and analogs having at least one common epitope with hCD47. The term “SIRPα” as used herein includes human SIRPα (hSIRPα), variants, isoforms, and species homologs of hSIRPα, and analogs having at least one common epitope with hSIRPα. CD47 signaling is involved in a range of cellular processes including apoptosis, proliferation, adhesion and migration. CD47 is overexpressed in many cancers and functions as “don't eat me” signal to macrophages. Blocking CD47 signaling through inhibitory anti-CD47 or anti-SIRPα antibodies enables macrophage phagocytosis of cancer cells and fosters the activation of cancer-specific T lymphocytes.

“Poliovirus receptor related immunoglobulin domain containing” (PVRIG, also known as CD112R) binds to “Poliovirus receptor-related 2” (PVRL2). PVRIG and PVRL2 are overexpressed in a number of cancers. PVRIG expression also induces TIGIT and PD-1 expression and PVRL2 and PVR (a TIGIT ligand) are co-overexpressed in several cancers. Blockade of the PVRIG signaling pathway results in increased T cell function and CD8+ T cell responses and, therefore, reduced immune suppression and elevated interferon responses. The term “PVRIG” as used herein includes human PVRIG (hPVRIG), variants, isoforms, and species homologs of hPVRIG, and analogs having at least one common epitope with hPVRIG. “PVRL2” as used herein includes hPVRL2, as defined above.

The “colony-stimulating factor 1” (CSF1) pathway is another checkpoint that can be targeted according to the disclosure. CSF1R is a myeloid growth factor receptor that binds CSF1. Blockade of the CSF1R signaling can functionally reprogram macrophage responses, thereby enhancing antigen presentation and anti-tumor T cell responses. The term “CSF1R” as used herein includes human CSF1R (hCSF1R), variants, isoforms, and species homologs of hCSF1R, and analogs having at least one common epitope with hCSF1R. The term “CSF1” as used herein includes human CSF1 (hCSF1), variants, isoforms, and species homologs of hCSF1, and analogs having at least one common epitope with hCSF1.

“Nicotinamide adenine dinucleotide phosphate NADPH oxidase” refers to an enzyme of the NOX family of enzymes of myeloid cells that generate immunosuppressive reactive oxygen species (ROS). Five NOX enzymes (NOX1 to NOX5) have been found to be involved in cancer development and immunosuppression. Elevated ROS levels have been detected in almost all cancers and promote many aspects of tumor development and progression. NOX produced ROS dampens NK and T cell functions and inhibition of NOX in myeloid cells improves anti-tumor functions of adjacent NK cells and T cells. The term “NOX” as used herein includes human NOX (hNOX), variants, isoforms, and species homologs of hNOX, and analogs having at least one common epitope with hNOX.

Another immune checkpoint that can be targeted according to the disclosure is the signal mediated by “tryptophan-2,3-dioxygenase” (TDO). TDO represents an alternative route to IDO in tryptophan degradation and is involved in immune suppression. Since tumor cells may catabolize tryptophan via TDO instead of IDO, TDO may represent an additional target for checkpoint blockade. Indeed, several cancer cell lines have been found to upregulate TDO and TDO may complement IDO inhibition. The term “TDO” as used herein includes human TDO (hTDO), variants, isoforms, and species homologs of hTDO, and analogs having at least one common epitope with hTDO.

Many of the immune checkpoints are regulated by interactions between specific receptor and ligand pairs, such as those described above. Thus, immune checkpoint proteins mediate immune checkpoint signaling. For example, checkpoint proteins directly or indirectly regulate T cell activation, T cell proliferation and/or T cell function. Cancer cells often exploit these checkpoint pathways to protect themselves from being attacked by the immune system. Hence, the function of checkpoint proteins is typically the regulation of T cell activation, T cell proliferation and/or T cell function. Immune checkpoint proteins thus regulate and maintain self-tolerance and the duration and amplitude of physiological immune responses. Many of the immune checkpoint proteins belong to the B7:CD28 family or to the tumor necrosis factor receptor (TNFR) super family and, by binding to specific ligands, activate signaling molecules that are recruited to the cytoplasmic domain (Suzuki et al., 2016, Jap J Clin One, 46:191-203).

The term “dysfunctional”, as used herein, refers to an immune cell that is in a state of reduced immune responsiveness to antigen stimulation. Dysfunctional includes unresponsive to antigen recognition and impaired capacity to translate antigen recognition into downstream T cell effector functions, such as proliferation, cytokine production (e.g., IL-2) and/or target cell killing.

The term “anergy”, as used herein, refers to the state of unresponsiveness to antigen stimulation resulting from incomplete or insufficient signals delivered through the T cell receptor (TCR). T cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T cells do not undergo clonal expansion and/or acquire effector functions.

The term “exhaustion”, as used herein, refers to immune cell exhaustion, such as T cell exhaustion as a state of T cell dysfunction that arises from sustained TCR signaling that occurs during many chronic infections and cancer. It is distinguished from anergy in that it arises not through incomplete or deficient signaling, but from sustained signaling. Exhaustion is defined by poor effector function, sustained expression of inhibitory receptors and a transcriptional state distinct from that of functional effector or memory T cells. Exhaustion prevents optimal control of diseases (e.g., infection and tumors). Exhaustion can result from both extrinsic negative regulatory pathways (e.g., immunoregulatory cytokines) as well as cell intrinsic negative regulatory pathways (inhibitory immune checkpoint pathways, such as described herein).

“Enhancing T cell function” means to induce, cause or stimulate a T cell to have a sustained or amplified biological function, or renew or reactivate exhausted or inactive T cells. Examples of enhancing T cell function include increased secretion of y-interferon from CD8+ T cells, increased proliferation, increased antigen responsiveness (e.g., tumor clearance) relative to such levels before the intervention. In one embodiment, the level of enhancement is as least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 200%, or more. Manners of measuring this enhancement are known to one of ordinary skill in the art.

The term “inhibitory nucleic acid” or “inhibitory nucleic acid molecule” as used herein refers to a nucleic acid molecule, e.g., DNA or RNA, that totally or partially reduces, inhibits, interferes with or negatively modulates one or more checkpoint proteins. Inhibitory nucleic acid molecules include, without limitation, oligonucleotides, siRNA, shRNA, antisense DNA or RNA molecules, and aptamers (e.g., DNA or RNA aptamers).

The term “oligonucleotide” as used herein refers to a nucleic acid molecule that is able to decrease protein expression, in particular expression of a checkpoint protein, such as the checkpoint proteins described herein. Oligonucleotides are short DNA or RNA molecules, typically comprising from 2 to 50 nucleotides. Oligonucleotides maybe single-stranded or double-stranded. A checkpoint inhibitor oligonucleotide may be an antisense-oligonucleotide.

Antisense-oligonucleotides are single-stranded DNA or RNA molecules that are complementary to a given sequence, in particular to a sequence of the nucleic acid sequence (or a fragment thereof) of a checkpoint protein. Antisense RNA is typically used to prevent protein translation of mRNA, e.g., of mRNA encoding a checkpoint protein, by binding to said mRNA. Antisense DNA is typically used to target a specific, complementary (coding or non-coding) RNA. If binding takes place, such a DNA/RNA hybrid can be degraded by the enzyme RNase H. Moreover, morpholino antisense oligonucleotides can be used for gene knockdowns in vertebrates. For example, Kryczek et al., 2006 (J Exp Med, 203:871-81) designed B7-14-specific morpholinos that specifically blocked B7-H4 expression in macrophages, resulting in increased T cell proliferation and reduced tumor volumes in mice with tumor associated antigen (TAA)-specific T cells.

The terms “siRNA” or “small interfering RNA” or “small inhibitory RNA” are used interchangeably herein and refer to a double-stranded RNA molecule with a typical length of 20-25 base pairs that interferes with expression of a specific gene, such as a gene coding for a checkpoint protein, with a complementary nucleotide sequence. In one embodiment, siRNA interferes with mRNA therefore blocking translation, e.g., translation of an immune checkpoint protein. Transfection of exogenous siRNA may be used for gene knockdown, however, the effect maybe only transient, especially in rapidly dividing cells. Stable transfection may be achieved, e.g., by RNA modification or by using an expression vector. Useful modifications and vectors for stable transfection of cells with siRNA are known in the art. siRNA sequences may also be modified to introduce a short loop between the two strands resulting in a “small hairpin RNA” or “shRNA”. shRNA can be processed into a functional siRNA by Dicer. shRNA has a relatively low rate of degradation and turnover. Accordingly, the immune checkpoint inhibitor may be a shRNA.

The term “aptamer” as used herein refers to a single-stranded nucleic acid molecule, such as DNA or RNA, typically in a length of 25-70 nucleotides that is capable of binding to a target molecule, such as a polypeptide. In one embodiment, the aptamer binds to an immune checkpoint protein such as the immune checkpoint proteins described herein. For example, an aptamer according to the disclosure can specifically bind to an immune checkpoint protein or polypeptide, or to a molecule in a signaling pathway that modulates the expression of an immune checkpoint protein or polypeptide. The generation and therapeutic use of aptamers is well known in the art (see, e.g., U.S. Pat. No. 5,475,096).

The terms “small molecule inhibitor” or “small molecule” are used interchangeably herein and refer to a low molecular weight organic compound, usually up to 1000 daltons, that totally or partially reduces, inhibits, interferes with, or negatively modulates one or more checkpoint proteins as described above. Such small molecular inhibitors are usually synthesized by organic chemistry, but may also be isolated from natural sources, such as plants, fungi, and microbes. The small molecular weight allows a small molecule inhibitor to rapidly diffuse across cell membranes. For example, various A2AR antagonists known in the art are organic compounds having a molecular weight below 500 daltons.

The term “cell based therapy” refers to the transplantation of cells (e.g., T lymphocytes, dendritic cells, or stem cells) expressing an immune checkpoint inhibitor into a subject for the purpose of treating a disease or disorder (e.g., a cancer disease).

The term “oncolytic virus” as used herein, refers to a virus capable of selectively replicating in and slowing the growth or inducing the death of a cancerous or hyperproliferative cell, either in vitro or in vivo, while having no or minimal effect on normal cells. An oncolytic virus for the delivery of an immune checkpoint inhibitor comprises an expression cassette that may encode an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. The oncolytic virus preferably is replication competent and the expression cassette is under the control of a viral promoter, e.g., synthetic early/late poxvirus promoter. Exemplary oncolytic viruses include vesicular stomatitis virus (VSV), rhabdoviruses (e.g., picornaviruses such as Seneca Valley virus; SVV-001), coxsackievirus, parvovirus, Newcastle disease virus (NDV), herpes simplex virus (HSV; OncoVEX GMCSF), retroviruses (e.g., influenza viruses), measles virus, reovirus, Sinbis virus, vaccinia virus, as exemplarily described in WO 2017/209053 (including Copenhagen, Western Reserve, Wyeth strains), and adenovirus (e.g., Delta-24, Delta-24-RGD, ICOVIR-5, ICOVIR-7, Onyx-015, ColoAd1, H101, AD5/3-D24-GMCSF). Generation of recombinant oncolytic viruses comprising a soluble form of an immune checkpoint inhibitor and methods for their use are disclosed in WO 2018/022831, herein incorporated by reference in its entirety. Oncolytic viruses can be used as attenuated viruses.

“Treatment cycle” is herein defined as the time period, within the effects of separate dosages of the binding agent add on due to its pharmacodynamics, or in other words the time period after the subject's body is essentially cleared from the administrated biding agent. Multiple small doses in a small time window, e.g. within 2-24 few hours, such as 2-12 hours or on the same day, might be equal to a larger single dose.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications. In one embodiment, “treatment” refers to the administration of an effective amount of a therapeutically active binding agent, such as of a therapeutically active antibody, of the present disclosure with the purpose of easing, ameliorating, arresting or eradicating (curing) symptoms or disease states.

The resistance to, failure to respond to and/or relapse from treatment with a binding agent of the present disclosure may be determined according to the Response Evaluation Criteria in Solid Tumors; version 1.1 (RECIST Criteria v1.1). The RECIST Criteria are set forth in the table below (LD: longest dimension).

TABLE 4
Definition of Response (RECIST Criteria v1.1)

| | Category | Criteria |
|---|---|---|
| Based on target | Complete Response | Disappearance of all target lesions. Any pathological lymph |
| lesions | (CR) | nodes must have reduction in short axis to <10 mm. |
| | Partial Response | ≥30% decrease in the sum of the LD of target lesions, |
| | (PR) | taking as reference the baseline sum LD. |
| | Stable Disease | Neither sufficient shrinkage to qualify for PR nor sufficient |
| | (SD) | increase to qualify for PD, taking as reference the smallest |
| | | sum of LDs since the treatment started. |
| | Progressive Disease | ≥20% increase in the sum of the LDs of target lesions, |
| | (PD) | taking as reference the smallest sum of the LDs recorded |
| | | since the treatment started or the appearance of one or more |
| | | new lesions. |
| Based on non- | CR | Disappearance of all non-target lesions and normalization of |
| target lesions | | tumor marker level. All lymph nodes must be non- |
| | | pathological in size (<10 mm short axis). |
| | SD | Persistence of one or more non-target lesion(s) or/and |
| | | maintenance of tumor marker level above the normal limits. |
| | PD | Appearance of one or more new lesions and/or unequivocal |
| | | progression of existing non-target lesions. |

The “best overall response” is the best response recorded from the start of the treatment until disease progression/recurrence (the smallest measurements recorded since the treatment started will be used as the reference for PD). Subjects with CR or PR are considered to be objective response. Subjects with CR, PR or SD are considered to be in disease control. Subjects with NE are counted as non-responders.

The best overall response is the best response recorded from the start of the treatment until disease progression/recurrence (the smallest measurements recorded since the treatment started will be used as the reference for PD). Subjects with CR, PR or SD are considered to be in disease control. Subjects with NE are counted as non-responders.

“Duration of response (DOR)” only applies to subjects whose confirmed best overall response is CR or PR and is defined as the time from the first documentation of objective tumor response (CR or PR) to the date of first PD or death due to underlying cancer.

“Progression-free survival (PFS)” is defined as the number of days from Day 1 in Cycle 1 to the first documented progression or death due to any cause.

“Overall survival (OS)” is defined as the number of days from Day 1 in Cycle 1 to death due to any cause. If a subject is not known to have died, then OS will be censored at the latest date the subject was known to be alive (on or before the cut-off date).

In the context of the present disclosure, the term “treatment regimen” refers to a structured treatment plan designed to improve and maintain health.

The term “effective amount” or “therapeutically effective amount” refers to an amount effective, at dosages and for periods of time necessary, to achieve a desired therapeutic result. A therapeutically effective amount of a binding agent, such as an antibody, like a multispecific antibody or monoclonal antibody, may vary according to factors such as the disease state, age, sex, and weight of the individual, and the ability of the binding agent to elicit a desired response in the individual. A therapeutically effective amount is also one in which any toxic or detrimental effects of the binding agent or a fragment thereof, are outweighed by the therapeutically beneficial effects. In the case that a reaction in a patient is insufficient with an initial dose, higher doses (or effectively higher doses achieved by a different, more localized route of administration) may be used. In case that unwanted side effects occur in a patient with a dose, lower doses (or effectively lower doses achieved by a different, more localized route of administration) may be used.

As used herein, the term “cancer” includes a disease characterized by aberrantly regulated cellular growth, proliferation, differentiation, adhesion, and/or migration. By “cancer cell” is meant an abnormal cell that grows by a rapid, uncontrolled cellular proliferation and continues to grow after the stimuli that initiated the new growth cease.

The term “cancer” according to the present disclosure comprises leukemias, seminomas, melanomas, sarcomas, myelomas, teratomas, lymphomas, mesotheliomas, neuroblastomas, gliomas, rectal cancer, endometrial cancer, kidney cancer, renal cancer, urothelial cancer, adrenal cancer, adrenocortical cancer, thyroid cancer, blood cancer, skin cancer, cancer of the brain, cervical cancer, intestinal cancer, liver cancer, colon cancer, stomach cancer, intestine cancer, head and neck cancer, gastric cancer, gastrointestinal cancer, lymph node cancer, esophagus cancer, colorectal cancer, pancreas cancer, ear, nose and throat (ENT) cancer, breast cancer, prostate cancer, penile cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above.

The term “cancer” according to the present disclosure also comprises cancer metastases. By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the present disclosure relates to “distant metastasis” which relates to a metastasis which is remote from the primary tumor and the regional lymph node system.

Terms such as “reduce”, “inhibit”, “interfere”, and “negatively modulate” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “increase” or “enhance” in one embodiment relate to an increase or enhancement by at least about 10%, at least about 20%, at least about 30%, at least about 40%, at least about 50%, at least about 80%, or at least about 100%.

“Physiological pH” as used herein refers to a pH of about 7.5.

As used in the present disclosure, “% by weight” refers to weight percent, which is a unit of concentration measuring the amount of a substance in grams (g) expressed as a percent of the total weight of the total composition in grams (g).

The term “freezing” relates to the solidification of a liquid, usually with the removal of heat.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “recombinant” in the context of the present disclosure means “made through genetic engineering”. In one embodiment, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and immunostimulants such as cytokines.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

According to the present disclosure, a part or fragment of a peptide or protein preferably has at least one functional property of the peptide or protein from which it has been derived. Such functional properties comprise a pharmacological activity, the interaction with other peptides or proteins, an enzymatic activity, the interaction with antibodies, and the selective binding of nucleic acids. E.g., a pharmacological active fragment of a peptide or protein has at least one of the pharmacological activities of the peptide or protein from which the fragment has been derived. A part or fragment of a peptide or protein preferably comprises a sequence of at least 6, in particular at least 8, at least 10, at least 12, at least 15, at least 20, at least 30 or at least 50, consecutive amino acids of the peptide or protein. A part or fragment of a peptide or protein preferably comprises a sequence of up to 8, in particular up to 10, up to 12, up to 15, up to 20, up to 30 or up to 55, consecutive amino acids of the peptide or protein.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch. 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P. BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference amino acid sequence consists of 200 amino acid residues, the degree of identity is given for at least about 100, at least about 120, at least about 140, at least about 160, at least about 180, or about 200 amino acid residues, in some embodiments continuous amino acid residues. In some embodiments, the degree of similarity or identity is given for the entire length of the reference sequence.

Homologous amino acid sequences exhibit according to the present disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. In a preferred embodiment, the binding agent used in the present disclosure is in substantially purified form.

The term “genetic modification” or simply “modification” includes the transfection of cells with nucleic acid. The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the present disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding antigen is transiently transfected into cells. RNA can be transfected into cells to transiently express its coded protein.

According to the present disclosure, an analog of a peptide or protein is a modified form of said peptide or protein from which it has been derived and has at least one functional property of said peptide or protein. E.g., a pharmacological active analog of a peptide or protein has at least one of the pharmacological activities of the peptide or protein from which the analog has been derived. Such modifications include any chemical modification and comprise single or multiple substitutions, deletions and/or additions of any molecules associated with the protein or peptide, such as carbohydrates, lipids and/or proteins or peptides. In one embodiment, “analogs” of proteins or peptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said proteins and peptides.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or protein, that reacts specifically with antibodies or T-lymphocytes (T-cells). According to the present disclosure, the term “antigen” comprises any molecule which comprises at least one epitope, such as a T cell epitope. Preferably, an antigen in the context of the present disclosure is a molecule which, optionally after processing, induces an immune reaction, which is preferably specific for the antigen (including cells expressing the antigen). In one embodiment, an antigen is a disease-associated antigen, such as a tumor antigen, a viral antigen, or a bacterial antigen, or an epitope derived from such antigen.

According to the present disclosure, any suitable antigen may be used, which is a candidate for an immune response, wherein the immune response may be both a humoral as well as a cellular immune response. In the context of some embodiments of the present disclosure, the antigen is preferably presented by a cell, preferably by an antigen presenting cell, in the context of MHC molecules, which results in an immune response against the antigen. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present disclosure, an antigen may correspond to a naturally occurring product, for example, a viral protein, or a part thereof.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In a preferred embodiment, the antigen is a tumor antigen, i.e., a part of a tumor cell, in particular those which primarily occur intracellularly or as surface antigens of tumor cells. In another embodiment, the antigen is a pathogen-associated antigen, i.e., an antigen derived from a pathogen, e.g., from a virus, bacterium, unicellular organism, or parasite, for example a viral antigen such as viral ribonucleoprotein or coat protein. In particular, the antigen should be presented by MHC molecules which results in modulation, in particular activation of cells of the immune system, preferably CD4⁺ and CD8⁺ lymphocytes, in particular via the modulation of the activity of a T-cell receptor.

The term “tumor antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, α1-fetoprotein, isoferritin, and fetal sulphoglycoprotein, α2-H-ferroprotein and γ-fetoprotein, as well as various virus tumor antigens. According to the present disclosure, a tumor antigen preferably comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to an antigenic determinant in a molecule such as an antigen, i.e., to a part in or fragment of the molecule that is recognized by the immune system, for example, that is recognized by antibodies T cells or B cells, in particular when presented in the context of MHC molecules. In one embodiment, “epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen-binding peptide (in other words, the amino acid residue is within the footprint of the specifically antigen-binding peptide).

An epitope of a protein preferably comprises a continuous or discontinuous portion of said protein and is preferably between about 5 and about 100, preferably between about 5 and about 50, more preferably between about 8 and about 0, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. It is particularly preferred that the epitope in the context of the present disclosure is a T cell epitope.

Terms such as “epitope”, “fragment of an antigen”, “immunogenic peptide” and “antigen peptide” are used interchangeably herein and preferably relate to an incomplete representation of an antigen which is preferably capable of eliciting an immune response against the antigen or a cell expressing or comprising and preferably presenting the antigen. Preferably, the terms relate to an immunogenic portion of an antigen. Preferably, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In one embodiment, vaccine antigen, i.e., an antigen whose inoculation into a subject induces an immune response, is recognized by an immune effector cell. Preferably, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell. In one embodiment, an antigen is presented by a diseased cell (such as tumor cell or an infected cell). In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g., performs and granzymes.

In one embodiment, an antigen receptor is an antibody or B cell receptor which binds to an epitope in an antigen. In one embodiment, an antibody or B cell receptor binds to native epitopes of an antigen.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by, e.g., antigen-specific antibodies added to the cells.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured. In certain embodiments of the present disclosure, the RNA (in particular mRNA) encodes at least one epitope.

The term “target” shall mean an agent such as a cell or tissue which is a target for an immune response such as a cellular immune response. Targets include cells that present an antigen or an antigen epitope, i.e., a peptide fragment derived from an antigen. In one embodiment, the target cell is a cell expressing an antigen and preferably presenting said antigen with class I MHC.

“Antigen processing” refers to the degradation of an antigen into processing products which are fragments of said antigen (e.g., the degradation of a protein into peptides) and the association of one or more of these fragments (e.g., via binding) with MHC molecules for presentation by cells, preferably antigen-presenting cells to specific T-cells.

By “antigen-responsive CTL” is meant a CD8 T-cell that is responsive to an antigen or a peptide derived from said antigen, which is presented with class I MHC on the surface of antigen presenting cells.

According to the present disclosure, CTL responsiveness may include sustained calcium flux, cell division, production of cytokines such as IFNγ and TNFα, up-regulation of activation markers such as CD44 and CD69, and specific cytolytic killing of tumor antigen expressing target cells. CTL responsiveness may also be determined using an artificial reporter that accurately indicates CTL responsiveness.

The terms “immune response” and “immune reaction” are used herein interchangeably in their conventional meaning and refer to an integrated bodily response to an antigen and preferably refers to a cellular immune response, a humoral immune response, or both. According to the present disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, preferably CD4⁺ and CD8⁺ T-lymphocytes, more preferably CD8⁺ T-lymphocytes, which may be detected in various proliferation or cytokine production tests in vitro.

The terms “inducing an immune response” and “eliciting an immune response” and similar terms in the context of the present disclosure refer to the induction of an immune response, preferably the induction of a cellular immune response, a humoral immune response, or both. The immune response may be protective/preventive/prophylactic and/or therapeutic. The immune response may be directed against any immunogen or antigen or antigen peptide, preferably against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. Preferably, after inducing an immune response in an individual, said individual is protected from developing a disease such as an infectious disease or a cancerous disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The terms “vaccination” and “immunization” describe the process of treating an individual for therapeutic or prophylactic reasons and relate to the procedure of administering one or more immunogen(s) or antigen(s) or derivatives thereof, in particular in the form of RNA (especially mRNA) coding therefor, as described herein to an individual and stimulating an immune response against said one or more immunogen(s) or antigen(s) or cells characterized by presentation of said one or more immunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular a tumor cell or an infected cell, or an antigen presenting cell presenting the antigen or an antigen peptide, either directly or following processing, in the context of MHC molecules, preferably MHC class I and/or MHC class II molecules, most preferably MHC class I molecules.

In the context of the present disclosure, the term “transcription” relates to a process, wherein the genetic code in a DNA sequence is transcribed into RNA (especially mRNA). Subsequently, the RNA (especially mRNA) may be translated into peptide or protein.

The term “expression” as used herein is defined as the transcription and/or translation of a particular nucleotide sequence. With respect to RNA, the term “expression” or “translation” relates to the process in the ribosomes of a cell by which a strand of mRNA directs the assembly of a sequence of amino acids to make a peptide or protein.

The term “optional” or “optionally” as used herein means that the subsequently described event, circumstance or condition may or may not occur, and that the description includes instances where said event, circumstance, or condition occurs and instances in which it does not occur.

As used herein “endogenous” refers to any material from or produced inside an organism, cell, tissue or system.

As used herein, the terms “linked”, “fused”, or “fusion” are used interchangeably. These terms refer to the joining together of two or more elements or components or domains.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably. Similarly, the term “method for preventing” in the context of progression of a disease, such as progression of a tumor or cancer, relates to any method that is intended to prevent the disease from progressing in an individual.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

### Aspects and Embodiments of the Present Disclosure

In a first aspect, the present disclosure provides a binding agent for use in a method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137.

As demonstrated in the present disclosure, a combination of (i) stimulation with a binding agent binding human CD40 and binding human CD137 and (ii) checkpoint inhibition (in particular inhibition of the PD-1/PD-L1 axis) amplifies the immune response. Without being bound to any theory, the rational behind this surprising finding could be as follows: CD137 is co-expressed on PD-1⁺ cells. Thus, blockade of PD-L1/PD-1 signals and co-stimulation through CD137 can synergize to enhance T-cell effector functions and improve the duration of the response. Through conditional activation of CD40 and CD137, a binding agent targeting CD40 and CD137 induces potent anti-tumor activity through enhanced T-cell priming, cytokine and chemokine production, and expansion and survival of antigen-experienced T cells. The PD-(L)1 pathway is expected to be activated during priming as well as during continuous antigen exposure, which may reduce the magnitude of the immune response induced by the binding agent targeting CD40 and CD137.

### Binding Agent Binding to CD40 and CD137

In one embodiment, CD40 is human CD40, in particular human CD40 comprising the sequence set forth in SEQ ID NO: 36. In one embodiment, CD137 is human CD137, in particular human CD137 comprising the sequence set forth in SEQ ID NO: 38. In one embodiment, CD40 is human CD40 and CD137 is human CD137. In one embodiment, CD40 is human CD40 comprising the sequence set forth in SEQ ID NO: 36, and CD137 is human CD137 comprising the sequence set forth in SEQ ID NO: 38.

In one embodiment of the binding agent according to the first aspect,
a) the first binding region binding to human CD40 comprises a heavy chain variable region (VH) comprising the CDR1, CDR2, and CDR3 sequences of SEQ ID NO: 7 or 9, and a light chain variable region (VL) comprising the CDR1, CDR2, and CDR3 sequences of SEQ ID NO: 8 or 10;
and
b) the second antigen-binding region binding to human CD137 comprises a heavy chain variable region (VH) comprising the CDR1, CDR2, and CDR3 sequences of SEQ ID NO: 17 or 19, and a light chain variable region (VL) comprising the CDR1, CDR2, and CDR3 sequences of SEQ ID NO: 18 or 20.

In one embodiment of the binding agent according to the first aspect,
a) the first binding region binding to human CD40 comprises a heavy chain variable region (VH) comprising the CDR1, CDR2, and CDR3 sequences set forth in: SEQ ID NO: 1, 2, and 3, respectively, and a light chain variable region (VL) comprising the CDR1, CDR2, and CDR3 sequences set forth in: SEQ ID NO: 4, 5, and 6, respectively;
and
b) the second antigen-binding region binding to human CD137 comprises a heavy chain variable region (VH) comprising the CDR1, CDR2, and CDR3 sequences set forth in: SEQ ID NO: 11, 12, and 13, respectively, and a light chain variable region (VL) comprising the CDR1, CDR2, and CDR3 sequences set forth in: SEQ ID NO: 14, 15, and 16, respectively.

In one embodiment of the binding agent according to the first aspect,
a) the first binding region binding to human CD40 comprises a heavy chain variable region (V H) comprising an amino acid sequence having at least 90%, at least 95%, at least 97%, at least 99%, or 100% sequence identity to SEQ ID NO: 7 or 9 and a light chain variable region (VL) region and comprising an amino acid sequence having at least 90%, at least 95%, at least 97%, at least 99%, or 100% sequence identity to SEQ ID NO: 8 or 10;
b) the second binding region binding to human CD137 comprises a heavy chain variable region (VH) comprising an amino acid sequence having at least 90%, at least 95%, at least 97%, at least 99%, or 25 100% sequence identity to SEQ ID NO: 17 or 19 and a light chain variable region (VL) region comprising an amino acid sequence having at least 90%, at least 95%, at least 97%, at least 99%, or 100% sequence identity to SEQ ID NO: 18 or 20.

In one embodiment of the binding agent according to the first aspect,
a) the first binding region binding to human CD40 comprises a heavy chain variable region (VH) comprising the amino acid sequence set forth in SEQ ID NO: 7 or 9 and a light chain variable region (VL) region comprising the amino acid sequence set forth in SEQ ID NO: 8 or 10;
and
b) the second binding region binding to human CD137 comprises a heavy chain variable region (VH) comprising the amino acid sequence set forth in SEQ ID NO: 17 or 19 and a light chain variable region (VL) region comprising the amino acid sequence set forth in SEQ ID NO: 18 or 20.

In one embodiment of the binding agent according to the first aspect,
a) the first binding region binding to human CD40 comprises a heavy chain variable region (VH) comprising the amino acid sequence set forth in SEQ ID NO: 9 and a light chain variable region (VL) region comprising the amino acid sequence set forth in SEQ ID NO: 10;
and
b) the second binding region binding to human CD137 comprises a heavy chain variable region (VH) comprising the amino acid sequence set forth in SEQ ID NO: 19 and a light chain variable region (VL) region comprising the amino acid sequence set forth in SEQ ID NO: 20.

The binding agent may in particular be an antibody, such as a multispecific antibody, e.g., a bispecific antibody. Also, the binding agent may be in the format of a full-length antibody or an antibody fragment.

It is further preferred that the binding agent is a human antibody or a humanized antibody.

Each variable region may comprise three complementarity determining regions (CDR1, CDR2, and CDR3) and four framework regions (FR1, FR2, FR3, and FR4).

The complementarity determining regions (CDRs) and the framework regions (FRs) may be arranged from amino-terminus to carboxy-terminus in the following order: FR1, CDR1, FR2, CDR2, FR3, CDR3, FR4.

In one embodiment of the first aspect, the binding agent comprises
i) a polypeptide comprising said first heavy chain variable region (VII) and a first heavy chain constant region (CH), and
ii) a polypeptide comprising said second heavy chain variable region (VH) and a second heavy chain constant region (CH).

In one embodiment of the first aspect, the binding agent comprises
i) a polypeptide comprising said first light chain variable region (VL) and further comprising a first light chain constant region (CL), and
ii) a polypeptide comprising said second light chain variable region (VL) and further comprising a second light chain constant region (CL).

In one embodiment of the first aspect, the binding agent is an antibody comprising a first binding arm and a second binding arm, wherein the first binding arm comprises
i) a polypeptide comprising said first heavy chain variable region (VH) and said first heavy chain constant region (CH), and
ii) a polypeptide comprising said first light chain variable region (VL) and said first light chain constant region (CL);
and the second binding arm comprises
iii) a polypeptide comprising said second heavy chain variable region (VH) and said second heavy chain constant region (CH), and
iv) a polypeptide comprising said second light chain variable region (VL) and said second light chain constant region (CL).

In one embodiment of the first aspect, the binding agent comprises i) a first heavy chain and light chain comprising said antigen-binding region capable of binding to CD40, the first heavy chain comprising a first heavy chain constant region and the first light chain comprising a first light chain constant region; and ii) a second heavy chain and light chain comprising said antigen-binding region capable of binding CD137, the second heavy chain comprising a second heavy chain constant region and the second light chain comprising a second light chain constant region.

Each of the first and second heavy chain constant regions (CH) may comprise one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

Each of the first and second heavy chain constant regions (CHs) may comprise a CH3 region, wherein the two CH3 regions comprise asymmetrical mutations. Asymmetrical mutations mean that the sequences of said first and second CH3 regions contain amino acid substitutions at non-identical positions. For example, one of said first and second CH3 regions contains a mutation at the position corresponding to position 405 in a human IgG1 heavy chain according to EU numbering, and the other of said first and second CH3 regions contains a mutation at the position corresponding to position 409 in a human IgG1 heavy chain according to EU numbering.

In said first heavy chain constant region (CH) at least one of the amino acids in a position corresponding to a position selected from the group consisting of T366, L368, K370, D399, F405, Y407, and K409 in a human IgG1 heavy chain according to EU numbering may have been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering may have been substituted. In particular embodiments, the first and the second heavy chains are not substituted in the same positions (i.e., the first and the second heavy chains contain asymmetrical mutations).

In one embodiment of the binding agent according to the first aspect, (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

In one embodiment of the first aspect, the binding agent induces Fc-mediated effector function to a lesser extent compared to another antibody comprising the same first and second antigen binding regions and two heavy chain constant regions (CHs) comprising human IgG1 hinge, CH2 and CH3 regions.

In one particular embodiment of the binding agent according to the first aspect, said first and second heavy chain constant regions (CHs) are modified so that the antibody induces Fe-mediated effector function to a lesser extent compared to an antibody which is identical except for comprising non-modified first and second heavy chain constant regions (CHs). In particular, each or both of said non-modified first and second heavy chain constant regions (CHs) may comprise, consists of or consist essentially of the amino acid sequence set forth in SEQ ID NO: 21 or 29.

The Fc-mediated effector function may be determined by measuring binding of the binding agent to Fcγ receptors, binding to C1q, or induction of Fc-mediated cross-linking of Fey receptors. In particular, the Fe-mediated effector function may be determined by measuring binding of the binding agent to C1q.

The first and second heavy chain constant regions of the binding agent may have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

In one embodiment of the binding agent according to the first aspect, in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

In one embodiment of the binding agent according to the first aspect, the positions corresponding to positions L234 and L235 in a human IgG1 heavy chain according to EU numbering may be F and E, respectively, in said first and second heavy chains.

In particular, the positions corresponding to positions L234, L235, and D265 in a human IgG1 heavy chain according to EU numbering may be F, E, and A, respectively, in said first and second heavy chain constant regions (HCs).

In one embodiment of the binding agent according to the first aspect, the positions corresponding to positions L234 and L235 in a human IgG1 heavy chain according to EU numbering of both the first and second heavy chain constant regions are F and E, respectively, wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

In one embodiment of the binding agent according to the first aspect, the positions corresponding to positions L234, L235, and D265 in a human IgG1 heavy chain according to EU numbering of both the first and second heavy chain constant regions are F, E, and A, respectively, wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

In one embodiment of the binding agent according to the first aspect, the constant region of said first and/or second heavy chain comprises an amino acid sequence selected from the group consisting of
a) the sequence set forth in SEQ ID NO: 21 or SEQ ID NO: 29 [IgG1-FC];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).

In one embodiment of the binding agent according to the first aspect, the constant region of said first or second heavy chain, such as the second heavy chain, comprises or consists essentially of or consists of an amino acid sequence selected from the group consisting of
a) the sequence set forth in SEQ ID NO: 22 or SEQ ID NO: 30 [IgG1-F405L];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).

In one embodiment of the binding agent according to the first aspect, the constant region of said first or second heavy chain, such as the first heavy chain comprises or consists essentially of or consists of an amino acid sequence selected from the group consisting of
a) the sequence set forth in SEQ ID NO: 23 or 31 [IgG1-F409R];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).

In one embodiment of the binding agent according to the first aspect, the constant region of said first and/or second heavy chain comprises or consists essentially of or consists of an amino acid sequence selected from the group consisting of
a) the sequence set forth in SEQ ID NO: 24 or SEQ ID NO: 32 [IgG1-Fc_FEA];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 7 substitutions, such as at the most 6 substitutions, at the most 5, at the most 4, at the most 3, at the most 2 or at the most 1 substitution compared to the amino acid sequence defined in a) or b).

In one embodiment of the binding agent according to the first aspect, the constant region of said first and/or second heavy chain, such as the second heavy chain, comprises or consists essentially of or consists of an amino acid sequence selected from the group consisting of
a) the sequence set forth in SEQ ID NO: 25 or SEQ ID NO: 33 [IgG1-Fc_FEAL];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4 substitutions, at the most 3, at the most 2 or at the most 1 substitution compared to the amino acid sequence defined in a) or b).

In one embodiment of the binding agent according to the first aspect, the constant region of said first and/or second heavy chain, such as the first heavy chain, comprises or consists essentially of or consists of an amino acid sequence selected from the group consisting of
a) the sequence set forth in SEQ ID NO: 26 or SEQ ID NO: 34 [IgG1-Fc_FEAR];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4, at the most 3, at the most 2 or at the most 1 substitution compared to the amino acid sequence defined in a) or b).

In one embodiment of the first aspect, the binding agent comprises a kappa (κ) light chain constant region.

In one embodiment of the first aspect, the binding agent comprises a lambda (λ) light chain constant region.

In one embodiment of the binding agent according to the first aspect, the first light chain constant region is a kappa (κ) light chain constant region or a lambda (λ) light chain constant region.

In one embodiment of the binding agent according to the first aspect, the second light chain constant region is a lambda (λ) light chain constant region or a kappa (κ) light chain constant region.

In one embodiment of the binding agent according to the first aspect, the first light chain constant region is a kappa (κ) light chain constant region and the second light chain constant region is a lambda (λ) light chain constant region or the first light chain constant region is a lambda (λ) light chain constant region and the second light chain constant region is a kappa (κ) light chain constant region.

In one embodiment of the binding agent according to the first aspect, the kappa (κ) light chain comprises an amino acid sequence selected from the group consisting of
a) the sequence set forth in SEQ ID NO: 27;
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

In one embodiment of the binding agent according to the first aspect, the lambda (λ) light chain comprises an amino acid sequence selected from the group consisting of
a) the sequence set forth in SEQ ID NO: 28;
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

The binding agent (in particular, antibody) according to the first aspect is of an isotype selected from the group consisting of IgG1, IgG2, IgG3, and IgG4. In particular, the binding agent may be a full-length IgG1 antibody. In preferred embodiments of the first aspect, the binding agent (in particular, antibody) is of the IgG1m(f) allotype.

Preferably, the binding agent is administered in a suitable amount, i.e., the amount of binding agent administered, e.g., in each dose and/or treatment cycle, may induce intracellular signaling when binding to CD137 expressed on another cell. Thus, a binding agent in a suitable amount according to the present disclosure is able to trans-activate two different cells. In humans, CD40 is expressed on a number of cells including antigen-presenting cells (APCs), such as dendritic cells, whereas CD137 is expressed on T cells and other cells. Thus, a binding agent binding to CD40 and CD137 in a suitable amount according to the present disclosure is able to bind simultaneously to an APC and a T cell expressing these receptors. Without being bound by theory, a binding agent may thus (i) mediate cell-to-cell interaction between APCs and T cells by receptor binding and (ii) activate both CD40 and CD137 at once, which is primarily induced by cross-linking and receptor clustering upon cell-to-cell interaction and not necessarily dependent on agonistic activity of the parental monospecific bivalent antibodies. Thus, these trans-activating binding agent exert co-stimulatory activity in the context of APC:T cell interactions, and can elicit a T cell response against tumor cells. As such, this mechanism of action can reflect natural T-cell activation via antigen-presentation by activated APCs, allowing for the presentation of a variety of tumor-specific antigens by the APCs to T cells. Without being limited to theory, the costimulatory activity may provide for one or more of (i) only specific T cells being activated (i.e., those that are in contact with an APC) as opposed to any T cell; (ii) re-activation of exhausted T cells, by strong co-stimulation via activated APCs and CD137 triggering; and (iii) the priming of T cells by inducing antigen presentation by activated APCs and at the same time triggering CD137.

The amount of binding agent administered in each dose and/or treatment cycle may in particular be in a range, wherein more than 5%, preferably more than 10%, more preferably more than 15%, even more preferably more than 20%, even more preferably more than 25%, even more preferably more than 30%, even more preferably more than 35%, even more preferably more than 40%, even more preferably more than 45%, most preferably more than 50% of said binding agents bind to both, CD40 and CD137.

In preferred embodiments, the amount of binding agent administered, e.g., in each dose and/or in each treatment cycle, is
a) about 0.01-2.5 (such as about 0.04-2.5) mg/kg body weight or about 1-200 (such as about 3-200) mg in total; and/or
b) about 0.07×10⁻⁹-16.9×10⁻⁹ (such as about 0.25×10⁻⁹-16.9×10⁻⁹) mol/kg body weight or about 8×10⁻⁹-1350×10⁻⁹ (such as about 20×10⁻⁹-1350×10⁻⁹) mol in total.

In some embodiments, the amount of binding agent administered, e.g., in each dose and/or in each treatment cycle, is
a) about 0.62-1.88 (such as about 1.0-1.5) mg/kg body weight or about 50-150 (such as about 80-120) mg in total; and/or
b) about 4.1×10⁻⁹-12.7×10⁻⁹ (such as about 6.7×10⁻⁹-10.1×10⁻⁹) mol/kg body weight or about 335×10⁻⁹-1020×10⁻⁹ (such as about 535×10⁻⁹-810×10⁻⁹) mol in total.

According to these embodiments, the dose defined in mg/kg may be converted to flat dose, and vice versa, based on the median body weight of the subjects to whom the binding agent is administered being 80 kg.

The binding agent may be administered in any manner and by any route known in the art. In a preferred embodiment, the binding agent is administered systemically, such as parenterally, in particular intravenously.

The binding agent may be administered in the form of any suitable pharmaceutical composition as described herein. In a preferred embodiment, the binding agent is administered in the form of an infusion.

The binding agent can be administered prior to, simultaneously with, or after administration of the checkpoint inhibitor.

In one embodiment, the binding agent is administered prior to the administration of the checkpoint inhibitor. For example, the gap between the end of the administration of the binding agent and the beginning of the administration of the checkpoint inhibitor can be at least about 10 min, such as at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min, and up to about 14 days (up to about 2 weeks), such as up to about 13 days, up to about 12 days, up to about 11 days, up to about 10 days, up to about 9 days, up to about 8 days, up to about 7 days (up to about 1 week), up to about 6 days, up to about 5 days, up to about 4 days, up to about 3 days, up to about 2 days, up to about 1 day (up to about 24 h), up to about 18 h, up to about 12 h, up to about 6 h, up to about 5 h, up to about 4 h, up to about 3 h, up to about 2.5 h, or up to about 2 h.

In one embodiment, the binding agent is administered after the administration of the checkpoint inhibitor. For example, the gap between the end of the administration of the checkpoint inhibitor and the beginning of the administration of the binding agent can be at least about 10 min, such as at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min, and up to about 14 days (up to about 2 weeks), such as up to about 13 days, up to about 12 days, up to about 11 days, up to about 10 days, up to about 9 days, up to about 8 days, up to about 7 days (up to about 1 week), up to about 6 days, up to about 5 days, up to about 4 days, up to about 3 days, up to about 2 days, up to about 1 day (up to about 24 h), up to about 18 h, up to about 12 h, up to about 6 h, up to about 5 h, up to about 4 h, up to about 3 h, up to about 2.5 h, or up to about 2 h.

In one embodiment, the binding agent is administered simultaneously with the checkpoint inhibitor. For example, the binding agent and the checkpoint inhibitor may be administered using a composition comprising both drugs. Alternatively, the binding agent may be administered into one extremity of the subject, and the checkpoint inhibitor may be administered into another extremity of the subject.

### Checkpoint Inhibitor

In one embodiment, the immune checkpoint inhibitor suitable for use in the methods disclosed herein, is an antagonist of inhibitory signals, e.g., an antibody which targets, for example, PD-1, PD-L1, CTLA-4, LAG-3, or TIM-3. These ligands and receptors are reviewed in Pardoll, D., Nature. 12: 252-264, 2012. Further immune checkpoint proteins that can be targeted according the disclosure are described herein.

In one embodiment, the immune checkpoint inhibitor prevents inhibitory signals associated with the immune checkpoint. In one embodiment, the immune checkpoint inhibitor is an antibody, or fragment thereof that disrupts or inhibits inhibitory signaling associated with the immune checkpoint. In one embodiment, the immune checkpoint inhibitor is a small molecule inhibitor that disrupts or inhibits inhibitory signaling. In one embodiment, the immune checkpoint inhibitor is a peptide-based inhibitor that disrupts or inhibits inhibitory signaling. In one embodiment, the immune checkpoint inhibitor is an inhibitory nucleic acid molecule that disrupts or inhibits inhibitory signaling.

Inhibiting or blocking of inhibitory immune checkpoint signaling, as described herein, results in preventing or reversing immune-suppression and establishment or enhancement of T cell immunity against cancer cells. In one embodiment, inhibition of immune checkpoint signaling, as described herein, reduces or inhibits dysfunction of the immune system. In one embodiment, inhibition of immune checkpoint signaling, as described herein, renders dysfunctional immune cells less dysfunctional. In one embodiment, inhibition of immune checkpoint signaling, as described herein, renders a dysfunctional T cell less dysfunctional.

In one embodiment, the immune checkpoint inhibitor prevents the interaction between checkpoint blocker proteins, e.g., the interaction between PD-1 and PD-L1 or PD-L2; the interaction between CTLA-4 and CD80 or CD86; the interaction between LAG-3 and one or more of its ligands; the interaction of one or more KIRs with their respective ligands; the interaction of TIM-3 with one or more of its ligands (such as Galectin-9, PtdSer, HMGB1 and CEACAM1); the interaction of TIGIT with one or more of its ligands (such as PVR, PVRL2 and PVRL3); the interaction of VISTA with one or more of its binding partners; the interaction of GARP with one or more of its ligands; the inhibitory signaling through CD39 and/or CD73 and/or the interaction of A2AR and/or A2BR with adenosine; the interaction of B7-H3 with its receptor and/or of B7-H4 with its receptor; the interaction of BTLA with its ligand HVEM; the interaction of CD94/NKG2A with HLA-E; the interaction of one or more Siglecs and their respective ligands; CD20 signaling; the interaction of CD47 with SIRPα, the interaction of PVRIG with PVRL2; the interaction of CSF1R with CSF1; NOX signaling; and/or IDO and/or TDO signaling.

The immune checkpoint inhibitor may be an antibody, an antigen-binding fragment thereof, or a construct thereof comprising an antibody portion with an antigen-binding fragment of the required specificity. Antibodies or antigen-binding fragments thereof are as described herein. Antibodies or antigen-binding fragments thereof that are immune checkpoint inhibitors include in particular antibodies or antigen-binding fragments thereof that bind to immune checkpoint proteins, such as immune checkpoint receptors or immune checkpoint receptor ligands. Antibodies or antigen-binding fragments may also be conjugated to further moieties, as described herein. In particular, antibodies or antigen-binding fragments thereof are chimerized, humanized or human antibodies. Preferably, immune checkpoint inhibitor antibodies or antigen-binding fragments thereof are antagonists of immune checkpoint receptors or of immune checkpoint receptor ligands.

In a preferred embodiment, an antibody that is an immune checkpoint inhibitor is an isolated antibody.

In one embodiment, the immune checkpoint inhibitor is an antibody, a fragment or construct thereof that prevents the interaction between checkpoint blocker proteins, e.g., an antibody, or fragment thereof that prevents the interaction between PD-1 and PD-L1 or PD-L2; an antibody, a fragment or construct thereof that prevents the interaction between CTLA-4 and CD80 or CD86; an antibody, a fragment or construct thereof that prevents the interaction between LAG-3 and its ligands; an antibody, a fragment or construct thereof that prevents the interaction of TIM-3 with one or more of its ligands Galectin-9, PtdSer, HMGB1 and CEACAM1; an antibody, a fragment or construct thereof that prevents the interaction of one or more KIRs with their respective ligands; an antibody, a fragment or construct thereof that prevents the interaction of TIGIT with one or more of its ligands PVR, PVRL2 and PVRL3; an antibody, a fragment or construct thereof that prevents the interaction of VISTA with one or more of its binding partners; an antibody, a fragment or construct thereof that prevents the interaction of GARP with one or more of its ligands; an antibody, a fragment or construct thereof that prevents inhibitory signaling through CD39 and/or CD73 and/or the interaction of A2AR and/or A2BR with adenosine; an antibody, a fragment or construct thereof that prevents interaction of B7-H3 with its receptor and/or of B7-H4 with its receptor; an antibody, a fragment or construct thereof that prevents the interaction of BTLA with its ligand HVEM; an antibody, a fragment or construct thereof that prevents the interaction of LAG-3 with one or more of its ligands; an antibody, a fragment or construct thereof that prevents the interaction of CD94/NKG2A with HLA-E; an antibody, a fragment or construct thereof that prevents the interaction of one or more Siglecs and their respective ligands; an antibody, a fragment or construct thereof that prevents CD20 signaling; an antibody, a fragment or construct thereof that prevents the interaction of CD47 with SIRPα; an antibody, a fragment or construct thereof that prevents the interaction of PVRIG with PVRL2; an antibody, a fragment or construct thereof that prevents the interaction of CSF1R with CSF1; an antibody, a fragment or construct thereof that prevents NOX signaling; and/or an antibody, a fragment or construct thereof that prevents IDO and/or TDO signaling.

The immune checkpoint inhibitor may be an inhibitory nucleic acid molecule, such as an oligonucleotide, siRNA, shRNA, an antisense DNA or RNA molecule, and an aptamer (e.g., DNA or RNA aptamer), in particular an antisense-oligonucleotide. In one embodiment, the immune checkpoint inhibitor being siRNA interferes with mRNA therefore blocking translation, e.g., translation of an immune checkpoint protein.

The checkpoint inhibitor may also be in the form of the soluble form of the molecules (or variants thereof) themselves, e.g., a soluble PD-L1 or PD-L1 fusion.

In the context of the disclosure, more than one checkpoint inhibitor can be used, wherein the more than one checkpoint inhibitors are targeting distinct checkpoint pathways or the same checkpoint pathway. Preferably, the more than one checkpoint inhibitors are distinct checkpoint inhibitors. Preferably, if more than one distinct checkpoint inhibitor is used, in particular at least 2, 3, 4, 5, 6, 7, 8, 9 or 10 distinct checkpoint inhibitors are used, preferably 2, 3, 4 or 5 distinct checkpoint inhibitors are used, more preferably 2, 3 or 4 distinct checkpoint inhibitors are used, even more preferably 2 or 3 distinct checkpoint inhibitors are used and most preferably 2 distinct checkpoint inhibitors are used.

In one embodiment, the inhibitory immunoregulator (immune checkpoint blocker) is a component of the PD-1/PD-L1 or PD-1/PD-L2 signaling pathway. Accordingly, in one embodiment of the disclosure, the checkpoint inhibitor is an inhibitor of the PD-1 signaling pathway. In certain embodiments, the checkpoint inhibitor of the PD-1 signaling pathway is a PD-1 inhibitor. In certain embodiments, the checkpoint inhibitor of the PD-1 signaling pathway is a PD-1 ligand inhibitor, such as a PD-L1 inhibitor or a PD-L2 inhibitor. In a preferred embodiment, the checkpoint inhibitor of the PD-1 signaling pathway is an antibody, an antigen-binding portion thereof or a construct thereof that disrupts or inhibits the interaction between the PD-1 receptor and one or more of its ligands, PD-L1 and/or PD-L2. Antibodies which bind to PD-1 and disrupt or inhibit the interaction between PD-1 and one or more of its ligands are known in the art. In certain embodiments, the antibody, antigen-binding portion thereof or a construct thereof binds specifically to PD-1. In certain embodiments, the antibody, antigen-binding portion thereof or a construct thereof binds specifically to PD-L1 and disrupts or inhibits its interaction with PD-1, thereby increasing immune activity. In certain embodiments, the antibody, antigen-binding portion thereof or a construct thereof binds specifically to PD-L2 and disrupts or inhibits its interaction with PD-1, thereby increasing immune activity.

In one embodiment, the inhibitory immunoregulator is a component of the CTLA-4 signaling pathway. Accordingly, in one embodiment of the disclosure, the checkpoint inhibitor is an inhibitor of the CTLA-4 signaling pathway. In certain embodiments, the checkpoint inhibitor of the CTLA-4 signaling pathway is a CTLA-4 inhibitor. In certain embodiments, the checkpoint inhibitor of the CTLA-4 signaling pathway is a CTLA-4 ligand inhibitor.

In one embodiment, the inhibitory immunoregulator is a component of the TIGIT signaling pathway. Accordingly, in one embodiment of the disclosure, the checkpoint inhibitor is an inhibitor of the TIGIT signaling pathway. In certain embodiments, the checkpoint inhibitor of the TIGIT signaling pathway is a TIGIT inhibitor. In certain embodiments, the checkpoint inhibitor of the TIGIT signaling pathway is a TIGIT ligand inhibitor.

In one embodiment, the inhibitory immunoregulator is a component of the B7 family signaling pathway. In certain embodiments, the B7 family members are B7-H3 and B7-H4. In one embodiment of the disclosure, the checkpoint inhibitor is an inhibitor of B7-H3 and/or B7-4. The B7 family does not have any defined receptors but these ligands are upregulated on tumor cells or tumor-infiltrating cells. Preclinical mouse models have shown that blockade of these ligands can enhance anti-tumor immunity.

In one embodiment, the inhibitory immunoregulator is a component of the BTLA signaling pathway. Accordingly, in one embodiment of the disclosure, the checkpoint inhibitor is an inhibitor of the BTLA signaling pathway. In certain embodiments, the checkpoint inhibitor of the BTLA signaling pathway is a BTLA inhibitor. In certain embodiments, the checkpoint inhibitor of the BTLA signaling pathway is a HVEM inhibitor.

In one embodiment, the inhibitory immunoregulator is a component of one or more KIR signaling pathways. Accordingly, in one embodiment of the disclosure, the checkpoint inhibitor is an inhibitor of one or more KIR signaling pathways. In certain embodiments, the checkpoint inhibitor of one or more KIR signaling pathways is a KIR inhibitor. In certain embodiments, the checkpoint inhibitor one or more KIR signaling pathways is a KIR ligand inhibitor. For example, the KIR inhibitor according to the present disclosure may be an anti-KIR antibody that binds to KIR2DL1, KIR2DL2, and/or KIR2DL3.

In one embodiment, the inhibitory immunoregulator is a component of the LAG-3 signaling pathway. Accordingly, in one embodiment of the disclosure, the checkpoint inhibitor is an inhibitor of LAG-3 signaling. In certain embodiments, the checkpoint inhibitor of the LAG-3 signaling pathway is a LAG-3 inhibitor. In certain embodiments, the checkpoint inhibitor of the LAG-3 signaling pathway is a LAG-3 ligand inhibitor.

In one embodiment, the inhibitory immunoregulator is a component of the TIM-3 signaling pathway. Accordingly, in one embodiment of the disclosure, the checkpoint inhibitor is an inhibitor of the TIM-3 signaling pathway. In certain embodiments, the checkpoint inhibitor of the TIM-3 signaling pathway is a TIM-3 inhibitor. In certain embodiments, the checkpoint inhibitor of the TIM-3 signaling pathway is a TIM-3 ligand inhibitor.

In one embodiment, the inhibitory immunoregulator is a component of the CD94/NKG2A signaling pathway. Accordingly, in one embodiment of the disclosure, the checkpoint inhibitor is an inhibitor of the CD94/NKG2A signaling pathway. In certain embodiments, the checkpoint inhibitor of the CD94/NKG2A signaling pathway is a CD94/NKG2A inhibitor. In certain embodiments, the checkpoint inhibitor of the CD94/NKG2A signaling pathway is a CD94/NKG2A ligand inhibitor.

In one embodiment, the inhibitory immunoregulator is a component of the IDO signaling pathway. Accordingly, in one embodiment of the disclosure, the checkpoint inhibitor is an inhibitor of the IDO signaling pathway, e.g., an IDO inhibitor.

In one embodiment, the inhibitory immunoregulator is a component of the adenosine signaling pathway. Accordingly, in one embodiment of the disclosure, the checkpoint inhibitor is an inhibitor of the adenosine signaling pathway. In certain embodiments, the checkpoint inhibitor of the adenosine signaling pathway is a CD39 inhibitor. In certain embodiments, the checkpoint inhibitor of the adenosine signaling pathway is a CD73 inhibitor. In certain embodiments, the checkpoint inhibitor of the adenosine signaling pathway is an A2AR inhibitor. In certain embodiments, the checkpoint inhibitor of the adenosine signaling pathway is an A2BR inhibitor.

In one embodiment, the inhibitory immunoregulator is a component of the VISTA signaling pathway. Accordingly, in one embodiment of the disclosure, the checkpoint inhibitor is an inhibitor of the VISTA signaling pathway. In certain embodiments, the checkpoint inhibitor of the VISTA signaling pathway is a VISTA inhibitor.

In one embodiment, the inhibitory immunoregulator is a component of one or more Siglec signaling pathways. Accordingly, in one embodiment of the disclosure, the checkpoint inhibitor is an inhibitor of one or more Siglec signaling pathways. In certain embodiments, the checkpoint inhibitor of one or more Siglec signaling pathways is a Siglec inhibitor. In certain embodiments, the checkpoint inhibitor of one or more Siglec signaling pathways is a Siglec ligand inhibitor.

In one embodiment, the inhibitory immunoregulator is a component of the CD20 signaling pathway. Accordingly, in one embodiment of the disclosure, the checkpoint inhibitor is an inhibitor of the CD20 signaling pathway. In certain embodiments, the checkpoint inhibitor of the CD20 signaling pathway is a CD20 inhibitor.

In certain embodiments, the inhibitory immunoregulator is a component of the GARP signaling pathway. Accordingly, in one embodiment of the disclosure, the checkpoint inhibitor is an inhibitor of the GARP signaling pathway. In certain embodiments, the checkpoint inhibitor of the GARP signaling pathway is a GARP inhibitor.

In one embodiment, the inhibitory immunoregulator is a component of the CD47 signaling pathway. Accordingly, in one embodiment of the disclosure, the checkpoint inhibitor is an inhibitor of the CD47 signaling pathway. In certain embodiments, the checkpoint inhibitor of the CD47 signaling pathway is a CD47 inhibitor. In certain embodiments, the checkpoint inhibitor of the CD47 signaling pathway is a SIRPα inhibitor.

In certain embodiments, the inhibitory immunoregulator is a component of the PVRIG signaling pathway. Accordingly, in one embodiment of the disclosure, the checkpoint inhibitor is an inhibitor of the PVRIG signaling pathway. In certain embodiments, the checkpoint inhibitor of the PVRIG signaling pathway is a PVRIG inhibitor. In certain embodiments, the checkpoint inhibitor of the PVRIG signaling pathway is a PVRIG ligand inhibitor.

In certain embodiments, the inhibitory immunoregulator is a component of the CSF1R signaling pathway. Accordingly, in one embodiment of the disclosure, the checkpoint inhibitor is an inhibitor of the CSF1R signaling pathway. In certain embodiments, the checkpoint inhibitor of the CSF1R signaling pathway is a CSF1R inhibitor. In certain embodiments, the checkpoint inhibitor of the CSF1R signaling pathway is a CSF1 inhibitor.

In certain embodiments, the inhibitory immunoregulator is a component of the NOX signaling pathway. Accordingly, in one embodiment of the disclosure, the checkpoint inhibitor is an inhibitor of the NOX signaling pathway, e.g., a NOX inhibitor.

In certain embodiments, the inhibitory immunoregulator is a component of the TDO signaling pathway. Accordingly, in one embodiment of the disclosure, the checkpoint inhibitor is an inhibitor of the TDO signaling pathway, e.g., a TDO inhibitor.

Exemplary PD-1 inhibitors include, without limitation, anti-PD-1 antibodies such as BGB-A317 (BeiGene; see U.S. Pat. No. 8,735,553, WO 2015/35606 and US 2015/0079109), lambrolizumab (e.g., disclosed as hPD109A and its humanized derivatives h409A1, h409A16 and h409A17 in WO2008/156712), AB137132 (Abeam), EH12.2H7 and RMP1-14 (#BE0146; Bioxcell Lifesciences Pvt. LTD.), MIH4 (Affymetrix eBioscience), nivolumab (OPDIVO, BMS-936558; Bristol Myers Squibb; see U.S. Pat. No. 8,008,449; WO 2013/173223; WO 2006/121168), pembrolizumab (KEYTRUDA; MK-3475; Merck; see WO 2008/156712), pidilizumab (CT-011; CureTech; see Hardy et al., 1994, Cancer Res., 54(22):5793-6 and WO 2009/101611), PDR001 (Novartis; see WO 2015/112900), MEDI0680 (AMP-514; AstraZeneca; see WO 2012/145493), TSR-042 (see WO 2014/179664), cemiplimab (REGN-2810; Regeneron; H4H7798N; cf. US 2015/0203579 and WO 2015/112800), JS001 (TAIZHOU JUNSHI PHARMA; see Si-Yang Liu et al., 2007, J. Hematol. Oncol. 70: 136), AMP-224 (GSK-2661380; ef. Li et al., 2016, Int J Mol Sci 17(7):1151 and WO 2010/027827 and WO 2011/066342), PF-06801591 (Pfizer), tislelizumab (BGB-A317; BeiGene; see WO 2015/35606, U.S. Pat. No. 9,834,606, and US 2015/0079109), BI 754091, SHR-1210 (see WO2015/085847), and antibodies 17D8, 2D3, 4H1, 4A11, 7D3, and 5F4 as described in WO 2006/121168, INCSHR1210 (Jiangsu Hengrui Medicine; also known as SHR-1210; see WO 2015/085847), TSR-042 (Tesaro Biopharmaceutical; also known as ANB011; see WO2014/179664), GLS-010 (Wuxi/Harbin Gloria Pharmaceuticals; also known as WBP3055; see Si-Yang et al., 2017, J. Hematol. Oncol. 70: 136), STI-1110 (Sorrento Therapeutics; see WO 2014/194302), AGEN2034 (Agenus; see WO 2017/040790), MGA012 (Macrogenics; see WO 2017/19846), IBI308 (Innovent; see WO 2017/024465, WO 2017/025016, WO 2017/132825, and WO 2017/133540), cetrelimab (JNJ-63723283; JNJ-3283; see Calvo et al., J. Clin. Oncol. 36, no. 5_suppl (2018) 58), genolimzumab (CBT-501; see Patel et al., J. ImmunoTher. Cancer, 2017, 5(Suppl 2):P242), sasanlimab (PF-06801591; see Youssef et al., Proc. Am. Assoc. Cancer Res. Ann. Meeting 2017; Cancer Res 2017; 77(13 Suppl):Abstract), toripalimab (JS-001; see US 2016/0272708), camrelizumab (SHR-1210; INCSHR-1210; see US 2016/376367; Huang et al., Clin. Cancer Res. 2018; 24(6):1296-1304), spartalizumab (PDR001; see WO 2017/106656; Naing et al., J. Clin. Oncol. 34, no. 15_suppl (2016) 3060-3060), BCD-100 (JSC BIOCAD, Russia; see WO 2018/103017), balstilimab (AGEN2034; see WO 2017/040790), sintilimab (IBI-308; see WO 2017/024465 and WO 2017/133540), ezabenlimab (BI-754091; see US 2017/334995; Johnson et al., J. Clin. Oncol. 36, no. 5_suppl (2018) 212-212), zimberelimab (GLS-010; see WO 2017/025051), LZM-009 (see US 2017/210806), AK-103 (see WO 2017/071625, WO 2017/166804, and WO 2018/036472), retifanlimab (MGA-012; see WO 2017/019846), Sym-021 (see WO 2017/055547), CS1003 (see CN107840887), the anti-PD1-antibody IgG1-PD1 disclosed herein (i.e., comprising the VH sequence as defined in SEQ ID NO: 43, the VL sequence as defined in SEQ ID NO: 44, the Fc sequence as defined in SEQ ID NO: 61, and the kappa sequence as defined in SEQ ID NO: 27), anti-PD-1 antibodies as described, e.g., in U.S. Pat. Nos. 7,488,802, 8,008,449, 8,168,757, WO 03/042402, WO 2010/089411 (further disclosing anti-PD-L1 antibodies), WO 2010/036959, WO 2011/159877 (further disclosing antibodies against TIM-3), WO 2011/082400, WO 2011/161699, WO 2009/014708, WO 03/099196, WO 2009/114335, WO 2012/145493 (further disclosing antibodies against PD-L1), WO 2015/035606, WO 2014/055648 (further disclosing anti-KIR antibodies), US 2018/0185482 (further disclosing anti-PD-L1 and anti-TIGIT antibodies), U.S. Pat. Nos. 8,008,449, 8,779,105, 6,808,710, 8,168,757, US 2016/0272708, and U.S. Pat. No. 8,354,509, small molecule antagonists to the PD-1 signaling pathway as disclosed, e.g., in Shaabani et al., 2018, Expert Op Ther Pat., 28(9):665-678 and Sasikumar and Ramachandra, 2018, BioDrugs, 32(5):481-497, siRNAs directed to PD-1 as disclosed, e.g., in WO 2019/000146 and WO 2018/103501, soluble PD-1 proteins as disclosed in WO 2018/222711 and oncolytic viruses comprising a soluble form of PD-1 as described, e.g., in WO 2018/022831.

In a certain embodiment, the PD-1 inhibitor is nivolumab (OPDIVO; BMS-936558), pembrolizumab (KEYTRUDA; MK-3475), pidilizumab (CT-011), PDR001, MEDI0680 (AMP-514), TSR-042, REGN2810, JS001, AMP-224 (GSK-2661380), PF-06801591, BGB-A317, BI 754091, or SHR-1210. In one embodiment, the PD-1 inhibitor is IgG1-PD1 as disclosed herein.

In certain embodiments, the inhibitory immunoregulator is an anti-PD-1 antibody or antigen-binding fragment thereof comprising the complementary determining regions (CDRs) of one of the anti-PD-1 antibodies or antigen-binding fragments described above, such as the CDRs of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021, CS1003, and IgG1-PD1.

In some embodiments, the CDRs of the anti-PD-1 antibody are delineated using the Kabat numbering scheme (Kabat, E. A., et al. (1991) Sequences of Proteins of Immunological Interest, Fifth Edition, U.S. Department of Health and Human Services, NTH Publication No. 91-3242).

In certain embodiments, the inhibitory immunoregulator is an anti-PD-1 antibody or antigen-binding fragment thereof comprising the heavy chain variable region and the light chain variable region of one of the anti-PD-1 antibodies or antigen-binding fragments described above, such as the heavy chain variable region and the light chain variable region of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021, CS1003, and IgG1-PD1.

In certain embodiments, the inhibitory immunoregulator is an anti-PD-1 antibody or antigen-binding fragment thereof selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021, CS1003, IgG1-PD1.

Anti-PD-1 antibodies of the disclosure are preferably monoclonal, and may be multispecific, human, humanized or chimeric antibodies, single chain antibodies, Fab fragments, F(ab′) fragments, fragments produced by a Fab expression library, and PD-1 binding fragments of any of the above. In some embodiments, an anti-PD-1 antibody described herein binds specifically to PD-1 (e.g., human PD-1). The immunoglobulin molecules of the disclosure can be of any isotype (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-PD-1 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-1 antibodies or antigen-binding fragments thereof are human, murine (e.g., mouse and rat), donkey, sheep, rabbit, goat, guinea pig, camelid, horse, or chicken.

The anti-PD-1 antibodies disclosed herein may be monospecific, bispecific, trispecific or of greater multi specificity. Multispecific antibodies may be specific for different epitopes of PD-1 or may be specific for both PD-1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

The anti-PD-1 antibodies disclosed herein may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003; 27(1):55-77 (“IMGT” numbering scheme); Honegger A and Plückthun A, “Yet another numbering scheme for immunoglobulin variable domains: an automatic modeling and analysis tool,” J Mol Biol, 2001; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a CDR or individual specified CDRs (e.g., CDR-H1, CDR-12, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-PD-1 antibodies or antigen-binding fragments thereof provided herein are according to the IMGT numbering scheme as described in Lefranc, M. P. et al., Dev. Comp. Immunol., 2003, 27, 55-77.

In some embodiments, the anti-PD-1 antibodies disclosed herein comprise the CDRs of the antibody nivolumab. See WO 2006/121168. In some embodiments, the CDRs of the antibody nivolumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-1 antibody or derivative thereof comprising a heavy or light chain variable domain, said variable domain comprising (a) a set of three CDRs, in which said set of CDRs are from the monoclonal antibody nivolumab, and (b) a set of four framework regions, in which said set of framework regions differs from the set of framework regions in the monoclonal antibody nivolumab, and in which said anti-PD-1 antibody or derivative thereof binds to PD-1. In certain embodiments, the anti-PD-1 antibody is nivolumab.

Anti-PD-1 antibodies disclosed herein may also be described or specified in terms of their binding affinity to PD-1 (e.g., human PD-1). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The anti-PD-1 antibodies also include derivatives and constructs that are modified, i.e., by the covalent attachment of any type of molecule to the antibody such that covalent attachment does not prevent the antibody from binding to PD-1. For example, but not by way of limitation, the anti-PD-1 antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative or construct may contain one or more non-classical amino acids.

Exemplary PD-1 ligand inhibitors are PD-L1 inhibitors and PD-L2 inhibitors and include, without limitation, anti-PD-L1 antibodies such as MEDI4736 (durvalumab; AstraZeneca; see WO 2011/066389), MSB-0010718C (see US 2014/0341917), YW243.55.S70 (see SEQ ID NO: 20 of WO 2010/077634 and U.S. Pat. No. 8,217,149), MIH1 (Affymetrix eBioscience; cf. EP 3 230 319), MDX-1105 (Roche/Genentech; see WO2013019906 and U.S. Pat. No. 8,217,149) STI-1014 (Sorrento; see WO2013/181634), CK-301 (Checkpoint Therapeutics), KN035 (3D Med/Alphamab; see Zhang et al., 2017, Cell Discov. 3:17004), atezolizumab (TECENTRIQ; RG7446; MPDL3280A; R05541267; see U.S. Pat. No. 9,724,413), BMS-936559 (Bristol Myers Squibb; see U.S. Pat. No. 7,943,743, WO 2013/173223), avelumab (bavencio; ef. US 2014/0341917), LY3300054 (Eli Lilly Co.), CX-072 (Proclaim-CX-072; also called CytomX; see WO2016/149201), FAZ053, KN035 (see WO2017020801 and WO2017020802), MDX-1105 (see US 2015/0320859), anti-PD-L1 antibodies disclosed in U.S. Pat. No. 7,943,743, including 3G10, 12A4 (also referred to as BMS-936559), 10A5, 5F8, 10H10, 1B12, 7H1, 11E6, 12B7, and 13G4, anti-PD-L1 antibodies as described in WO 2010/077634, U.S. Pat. No. 8,217,149, WO 2010/036959, WO 2010/077634, WO 2011/066342, U.S. Pat. Nos. 8,217,149, 7,943,743, WO 2010/089411, U.S. Pat. Nos. 7,635,757, 8,217,149, US 2009/0317368, WO 2011/066389, WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO 2018/222711, WO2015/112805, WO2015/061668, WO2014/159562, WO2014/165082, WO2014/100079.

In a certain embodiment, the PD-L1 inhibitor is atezolizumab (TECENTRIQ; RG7446; MPDL3280A; R05541267; see U.S. Pat. No. 9,724,413).

In certain embodiments, the inhibitory immunoregulator is an anti-PD-L1 antibody or antigen-binding fragment thereof comprising the complementary determining regions (CDRs) of one of the anti-PD-L1 antibodies or antigen-binding fragments described above, such as the CDRs of atezolizumab or an antigen-binding fragment thereof.

In some embodiments, the CDRs of the anti-PD-L1 antibody are delineated using the Kabat numbering scheme (Kabat, E. A., et al. (1991) Sequences of Proteins of Immunological Interest, Fifth Edition, U.S. Department of Health and Human Services, NTH Publication No. 91-3242).

In certain embodiments, the inhibitory immunoregulator is an anti-PD-L1 antibody or antigen-binding fragment thereof comprising the heavy chain variable region and the light chain variable region of one of the anti-PD-L1 antibodies or antigen-binding fragments described above, such as the heavy chain variable region and the light chain variable region of atezolizumab or antigen-binding fragments thereof.

Anti-PD-L1 antibodies of the disclosure are preferably monoclonal, and may be multispecific, human, humanized or chimeric antibodies, single chain antibodies, Fab fragments, F(ab′) fragments, fragments produced by a Fab expression library, and PD-L1 binding fragments of any of the above. In some embodiments, an anti-PD-L1 antibody described herein binds specifically to PD-L1 (e.g., human PD-L1). The immunoglobulin molecules of the disclosure can be of any isotype (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-PD-L1 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-L1 antibodies or antigen-binding fragments thereof are human, murine (e.g., mouse and rat), donkey, sheep, rabbit, goat, guinea pig, camelid, horse, or chicken.

The anti-PD-L1 antibodies disclosed herein may be monospecific, bispecific, trispecific or of greater multi specificity. Multispecific antibodies may be specific for different epitopes of PD-L1 or may be specific for both PD-L1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

The anti-PD-L1 antibodies disclosed herein may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefrane M P et al., “IMGT unique numbering for immunoglobulin and T cell receptor variable domains and Ig superfamily V-like domains,” Dev Comp Immunol, 2003; 27(1):55-77 (“IMGT” numbering scheme); Honegger A and Plükthun A, “Yet another numbering scheme for immunoglobulin variable domains: an automatic modeling and analysis tool,” J Mol Biol, 2001; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a CDR or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-PD-L1 antibodies or antigen-binding fragments thereof provided herein are according to the IMGT numbering scheme as described in Lefranc, M. P. et al., Dev. Comp. Immunol., 2003, 27, 55-77.

In some embodiments, the anti-PD-L1 antibodies disclosed herein comprise the CDRs of the antibody atezolizumab. See U.S. Pat. No. 9,724,413. In some embodiments, the CDRs of the antibody atezolizumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-L1 antibody or derivative thereof comprising a heavy or light chain variable domain, said variable domain comprising (a) a set of three CDRs, in which said set of CDRs are from the monoclonal antibody atezolizumab, and (b) a set of four framework regions, in which said set of framework regions differs from the set of framework regions in the monoclonal antibody atezolizumab, and in which said anti-PD-L1 antibody or derivative thereof binds to PD-L1. In certain embodiments, the anti-PD-L1 antibody is atezolizumab.

Anti-PD-L1 antibodies disclosed herein may also be described or specified in terms of their binding affinity to PD-L1 (e.g., human PD-L1). Preferred binding affinities include those with a dissociation constant or Kd less than 5×10⁻² M, 10⁻² M, 5×10⁻³ M, 10⁻⁴ M, 5×10⁻⁴ M, 10⁻⁴ M, 5×10⁻⁴ M, 10⁻⁵ M, 5×10⁻⁶ M, 10⁻⁶ M, 5×10⁻⁷ M, 10⁻⁸ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹⁴ M, 5×10⁻¹⁴ M, 10⁻¹⁴ M, 5×10⁻¹⁵ M, or 10⁻¹⁵ M.

The anti-PD-L1 antibodies also include derivatives and constructs that are modified, i.e., by the covalent attachment of any type of molecule to the antibody such that covalent attachment does not prevent the antibody from binding to PD-L1. For example, but not by way of limitation, the anti-PD-L1 antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative or construct may contain one or more non-classical amino acids.

Exemplary CTLA-4 inhibitors include, without limitation, the monoclonal antibodies ipilimumab (Yervoy; Bristol Myers Squibb) and tremelimumab (Pfizer/Medlmmune), trevilizumab, AGEN-1884 (Agenus) and ATOR-1015, the anti-CTLA4 antibodies disclosed in WO 2001/014424, US 2005/0201994, EP 1212422, U.S. Pat. Nos. 5,811,097, 5,855,887, 6,051,227, 6,682,736, 6,984,720, WO 01/14424, WO 00/37504, US 2002/0039581, US 2002/086014, WO 98/42752, U.S. Pat. Nos. 6,207,156, 5,977,318, 7,109,003, and 7,132,281, the dominant negative proteins abatacept (Orencia; see EP 2 855 533), which comprises the Fe region of IgG1 fused to the CTLA-4 ECD, and belatacept (Nulojix; see WO 2014/207748), a second generation higher-affinity CTLA-4-Ig variant with two amino acid substitutions in the CTLA-4 ECD relative to abatacept, soluble CTLA-4 polypeptides, e.g., RG2077 and CTLA4-IgG4m (see U.S. Pat. No. 6,750,334), anti-CTLA-4 aptamers and siRNAs directed to CTLA-4, e.g., as disclosed in US 2015/203848. Exemplary CTLA-4 ligand inhibitors are described in Pile et al., 2015 (Encyclopedia of Inflammatory Diseases, M. Parnham (ed.), doi: 10.1007/978-3-0348-0620-6_20).

Exemplary checkpoint inhibitors of the TIGIT signaling pathway include, without limitation, anti-TIGIT antibodies, such as BMS-986207, COM902 (CGEN-15137; Compugen), AB154 (Arcus Biosciences) or etigilimab (OMP-313M32; OncoMed Pharmaceuticals), or the antibodies disclosed in WO2017/059095, in particular “MAB10”, US 2018/0185482, WO 2015/009856, and US 2019/0077864.

Exemplary checkpoint inhibitors of B7-H3 include, without limitation, the Fe-optimized monoclonal antibody enoblituzumab (MGA271; Macrogenics; see US 2012/0294796) and the anti-B7-H3 antibodies MGD009 (Macrogenics) and pidilizumab (see U.S. Pat. No. 7,332,582).

Exemplary B7-H4 inhibitors include, without limitation, antibodies as described in Dangaj et al., 2013 (Cancer Research 73:4820-9) and in Smith et al., 2014 (Gynecol Oncol, 134:181-189), WO 2013/025779 (e.g., 2D1 encoded by SEQ ID NOs: 3 and 4, 2H9 encoded by SEQ ID NO: 37 and 39, and 2E11 encoded by SEQ ID NOs: 41 and 43) and in WO 2013/067492 (e.g., an antibody with an amino acid sequence selected from SEQ ID NOs: 1-8), morpholino antisense oligonucleotides, e.g., as described by Kryczek et al., 2006 (J Exp Med, 203:871-81), or soluble recombinant forms of B7-14, such as disclosed in US 2012/0177645.

Exemplary BTLA inhibitors include, without limitation, the anti-BTLA antibodies described in Crawford and Wherry, 2009 (J Leukocyte Biol 86:5-8), WO 2011/014438 (e.g., 4C7 or an antibody comprising heavy and light chains according to SEQ ID NOs: 8 and 15 and/or SEQ ID NOs: 11 and 18), WO 2014/183885 (e.g., the antibody deposited under the number CNCM I-4752) and US 2018/155428.

Exemplary inhibitors of KIR signaling include, without limitation, the monoclonal antibodies lirilumab (1-7F9; IPH2102; see U.S. Pat. No. 8,709,411), IPH4102 (Innate Phanna; see Marie-Cardine et al., 2014, Cancer 74(21): 6060-70), anti-KIR antibodies as disclosed, e.g., in US 2018/208652, US 2018/117147, US 2015/344576, WO 2005/003168, WO 2005/009465, WO 2006/072625, WO 2006/072626, WO 2007/042573, WO 2008/084106 (e.g., an antibody comprising heavy and light chains according to SEQ ID NOs: 2 and 3), WO 2010/065939, WO 2012/071411, WO 2012/160448 and WO 2014/055648.

Exemplary LAG-3 inhibitors include, without limitation, the anti-LAG-3 antibodies BMS-986016 (Bristol-Myers Squibb; see WO 2014/008218 and WO 2015/116539), 25F7 (see US2011/0150892), IMP731 (see WO 2008/132601), H5L7BW (cf. WO2014140180), MK-4280 (28G-10; Merck; see WO 2016/028672), REGN3767 (Regneron/Sanofi), BAP050 (see WO 2017/019894), IMP-701 (LAG-525; Novartis) Sym022 (Symphogen), TSR-033 (Tesaro), MGD013 (a bispecific DART antibody targeting LAG-3 and PD-1 developed by MacroGenics), BI754111 (Boehringer Ingelheim), FS118 (a bispecific antibody targeting LAG-3 and PD-1 developed by F-star), GSK2831781 (GSK) and antibodies as disclosed in WO 2009/044273, WO 2008/132601, WO 2015/042246, EP 2 320 940, US 2019/169294, US 2019/169292, WO 2016/028672, WO 2016/126858, WO 2016/200782, WO 2015/200119, WO 2017/220569, WO 2017/087589, WO 2017/219995, WO 2017/019846, WO 2017/106129, WO 2017/062888, WO 2018/071500, WO 2017/087901, US 2017/0260271, WO 2017/198741, WO2017/220555, WO2017/015560, WO2017/025498, WO2017/149143, WO 2018/069500, WO2018/083087, WO2018/034227 WO2014/140180, the LAG-3 antagonistic protein AVA-017 (Avacta), the soluble LAG-3 fusion protein IMP321 (eftilagimod alpha; Immutep; see EP 2 205 257 and Brignone et al., 2007, J. Immunol., 179: 4202-4211), and soluble LAG-3 proteins disclosed in WO 2018/222711.

Exemplary TIM-3 inhibitors include, without limitation, antibodies targeting TIM-3 such as F38-2E2 (BioLegend), cobolimab (TSR-022; Tesaro), LY3321367 (Eli Lilly), MBG453 (Novartis) and antibodies as disclosed in, e.g., WO 2013/006490, WO 2018/085469 (e.g., antibodies comprising heavy and light chain sequences encoded by nucleic acid sequences according to SEQ ID NOs: 3 and 4), WO 2018/106588, WO 2018/106529 (e.g., an antibody comprising heavy and light chain sequences according to SEQ ID NOs: 8-11).

Exemplary TIM-3 ligand inhibitors include, without limitation, CEACAM1 inhibitors such as the anti-CEACAM1 antibody CM10 (cCAM Biotherapeutics; see WO 2013/054331), antibodies disclosed in WO 2015/075725 (e.g., CM-24, 26H7, 5F4, TEC-11, 12-140-4, 4/3/17, COL-4, F36-54, 34B1, YG-C28F2, D14HD11, M8.7.7, DII-AD11, HEA81, B 1.1, CLB-gran-10, F34-187, T84.1, B6.2, B 1.13, YG-C94G7, 12-140-5, scFv DIATHIS1, TET-2; cCAM Biotherapeutics), antibodies described by Watt et al., 2001 (Blood, 98: 1469-1479) and in WO 2010/12557 and PtdSer inhibitors such as bavituximab (Peregrine).

Exemplary CD94/NKG2A inhibitors include, without limitation, monalizumab (IPH2201; Innate Pharma) and the antibodies and method for their production as disclosed in U.S. Pat. No. 9,422,368 (e.g., humanized Z199; see EP 2 628 753), EP 3 193 929 and WO2016/032334 (e.g., humanized Z270; see EP 2 628 753).

Exemplary IDO inhibitors include, without limitation, exiguamine A, epacadostat (INCB024360; InCyte; see U.S. Pat. No. 9,624,185), indoximod (Newlink Genetics; CAS #: 110117-83-4), NLG919 (Newlink Genetics/Genentech; CAS #: 1402836-58-1), GDC-0919 (Newlink Genetics/Genentech; CAS #: 1402836-58-1), F001287 (Flexus Biosciences/BMS; CAS #: 2221034-29-1), KHK2455 (Cheong et al., 2018, Expert Opin Ther Pat. 28(4):317-330), PF-06840003 (see WO 2016/181348), navoximod (RG6078, GDC-0919, NLG919; CAS #: 1402837-78-8), linrodostat (BMS-986205; Bristol-Myers Suibb; CAS #: 1923833-60-6), small molecules such as 1-methyl-tryptophan, pyrrolidine-2,5-dione derivatives (see WO 2015/173764) and the IDO inhibitors disclosed by Sheridan, 2015, Nat Biotechnol 33:321-322.

Exemplary CD39 inhibitors include, without limitation. A001485 (Arcus Biosciences), PSB 069 (CAS #: 78510-31-3) and the anti-CD39 monoclonal antibody IPH5201 (Innate Pharma; see Perrot et al., 2019, Cell Reports 8:2411-2425.E9).

Exemplary CD73 inhibitors include, without limitation, anti-CD73 antibodies such as CPI-006 (Corvus Pharmaceuticals), MEDI9447 (Medlmmune; see WO2016075099), IPH5301 (Innate Pharma; see Perrot et al., 2019, Cell Reports 8:2411-2425.E9), the anti-CD73 antibodies described in WO2018/110555, the small molecule inhibitors PBS 12379 (Tocris Bioscience; CAS #: 1802226-78-3), A000830, A001 190 and A001421 (Arcus Biosciences; see Becker et al., 2018, Cancer Research 78(13 Supplement):3691-3691, doi: 10.1158/1538-7445.AM2018-3691), CB-708 (Calithera Biosciences) and purine cytotoxic nucleoside analogue-based diphosphonates as described by Allard et al., 2018 (Immunol Rev., 276(1):121-144).

Exemplary A2AR inhibitors include, without limitation, small molecule inhibitors such as istradefylline (KW-6002; CAS #: 155270-99-8), PBF-509 (Palobiopharma), ciforadenant (CPI-444: Corvus Pharma/Genentech; CAS #: 1202402-40-1), ST1535 ([2butyl-9-methyl-8-(2H-1,2,3-triazol 2-yl)-9H-purin-6-xylamine]; CAS #: 496955-42-1), ST4206 (see Stasi et al., 2015, Europ J Pharm 761:353-361; CAS #: 1246018-36-9), tozadenant (SYN115; CAS #: 870070-55-6), V81444 (see WO 2002/055082), preladenant (SCH420814; Merck; CAS #: 377727-87-2), vipadenant (BIIB014; CAS #: 442908-10-3), ST1535 (CAS #: 496955-42-1), SCH412348 (CAS #: 377727-26-9), SCH442416 (Axon 2283; Axon Medchem; CAS #: 316173-57-6), ZM241385 (4-(2-(7-amino-2-(2-furyl)-(1,2,4)triazolo(2,3-a)-(1,3,5)triazin-5-yl-amino)ethyl)phenol; Cas #: 139180-30-6), AZD4635 (AstraZeneca), AB928 (a dual A2AR/A2BR small molecule inhibitor; Arcus Biosciences) and SCH58261 (see Popoli et al., 2000, Neuropsychopharm 22:522-529; CAS #: 160098-96-4).

Exemplary A2BR inhibitors include, without limitation, AB928 (a dual A2AR/A2BR small molecule inhibitor; Arcus Biosciences), MRS 1706 (CAS #: 264622-53-9), GS6201 (CAS #: 752222-83-6) and PBS 1115 (CAS #: 152529-79-8).

Exemplary VISTA inhibitors include, without limitation, anti-VISTA antibodies such as JNJ-61610588 (onvatilimab; Janssen Biotech) and the small molecule inhibitor CA-170 (anti-PD-L1/L2 and anti-VISTA small molecule; CAS #: 1673534-76-3).

Exemplary Siglec inhibitors include, without limitation, the anti-Sigle-7 antibodies disclosed in US 2019/023786 and WO 2018/027203 (e.g., an antibody comprising a variable heavy chain region according to SEQ ID NO: 1 and a variable light chain region according to SEQ ID NO: 15), the anti-Siglec-2 antibody inotuzumab ozogamicin (Besponsa; see U.S. Pat. Nos. 8,153,768 and 9,642,918), the anti-Siglec-3 antibody gemtuzumab ozogamicin (Mylotarg; see U.S. Pat. No. 9,359,442) or the anti-Siglec-9 antibodies disclosed in US 2019/062427, US 2019/023786, WO 2019/011855, WO 2019/011852 (e.g., an antibody comprising the CDRs according to SEQ ID NOs: 171-176, or 3 and 4, or 5 and 6, or 7 and 8, or 9 and 10, or 11 and 12, or 13 and 14, or 15 and 16, or 17 and 18, or 19 and 20, or 21 and 22, or 23 and 24, or 25 and 26), US 2017/306014 and EP 3 146 979.

Exemplary CD20 inhibitors include, without limitation, anti-CD20 antibodies such as rituximab (RITUXAN; IDEC-102; IDEC-C2B8; see U.S. Pat. No. 5,843,439), ABP 798 (rituximab biosimilar), ofatumumab (2F2; see WO2004/035607), obinutuzumab, ocrelizumab (2h7; see WO 2004/056312), ibritumomab tiuxetan (Zevalin), tositumomab, ublituximab (LFB-R603; LFB Biotechnologies) and the antibodies disclosed in US 2018/0036306 (e.g., an antibody comprising light and heavy chains according to SEQ ID NOs: 1-3 and 4-6, or 7 and 8, or 9 and 10).

Exemplary GARP inhibitors include, without limitation, anti-GARP antibodies such as ARGX-115 (arGEN-X) and the antibodies and methods for their production as disclosed in US 2019/127483, US 2019/016811, US 2018/327511, US 2016/251438, EP 3 253 796.

Exemplary CD47 inhibitors include, without limitation, anti-CD47 antibodies such as HuF9-G4 (Stanford University/Forty Seven), CC-90002/INBRX-103 (Celgene/Inhibrx), SRF231 (Surface Oncology), IB1188 (Innovent Biologics), AO-176 (Arch Oncology), bispecific antibodies targeting CD47 including TG-1801 (NI-1701; bispecific monoclonal antibody targeting CD47 and CD19; Novimmune/TG Therapeutics) and NI-1801 (bispecific monoclonal antibody targeting CD47 and mesothelin; Novimmune), and CD47 fusion proteins such as ALX148 (ALX Oncology; see Kauder et al., 2019, PLoS One, doi: 10.1371/journal.pone.0201832).

Exemplary SIRPα inhibitors include, without limitation, anti-SIRPα antibodies such as OSE-172 (Boehringer Ingelheim/OSE), FSI-189 (Forty Seven), anti-SIRPα fusion proteins such as TTI-621 and TTI-662 (Trillium Therapeutics; see WO 2014/094122).

Exemplary PVRIG inhibitors include, without limitation, anti-PVRIG antibodies such as COM701 (CGEN-15029) and antibodies and method for their manufacture as disclosed in, e.g., WO 2018/033798 (e.g., CHA.7.518.1H4(S24IP), CHA.7.538.1.2.H4(S241P), CPA.9.086H4(S241P), CPA.9.083H4(S241P), CHA.9.547.7.H4(S241P), CHA.9.547.13.114(S241P) and antibodies comprising a variable heavy domain according to SEQ ID NO: 5 and a variable light domain according to SEQ ID NO: 10 of WO 2018/033798 or antibodies comprising a heavy chain according to SEQ ID NO:9 and a light chain according to SEQ ID NO: 14; WO 2018/033798 further discloses anti-TIGIT antibodies and combination therapies with anti-TIGIT and anti-PVRIG antibodies), WO2016134333, WO2018017864 (e.g., an antibody comprising a heavy chain according to SEQ ID NOs: 5-7 having at least 90% sequence identity to SEQ ID NO: 11 and/or a light chain according to SEQ ID NOs: 8-10 having at least 90% sequence identity to SEQ ID NO: 12, or an antibody encoded by SEQ ID NOs: 13 and/or 14 or SEQ ID NOs: 24 and/or 29, or another antibody disclosed in WO 2018/017864) and anti-PVRIG antibodies and fusion peptides as disclosed in WO 2016/134335.

Exemplary CSF1R inhibitors include, without limitation, anti-CSF1R antibodies cabiralizumab (FPA008; FivePrime; see WO 2011/140249, WO 2013/169264 and WO 2014/036357), IMC-CS4 (EiiLilly), emactuzumab (R05509554; Roche), RG7155 (WO 2011/70024, WO 2011/107553, WO 2011/131407, WO 2013/87699, WO 2013/119716, WO 2013/132044) and the small molecule inhibitors BLZ945 (CAS #: 953769-46-5) and pexidartinib (PLX3397; Selleckehem; CAS #: 1029044-16-3).

Exemplary CSF1 inhibitors include, without limitation, anti-CSF1 antibodies disclosed in EP 1 223 980 and Weir et al., 1996 (J Bone Mineral Res 11: 1474-1481), WO 2014/132072, and antisense DNA and RNA as disclosed in WO 2001/030381.

Exemplary NOX inhibitors include, without limitation, NOX1 inhibitors such as the small molecule ML171 (Gianni et al., 2010, ACS Chem Biol 5(10):981-93, NOS31 (Yamamoto et al., 2018, Biol Pharm Bull. 41(3):419-426), NOX2 inhibitors such as the small molecules ceplene (histamine dihydrochloride; CAS #: 56-92-8), BJ-1301 (Gautam et al., 2017, Mol Cancer Ther 16(10):2144-2156; CAS #: 1287234-48-3) and inhibitors described by Lu et al., 2017, Biochem Pharmacol 143:25-38, NOX4 inhibitors such as the small molecule inhibitors VAS2870 (Altenhofer et al., 2012, Cell Mol Life Sciences 69(14):2327-2343), diphenylene iodonium (CAS #: 244-54-2) and GKT137831 (CAS #: 1218942-37-0; see Tang et al., 2018, 19(10):578-585).

Exemplary TDO inhibitors include, without limitation, 4-(indol-3-yl)-pyrazole derivatives (see U.S. Pat. No. 9,126,984 and US 2016/0263087), 3-indol substituted derivatives (see WO 2015/140717, WO 2017/025868, WO 2016/147144), 3-(indol-3-yl)-pyridine derivatives (see US 2015/0225367 and WO 2015/121812), dual IDO/TDO antagonist, such as small molecule dual IDO/TDO inhibitors disclosed in WO 2015/150097, WO 2015/082499, WO 2016/026772, WO 2016/071283, WO 2016/071293, WO 2017/007700, and the small molecule inhibitor CB548 (Kim, C, et al., 2018, Annals Oncol 29 (suppl_8): viii400-viii441).

According to the disclosure, the immune checkpoint inhibitor is an inhibitor of an inhibitory checkpoint protein but preferably not an inhibitor of a stimulatory checkpoint protein.

In a preferred embodiment, the immune checkpoint inhibitor is an antibody, in particular an antagonistic or blocking antibody, which disrupts or inhibits one of the inhibitory immune checkpoint signaling pathways described herein, particularly one of the inhibitory immune checkpoint signaling pathways selected from the group consisting of the PD-1 pathway (interaction of PD-1 with one or more of its ligands (such as PD-L1 and/or PD-L2)), the CTLA-4 pathway (interaction of CTLA-4 with one or more of its ligands (such as CD80 or CD86)), the TIM-3 pathway (interaction of TIM-3 with one or more of its ligands (such as Galectin-9, PtdSer, HMGB1 and CEACAM1)), the KIR pathway (interaction of KIR with one or more of its ligands), the LAG-3 pathway (interaction of LAG-3 with one or more of its ligands), the TIGIT pathway (interaction of TIGIT with one or more of its ligands (such as PVR, PVRL2 and PVRL3)), the VISTA pathway (interaction of VISTA with one or more of its ligands), and the GARP pathway (interaction of GARP with one or more of its ligands). In one preferred embodiment, the immune checkpoint inhibitor is an antibody, in particular an antagonistic or blocking antibody, which disrupts or inhibits one of the inhibitory immune checkpoint signaling pathways selected from the group consisting of the PD-1 pathway (interaction of PD-1 with one or more of its ligands (such as PD-L1 and/or PD-L2)), the CTLA-4 pathway (interaction of CTLA-4 with one or more of its ligands (such as CD80 or CD86)). In one preferred embodiment, the immune checkpoint inhibitor is an antibody, in particular an antagonistic or blocking antibody, which disrupts or inhibits the PD-1 pathway (interaction of PD-1 with one or more of its ligands (such as PD-L1 and/or PD-L2)). In one preferred embodiment, the immune checkpoint inhibitor is an antibody, in particular an antagonistic or blocking antibody, which disrupts or inhibits the interaction between PD-1 and PD-L1.

Checkpoint inhibitors may be administered in the form of nucleic acid, such DNA or RNA molecules, encoding an immune checkpoint inhibitor, e.g., an inhibitory nucleic acid molecule or an antibody or fragment thereof. For example, antibodies can be delivered encoded in expression vectors, as described herein. Nucleic acid molecules can be delivered as such, e.g., in the form of a plasmid or mRNA molecule, or complexed with a delivery vehicle, e.g., a liposome, lipoplex or nucleic-acid lipid particles. Checkpoint inhibitors may also be administered via an oncolytic virus comprising an expression cassette encoding the checkpoint inhibitor. Checkpoint inhibitors may also be administered by administration of endogeneic or allogeneic cells able to express a checkpoint inhibitor, e.g., in the form of a cell based therapy.

In one embodiment, the cell based therapy comprises genetically engineered cells. In one embodiment, the genetically engineered cells express an immune checkpoint inhibitor, such as described herein. In one embodiment, the genetically engineered cells express an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. Genetically engineered cells may also express further agents that enhance T cell function. Such agents are known in the art. Cell based therapies for the use in inhibition of immune checkpoint signaling are disclosed, e.g., in WO 2018/222711, herein incorporated by reference in its entirety.

Preferably, the checkpoint inhibitor is administered in a suitable amount, i.e., the amount of checkpoint inhibitor administered, e.g., in each dose and/or treatment cycle, may totally or partially reduce, inhibit, interfere with or negatively modulate one or more checkpoint proteins or may totally or partially reduce, inhibit, interfere with or negatively modulate expression of one or more checkpoint proteins. Thus, a checkpoint inhibitor in a suitable amount according to the present disclosure is able to totally or partially reduce, inhibit, interfere with or negatively modulate one or more checkpoint proteins or is able to totally or partially reduce, inhibit, interfere with or negatively modulate expression of one or more checkpoint proteins. Therefore, the checkpoint inhibitors preferably prevent inhibitory signals associated with the immune checkpoint resulting in preventing or reversing immune-suppression and establishment or enhancement of T cell immunity against cancer cells.

The amount of checkpoint inhibitor administered in each dose and/or treatment cycle may in particular be in a range, wherein more than 5%, preferably more than 10%, more preferably more than 15%, even more preferably more than 20%, even more preferably more than 25%, even more preferably more than 30%, even more preferably more than 35%, even more preferably more than 40%, even more preferably more than 45%, most preferably more than 50% of said checkpoint inhibitors bind to the checkpoint protein.

In preferred embodiments, the amount of checkpoint inhibitor administered, e.g., in each dose and/or in each treatment cycle, is
a) about 100-200 mg in total; and/or
b) about 0.20×10⁻⁹-1350×10⁻⁹ mol in total.

Checkpoint inhibitors may be administered in any manner and by any route known in the art. The mode and route of administration will depend on the type of checkpoint inhibitor to be used. In a preferred embodiment, the checkpoint inhibitor is administered systemically, such as parenterally, in particular intravenously.

Checkpoint inhibitors may be administered in the form of any suitable pharmaceutical composition as described herein. In a preferred embodiment, the checkpoint inhibitor is administered in the form of an infusion.

### Additional Therapeutic Agents

Besides the binding agent and the checkpoint inhibitor, the treatment regimen according to the first aspect of the present disclosure may further comprises administering to the subject one or more additional therapeutic agents.

In one embodiment, the one or more additional therapeutic agents comprise one or more chemotherapeutic agents, in particular, those chemotherapeutic agents which are commonly used in the treatment of a tumor or cancer as described herein. For example, the one or more chemotherapeutic agents include platinum-based compounds (e.g., cisplatin, oxaliplatin, and carboplatin), taxane-based compounds (e.g., paclitaxel and nab-paclitaxel), nucleoside analogs (e.g., 5-fluorouracil and gemcitabine), and combinations thereof (e.g., cisplatin/carboplatin+5-fluorouracil or nab-paclitaxel+gemcitabine).

### Subject and Tumor or Cancer to be Treated

The subject to be treated according to the present disclosure is preferably a human subject.

The tumor or cancer to be treated may be any tumor or cancer. Examples of tumors/cancers include, but are not limited to, carcinoma, lymphoma, blastoma, sarcoma, and leukemia, such as bone cancer, blood cancer, lung cancer, liver cancer, pancreatic cancer, skin cancer, cancer of the head or neck, cutaneous or intraocular melanoma, uterine cancer, ovarian cancer, rectal cancer, colorectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma.

In one embodiment, the tumor or cancer to be treated is a non-central nervous system (CNS) tumor or cancer, such as a non-CNS malignant tumor.

Preferably, the tumor or cancer may be selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, endometrial cancer, prostate cancer, penile cancer, cervical cancer, Hodgkin's lymphoma, non-Hodgkin's lymphoma, Merkel cell carcinoma and mesothelioma. More preferably, the tumor or cancer is selected from the group consisting of melanoma, lung cancer, colorectal cancer, pancreatic cancer, and head and neck cancer.

In one preferred embodiment, the tumor or cancer to be treated is a solid tumor or cancer. In one embodiment, the tumor or cancer to be treated is a non-CNS solid tumor or cancer, such as a non-CNS solid malignant tumor.

The tumor or cancer may in particular be a melanoma. Melanoma of skin is the seventeenth most common malignancy with an estimated age-standardized incidence rate of 3.4 per 100,000. Worldwide, approximately 324,635 new cases of melanoma of skin and 57,043 deaths are estimated in 2020 (GLOBOCAN, 2020). Five-year survival outcomes for patients with regional or distant disease are approximately 66% and 27%, respectively (SEER, 2018). In the first-line (I L) setting, targeted therapies and immune checkpoint (ICP) inhibitors have been approved for the treatment of advanced or metastatic melanoma alone or in combination. While improved outcomes are associated with combination therapy such as a programmed cell death protein 1 (PD-1) plus cytotoxic T-lymphocyte-associated protein 4 (CTLA-4) inhibitor, patients experience more frequent and severe immune-related adverse events (irAEs) (NCCN, 2021c). Novel combination approaches aimed to enhance efficacy and limit toxicity offer an opportunity to improve on existing standard of care (SOC). Patients with advanced or metastatic melanoma who have progressed on targeted therapy or immunotherapy typically receive cytotoxic therapies with modest response rates; therefore, there is also high unmet medical need in the second-line (2L) and later (2L+) setting (NCCN, 2021c).

In one embodiment, wherein the tumor or cancer is melanoma, the tumor or cancer is not an ocular (uveal) or mucosal melanoma. In one embodiment, the tumor or cancer is cutaneous or acral melanoma.

In one embodiment, wherein the tumor or cancer is melanoma, the melanoma is unresectable melanoma, in particular unresectable Stage III or Stage IV melanoma (preferably, according to the staging system of the American Joint Committee on Cancer (AJCC; version 8)).

In one embodiment, wherein the tumor or cancer is melanoma, the subject has not received prior systemic anticancer treatment for unresectable or metastatic melanoma, i.e., before the treatment according to the first aspect, the subject has not received systemic anticancer treatment for unresectable or metastatic melanoma.

In one embodiment, wherein the tumor or cancer is melanoma, the subject has a known tumor BRAF mutation status as per local standard testing (preferably an FDA-approved test). For such a subject, in particular for a subject with BRAF V600E mutant melanoma, preferably one or more (preferably all) of the following criteria are met: (i) lactate dehydrogenase<local upper limit of normal; (ii) no clinically significant tumor related symptoms in the judgment of the investigator; and (iii) absence of rapidly progressing metastatic melanoma in the judgment of the investigator.

In one embodiment, wherein the tumor or cancer is melanoma, the subject has not received prior treatment with an immune checkpoint (ICP) inhibitor, i.e., before the treatment according to the first aspect, the subject has not received treatment with an ICP inhibitor (in other words, the subject is naive to ICP inhibitor (CPI-naive)).

The tumor or cancer may in particular be a colorectal cancer. Colorectal cancer (CRC) is the third most commonly diagnosed cancer in males and the second in females. Worldwide, approximately 1,931,590 new cases of CRC and 935,173 deaths are estimated in 2020 (GLOBOCAN, 2020). Five-year relative survival rates in the US are 71% for patients with regional disease at diagnosis and 14% for patients with distant disease at diagnosis (SEER, 2018). Recommended initial therapy options for advanced or metastatic disease depend on whether the patient is a candidate for intensive therapy. The more intensive initial therapy options include 5-fluorouracil (5-FU)/leucovorin with oxaliplatin (FOLFOX), 5-FU/leucovorin with irinotecan (FOLFIRI), capecitabine with oxaliplatin, and 5-FU, oxaliplatin, and irinotecan (FOLFOXIRI). Addition of a biological agent (e.g., bevacizumab, cetuximab, panitumumab) is also an option in combination with some of these regimens (NCCN, 2021a). While the approval of targeted agents such as bevacizumab and cetuximab has improved outcomes for patients with metastatic CRC, all targeted agents currently approved either target the VEGF pathway or the EGFR pathway. Thus, there remains a need for new agents with novel mechanisms of action (MoAs), especially for patients whose tumors harbor RAS (KRAS, NRAS) or BRAF mutations, and for patients whose disease has progressed following available treatment options.

In one embodiment, wherein the tumor or cancer is CRC, the subject has not received prior treatment with an immune checkpoint (ICP) inhibitor, i.e., before the treatment according to the first aspect, the subject has not received treatment with ICP inhibitor.

The tumor or cancer may in particular be a lung cancer. The lung cancer may be a non-small cell lung cancer (NSCLC), such as a squamous or a non-squamous NSCLC. Lung cancer is the second most common malignancy with an estimated age-standardized incidence rate of 22.4 per 100,000 and a leading cause of cancer death for both men and women (Kantar, 2021). Worldwide, approximately 2,206,771 new cases of lung cancer and 1,796,144 deaths are estimated in 2020 (GLOBOCAN, 2020). Non-small-cell lung cancer (NSCLC) accounts for 85% to 90% of all cases, with a 5-year survival rate of approximately 18% across all stages of the disease, and only 3.5% for metastatic disease (Jemal et al., 2011) (Kantar, 2021; SEER, 2018). In the IL setting, treatment typically consists of platinum-based chemotherapy in combination with immunotherapy, or a targeted therapy, depending on molecular and biomarker analysis and the histology of the tumor (NCCN, 2021d). More recently, the advent of PD-1 and programmed death ligand 1 (PD-L1) inhibitors have improved outcomes for patients without driver mutations (approximately 62% of the non-squamous population and 77% of the squamous population (Kantar, 2021)). More treatment alternatives are needed for patients whose tumors do not harbor certain oncogenic mutations or do not express the biomarker for checkpoint inhibitor (CPI) options. Novel combinations with complementary approaches to enhance response may further address the unmet need in this population. For patients in the 2L setting, SOC is limited to platinum-based chemotherapy, a CPI monotherapy or docetaxel with or without ramucirumab depending on the previous therapy received. For patients in the third-line (3L) setting, chemotherapy monotherapy is the standard. Novel therapies are needed to limit toxicity and potentially enhance efficacy in this population (NCCN, 2021d).

In one embodiment, wherein the tumor or cancer is lung cancer, this tumor or cancer is a non-small cell lung cancer (NSCLC), such as a squamous or non-squamous NSCLC.

In one embodiment, wherein the tumor or cancer is lung cancer, in particular NSCLC, the tumor or cancer does not have an epidermal growth factor (EGFR)-sensitizing mutation and/or anaplastic lymphoma (ALK) translocation/ROS1 rearrangement. For subjects who are known to have a tumor of predominantly squamous histology, molecular testing for EGFR mutation and ALK translocation will not be required if this is per local SOC.

In one embodiment, wherein the tumor or cancer is lung cancer, in particular NSCLC, the tumor or cancer comprises cancer cells and PD-L1 is expressed in ≥1% of the cancer cells. Such expression may be determined by any means and method known to the skilled person, such as by immunohistochemistry (IHC) determined by a local SOC testing (preferably an FDA-approved test) or at a central laboratory.

In one embodiment, wherein the tumor or cancer is lung cancer, the subject has a histologically confirmed diagnosis of Stage IV metastatic or recurrent NSCLC (AJCC version 8), with no prior systemic anticancer therapy given as primary therapy for advanced or metastatic disease.

In one embodiment, wherein the tumor or cancer is lung cancer, the subject has not received prior treatment with an immune checkpoint (ICP) inhibitor, i.e., before the treatment according to the first aspect, the subject has not received treatment with ICP inhibitor.

The tumor or cancer may in particular be a head and neck cancer. Over 600,000 cases of head and neck squamous cell carcinoma (H4NSCC) are diagnosed annually worldwide. In 2020, approximately 65,630 new cases of oral cavity, pharyngeal, and laryngeal cancers and an estimated 14,500 deaths will occur over the same period in the US (NCCN, 2021b). Tobacco use, alcohol use, and human papillomavirus (HPV) infection increase the risk of developing HNSCC. Patients with locally HPV-positive HNSCC have improved treatment outcomes compared with patients with HPV-negative disease. For patients with recurrent or metastatic HNSCC, pembrolizumab/platinum (cisplatin or carboplatin)/5-FU and pembrolizumab monotherapy (for patients with PD-L1 combined positive score [CPS]≥20 or ≥1) are recommended 1L regimens; however, the median overall survival (mOS) is less than 15 months (NCCN, 2021b). Therefore, HNSCC remains an area of high unmet medical need and further opportunity exists to improve outcomes with novel treatment approaches.

In one embodiment, wherein the tumor or cancer is head and neck cancer, the tumor or cancer is squamous cell carcinoma (HNSCC).

In one embodiment, wherein the tumor or cancer is head and neck cancer, histologically or cytologically-confirmed recurrent or metastatic HNSCC is considered incurable by local therapies.

In one embodiment, wherein the tumor or cancer is head and neck cancer, the subject has not had prior systemic therapy administered in the recurrent or metastatic setting. Systemic therapy which was completed more than 6 months prior to signing consent if given as part of multimodal treatment for locally advanced disease is allowed.

In one embodiment, wherein the tumor or cancer is head and neck cancer, the eligible primary tumor locations are oropharynx, oral cavity, hypopharynx, and larynx.

In one embodiment, wherein the tumor or cancer is head and neck cancer, the subject does not have a primary tumor site of nasopharynx (any histology).

In one embodiment, wherein the tumor or cancer is head and neck cancer, the subject has tumor PD-L1 IHC combined positive score (CPS)≥1 (which may be determined by local (preferably an FDA-approved test) or central laboratory testing (central testing is mandated for the expansion phase)).

In one embodiment, wherein the tumor or cancer is oropharyngeal cancer, the subject has human papillomavirus (HPV) p16 test results (preferably available per local SOC). Oral cavity, hypopharynx, and larynx cancer are not required to undergo HPV testing by p16 IHC as by convention these tumor locations are assumed to be HPV negative.

In one embodiment, wherein the tumor or cancer is head and neck cancer, the subject has not received treatment with an immune checkpoint (ICP) inhibitor, i.e., before the treatment according to the first aspect, the subject has not received treatment with ICP inhibitor.

The tumor or cancer may in particular be a Pancreatic Ductal Adenocarcinoma. Pancreatic ductal adenocarcinoma (PDAC) is the third leading cause of cancer-related deaths in the US. Approximately 60,430 new cases of pancreatic cancer and 48,220 deaths are estimated to occur in the US in 2021 (Siegal, 2021). For patients with metastatic disease at diagnosis, the prognosis is dismal, with a mOS of <1 year. FOLFOXIRI and gemcitabine alone or in combination with albumin-bound paclitaxel are the predominant systemic therapeutic regimens used as 1L treatments in this setting, although other regimens containing agents such as irinotecan liposome injection (combined with 5-FU and leucovorin), bevacizumab, or erlotinib and FOLFOX may be utilized as 2L+treatments (NCCN, 2021e). Despite the increased number of treatments available in this setting, the significant toxicity and lack of survival benefit with current chemotherapy and combined modalities indicate that clinical trials are crucial options for newly diagnosed, patients with late stage disease.

In one embodiment, wherein the tumor or cancer is pancreatic cancer, the tumor or cancer is not pancreatic endocrine cancer.

In one embodiment, wherein the tumor or cancer is pancreatic cancer, the tumor or cancer is pancreatic ductal adenocarcinoma (PDAC).

In one embodiment, wherein the tumor or cancer is pancreatic cancer, the subject has not received prior treatment of metastatic disease by radiotherapy, surgery, chemotherapy, or investigational therapy, i.e., before the treatment according to the first aspect, the subject has not received treatment of metastatic disease by radiotherapy, surgery, chemotherapy, or investigational therapy.

In one embodiment, wherein the tumor or cancer is pancreatic cancer, the subject has not received prior treatment with a checkpoint inhibitor, i.e., before the treatment according to the first aspect, the subject has not received treatment with ICP inhibitor.

In one embodiment, wherein the tumor or cancer is pancreatic cancer, the tumor or cancer does not have actionable gene alterations such as BRCA 1/2 or PALB2 mutations.

### Treatment Regimen

The binding agent and the checkpoint inhibitor can be administered by any suitable way, such as intravenously, intraarterially, subcutaneously, intradermally, intramuscularly, intranodally, or intratumorally.

In one embodiment of the first aspect, the binding agent is in particular administered to the subject by systemic administration. Preferably, the binding agent is administered to the subject by intravenous injection or infusion. In one embodiment, the binding agent is administered in at least one treatment cycle.

In one embodiment, the checkpoint inhibitor is in particular administered to the subject by systemic administration. Preferably, the checkpoint inhibitor is administered to the subject by intravenous injection or infusion. In one embodiment, the checkpoint inhibitor is administered in at least one treatment cycle.

In one embodiment, the binding agent and the checkpoint inhibitor are in particular administered to the subject by systemic administration. Preferably, the binding agent and the checkpoint inhibitor are administered to the subject by intravenous injection or infusion. In one embodiment, the binding agent and the checkpoint inhibitor are administered in at least one treatment cycle.

In one embodiment, each treatment cycle is about two weeks (14 days), three weeks (21 days) or four weeks (28 days), preferably three weeks (21 days).

In particular embodiments, each dose is administered or infused every second week (1Q2W), every third week (1Q3W) or every fourth week (1Q4W), preferably every third week (1Q3W).

In some embodiments, one dose or each dose is administered or infused on day 1 of each treatment cycle. For example, one dose of the binding agent and one dose of the checkpoint inhibitor may be administered on day 1 of each treatment cycle.

Each dose may be administered or infused over a minimum of 30 minutes, such as over a minimum of 60 minutes, a minimum of 90 minutes, a minimum of 120 minutes or a minimum of 240 minutes.

The binding agent and the checkpoint inhibitor may be administered simultaneously. In an alternative preferred embodiment, the binding agent and the checkpoint inhibitor are administered separately.

In one embodiment, wherein the method further comprises administering to the subject one or more additional therapeutic agents, the one or more additional therapeutic agents preferably comprise one or more chemotherapeutic agents, such as platinum-based compounds (e.g., cisplatin, oxaliplatin, and carboplatin), taxane-based compounds (e.g., paclitaxel and nab-paclitaxel), nucleoside analogs (e.g., 5-fluorouracil and gemcitabine), and combinations thereof (e.g., cisplatin/carboplatin+5-fluorouracil or nab-paclitaxel+gemeitabine). In this embodiment, it is preferred that the one or more additional therapeutic agents are administered in at least one treatment cycle, wherein each treatment cycle preferably is three weeks (21 days). For example, one dose of the one or more additional therapeutic agents is administered at least every third week (1Q3W) for at least the first treatment cycle, such as twice every third week (2Q3W) for at least the first treatment cycle. In one embodiment, one dose of the one or more additional therapeutic agents is administered at least on day 1 of at least the first treatment cycle, such as on days 1 and 8 of at least the first treatment cycle.

The binding agent, the checkpoint inhibitor and, if present, the one or more additional therapeutic agents may be administered in any suitable form (e.g., naked as such). However, it is preferred that the binding agent, the checkpoint inhibitor, and, if present, the one or more additional therapeutic agents are administered in the form of any suitable pharmaceutical composition as described herein. In one embodiment, at least the binding agent and the checkpoint inhibitor are administered in the form of separate pharmaceutical compositions (i.e., one pharmaceutical composition for the binding agent and one pharmaceutical composition for the checkpoint inhibitor), preferably the binding agent, the checkpoint inhibitor and, if present, the one or more additional therapeutic agents are administered in the form of separate pharmaceutical compositions (i.e., one pharmaceutical composition for the binding agent, one pharmaceutical composition for the checkpoint inhibitor, and at least one pharmaceutical composition for the one or more additional therapeutic agents).

A composition or pharmaceutical composition may be formulated with a carrier, excipient and/or diluent as well as any other components suitable for pharmaceutical compositions, including known adjuvants, in accordance with conventional techniques such as those disclosed in Remington: The Science and Practice of Pharmacy, 19^{th} Edition, Gennaro, Ed., Mack Publishing Co., Easton, PA, 1995. The pharmaceutically acceptable carriers or diluents as well as any known adjuvants and excipients should be suitable for the binding agent and/or the checkpoint inhibitor and/or, if present, the one or more additional therapeutic agents and the chosen mode of administration. Suitability for carriers and other components of pharmaceutical compositions is determined based on the lack of significant negative impact on the desired biological properties of the chosen compound or pharmaceutical composition (e.g., less than a substantial impact [10% or less relative inhibition, 5% or less relative inhibition, etc.] upon antigen binding).

A composition, in particular the pharmaceutical composition of the binding agent, the pharmaceutical composition of the checkpoint inhibitor, and, if present, the at least one pharmaceutical composition of the one or more additional therapeutic agents, may include diluents, fillers, salts, buffers, detergents (e.g., a nonionic detergent, such as Tween-20 or Tween-80), stabilizers (e.g., sugars or protein-free amino acids), preservatives, solubilizers, and/or other materials suitable for inclusion in a pharmaceutical composition.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

Pharmaceutically acceptable carriers include any and all suitable solvents, dispersion media, coatings, antibacterial and antifungal agents, isotonicity agents, antioxidants and absorption-delaying agents, and the like that are physiologically compatible with the active compound, in particular a binding agent, the checkpoint inhibitor and/or, if present, the one or more additional therapeutic agents as used herein.

Examples of suitable aqueous and non-aqueous carriers which may be employed in the (pharmaceutical) compositions include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the (pharmaceutical) compositions is contemplated.

The term “excipient” as used herein refers to a substance which may be present in a (pharmaceutical) composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water A (pharmaceutical) composition may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

A (pharmaceutical) composition may also comprise isotonicity agents, such as sugars, polyalcohols, such as mannitol, sorbitol, glycerol or sodium chloride in the composition.

A (pharmaceutical) composition may also contain one or more adjuvants appropriate for the chosen route of administration such as preservatives, wetting agents, emulsifying agents, dispersing agents, preservatives or buffers, which may enhance the shelf life or effectiveness of the composition. The composition as used herein may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho esters, and polylactic acid alone or with a wax, or other materials well known in the art. Methods for the preparation of such formulations are generally known to those skilled in the art, see e.g. Sustained and Controlled Release Drug Delivery Systems, J. R. Robinson, ed., Marcel Dekker, Inc., New York, 1978.

“Pharmaceutically acceptable salts” comprise, for example, acid addition salts which may, for example, be formed by using a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, suitable pharmaceutically acceptable salts may include alkali metal salts (e.g., sodium or potassium salts); alkaline earth metal salts (e.g., calcium or magnesium salts); ammonium (NH₄⁺); and salts formed with suitable organic ligands (e.g., quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, arginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, galactate, galacturonate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isobutyrate, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, phthalate, picrate, pivalate, polygalacturonate, propionate, salicylate, stearate, sulfate, suberate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)). Salts which are not pharmaceutically acceptable may be used for preparing pharmaceutically acceptable salts and are included in the present disclosure.

In one embodiment, the binding agent, the checkpoint inhibitor, and, if present, the one or more additional therapeutic agents used herein may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Except in so far as any conventional media or agent is incompatible with the active compound, use thereof in the compositions is contemplated. Other active or therapeutic compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compounds in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

In a second aspect, the present disclosure provides a kit comprising (i) a binding agent comprising a first binding region binding to CD40 and a second binding region binding to CD137, (ii) a checkpoint inhibitor, and optionally (iii) one or more additional therapeutic agents. The embodiments disclosed herein with respect to the first aspect (in particular regarding the binding agent, the checkpoint inhibitor, and the optional one or more additional therapeutic agents) also apply to the kit of the second aspect. In one embodiment, the kit comprises at least two containers, wherein one thereof contains the binding agent (as such or in the form of a (pharmaceutical) composition) and the second container contains the checkpoint inhibitor (as such or in the form of a (pharmaceutical) composition). If the kit also comprises one or more additional therapeutic agents, it is preferred that the kit comprises at least three containers, one containing the binding agent (as such or in the form of a (pharmaceutical) composition), one containing the checkpoint inhibitor (as such or in the form of a (pharmaceutical) composition), and at least a third container containing the one or more additional therapeutic agents (as such or in the form of (a) (pharmaceutical) composition(s)).

In a third aspect, the present disclosure provides a kit of the second aspect for use in a method for reducing or preventing progression of a tumor or treating cancer in a subject. The embodiments disclosed herein with respect to the first aspect (in particular regarding the binding agent, the checkpoint inhibitor, the optional one or more additional therapeutic agents, the treatment regimen, the specific tumor/cancer, and the subject) and/or the second aspect also apply to the kit for use of the third aspect.

In a fourth aspect, the present disclosure provides a method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137. The embodiments disclosed herein with respect to the first aspect (in particular regarding the binding agent, the checkpoint inhibitor, the optional one or more additional therapeutic agents, the treatment regimen, the specific tumor/cancer, and the subject) also apply to the method of the fourth aspect.

In a further aspect, the present disclosure provides a checkpoint inhibitor for use in a method for reducing or preventing progression of a tumor or treating cancer in a subject, said method comprising administering to said subject the checkpoint inhibitor prior to, simultaneously with, or after administration of a binding agent inhibitor, wherein the binding agent comprises a first binding region binding to CD40 and a second binding region binding to CD137. The embodiments disclosed herein with respect to the first aspect (in particular regarding the binding agent, the checkpoint inhibitor, the optional one or more additional therapeutic agents, the treatment regimen, the specific tumor/cancer, and the subject) also apply to the checkpoint inhibitor for use of this further aspect.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Itemized Claims

1. A binding agent for use in a method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137.

2. The binding agent for use of item 1, wherein CD40 is human CD40, in particular human CD40 comprising the sequence set forth in SEQ ID NO: 36, and/or CD137 is human CD137, in particular human CD137 comprising the sequence set forth in SEQ ID NO: 38.

3. The binding agent for use of item 1 or 2, wherein the checkpoint inhibitor is at least one selected from the group consisting of PD-1 inhibitors, PD-L1 inhibitors, PD-L2 inhibitors, CTLA-4 inhibitors, TIM-3 inhibitors, KIR inhibitors, LAG-3 inhibitors, TIGIT inhibitors, VISTA inhibitors, and GARP inhibitors.

4. The binding agent for use of any one of items 1 to 3, wherein the checkpoint inhibitor is an antibody, such as a PD-1 blocking antibody, in particular pembrolizumab.

4a. The binding agent for use of any one of items 1 to 4, wherein the checkpoint inhibitor is an anti-PD-1 antibody or antigen-binding fragment thereof comprising the complementary determining regions (CDRs) of one of the anti-PD-1 antibodies or antigen-binding fragments described herein, such as the CDRs of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

4b. The binding agent for use of any one of items 1 to 4a, wherein the checkpoint inhibitor is an anti-PD-1 antibody or antigen-binding fragment thereof comprising the heavy chain variable region and the light chain variable region of one of the anti-PD-1 antibodies or antigen-binding fragments described herein, such as the heavy chain variable region and the light chain variable region of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IB1-308, BI-754091, GLS-010, LZM-009, AK-103, MGA-012, Sym-021, CS1003, and IgG1-PD1.

4b1. The binding agent for use of any one of items 1 to 4a, wherein the checkpoint inhibitor is an anti-PD-1 antibody or antigen-binding fragment thereof comprising the heavy chain variable region as defined in SEQ ID NO: 43 and the light chain variable region as defined in SEQ ID NO: 44.

4c. The binding agent for use of any one of items 1 to 4b, wherein the checkpoint inhibitor is an anti-PD-1 antibody or antigen-binding fragment thereof selected from the group consisting of nivolumab, Amp-514, tislelizumab, cemiplimab, TSR-042, JNJ-63723283, CBT-501, PF-06801 591, JS-001, camrelizumab, PDR001, BCD-100, AGEN2034, IBI-308, BI-754091, GLS-010, LZM-009, AK-103, MGA-012, Sym-021, CS1003, and IgG1-PD1.

4c1. The binding agent for use of any one of items 1 to 4b1, wherein the checkpoint inhibitor is an anti-PD-1 antibody or antigen-binding fragment thereof, wherein the anti-PD-1 antibody comprises the VH sequence as defined in SEQ ID NO: 43, the VL sequence as defined in SEQ ID NO: 44, the Fc sequence as defined in SEQ ID NO: 61, and optionally the kappa sequence as defined in SEQ ID NO: 27.

4d. The binding agent for use of any one of items 1 to 4, wherein the checkpoint inhibitor is an anti-PD-L1 antibody or antigen-binding fragment thereof comprising the complementary determining regions (CDRs) of one of the anti-PD-L1 antibodies or antigen-binding fragments described herein, such as the CDRs of atezolizumab or an antigen-binding fragment thereof.

4e. The binding agent for use of any one of items 1 to 4 and 4d, wherein the checkpoint inhibitor is an anti-PD-L1 antibody or antigen-binding fragment thereof comprising the heavy chain variable region and the light chain variable region of one of the anti-PD-L1 antibodies or antigen-binding fragments described herein, such as the heavy chain variable region and the light chain variable region of atezolizumab or an antigen-binding fragment thereof.

5. The binding agent for use of any one of the preceding items, wherein one or both of the binding agent and the checkpoint inhibitor is/are administered systemically, preferably intravenously.

6. The binding agent for use of any one of the preceding items, wherein
a) the first binding region comprises a heavy chain variable region (VH) comprising the CDR1, CDR2, and CDR3 sequences of SEQ ID NO: 7 or 9, and a light chain variable region (VL) comprising the CDR1, CDR2, and CDR3 sequences of SEQ ID NO: 8 or 10;
and
b) the second antigen-binding region comprises a heavy chain variable region (VH) comprising the CDR1, CDR2, and CDR3 sequences of SEQ ID NO: 17 or 19, and a light chain variable region (VL) comprising the CDR1, CDR2, and CDR3 sequences of SEQ ID NO: 18 or 20.

7. The binding agent for use of any one of the preceding items, wherein
a) the first binding region comprises a heavy chain variable region (VH) comprising the CDR1, CDR2, and CDR3 sequences set forth in: SEQ ID NO: 1, 2, and 3, respectively, and a light chain variable region (VL) comprising the CDR1, CDR2, and CDR3 sequences set forth in: SEQ ID NO: 4, 5, and 6, respectively;
and
b) the second antigen-binding region comprises a heavy chain variable region (VH) comprising the CDR1, CDR2, and CDR3 sequences set forth in: SEQ ID NO: 11, 12, and 13, respectively, and a light chain variable region (VL) comprising the CDR1. CDR2, and CDR3 sequences set forth in: SEQ ID NO: 14, 15, and 16, respectively.

8. The binding agent for use of any one of the preceding items, wherein
a) the first binding region comprises a heavy chain variable region (VH) comprising an amino acid sequence having at least 90%, at least 95%, at least 97%, at least 99%, or 100% sequence identity to SEQ ID NO: 7 or 9 and a light chain variable region (VL) region and comprising an amino acid sequence having at least 90%, at least 95%, at least 97%, at least 99%, or 100% sequence identity to SEQ ID NO: 8 or 10;
b) the second binding region comprises a heavy chain variable region (VII) comprising an amino acid sequence having at least 90%, at least 95%, at least 97%, at least 99%, or 25 100% sequence identity to SEQ ID NO: 17 or 19 and a light chain variable region (VL) region comprising an amino acid sequence having at least 90%, at least 95%, at least 97%, at least 99%, or 100% sequence identity to SEQ ID NO: 18 or 20.

9. The binding agent for use of any one of the preceding items, wherein
a) the first binding region comprises a heavy chain variable region (VH) comprising the amino acid sequence set forth in SEQ ID NO: 7 or 9 and a light chain variable region (VL) region comprising the amino acid sequence set forth in SEQ ID NO: 8 or 10;
and
b) the second binding region comprises a heavy chain variable region (VH) comprising the amino acid sequence set forth in SEQ ID NO: 17 or 19 and a light chain variable region (VL) region comprising the amino acid sequence set forth in SEQ ID NO: 18 or 20.

10. The binding agent for use of any one of the preceding items, wherein
a) the first binding region comprises a heavy chain variable region (VH) comprising the amino acid sequence set forth in SEQ ID NO: 9 and a light chain variable region (VL) region comprising the amino acid sequence set forth in SEQ ID NO: 10;
and
b) the second binding region comprises a heavy chain variable region (VH) comprising the amino acid sequence set forth in SEQ ID NO: 19 and a light chain variable region (VL) region comprising the amino acid sequence set forth in SEQ ID NO: 20.

11. The binding agent for use of any one of the preceding items, wherein the binding agent is a multispecific antibody, such as a bispecific antibody.

12. The binding agent for use of any one of the preceding items, wherein the binding agent is in the format of a full-length antibody or an antibody fragment.

13. The binding agent for use of any one of items 6-12, wherein each variable region comprises three complementarity determining regions (CDR1, CDR2, and CDR3) and four framework regions (FR1, FR2, FR3, and FR4).

14. The binding agent for use of item 13, wherein said complementarity determining regions and said framework regions are arranged from amino-terminus to carboxy-terminus in the following order: FR1, CDR1, FR2, CDR2, FR3, CDR3, FR4.

15. The binding agent for use of any one of items 6-14, which comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

16. The binding agent for use of any one of items 6-15, which comprises
i) a polypeptide comprising said first light chain variable region (VL) and further comprising a first light chain constant region (CL), and
ii) a polypeptide comprising said second light chain variable region (VL) and further comprising a second light chain constant region (CL).

17. The binding agent for use of any one of items 6-16, wherein the binding agent is an antibody comprising a first binding arm and a second binding arm, wherein the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).

18. The binding agent for use of any one of the preceding items, which comprises
i) a first heavy chain and light chain comprising said antigen-binding region capable of binding to CD40, and
ii) a second heavy chain and light chain comprising said antigen-binding region capable of binding CD137.

19. The binding agent for use of any one of the preceding items, wherein said binding agent comprises
i) a first heavy chain and light chain comprising said antigen-binding region capable of binding to CD40, the first heavy chain comprising a first heavy chain constant region and the first light chain comprising a first light chain constant region; and
ii) a second heavy chain and light chain comprising said antigen-binding region capable of binding CD137, the second heavy chain comprising a second heavy chain constant region and the second light chain comprising a second light chain constant region.

20. The binding agent for use of any one of items 15-19, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

21. The binding agent for use of any one of items 15-20, wherein each of the first and second heavy chain constant regions (CHs) comprises a CH3 region and wherein the two CH3 regions comprise asymmetrical mutations.

22. The binding agent for use of any one of items 15-21, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

23. The binding agent for use of item 22, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

24. The binding agent for use of any of the preceding items, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.

25. The binding agent for use of item 24, wherein said first and second heavy chain constant regions (CHs) are modified so that the antibody induces Fe-mediated effector function to a lesser extent compared to an antibody which is identical except for comprising non-modified first and second heavy chain constant regions (CHs).

26. The binding agent for use of item 25, wherein each of said non-modified first and second heavy chain constant regions (CHs) comprises the amino acid sequence set forth in SEQ ID NO: 21 or 29.

27. The binding agent for use of item 25 or 26, wherein said Fe-mediated effector function is measured by binding to Fcγ receptors, binding to C1q, or induction of Fe-mediated crosslinking of Fey receptors.

28. The binding agent for use of item 27, wherein said Fe-mediated effector function is measured by binding to C1q.

29. The binding agent for use of any one of items 24-28, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

30. The binding agent for use of any one of items 15-29, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

31. The binding agent for use of item 30, wherein the positions corresponding to positions L234 and L235 in a human IgG1 heavy chain according to EU numbering are F and E, respectively, in said first and second heavy chains.

32. The binding agent for use of item 30 or 31, wherein the positions corresponding to positions L234, L235, and D265 in a human IgG1 heavy chain according to EU numbering are F, E, and A, respectively, in said first and second heavy chain constant regions (HCs).

33. The binding agent for use of any one of items 30-32, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

34. The binding agent for use of any one of items 30-33, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

35. The binding agent for use of any one of items 15-34, wherein the constant region of said first and/or second heavy chain comprises or consists essentially of or consists of an amino acid sequence selected from the group consisting of
a) the sequence set forth in SEQ ID NO: 21 or 29 [IgG1-FC];
b) a subsequence of the sequence in a), such as a subsequence, wherein 1, 2, 3, 4, 5, 6, 7, 8, 9 or consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4, at most 3, at most 2 substitutions or at most 1 substitution, compared to the amino acid sequence defined in a) or b).

36. The binding agent for use of any one of items 15-34, wherein the constant region of said first or second heavy chain, such as the second heavy chain, comprises or consists essentially of or consists of an amino acid sequence selected from the group consisting of
a) the sequence set forth in SEQ ID NO: 22 or 30 [IgG1-F405L];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 9 substitutions, such as at most 8, at most 7, at most 6, at most 5, at most 4, at most 3, at most 2 substitutions or at most 1 substitution, compared to the amino acid sequence defined in a) or b).

37. The binding agent for use of any one of items 15-34, wherein the constant region of said first or second heavy chain, such as the first heavy chain comprises or consists essentially of or consists of an amino acid sequence selected from the group consisting of
a) the sequence set forth in SEQ ID NO: 23 or 31 [IgG1-F409R];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

38. The binding agent for use of any one of items 15-34, wherein the constant region of said first and/or second heavy chain comprises or consists essentially of or consists of an amino acid sequence selected from the group consisting of
a) the sequence set forth in SEQ ID NO: 24 or 32 [IgG1-Fc_FEA];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 7 substitutions, such as at most 6 substitutions, at most 5, at most 4, at most 3, at most 2 substitutions or at most 1 substitution, compared to the amino acid sequence defined in a) or b).

39. The binding agent for use of any one of items 15-38, wherein the constant region of said first and/or second heavy chain, such as the second heavy chain, comprises or consists essentially of or consists of an amino acid sequence selected from the group consisting of
a) the sequence set forth in SEQ ID NO: 25 or 33 [IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).

40. The binding agent for use of any one of items 15-39, wherein the constant region of said first and/or second heavy chain, such as the first heavy chain, comprises or consists essentially of or consists of an amino acid sequence selected from the group consisting of
a) the sequence set forth in SEQ ID NO: 26 or 34 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).

41. The binding agent for use of any one of the preceding items, wherein said binding agent comprises a kappa (κ) light chain constant region.

42. The binding agent for use of any one of the preceding items, wherein said binding agent comprises a lambda (λ) light chain constant region.

43. The binding agent for use of any one of the preceding items, wherein said first light chain constant region is a kappa (κ) light chain constant region or a lambda (λ) light chain constant region.

44. The binding agent for use of any one of the preceding items, wherein said second light chain constant region is a lambda (λ) light chain constant region or a kappa (κ) light chain constant region.

45. The binding agent for use of any one of the preceding items, wherein said first light chain constant region is a kappa (κ) light chain constant region and said second light chain constant region is a lambda (λ) light chain constant region or said first light chain constant region is a lambda (λ) light chain constant region and said second light chain constant region is a kappa (κ) light chain constant region.

46. The binding agent for use of any one of items 41-45, wherein the kappa (κ) light chain comprises an amino acid sequence selected from the group consisting of
a) the sequence set forth in SEQ ID NO: 27,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

47. The binding agent for use of any one of items 42-46, wherein the lambda (λ) light chain comprises an amino acid sequence selected from the group consisting of
a) the sequence set forth in SEQ ID NO: 28,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

48. The binding agent for use of any one of the preceding items, wherein the binding agent is of an isotype selected from the group consisting of IgG1, IgG2, IgG3, and IgG4.

49. The binding agent for use of any one of the preceding items, wherein the binding agent is a full-length IgG1 antibody.

50. The binding agent for use of any one of the preceding items, wherein the binding agent is an antibody of the IgG1m(f) allotype.

51. The binding agent for use of any one of the preceding items, wherein the subject is a human subject.

52. The binding agent for use of any one of the preceding items, wherein the tumor or cancer is a solid tumor or cancer.

53. The binding agent for use of any one of the preceding items, wherein the tumor or cancer is selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, endometrial cancer, prostate cancer, penile cancer, cervical cancer, Hodgkin's lymphoma, non-Hodgkin's lymphoma, Merkel cell carcinoma and mesothelioma.

54. The binding agent for use of any one of the preceding items, wherein the tumor or cancer is selected from the group consisting of melanoma, lung cancer, colorectal cancer, pancreatic cancer, and head and neck cancer.

55. The binding agent for use of item 53 or 54, wherein the tumor or cancer is melanoma, such as cutaneous or acral melanoma.

56. The binding agent for use of item 55, wherein the melanoma is unresectable melanoma, in particular unresectable Stage III or Stage IV melanoma.

57. The binding agent for use of item 55 or 56, wherein the subject has not received prior treatment with a checkpoint inhibitor.

58. The binding agent for use of any one of items 55-57, wherein the subject has not received prior systemic anticancer treatment for unresectable or metastatic melanoma.

59. The binding agent for use of item 53 or 54, wherein the tumor or cancer is lung cancer, in particular a non-small cell lung cancer (NSCLC), such as a squamous or non-squamous NSCLC.

60. The binding agent for use of item 59, wherein the lung cancer, in particular NSCLC, does not have an epidermal growth factor (EGFR)-sensitizing mutation and/or anaplastic lymphoma (ALK) translocation/ROS1 rearrangement.

61. The binding agent for use of item 59 or 60, wherein the lung cancer, in particular NSCLC, comprises cancer cells and PD-L1 is expressed in ≥1% of the cancer cells.

62. The binding agent for use of any one of items 59-61, wherein the subject has not received prior treatment with a checkpoint inhibitor.

63. The binding agent for use of item 53 or 54, wherein the tumor or cancer is head and neck cancer, in particular head and neck squamous cell carcinoma (HNSCC).

64. The binding agent for use of item 63, wherein the subject has not received prior treatment with a checkpoint inhibitor.

65. The binding agent for use of item 53 or 54, wherein the tumor or cancer is pancreatic cancer, in particular pancreatic ductal adenocarcinoma (PDAC).

66. The binding agent for use of item 65, wherein the subject has not received prior treatment of metastatic disease by radiotherapy, surgery, chemotherapy, or investigational therapy.

67. The binding agent for use of item 65 or 66, wherein the subject has not received prior treatment with a checkpoint inhibitor.

68. The binding agent for use of item 53 or 54, wherein the tumor or cancer is colorectal cancer.

69. The binding agent for use of item 68, wherein the subject has not received prior treatment with a checkpoint inhibitor.

70. The binding agent for use of any one of the preceding items, wherein the binding agent and the checkpoint inhibitor are administered in at least one treatment cycle, each treatment cycle being three weeks (21 days).

71. The binding agent for use of any one of the preceding items, wherein one dose of the binding agent and one dose of the checkpoint inhibitor are administered every third week (1Q3W).

72. The binding agent for use of any one of the preceding items, wherein one dose of the binding agent and one dose of the checkpoint inhibitor are administered on day 1 of each treatment cycle.

73. The binding agent for use of any one of the preceding items, wherein the method further comprises administering to said subject one or more additional therapeutic agents.

74. The binding agent for use of item 73, wherein the one or more additional therapeutic agents comprise one or more chemotherapeutic agents, such as platinum-based compounds (e.g., cisplatin, oxaliplatin, and carboplatin), taxane-based compounds (e.g., paclitaxel and nab-padlitaxel), nucleoside analogs (e.g., 5-fluorouracil and gemcitabine), and combinations thereof (e.g., cisplatin/carboplatin+5-fluorouracil or nab-paclitaxel+gemcitabine).

75. The binding agent for use of item 73 or 74, wherein the one or more additional therapeutic agents are administered in at least one treatment cycle, each treatment cycle being three weeks (21 days).

76. The binding agent for use of any one of items 73-75, wherein one dose of the one or more additional therapeutic agents is administered at least every third week (1Q3W) for at least the first treatment cycle, such as twice every third week (2Q3W) for at least the first treatment cycle.

77. The binding agent for use of any one of items 73-76, wherein one dose of the one or more additional therapeutic agents is administered at least on day 1 of at least the first treatment cycle, such as on days 1 and 8 of at least the first treatment cycle.

78. A kit comprising (i) a binding agent comprising a first binding region binding to CD40 and a second binding region binding to CD137, (ii) a checkpoint inhibitor, and optionally (iii) one or more additional therapeutic agents.

79. The kit according to item 78, wherein the binding agent and/or the checkpoint inhibitor and/or the one or more additional therapeutic agents is/are as defined in any one of items 1-50 and 74.

80. The kit according to item 78 or 79, wherein the binding agent, the checkpoint inhibitor, and, if present, the one or more additional therapeutic agents are for systemic administration, in particular for injection or infusion, such as intravenous injection or infusion.

81. The kit according to any one of items 78-80 for use in a method for reducing or preventing progression of a tumor or treating cancer in a subject.

82. The kit for use according to item 81, wherein the tumor or cancer and/or the subject and/or the method is/are as defined in any one of items 51-77.

83. A method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137.

84. The method of item 83, wherein the tumor or cancer and/or the subject and/or the method and/or the binding agent and/or the checkpoint inhibitor is/are as defined in any one of items 1-77.

Further aspects of the present disclosure are disclosed herein.

### EXAMPLES

### Example 1: Effect of bsIgG1-CD40×4-1BB and DuoBody-CD40×4-1BB in Combination with Pembrolizumab on IFNγ Secretion in an Allogeneic MLR Assay

To analyze if the combination of bsIgG1-CD40×4-1BB (Fc active) or DuoBody-CD40×4-1BB (Fc inactive) with pembrolizumab could result in potentiation of cytokine production in a mixed lymphocyte reaction (MLR) assay compared to single agent activity, five unique, allogeneic pairs of human mature dendritic cells (mDCs) and CD8+ T cells were co-cultured in the presence of bsIgG1-CD40×4-1BB alone, DuoBody-CD40×4-1BB alone, pembrolizumab alone or a combination of either CD40×4-1BB bispecific with pembrolizumab. Interferon (IFN)γ secretion was assessed in the supernatants of the co-cultures using an IFNγ-specific immunoassay.

### Methods

Monocyte and T Cells from Healthy Donors

CD14+ monocytes and purified CD8+ T cells were obtained from Precision Medicine or BiolVT. Allogeneic donor pairs were used for the MLR assay.

### Differentiation of Monocytes to Immature Dendritic Cells

Human CD14⁺ monocytes were obtained from healthy donors (see above). For differentiation into immature dendritic cells (iDCs), 1-1.5×10⁶ monocytes/mL were cultured for six days in Roswell Park Memorial Institute (RPMI) 1640 complete medium (ATCC modification formula; ThermoFisher, cat. no. A1049101) supplemented with 10% heat-inactivated Fetal Bovine Serum (FBS; Gibco, cat. no. 16140071), 100 ng/ml, granulocyte-macrophage colony-stimulating factor (GM-CSF; BioLegend, cat. no. 766106) and 300 ng/mL interleukin-4 (IL-4; BioLegend, cat. no. 766206) in T25 culture flasks (Falcon, cat. no. 353108) at 37° C. Once during these six days, the medium was replaced with fresh medium with supplements.

Maturation of iDCs

To mature the iDCs, the cells were harvested by collecting non-adherent cells, counted, incubated at 1-1.5×10⁶ cells/mL in RPMI 1640 complete medium supplemented with 10% FBS, 100 ng/mL GM-CSF, 300 ng/mL IL-4 and 1× with lipopolysaccharide (LPS; ThermoFisher, cat. no. 00-4976-93) for 24 h prior to start of the mixed lymphocyte reaction (MLR) assay at 37° C.

### Mixed Lymphocyte Reaction (MLR)

One day prior to the start of an MLR assay, purified CD8+ T cells obtained from allogeneic healthy donors were thawed. Cells were resuspended at 1×10⁶ cells/mL in RPMI 1640 complete medium supplemented with 10% FBS and 10 ng/mL IL-2 (BioLegend, cat. no. 589106) and incubated O/N at 37° C.

The next day, the LPS-matured dendritic cells (mDCs, see Maturation of iDCs) and allogeneic purified CD8+ T cells were harvested and resuspended in AIM-V medium (ThermoFisher, cat. no. 12055091) at 4×10⁵ cells/mL and 4×10⁶ cells/mL, respectively.

In co-cultures, 20,000 mDCs were incubated with 200,000 allogeneic purified CD8+ T cells (DC:T cell ratio of 1:10) in the presence of DuoBody-CD40×4-1BB (0.001-30 μg/mL) either alone or in combination with pembrolizumab (0.1-30 μg/mL or 0.1-100 μg/mL; non-clinical/research-grade version of the clinical product pembrolizumab; Selleckchem, cat. no. A2005), bsIgG1-CD40×4-1BB (0.001-30 μg/mL) either alone or in combination with pembrolizumab (0.1-30 μg/mL), pembrolizumab (0.1-30 μg/mL or 0.1-100 μg/mL), bsIgG1-CD40×ctrl (30 μg/mL), bsIgG1-ctrl×4-1BB (30 μg/mL), IgG4 (100 μg/mL; Biolegend, cat. no. 403702), IgG1-ctrl-FEAL (30 μg/mL) or IgG1-ctrl (0.001-30 μg/mL) in AIM-V medium in a 96-well round-bottom plate (Falcon, cat. no. 353227) at 37° C. After 5 days, the plates were centrifuged at 500×g for 5 min and the supernatant was carefully transferred from each well to a new 96-well round bottom plate.

The collected supernatants from the MLR assay were analyzed for interferon (IFN)γ levels by enzyme-linked immunosorbent assay (ELISA) using an Alpha Lisa IFNγ kit (Perkin Elmer, cat. no. AL217) on an Envision instrument, according to the manufacturer's instructions.

### Antibodies

Antibodies listed below were expressed as IgG1,κ. When applicable, specific mutations were introduced by gene synthesis at GeneArt. Antibodies were purified from the culture supernatant by Protein A affinity chromatography. Bispecific antibodies (DuoBody molecules) were obtained by controlled Fab-arm exchange (WO2011/131746). Briefly, two parental antibodies, containing single matched point mutations in the CH3 domain (F405L in one and K409R in the other [EU numbering (Kabat, NIH publication no 91-3242, 5th edition ed. National Institutes of Public Health, Bethesda, MD, USA. 662, 680, 689)]), were produced separately, mixed and subjected to controlled reducing conditions. The reduction breaks down the inter-chain disulfide bonds of the molecule, while the matched CH3 domains (containing the F405L and K409R) drive heterodimerization of the Fab arms and formation of bispecific molecules. Subsequent re-oxidation of the disulfide bonds yields highly pure bispecific antibody preparations with a regular IgG1 architecture. IgG concentration was measured by absorbance at 280 nm. Purified antibodies were stored at 4° C.

TABLE 5

| Test compound | Supplier, cat. no. | Comprising SEQ ID NOs |
|---|---|---|
| DuoBody-CD40 × 4-1BB | N/A | SEQ ID NOs: 9, 10, 19, 20, 25, 26, 27 |
| bsIgG1-CD40 × 4-1BB¹ | N/A | SEQ ID NO: 9, 10, 19, 20, 22, 23, 27 |
| bsIgG1-CD40 × ctrl² | N/A | SEQ ID NO: 9, 10, 25, 26, 27 |
| bsIgG1-ctrl × 4-1BB² | N/A | SEQ ID NO: 19, 20, 25, 26, 27 |
| Pembrolizumab | Selleckchem, cat. no. A2005 (non- | N/A |
| | clinical/research-grade version of | |
| | the clinical product pembrolizumab; | |
| | Lot no. A200504) | |
| IgG1-ctrl² | N/A | SEQ ID NO: 21, 27 |
| IgG1-ctrl-FEAL² | N/A | SEQ ID NO: 25, 27 |

¹This antibody comprises the CD40-specific and 4-1BB-specific Fab arms presented in Table 1 in an Fc-active IgG1 backbone (i.e. without the Fc-silencing L234F, L235E, D265A mutations).
²Control binding moiety based on anti-HIV gp120 antibody IgG1-b12 (Barbas et al., J Mol Biol 1993, 230: 812-823)

### Results

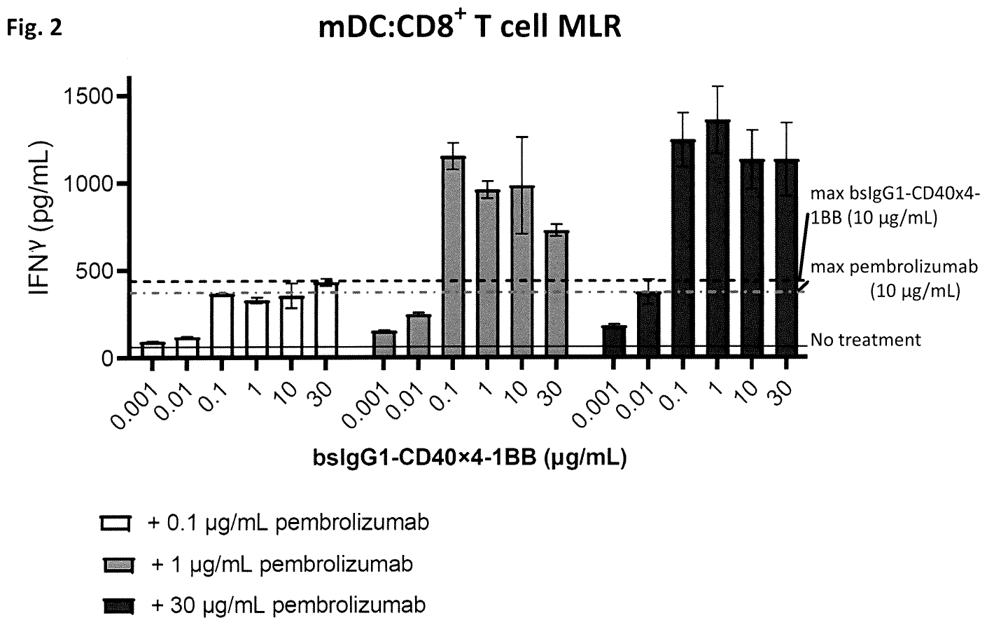

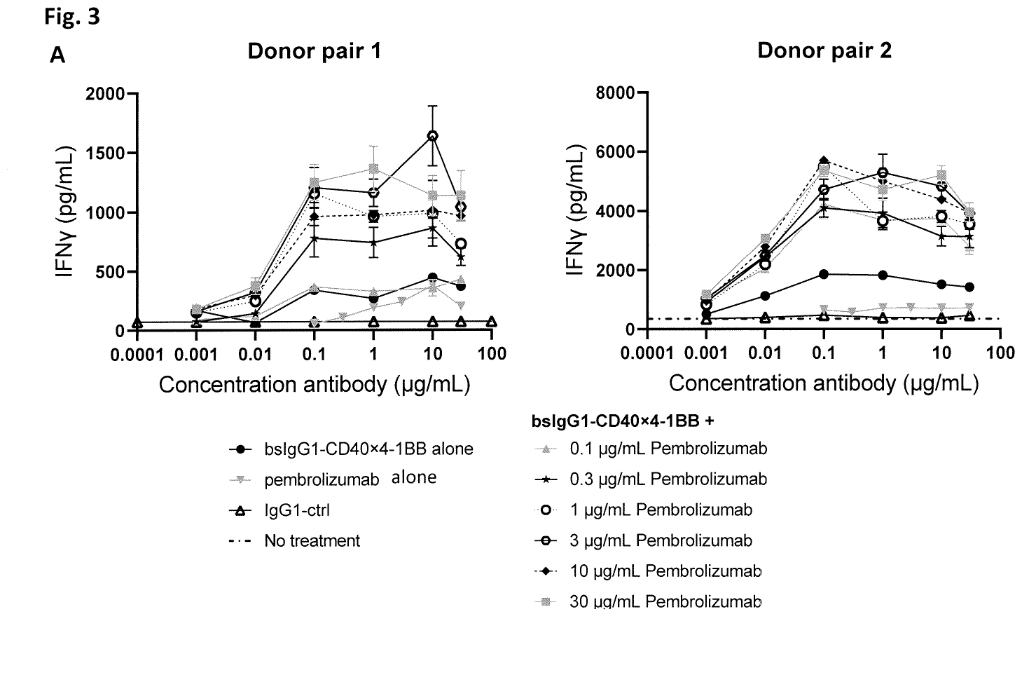

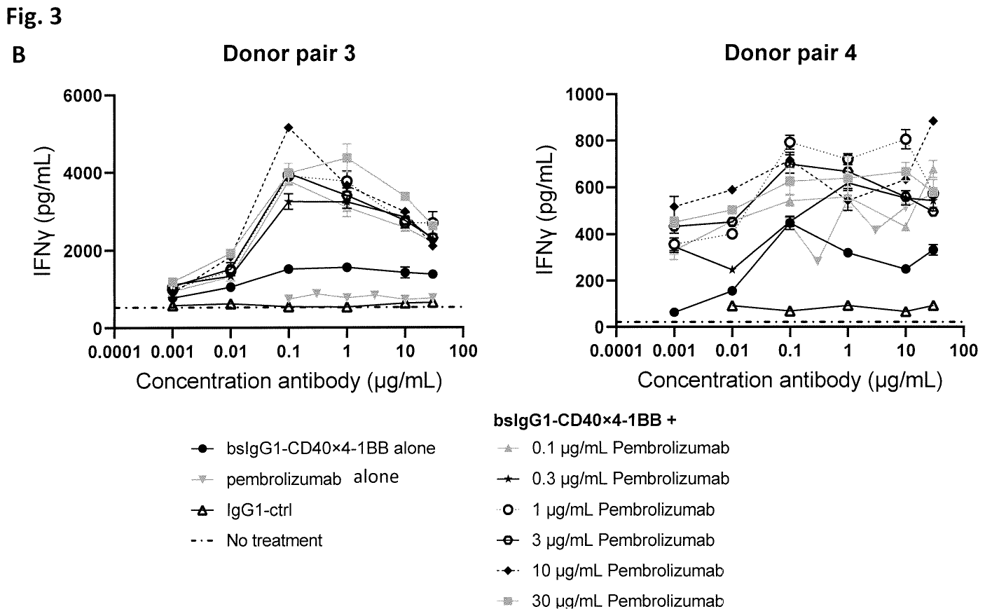

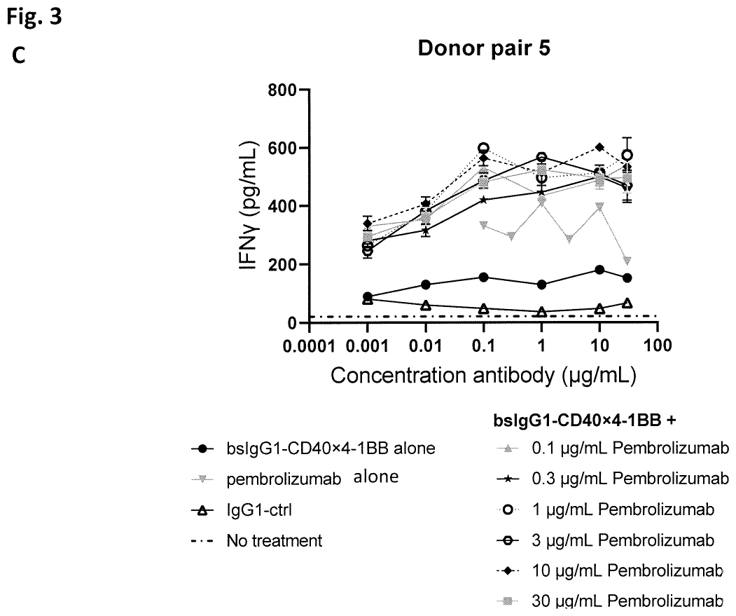

In the first experiment, BsIgG1-CD40×4-1BB enhanced IFNγ secretion in co-cultures of purified CD8+ T cells and allogeneic mDCs compared to IgG1-ctrl in five donor pairs (see FIGS. 2 & 3, Table 6). Concurrent exposure to bsIgG1-CD40×4-1BB and pembrolizumab induced a 1.89 to 2.73-fold increase in IFNγ compared to bsIgG1-CD40×4-1BB alone (FIG. 2, Table 7) and a 2.14 to 3.09-fold increase compared to pembrolizumab alone (FIG. 2, Table 8). Highest IFNγ concentrations were observed upon treatment with the combination of bsIgG1-CD40×4-1BB with >1 μg/mL pembrolizumab.

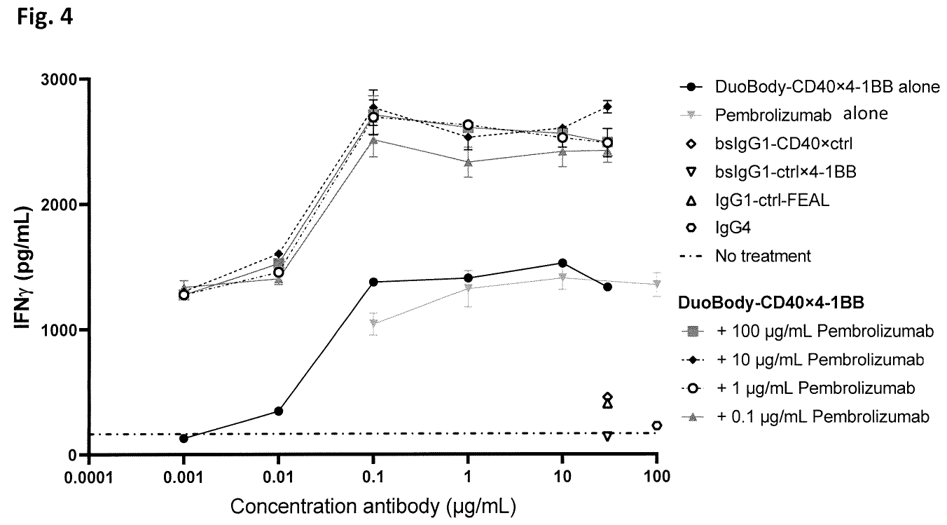

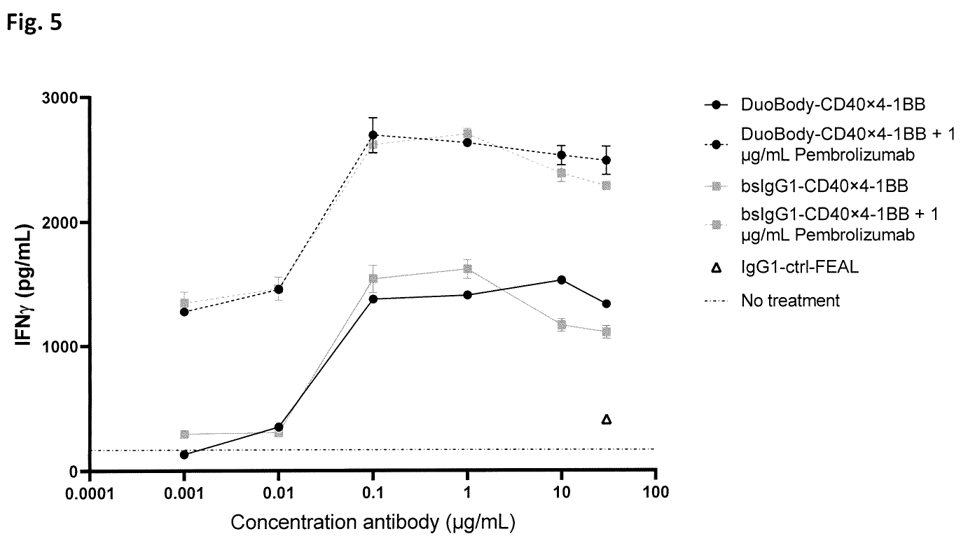

In a second experiment, DuoBody-CD40×4-1BB enhanced IFNγ secretion in co-cultures of purified CD8+ T cells and allogeneic mDCs compared to IgG1-ctrl-FEAL and monovalent control antibodies bsIgG1-CD40×ctrl or bsIgG1-ctrl×4-1BB (FIG. 4). Concurrent exposure to DuoBody-CD40×4-1BB and pembrolizumab induced an increase in IFNγ compared to DuoBody-CD40×4-1BB alone and compared to pembrolizumab alone at all concentrations tested (FIG. 4). Comparable levels of IFNγ were observed when DuoBody-CD40×4-1BB was combined with 0.1, 1, 10 or 100 μg/mL pembrolizumab (FIG. 4). When comparing DuoBody-CD40×4-1BB and bsIgG1-CD40×4-1BB, both bispecific antibodies induced comparable levels of IFNγ either alone or in combination with 1 μg/mL pembrolizumab (FIG. 5).

### Conclusion

Together, these results indicate that combining DuoBody-CD40×4-1BB or bsIgG1-CD40×4-1BB with pembrolizumab potentiates IFNγ secretion in a mature DC/CD8⁻ T cell MLR assay. This data suggests that the magnitude of immune response can be amplified through targeting of the PD-1/PD-L1 axis in combination with CD40 and 4-1BB co-stimulation.

TABLE 6
IFNy secretion induced by bsIgG1-CD40 × 4-1BB either alone or in combination with PD-1 mAb

| | | | | | | bsIgG1-CD40 × 4- | | bsIgG1-CD40 × 4- | | bsIgG1-CD40 × 4- | |
|---|---|---|---|---|---|---|---|---|---|---|---|
| | No | | | bsIgG1- | | 1BB + 0.1 μg/mL | | 1BB + 0.3 μg/mL | | 1BB + 1 μg/mL | |
| | treatment | IgG1-ctrl | | CD40 × 4-1BB | | pembrolizumab | | pembrolizumab | | pembrolizumab | |
| Donor | Baseline | ymax | Fold | ymax | Fold | ymax | Fold | ymax | Fold | ymax | Fold |
| pair | (pg/mL) | (pg/mL) | change | (pg/mL) | change | (pg/mL) | change | (pg/mL) | change | (pg/mL) | change |
| 1 | 74 | | | 441 | 6.0 | 429 | 5.8 | 861 | 11.6 | 1154 | 15.6 |
| 2 | 356 | 475 | 1.3 | 1858 | 5.2 | 4232 | 11.9 | 4102 | 11.5 | 5493 | 15.4 |
| 3 | 534 | 657 | 1.2 | 1562 | 2.9 | 3781 | 7.1 | 3250 | 6.1 | 3905 | 7.3 |
| 4 | 21 | 93 | 4.4 | 448 | 21.3 | 678 | 32.3 | 618 | 29.4 | 806 | 38.4 |
| 5 | 21 | 408 | 19.4 | 179 | 8.5 | 539 | 25.7 | 499 | 23.8 | 597 | 28.4 |

| | bsIgG1-CD40 × 4- | | bsIgG1-CD40 × 4- | | bsIgG1-CD40 × 4- | | | |
|---|---|---|---|---|---|---|---|---|
| | 1BB + 3 μg/mL | | 1BB + 10 μg/mL | | 1BB + 30 μg/mL | | | |
| | pembrolizumab | | pembrolizumab | | pembrolizumab | | Pembrolizumab | |
| Donor | ymax | Fold | ymax | Fold | ymax | Fold | ymax | Fold |
| pair | (pg/mL) | change | (pg/mL) | change | (pg/mL) | change | (pg/mL) | change |
| 1 | 1637 | 22.1 | 1013 | 13.7 | 1361 | 18.4 | 376 | 5.1 |
| 2 | 5300 | 14.9 | 5700 | 16.0 | 5361 | 15.1 | 726 | 2.0 |
| 3 | 3969 | 7.4 | 5156 | 9.6 | 4370 | 8.2 | 882 | 1.6 |
| 4 | 700 | 33.3 | 885 | 42.1 | 667 | 31.8 | 574 | 27.3 |
| 5 | 567 | 27.0 | 600 | 28.6 | 523 | 24.9 | 408 | 19.4 |

TABLE 7
Fold increase in ymax compared to bslgG1-CD40 × 4-1BB

| | bsIgG1-CD40 × | bsIgG1-CD40 × | bsIgG1-CD40 × | bsIgG1-CD40 × | bsIgG1-CD40 × | bsigG1-CD40 × |
|---|---|---|---|---|---|---|
| | 4-1BB + 0.1 μg/mL | 4-1BB + 0.3 μg/mL | 4-1BB + 1 μg/mL | 4-1BB + 3 μg/mL | 4-1BB + 10 μg/mL | 4-1BB + 30 μg/mL |
| Donor pair | pembrolizumab | pembrolizumab | pembrolizumab | pembrolizumab | pembrolizumab | pembrolizumab |
| 1 | 0.97 | 1.95 | 2.61 | 3.70 | 2.29 | 3.08 |
| 2 | 2.28 | 2.21 | 2.96 | 2.85 | 3.07 | 2.89 |
| 3 | 2.42 | 2.08 | 2.50 | 2.54 | 3.30 | 2.80 |
| 4 | 1.51 | 1.38 | 1.80 | 1.56 | 1.97 | 1.49 |
| 5 | 3.01 | 2.78 | 3.33 | 3.16 | 3.35 | 2.92 |
| Geomean ± SD | 1.89 ± 0.71 | 2.02 ± 0.45 | 2.58 ± 0.51 | 2.65 ± 0.71 | 2.73 ± 0.56 | 2.55 ± 0.58 |

TABLE 8
Fold increase in ymax compared to pembrolizumab

| | bsIgG1-CD40 × | bsIgG1-CD40 × | bsIgG1-CD40 × | bsIgG1-CD40 × | bsIgG1-CD40 × | bsIgG1-CD40 × |
|---|---|---|---|---|---|---|
| | 4-1BB + 0.1 μg/mL | 4-1BB + 0.3 μg/mL | 4-1BB + 1 μg/mL | 4-1BB + 3 μg/mL | 4-1BB + 10 μg/mL | 4-1BB + 30 μg/mL |
| Donor pair | pembrolizumab | pembrolizumab | pembrolizumab | pembrolizumab | pembrolizumab | pembrolizumab |
| 1 | 1.14 | 2.29 | 3.06 | 4.35 | 2.69 | 3.61 |
| 2 | 5.83 | 5.65 | 7.57 | 7.30 | 7.85 | 7.38 |
| 3 | 4.29 | 3.69 | 4.43 | 4.50 | 5.85 | 4.96 |
| 4 | 1.18 | 1.08 | 1.41 | 1.22 | 1.54 | 1.16 |
| 5 | 1.32 | 1.22 | 1.46 | 1.38 | 1.47 | 1.28 |
| Geomean ± SD | 2.14 ± 1.95 | 2.29 ± 1.71 | 2.92 ± 2.28 | 3.00 ± 2.26 | 3.09 ± 2.54 | 2.88 ± 2.34 |

### Example 2: Clinical Trial Design

### Trial Design

This is a first-in-human, open-label, multicenter phase 1/2 trial of GEN1042 in subjects with solid malignant tumors. The trial consists of 4 parts: a GEN1042 monotherapy dose escalation (phase 1a), a GEN 1042 monotherapy expansion (phase 2a), a combination therapy safety run-in (phase 1b), and a combination therapy expansion (phase 2).

The dose escalation for monotherapy (phase 1a) will evaluate GEN1042 in subjects with non-central nervous system (CNS) solid malignant tumors to determine the MTD or maximum administered dose and/or RP2D. The phase 1b safety run-in will evaluate the GEN1042 monotherapy RP2D from dose escalation in combination with 1 or more therapies in select tumor types following as described in detail below. The RP2D of GEN1042 determined during the safety run-in will be further evaluated in phase 2.

### Treatment Schedule

### Safety Run-In for Combination Therapy—Phase 1b

The “3+3” design is conventional for phase 1 oncology studies. The combination safety run-in part of this trial will follow “3+3” to allow the safety of each dose of GEN1042 administered+/−pembrolizumab+/−chemotherapy in 3 subjects to be assessed before treating additional subjects with the same or next doses. Subjects will not be randomized; they will be assigned to the cohort that is being filled at the time the subject is ready to enter the trial.

In detail, safety combination cohorts will receive GEN1042+pembrolizumab OR GEN1042+chemotherapy+/−pembrolizumab. Enrollment in phase 1b will begin after the RP2D of GEN1042 monotherapy has been determined from the dose escalation part (phase 1a).

Three parallel regimens are planned during the combination safety run-in:
1. GEN1042+pembrolizumab Q3W; continued treatment until PD, undue toxicity, withdrew consent or up to 35 cycles (total 2 years)
   NSCLC (CPI-naive, PD-L1 expressing, TPS≥1% per local lab testing)
   HNSCC (CPI-naive, PD-L1 expressing, CPS≥1 per local lab testing)
   Melanoma (CPI-naive, regardless of PD-L1 expressing)
2. GEN 1042+pembrolizumab+cis-/carboplatin+5-FU Q3W for 6 cycles followed by GEN1042+pembrolizumab Q3W; continued treatment until PD, undue toxicity, withdrew consent or up to additional 29 cycles (total 2 years)
   HNSCC (CPI-naive, PD-L1 expressing, CPS≥1 per local lab testing)
3. PDAC (Regardless of PD-L1 expression):
   Regimen 3a: GEN1042 Q3W+gemcitabine+nab-paclitaxel 2Q3W for a total of 8 cycles
   Regimen 3b: GEN1042+pembrolizumab Q3W+gemcitabine+nab-paclitaxel 2Q3W for 8 cycles followed by GEN1042+pembrolizumab Q3W continued treatment until PD, undue toxicity, withdrew consent or up to an additional 27 cycles (total 2 years)

The above respective safety combination regimen will be evaluated following a 3+3 dose de-escalation design. Cohorts of 3-6 subjects will be entered sequentially into de-escalating dosage tiers. The starting dose for GEN1042 is 100 mg 1Q3W (DL1) for regimens 1, 2 and 3a. The next dose level will be 60 mg (DL2) of GEN1042. The dose of GEN1042 determined through Regimen 3a will be the starting dose for Regimen 3b. The approved dose of pembrolizumab and chemotherapy will be used following the SOC practice.
Each regimen will be tested starting with three subjects using the RP2D GEN1042 dose from phase 1a (100 mg 1Q3W) for regimens 1, 2 and 3a, OR using the safe and tolerable dose from Regimen 3a for Regimen 3b, administered in combination with the designated therapy(ies) shown above.
If none of the three subjects in a cohort experiences a DLT, the regimen will be deemed safe, and this regimen will be further tested in the expansion arm.
If one of the first three subjects experiences a DLT, three more subjects will be treated at the same dose level. If at most one out of six subjects experienced DLT, the regimen will also be considered safe to move forward into the expansion part.
If at least two subjects among a cohort of three to six subjects experience DLTs (ie, ≥33% of subjects with a DLT at that dose level), de-escalate to the next lower dose level, eg, 60 mg of GEN1042.
The goal in Regimen 3 is to identify a safe and tolerable dose of GEN1042 in combination with pembrolizumab, gemcitabine and nab-paclitaxel.

Doses below the highest dose level which is deemed safe in the dose escalation part (eg, 300 mg, 30 mg, etc.) may also be tested in the expansion part upon agreement between the investigator and the sponsor. If there is no tolerable dose identified, the safety run-in and its associated expansion arm will be terminated.

DLT will be evaluated after each subject completed DLT observation period, that is 1 cycle (21 days) for GEN1042+pembrolizumab OR 2 cycles (21 days) for GEN1042+chemotherapy+/−pembrolizumab, respectively. Determination of RP2D of GEN1042 for the combination regimen will be based on the totality of the data, taking into consideration the DLT and the overall safety profile, anti-tumor activity, PK, and biomarker data if available.

### Expansion Part—Combination Therapy Cohorts—Phase 2

For the combination therapy expansion, the selected RP2D GEN1042 dose determined from phase 1b combination safety run-in will be administered, in combination with 1 or more therapies as shown below. The combination therapy will be administered as the 1L treatment setting. Five parallel arms in four indications are planned.

TABLE 9

| Combination | | |
|---|---|---|
| Expansion | | |
| Arms | Indications | Treatment Regimen |
| 1 | 1L melanoma, | GEN1042 + pembrolizumab Q3W; continued treatment |
| | CPI-naive, regardless | until PD, undue toxicity, withdrew consent or up to 35 |
| | of PD-L1 expression | cycles (total 2 years) |
| 2a | 1L NSCLC, | GEN1042 + pembrolizumab Q3W; continued treatment |
| | CPI-naive, PD-L1 | until PD, undue toxicity, withdrew consent or up to 35 |
| | TPS ≥1-49% | cycles (total 2 years) |
| 2b | 1L NSCLC, | GEN1042 + pembrolizumab Q3W; continued treatment |
| | CPI-naive, PD-L1 | until PD, undue toxicity, withdrew consent or up to 35 |
| | TPS ≥50% | cycles (total 2 years) |
| 3 | 1L HNSCC, | GEN1042 + pembrolizumab Q3W; continued treatment |
| | CPI-naive, PD-L1 | until PD, undue toxicity, withdrew consent or up to 35 |
| | CPS ≥20 | cycles (total 2 years) |
| 4 | 1L HNSCC, | GEN1042 + pembrolizumab + cis-/carboplatin + 5-FU, |
| | CPI-naive, PD-L1 | Q3W for 6 cycles → GEN1042 + pembrolizumab Q3W; |
| | CPS 1-19 | continued treatment until PD, undue toxicity, withdrew |
| | | consent or up to an additional 29 cycles (total 2 years) |
| 5 | 1L PDAC, regardless | GEN1042 + pembrolizumab Q3W + gemcitabine + nab- |
| | of PD-L1 expression | paclitaxel 2Q3W for 8 cycles → GEN1042 + |
| | | pembrolizumab Q3W continued treatment until PD, |
| | | undue toxicity, withdrew consent or up to an additional 27 |
| | | cycles (total 2 years) |

Abbreviations: 1L = first line; 2Q3W = Twice every 3 weeks (weekly for 2 weeks followed by a week of rest); 5-FU = 5-fluorouracil; Cis/Carbo = cisplatin/carboplatin; CPI = checkpoint inhibitor; CPS = combined positive score; HSNCC = head and neck squamous cell carcinoma; NSCLC = non-small-cell lung cancer; PD = progressive disease; PDAC = pancreatic ductal adenocarcinoma; PD-L1 = programmed death-ligand 1; Q3W = every 3 weeks; TPS = tumor proportion score.

TABLE 10
Dosage and Administration

| | | | | | | |
|---|---|---|---|---|---|---|
| Drug | | | | | | |
| Name/Treatment | | | | | | |
| Regimen | Week 1 | Week 2 | Week 3 | Week 4 | Week 5 | Week 6 |

| | | | | |
|---|---|---|---|---|
| GEN1042 | Day 1 | | Day 1 | |
| Monotherapy | GEN1042 | | GEN1042 | |
| GEN1042 + | Day 1 | | Day 1 | |
| Pembrolizumab | Pembrolizumab + | | Pembrolizumab + | |
| 200 mg | GEN1042 | | GEN1042 | |
| GEN1042 + | Day 1 | | Day 1 | |
| Pembrolizumab | Pembrolizumab + | | Pembrolizumab+ | |
| 200 mg + | GEN1042 + | | GEN1042 + | |
| Cisplatin | Cisplatin + | | Cisplatin + | |
| 100 mg/m² + 5-FU | 5-FU (for 4 days, | | 5-FU (for 4 days, | |
| 1000 mg/m²/day^{a} | day 1, 2, 3, 4) | | day 1, 2, 3, 4) | |
| GEN1042 + | Day 1 | | Day 1 | |
| Pembrolizumab | Pembrolizumab + | | Pembrolizumab + | |
| 200 mg + | GEN1042 + | | GEN1042 + | |
| Carboplatin | Carboplatin + | | Carboplatin + | |
| AUC 5 + 5-FU | 5-FU (for 4 days, | | 5-FU (for 4 days, | |
| 1000 mg/m²/day^{a} | day 1, 2, 3, 4) | | day 1, 2, 3, 4) | |
| GEN1042 + nab- | Day 1 | Day 1 | Day 1 | Day 1 |
| paclitaxel | GEN1042 + | nab-paclitaxel + | GEN1042 + | nab-paclitaxel + |
| 125 mg/m² + | nab-paclitaxel + | gemcitabine | nab-paclitaxel + | gemcitabine |
| gemcitabine | gemcitabine | | gemcitabine | |
| 1000 mg/m² | | | | |
| GEN1042 + | Day 1 | Day 1 | Day 1 | Day 1 |
| Pembrolizumab | Pembrolizumab + | nab-paclitaxel + | Pembrolizumab + | nab-paclitaxel + |
| 200 mg + nab- | GEN1042 + | gemcitabine | GEN1042 + | gemcitabine |
| paclitaxel | nab-paclitaxel + | | nab-paclitaxel + | |
| 125 mg/m² + | gemcitabine | | gemcitabine | |
| gemcitabine | | | | |
| 1000 mg/m^{2b} | | | | |

Abbreviations: 5-FU = 5-fluorouracil; Q3W = every 3 weeks.
^{a}Starting from Cycle 7, subjects without disease progression will receive GEN1042 + pembrolizumab Q3W continuously
^{b}Starting from Cycle 9, subjects without disease progression will receive GEN1042 + pembrolizumab Q3W continuously

For combination therapy expansion, treatment will be administered in the order presented below: Pembrolizumnab infusion will be administered first followed by GEN1042 followed by SOC chemotherapy. Based on convenience for the subject and institution, the gap between drugs can range from 30 min to 2 hours (meal breaks, short walks, managing infusion-related reactions [IRRs], etc.) as long as the start times of every component of combination regimens are duly recorded.

### Generation of Bispecific Antibodies

The bispecific anti-CD40 anti-4-1BB (herein after referred to as GEN1042 or DuoBody-CD40×4-1BB) was produced with the humanized VH and VL sequences, the human kappa light chain, and a human IgG1 heavy chain described in Table 1. The CD40 binding arm has been produced with the human IgG1 heavy chain containing the following amino acid mutations: L234F, L235E, D265A and F405L (FEAL), wherein the amino acid position number is according to EU numbering (corresponding to SEQ ID NO: 33). The CD137 binding arm has been produced with a human IgG1 heavy chain containing the following amino acid mutations: 1,234F, L235E, D265A and K409R (FEAR), wherein the amino acid position number is according to EU numbering (correspond to SEQ ID NO: 34).

Bispecific IgG1 antibodies were generated by Fab-arm-exchange under controlled reducing conditions. The basis for this method is the use of complementary CH3 domains, which promote the formation of heterodimers under specific assay conditions as described in WO2011/131746. The F405L and K409R EU numbering) mutations were introduced into the relevant antibodies to create antibody pairs with complementary CH3 domains.

To generate bispecific antibodies, the two parental complementary antibodies, each antibody at a final concentration of 0.5 mg/ml, were incubated with 75 mM 2-mercaptoethylamine-HCI (2-MEA) in a total volume of 100 μL PBS at 31° C. for 5 hours. The reduction reaction was stopped by removing the reducing agent 2-MEA using spin columns (Microcon centrifugal filters, 30k, Millipore) according to the manufacturer's protocol.

### Inclusion Criteria for Combination Therapy

The subjects must be ≥18 years of age; have measurable disease according to RECIST 1.1; life expectancy ≥3 months; have an Eastern Cooperative Oncology Group (ECOG) Performance Status of 0-1; adequate organ, bone marrow, liver, coagulation, and renal function; and not received prior therapy with an anti-PD-1, anti-PD-L1, or anti-programmed death-ligand 2 agent or with an agent directed to another stimulatory or co-inhibitory T-cell receptor (eg, CTLA-4, OX-40, CD40 or 4-1BB). Additional criteria for each cohort are as follows:

### Melanoma

a. Histologically confirmed unresectable Stage III or Stage IV melanoma, as per American Joint Committee on Cancer (AJCC; version 8) staging system. Primary ocular or mucosal melanoma is excluded.
b. No prior systemic anticancer therapy for unresectable or metastatic melanoma.
c. With a known tumor BRAF mutation status as per local standard testing (preferably an FDA-approved test).
d. For subjects with BRAF V600E mutant melanoma, the following additional criteria should be met:
   i. Lactate dehydrogenase<local upper limit of normal
   ii. No clinically significant tumor related symptoms in the judgment of the investigator
   iii. Absence of rapidly progressing metastatic melanoma in the judgment of the investigator

### NSCLC

a. Have a histologically confirmed diagnosis of Stage IV metastatic or recurrent NSCLC (AJCC version 8), with no prior systemic anticancer therapy given as primary therapy for advanced or metastatic disease.
b. Tumor does not have an actionable EGFR activating mutation or ALK translocation. For subjects who are known to have a tumor of predominantly squamous histology, molecular testing for EGFR mutation and ALK translocation will not be required if this is per local SOC.
c. Tumor demonstrates PD-L1 expression in ≥1% of tumor cells (TPS≥1%) as assessed by immunohistochemistry (IHC) determined by a local SOC testing (preferably an FDA-approved test) or at a central laboratory. Central laboratory testing is mandated for the expansion phase.

### HNSCC

a. Histologically or cytologically-confirmed recurrent or metastatic HNSCC that is considered incurable by local therapies.
b. Subjects should not have had prior systemic therapy administered in the recurrent or metastatic setting. Systemic therapy which was completed more than 6 months prior to signing consent if given as part of multimodal treatment for locally advanced disease is allowed.
c. The eligible primary tumor locations are oropharynx, oral cavity, hypopharynx, and larynx.
d. Subjects should not have a primary tumor site of nasopharynx (any histology).
e. Have tumor PD-L1 IHC CPS≥1 per local (preferably an FDA-approved test) or central laboratory testing (central testing is mandated for the expansion phase).
f. Human papillomavirus (HPV) p16 test results available per local SOC for participants with oropharyngeal disease. Note: Oral cavity, hypopharynx, and larynx cancer are not required to undergo HPV testing by p16 IHC as by convention these tumor locations are assumed to be HPV negative.

### PDAC

a. Histologically or cytologically confirmed metastatic pancreatic adenocarcinoma. Pancreatic endocrine cancer is excluded.
b. No actionable gene alterations such as BRCA 1/2 or PALB2 mutations
c. No previous radiotherapy, surgery, chemotherapy, or investigational therapy for the treatment of metastatic disease
d. If a subject has had adjuvant/neoadjuvant therapy and/or therapy for locally advanced disease (chemotherapy for non-metastatic pancreatic cancer in combination with or without radiation therapy), all toxicities must have returned to baseline or ≤grade 1.

### Results

In the monotherapy dose escalation part of the Phase 1/2 GCT1042-01 trial, 50 subjects with metastatic or unresectable, non-central nervous system (CNS) solid tumors who exhausted standard of care therapy were treated at doses ranging from 0.1 mg to 400 mg Q3W. Two of 50 subjects treated in the dose escalation part had a confirmed partial response (4.0%) per RECIST v1.1, including 1 subject with neuroendocrine lung carcinoma and 1 subject with melanoma. Disease control was achieved in 50% of subjects. Disease control was defined as having a best overall response of complete, partial, and stable disease per RECIST v1.1.

Following the dose escalation part, 100 mg Q3W was selected for further evaluation in heavily pre-treated post-checkpoint inhibitor (CPI) NSCLC and melanoma subjects. Of the 22 post-CPI NSCLC subjects treated, 9 subjects (40.9%) achieved a best response of stable disease. No subjects in the post-CPI NSCLC cohort experienced a confirmed or partial response, and 6 subjects (27.3%) were not evaluable for response per RECIST v1.1. At the time of the data cut-off on 21-May-22, 1 subject in the post-CPI melanoma monotherapy expansion cohort treated with 100 mg of GEN 1042 Q3W was evaluable for response. The subject achieved a best overall response of stable disease.

Following the dose escalation part, GEN1042 100 mg Q3W was explored in combination with pembrolizumab 200 mg Q3W. As of the data cutoff of 21-May-22, a total of 10 subjects with metastatic melanoma, NSCLC, or HNSCC with no prior systemic anticancer therapy for metastatic disease were enrolled in the trial. The Objective Response Rate (ORR) per RECIST v 0.1 for all subjects treated was 40% ( 4/10). Two subjects experienced a complete response, and 2 subjects experienced a partial response. Disease control was achieved in 70% of subjects and was defined as having a best overall response of complete, partial, and stable disease per RECIST v1.1.

### Example 3: Effect of bsIgG1-CD40×4-1BB in Combination with Nivolumab on IFNγ Secretion in an Allogeneic MLR Assay

To analyze if the combination of bsIgG1-CD40×4-1BB (Fe active) with nivolumab could result in potentiation of cytokine production compared to single agent activity, an allogeneic MLR assay was performed in which co-cultures of one allogeneic pair of human mature dendritic cells (mDCs) and CD8+ T cells were incubated in the presence of bsIgG1-CD40×4-1BB alone, nivolumab alone or a combination of both antibodies. IFNγ secretion was assessed in the supernatants of the co-cultures using an IFNγ-specific immunoassay.

### Methods

Monocyte and T Cells from Healthy Donors

CD14+ monocytes and purified CD8+ T cells were obtained from Precision Medicine or BioIVT. Allogeneic donor pairs were used for the MLR assay.

### Differentiation of Monocytes to Immature Dendritic Cells

Human CD14+ monocytes were obtained from healthy donors. For differentiation into immature dendritic cells (iDCs), 1-1.5×10⁶ monocytes/mL were cultured for six days in Roswell Park Memorial Institute (RPMI) 1640 complete medium (ATCC modification formula; ThermoFisher, cat. no. A1049101) supplemented with 10% heat-inactivated Fetal Bovine Serum (FBS; Gibco, cat. no. 16140071), 100 ng/mL granulocyte-macrophage colony-stimulating factor (GM-CSF; BioLegend, cat. no. 766106) and 300 ng/mL interleukin-4 (IL-4; BioLegend, cat. no. 766206) in T25 culture flasks (Falcon, cat. no. 353108) at 37° C. Once during these six days, the medium was replaced with fresh medium with supplements.

Maturation of iDCs

To mature the iDCs, the cells were harvested by collecting non-adherent cells, counted, incubated at 1-1.5×10⁶ cells/mL in RPMI 1640 complete medium supplemented with 10% FBS, 100 ng/mL GM-CSF, 300 ng/mL IL-4 and 1× with lipopolysaccharide (LPS; ThermoFisher, cat. no. 00-4976-93) for 24 h prior to start of the mixed lymphocyte reaction (MLR) assay at 37° C.

### Mixed Lymphocyte Reaction (MLR)

One day prior to the start of an MLR assay, purified CD8+ T cells obtained from allogeneic healthy donors were thawed. Cells were resuspended at 1×10⁶ cells/mL in RPMI 1640 complete medium supplemented with 10% FBS and 10 ng/mL IL-2 (BioLegend, cat. no. 589106) and incubated O/N at 37° C.

The next day, the LPS-matured dendritic cells (mDCs, see Maturation of iDCs) and allogeneic purified CD8+ T cells were harvested and resuspended in AIM-V medium (ThermoFisher, cat. no. 12055091) at 4×10⁵ cells/mL and 4×10⁶ cells/mL, respectively.

In cocultures, 20,000 mDCs were incubated with 200,000 allogeneic purified CD8+ T cells (DC:T cell ratio of 1:10) in the presence of bsIgG1-CD40×4-1BB (0.001-10 μg/mL) either alone or in combination with the nivolumab MDX-1106 (0.0005-5 μg/mL), and IgG1-ctrl (0.001-10 μg/mL) in AIM-V medium in a 96-well round-bottom plate (Falcon, cat. no. 353227) at 37° C. After 5 days, the plates were centrifuged at 500×g for 5 min and the supernatant was carefully transferred from each well to a new 96-well round bottom plate.

The collected supernatants from the MLR assay were analyzed for interferon γ (IFNγ) levels by enzyme-linked immunosorbent assay (ELISA) using an Alpha Lisa IFNγ kit (Perkin Elmer, cat. no. AL217) on an Envision instrument, according to the manufacturer's instructions.

TABLE 11
Antibodies

| Test compound | SEQ IDs |
|---|---|
| bsIgG1-CD40 × 4-1BB¹ | SEQ ID: 9, 10, 19, 20, 30, 31, 27 |
| Nivolumab IgG1-PD1- | SEQ ID: 57, 58, 29, 25, 27 |
| MDX-1106-FEAL² | |
| IgG1-ctrl³ | SEQ ID: 59, 60, 21, 27 |

¹This antibody comprises the CD40-specific and 4-1BB-specific Fab arms presented in Table 1 in an Fc-active IgG1 backbone (i.e. without the Fc-silencing L234F, L235E, D265A mutations).
²This antibody comprises the CDR sequences of nivolumab (MDX-1106) presented in Table 1 in an Fc-inert IgG1 backbone (i.e. with the Fc-silencing L234F, L235E, D265A mutations and DuoBody mutation F405L).
³Based on anti-HIV gp120 antibody IgG1-b12 (Barbas et al., J Mol Biol 1993, 230: 812-823).

### Results

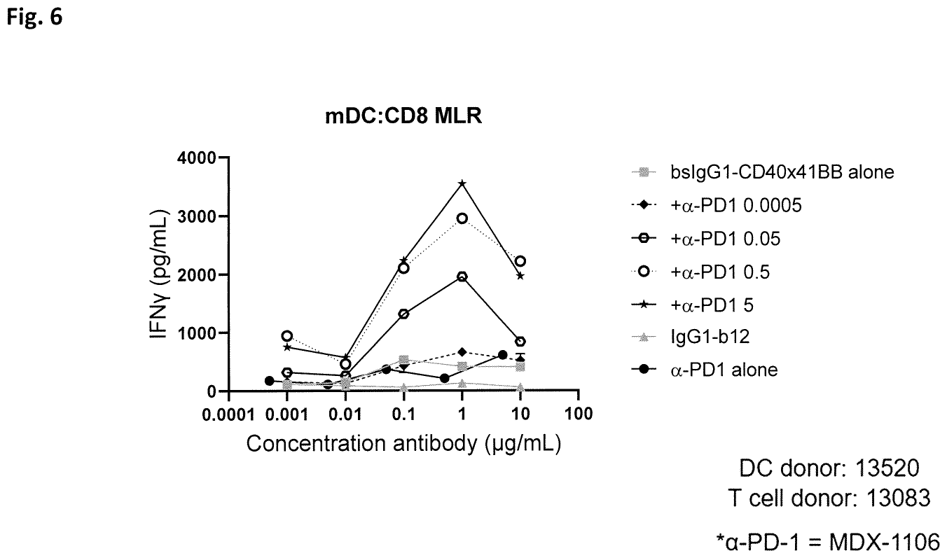

BsIgG1-CD40×4-1BB enhanced IFNγ secretion in co-cultures of purified CD8⁺ T cells and allogeneic mDCs compared to IgG1-ctrl (see FIG. 6). Concurrent exposure to bsIgG1-CD40×4-1BB and 0.05-5 μg/mL nivolumab (IgG1-PD1-MDX1106-FEAL, α-PD1) induced a dose-dependent increase in IFNγ compared to either bsIgG1-CD40×4-1BB alone or the nivolumab alone (FIG. 6).

### Conclusion

This data suggests that the magnitude of immune response can be amplified through targeting of the PD-1/PD-L1 axis by using anti-PD1 antibodies in combination with CD40 and 4-1BB co-stimulation.

### Example 4: Effect of DuoBody-CD40×4-1BB in Combination with IgG1-PD1 on IFNγ Secretion in an Allogeneic MLR Assay

To analyze if the combination of DuoBody-CD40×4-1BB with IgG1-PD1 (an Fe inert anti-PD-1 monoclonal antibody of the IgG1 isotype) could result in potentiation of cytokine production in a mixed lymphocyte reaction (MLR) assay compared to single agent activity, two unique, allogeneic pairs of human mature dendritic cells (mDCs) and CD8+ T cells were co-cultured in the presence of DuoBody-CD40×4-1B13 alone, IgG1-PD1 alone or a combination of both antibodies. IFNγ secretion was assessed in the supernatants of the co-cultures using an IFNγ-specific immunoassay.

### Methods

Monocytes and T Cells from Healthy Donors

CD14+ monocytes and purified CD8+ T cells were obtained from BioIVT. Two unique allogeneic donor pairs were used for the MLR assay.

### Differentiation of Monocytes to Immature Dendritic Cells

Human CD14+ monocytes were obtained from healthy donors. For differentiation into immature dendritic cells (iDCs), 1-1.5×10⁶ monocytes/mL were cultured for six days in Roswell Park Memorial Institute (RPMI) 1640 complete medium (ATCC modification formula; ThermoFisher, cat. no. A1049101) supplemented with 10% heat-inactivated Fetal Bovine Serum (FBS; Gibco, cat. no. 16140071), 100 ng/mL granulocyte-macrophage colony-stimulating factor (GM-CSF; BioLegend, cat. no. 766106) and 300 ng/mL interleukin-4 (IL-4; BioLegend, cat. no. 766206) in T25 culture flasks (Falcon, cat. no. 353108) at 37° C. After four days, the medium was replaced with fresh medium and supplements.

Differentiation of iDCs to mDCs

Prior to start of the MLR assay, iDCs were harvested by collecting non-adherent cells and differentiated to mature DCs (mDCs) by incubating 1-1.5×10⁶ cells/mL in RPMI 1640 complete medium supplemented with 10% FBS, 100 ng/mL GM-CSF, 300 ng/mL IL-4 and 5 μg/mL lipopolysaccharide (LPS; ThermoFisher, cat. no. 00-4976-93) for 24 h at 37° C.

### Mixed Lymphocyte Reaction (MLR)

One day prior to the start of an MLR assay, purified CD8+ T cells obtained from allogeneic healthy donors were thawed, resuspended at 1×10⁶ cells/mL in RPMI 1640 complete medium supplemented with 10% FBS and 10 ng/mL IL-2 (BioLegend, cat. no. 589106) and incubated O/N at 37° C. The next day, the LPS-matured dendritic cells (mDCs, see Maturation of iDCs) and allogeneic purified CD8+ T cells were harvested and resuspended in AIM-V medium (ThermoFisher, cat. no. 12055091) at 4×10⁵ cells/mL and 4×10⁶ cells/mL, respectively. Co-cultures were seeded at a DC:T cell ratio of 1:10, corresponding to 20,000 mDCs incubated with 200,000 allogeneic purified CD8+ T cells, and cultured in the presence of IgG1-PD1 (0.001-100 μg/mL) as single agent, DuoBody-CD40×4-1BB (0.001-30 μg/mL) as single agent, or both agents combined corresponding to a dose-response matrix of 7×7 combinations in AIM-V medium in a 96-well round-bottom plate (Falcon, cat. no. 353227) at 37° C. for 5 days. Co-cultures treated with IgG1-ctrl-FERR (100 μg/mL), bsIgG1-CD40×ctrl (30 μg/mL), bsIgG1-ctrl×4-1BB (30 μg/mL), and IgG1-ctrl-FEAL (30 μg/mL) were included as controls. After 5 days, the plates were centrifuged at 500×g for 5 min and the supernatant was carefully transferred from each well to a new 96-well round bottom plate.

The collected supernatants from the MLR assay were analyzed for IFNγ levels by enzyme-linked immunosorbent assay (ELISA) using an Alpha Lisa IFNγ kit (Perkin Elmer, cat. no. AL217) on an Envision instrument, according to the manufacturer's instructions.

TABLE 12

| Test compound | Comprising SEQ ID NOs |
|---|---|
| DuoBody-CD40 × 4-1BB | SEQ ID NO: 9, 10, 19, 20, 25, 26, 27 |
| bsIgG1-CD40 × ctrl¹ | SEQ ID NO: 9, 10, 25, 26, 27 |
| bsIgG1-ctrl × 4-1BB¹ | SEQ ID NO: 19, 20, 25, 26, 27 |
| IgG1-PD1 | SEQ ID NO: 27, 43, 44, 61 |
| IgG1-ctrl-FEAL¹ | SEQ ID NO: 25, 27 |
| IgG1-ctrl-FERR¹ | SEQ ID NO: 27, 59, 60, 62 |

¹Control binding moiety based on anti-HIV gp120 antibody IgG1-b12 (Barbas et al., J Mol Biol 1993, 230: 812-823)

### Synergy Analysis

In the synergy analysis, data was processed for each donor pair separately. The concentrations of IFNγ (μg/mL) in each treatment condition were normalized by subtracting the control values (no treatment control wells) and expressed as percentage of the maximal value in the assay (IFNγ induction). The IFNγ induction values represent an average of two replicates. The interaction between the two antibodies combined was analyzed using the SynergyFinder package (v 3.2.2; Zheng et al., 2021 bioRxiv https://doi.org/10.1101/2021.06.01.446564) in R (v 4.1.0). The synergistic effect was defined as the excess of observed effect over expected effect as calculated by two reference models (synergy scoring models): Highest Single Agent (HSA; Berenbaum, 1989 Pharmacol Rev. 41: 93-141) and Bliss (Bliss, 1939 Annals of Applied Biol. 26:585-615).

### Results & Conclusion

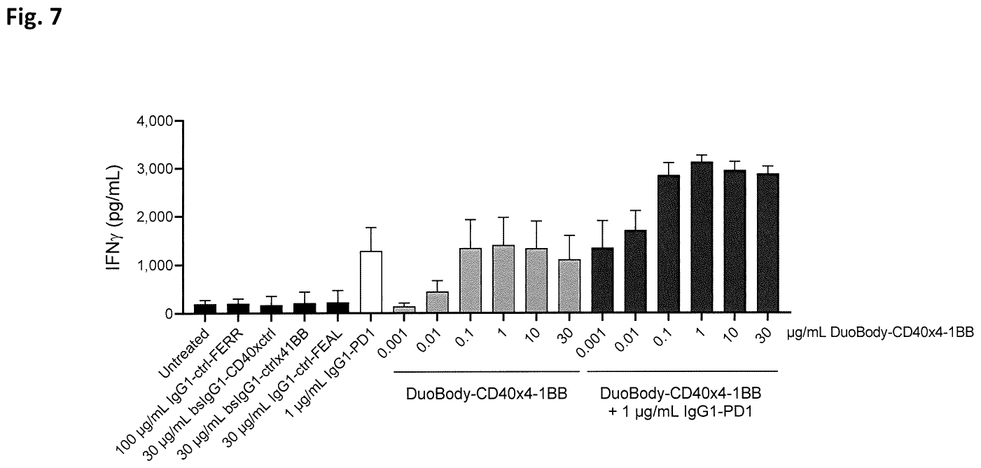

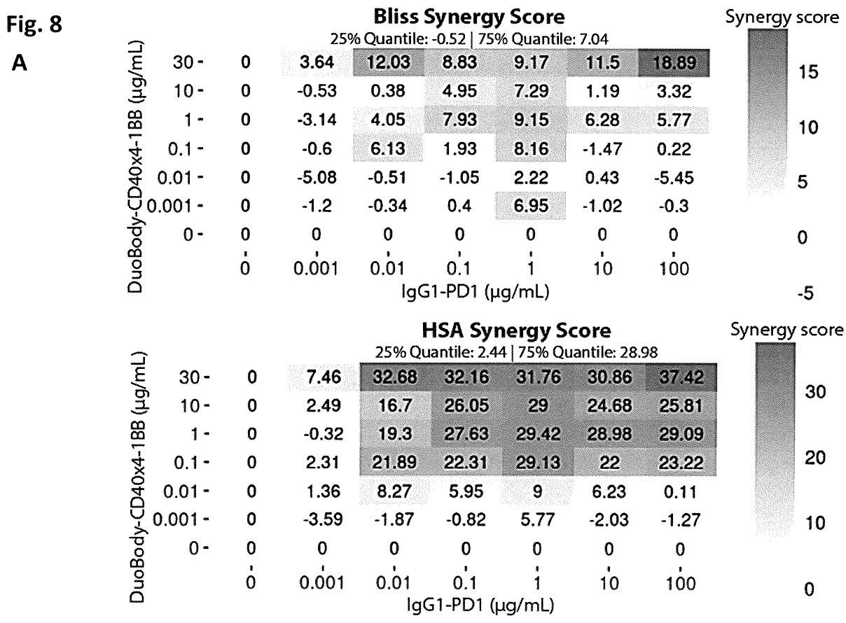

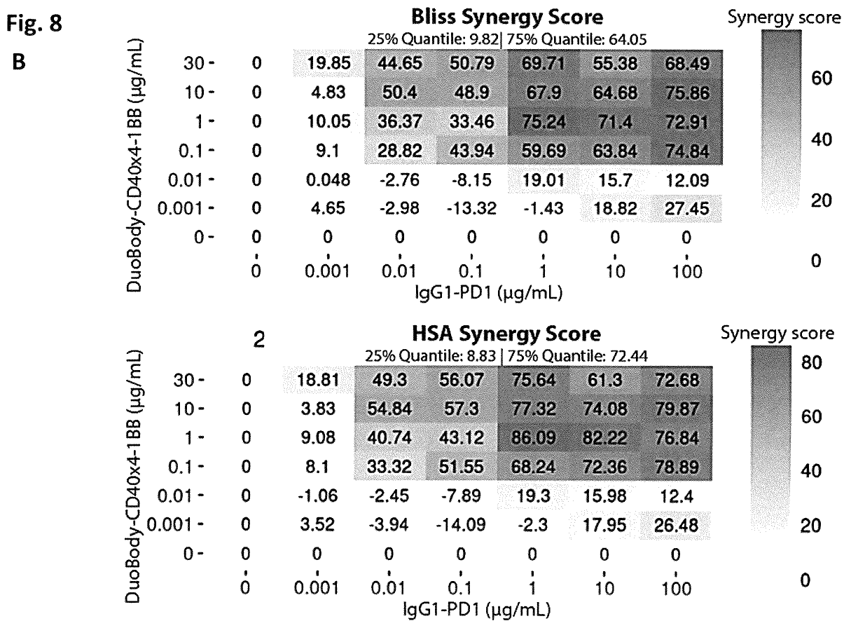

Treatment with either DuoBody-CD40×4-1BB or IgG1-PD1 alone enhanced the secretion of IFNγ in the MLR assay; combination of DuoBody-CD40×4-1BB with 1 μg/mL IgG1-PD1 further potentiated secretion of IFNγ compared to single-agent activity (FIG. 7). For both unique donor pairs, combined treatment with DuoBody-CD40×4-1BB and IgG1-PD1 resulted in a synergistic effect across a range of concentration combinations as determined using the Bliss and HSA synergy scoring models (FIG. 8).

### Example 5: Antigen-Specific Stimulation Assay to Determine the Capacity of IgG1-PD1 in Combination with DuoBody-CD40×4-1BB to Enhance T-Cell Proliferation and Cytokine Secretion

To determine the combinatorial effect of DuoBody-CD40×4-1BB and IgG1-PD1 on T-cell proliferation and cytokine production compared to single-agent activity, an antigen-specific stimulation assay was conducted using co-cultures of PD-1-overexpressing human CD8+ T cells and cognate antigen-expressing immature dendritic cells (iDCs).

### Methods

### Isolation of Cells and Differentiation of Monocytes to Immature Dendritic Cells

HLA-A*02⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were cryopreserved for T-cell isolation. For differentiation into iDCs, 1×10⁶ monocytes/mL were cultured for five days in RPMI 1640 (Life Technologies GmbH, cat. no. 61870-010) containing 5% pooled human serum (One Lambda Inc., cat. no. A25761), 1 mM sodium pyruvate (Life technologies GmbH, cat. no. 11360-039), 1× non-essential amino acids (Life Technologies GmbH, cat. no. 11140-035), 200 ng/mL granulocyte-macrophage colony-stimulating factor (GM-CSF; Miltenyi, cat. no. 130-093-868) and 200 ng/mL interleukin-4 (IL-4; Miltenyi, cat. no. 130-093-924). On day 3, half of the medium was replaced with fresh medium containing supplements. iDCs were harvested by collecting non-adherent cells and adherent cells were detached by incubation with Dulbecco's phosphate-buffered saline (DPBS) containing 2 mM EDTA for 10 min at 37° C. After washing with DPBS iDCs were cryopreserved in FBS (Sigma-Aldrich, cat. no. F7524) containing 10% DMSO (AppliChem GmbH, cat. no A3672,0050) for future use in antigen-specific T cell assays.

Electroporation of iDCs and CD8+ T Cells and CFSE-Labeling

One day prior to the start of an antigen-specific CD8+ T cell stimulation assay, frozen PBLs and iDCs from the same donor were thawed. CD8+ T cells were isolated from PBLs by MACS technology using anti-CD8 MicroBeads (Miltenyi, cat. no. 130-045-201), according to the manufacturer's instructions. About 10×10⁶ to 15×10⁶ CD8+ T cells were electroporated with each 10 μg of in vitro transcribed (IVT)-RNA encoding the alpha and beta chains of a murine TCR specific for human claudin-6 (CLDN6; HLA-A*02-restricted; described in WO 2015150327 A1) plus 10 μg IVT-RNA encoding human PD-1 (UniProt Q15116) in 250 μL X-Vivol5 medium (Lonza, cat. no. BE02-060Q). The cells were transferred to a 4-mm electroporation cuvette (VWR International GmbH, cat. no. 732-0023) and electroporated using the BTX ECM® 830 Electroporation System (BTX; 500 V, 3 ms pulse). Immediately after electroporation, cells were transferred into fresh IMDM GlutaMAX medium (Life Technologies GmbH, cat. no. 319800-030) containing 5% pooled human serum and rested at 37° C., 5% CO₂ for at least 1 hour. T cells were labeled using 0.8 μM carboxyfluorescein succinimidyl ester (CFSE; Life Technologies GmbH, cat. No V12883) in PBS according to the manufacturer's instructions and incubated in IMDM medium supplemented with 5% human AB serum overnight.

Up to 5×10⁶ thawed iDCs were electroporated with 2 μg IVT-RNA encoding full-length human CLDN6 (WO 2015150327 A1), in 250 μL X-Vivol5 medium, using the electroporation system as described above (300 V, 12 ms pulse) and incubated in IMDM medium supplemented with 5% pooled human serum overnight.

The next day, cells were harvested. Cell-surface expression of CLDN6 on iDCs, as well as cell-surface expression of the CLDN6-specific TCR and PD-1 on T cells was confirmed by flow cytometry. To this end, iDCs were stained with a fluorescently labeled CLDN6-specific antibody (non-commercially available; in-house production). T cells were stained with a brilliant violet (BV)421-conjugated anti-mouse TCR-β chain antibody (Becton Dickinson GmbH, cat. no. 562839) and an allophycocyanin (APC)-conjugated anti-human PD-1 antibody (Thermo Fisher Scientific, cat. no. 17-2799-42).

### Antigen-Specific In Vitro T-Cell Stimulation Assay

Electroporated iDCs were incubated with electroporated, CFSE-labeled T cells at a ratio of 1:10 in the presence of IgG1-PD1 (0.8 μg/mL), pembrolizumab (Keytruda®, Merck Sharp & Dohme GmbH, PZN 10749897) (0.8 μg/mL), or the negative control antibody IgG1-ctrl-FERR (0.8 μg/mL), either alone or in combination with DuoBody-CD40×4-1BB (0.0022, 0.0067, or 0.2 μg/mL), in IMDM medium containing 5% pooled human serum in a 96-well round-bottom plate. After 4 days of culture, the cells were stained with an APC-conjugated anti-human CD8 antibody. T-cell proliferation was evaluated by flow cytometry analysis of CFSE dilution in CD8+ T cells using a BD FACSCelesta™ flow cytometer (Becton Dickinson GmbH).

Flow cytometry data was analyzed using FlowJo software version 10.7.1. CFSE label dilution of CD8+ T cells was assessed using the proliferation modeling tool in FlowJo, and expansion indices calculated using the integrated formula.

### Determination of Cytokine Concentrations

Cytokine concentrations in supernatants that had been collected from T cell/iDC co-cultures after 4 days were determined by multiplexed electrochemiluminescence immunoassay using a custom-made U-Plex biomarker group 1 (human) assay for the detection of panel of 10 human cytokines (GM-CSF, IL-2, IL-8, IL-10, IL-12p70, IL-13, interferon [IFN]γ, IFNγ-inducible protein [IP]-10 [also known as C-X-C motif chemokine ligand 10], macrophage chemoattractant protein [MCP]1, and tumor necrosis factor [TNF]α; Meso Scale Discovery, cat. No. K15067L-2) following the manufacturer's protocol.

TABLE 13
Antibodies

| Test compound | Supplier, cat. no. | Comprising SEQ ID NOs |
|---|---|---|
| DuoBody-CD40 × 4-1BB | N/A | SEQ ID NO: 9, 10, 19, 20, 25, 26, 27 |
| IgG1-PD1 | N/A | SEQ ID NO: 27, 43, 44, 61 |
| IgG1-ctrl-FERR¹ | N/A | SEQ ID NO: 27, 59, 60, 62 |
| pembrolizumab | Keytruda ®, Merck Sharp & | N/A |
| | Dohme GmbH, PZN 10749897 | |

¹Control binding moiety based on anti-HIV gp120 antibody IgG1-b12 (Barbas et al., J Mol Biol 1993, 230: 812-823)

### Results

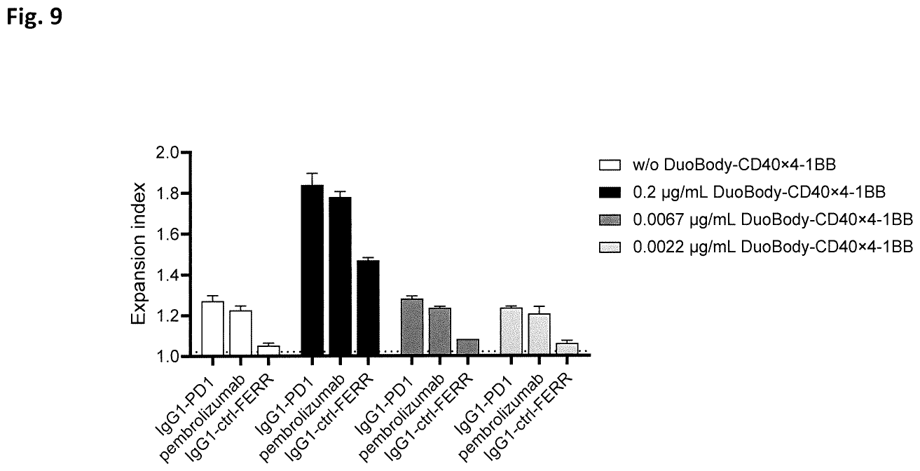

Combination treatment with IgG1-PD1 and DuoBody-CD40×4-1BB potentiated CD8+ T-cell proliferation, compared to DuoBody-CD40×4-1BB combined with non-binding control antibody IgG1-ctrl-FERR and compared to IgG1-PD1 as single treatment (FIG. 9). An increase in proliferation was seen when a high concentration of DuoBody-CD40×4-1BB (0.2 μg/mL) was used in combination treatment, compared to both single-agent treatments. Combination of IgG1-PD1 with low (0.0022 μg/mL) or intermediate (0.0067 μg/mL) concentrations of DuoBody-CD40×4-1BB had minimal or no effect on proliferation, compared to IgG1-PD1 alone. Combination treatment with pembrolizumab and a high concentration of DuoBody-CD40×4-1BB also enhanced proliferation compared to both compounds as single agents.

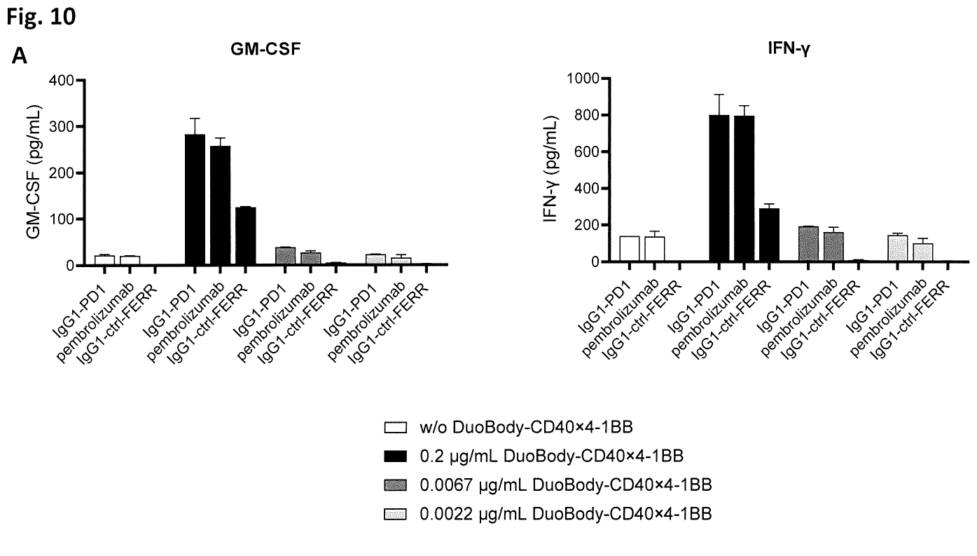

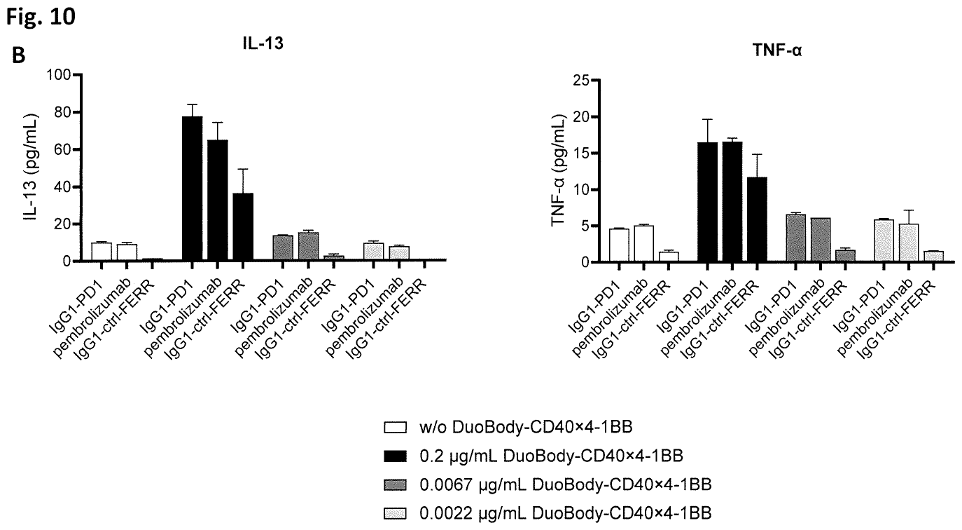

Combination treatment with IgG1-PD1 and DuoBody-CD40×4-1BB enhanced the secretion of the proinflammatory cytokines GM-CSF, IFNγ, IL-13, and TNFα, compared to DuoBody-CD40×4-1BB combined with IgG1-ctrl-FERR and compared to IgG1-PD1 as single treatment (FIG. 10). An increase in cytokine secretion was seen when a high concentration of DuoBody-CD40×4-1BB (0.2 μg/mL) was used in combination treatment, compared to both single-agent treatments. Combination of IgG1-PD1 with low (0.0022 μg/mL) or intermediate (0.0067 μg/mL) concentrations of DuoBody-CD40×4-1BB had minimal or no effect on cytokine secretion, compared to IgG1-PD1 alone. Combination treatment with pembrolizumab and a high concentration of DuoBody-CD40×4-1BB also enhanced cytokine secretion compared to both compounds as single agents. Secretion of other cytokines tested were not consistently enhanced, or not enhanced, compared to single-agent treatments.

### Example 6: Generation of IgG1-PD1 and Screening Materials

### PD-1 and FcγR Constructs

Plasmids encoding various full-length PD-1 variants were generated: human (Homo sapiens; UniProtKB ID: Q15116), cynomolgus monkey (Macaca fascicularis; UniProtKB ID: B0LAJ3), dog (Canis familiaris; UniProtKB ID: E2RPS2), rabbit (Oryctolagus cuniculus; UniProtKB ID: G1SUF0), pig (Sus scrofa; UniProtKB ID: A0A287A1C3), rat (Rattus norvegicus; UniProtKB ID: D3ZIN8), and mouse (Mus musculus; UniProtKB ID: Q02242), as well as a plasmid encoding human FcγRIa (UniProt KB ID: P12314).

### Generation of CHO-S Cell Lines Transiently Expressing Full-Length PD-1 or FcγR Variants

CHO-S cells (a subclone of CHO cells adapted to suspension growth; ThermoFisher Scientific, cat. no. R800-07) were transfected with PD-1 or FcγR plasmids using FreeStyle™ MAX Reagent (ThermoFisher Scientific, cat. no. 16447100) and OptiPRO™ serum-free medium (ThermoFisher Scientific, cat. no. 12309019), according to the manufacturer's instructions.

### Production of Antibody Variants

### IgG1-PD1

Three New Zealand White rabbits were immunized with recombinant human His-tagged PD-1 protein (R&D Systems, cat. no. 8986-PD). Single B cells from blood were sorted and supernatants screened for production of PD-1 specific antibodies by human PD-1 enzyme-linked immunosorbent assay (ELISA), cellular human PD-1 binding assay and by human PD-1/PD-L1 blockade bioassay. From screening-positive B cells, RNA was extracted, and sequencing was performed. The variable regions of heavy and light chain were gene synthesized and cloned N-terminal of human immunoglobulin constant parts (IgG1/K) containing mutations L234A and L235A (LALA) wherein the amino acid position number is according to EU numbering (SEQ ID NO: 70) to minimize interactions with Fey receptors.

### Transient Transfections of HEK293-FreeStyle Cells Using 293-Free Transfection Reagent

(Novagen/Merck) were executed by Tecan Freedom Evo device. Produced chimeric antibodies were purified from cell supernatant using protein-A affinity chromatography on a Dionex Ultimate 3000 HPLC with plate autosampler. Purified antibodies were used for further analysis in particular retesting by human PD-1 ELISA, cellular human PD-1 binding assay, human PD-1/PD-L1 blockade bioassay, and T-cell proliferation assay. The chimeric rabbit antibody MAB-19-0202 (SEQ ID NO: 71 and 72) was identified as best performing clone and subsequently humanized.

The variable region sequences of the chimeric PD-1 antibody MAB-19-0202 are shown in the following tables. Table 14 shows the variable regions of the heavy chain, while Table 15 shows the variable regions of the light chain. In both cases the framing regions (FRs) as well as the complementarity determining regions (CDRs) according to Kabat numbering are defined. The underlined amino acids indicate the CDRs according to the IMGT numbering. The bold letters indicate the intersection of Kabat and IMGT numbering.

TABLE 14
Framing and variable regions of the heavy
chain of MAB-19-0202 (MAB-19-0202-HC; SEQ
ID NO: 71) (including CDRs according to
Kabat (bold letters) or IMGT (underlined)
numbering)

| | CDR1 | | CDR2 | | CDR3 | |
|---|---|---|---|---|---|---|
| | (SEQ | | (SEQ | | (SEQ | |
| | ID | | ID | | ID | |
| FR1 | NO) | FR2 | NO) | FR3 | NO) | FR4 |
| aa | SYNM | aa | IISG | aa | AFYDD | aa |
| 1 - 29 | G | 35 - 48 | GTIG | 65 - 95 | YDYNV | 106 - 116 |
| | (64) | | HYAS | | (47) | |
| | | | WAKG | | | |
| | | | (66) | | | |

TABLE 15
Framing and variable regions of the light
chain of MAB-19-0202 (MAB-19-0202-LC; SEQ
ID NO: 72) (including CDRs according to
Kabat (bold letters) or IMGT (underlined)
numbering)

| | CDR1 | | CDR2 | | CDR3 | |
|---|---|---|---|---|---|---|
| | (SEQ | | (SEQ | | (SEQ | |
| | ID | | ID | | ID | |
| FR1 | NO) | FR2 | NO) | FR3 | NO) | FR4 |
| aa | QSSQ | aa | QASK | aa | AGGY | aa |
| 1 - 23 | SVYG | 37 - 51 | LET | 59 - 90 | SSSS | 102 - 111 |
| | NNQL | | (69) | | DTT | |
| | S | | | | (50) | |
| | (68) | | | | | |

Humanized heavy and light chain variable region antibody sequences were generated by structural modelling-assisted CDR grafting, gene synthesized and cloned N-terminal of human immunoglobulin constant parts (IgG1/x with LALA mutations). Humanized antibodies were used for further analysis in particular retesting by human PD-1 ELISA, cellular human PD-1 binding assay, human PD-1/PD-L1 blockade bioassay, and the T-cell proliferation assay. The humanized antibody MAB-19-0618 (SEQ ID NO: 43 and 44) was identified as best performing clone.

The allocation of the humanized light and heavy chains to antibody ID of the recombinant humanized sequences are listed in Table 16. The variable region sequences of the humanized light and heavy chains are shown in Table 17 and 18. Table 17 shows the variable regions of the heavy chain, while Table 18 shows the variable regions of the light chain. In both cases the framing regions (FRs) as well as the complementarity determining regions (CDRs) according to Kabat numbering are defined. The underlined amino acids indicate the CDRs according to the IMGT numbering.

TABLE 16
Allocation of the humanized light and heavy chains to
antibody ID of the recombinant humanized sequences

| | Light chain | Heavy chain |
|---|---|---|
| Antibody ID | (SEQ ID NO: 44) | (SEQ ID NO: 43) |
| MAB-19-0618 | humanized variant derived | humanized variant derived |
| | from MAB-19-0202-L4 | from MAB-19-0202-H5 |

TABLE 17
Framing and variable regions of the heavy
chain of MAB-19-0618 (SEQID NO: 43, derived
from MAB-19-0202-H5), including CDRs
according to Kabat (bold letters) or IMGT
(underlined) numbering

| | CDR1 | | CDR2 | | CDR3 | |
|---|---|---|---|---|---|---|
| | (SEQ | | (SEQ | | (SEQ | |
| | ID | | ID | | ID | |
| FR1 | NO) | FR2 | NO) | FR3 | NO) | FR4 |
| aa | SYNMG | aa | IISG | aa | AFYD | aa |
| 1 - 30 | (45; | 36 - 49 | GTIG | 66 - 97 | DYD | 108 - 118 |
| | 63; | | HYAS | | YNV | |
| | 64) | | WAKG | | (47; | |
| | | | (46; | | 67) | |
| | | | 65; | | | |
| | | | 66) | | | |

TABLE 18
Framing and variable regions of the light
chain of MAB-19-0618 (SEQID NO: 44, derived
from MAB-19-0202-L4), including CDRs
according to Kabat (bold letters) or IMGT
(underlined )numbering

| | CDR1 | | CDR2 | | CDR3 | |
|---|---|---|---|---|---|---|
| | (SEQ | | (SEQ | | (SEQ | |
| | ID | | ID | | ID | |
| FR1 | NO) | FR2 | NO) | FR3 | NO) | FR4 |
| aa | QSSQ | aa | QASK | aa | AGGY | aa |
| 1 - 23 | SVYG | 37 - 51 | LET | 59 - 90 | SSSS | 102 - 111 |
| | NNQL | | (49, | | DTT | |
| | S | | 69) | | (50) | |
| | (48; | | | | | |
| | 68) | | | | | |

The sequences of the variable regions of the heavy and light chains of MAB-19-0618 were gene synthesized and cloned by ligation-independent cloning (LIC) into expression vectors with codon-optimized sequences encoding the human IgG1m(f) heavy chain constant domain containing the Fc-silencing mutations L234F, L235E and G236R (FER) wherein the amino acid position number is according to EU numbering (SEQ ID NO: 61) and the human kappa light chain constant domain (SEQ ID NO: 27). The resulting antibody was designated IgG1-PD1.

The GS Xceed® Expression System (Lonza) was used to generate a stable cell line expressing IgG1-PD1. The sequences encoding the heavy and light chain of IgG1-PD1 were cloned into the expression vectors pXC-18.4 and pXC-Kappa (containing the glutamine synthetase [GS] gene), respectively, by Lonza Biologics plc. Next, a double gene vector (DGV) encoding both the heavy and light chain of IgG1-PD1 was constructed by ligating the complete expression cassette from the heavy chain vector into the light chain vector. The DNA of this DGV was linearized with the restriction enzyme Pvul-HF (New England Biolabs, R3150L) and used for stable transfection of CHOK1SV® GS-KO® cells. IgG1-PD1 was purified for functional characterization.

### IgG1-CD52-E430G

A human IgG1 antibody with an E430G hexamerization-enhancing mutation (WO 2013/004842 A2) in the Fc domain (SEQ ID NO: 73) and antigen-binding domains identical to CAMPATH-1H, a CD52-specific antibody, was used as a positive control in C1q binding experiments (Crowe et al., 1992 Clin Exp Immunol. 87(1):105-110) (SEQ ID NO. 74 and 75).

### Control Antibodies

Human IgG1 antibodies with antigen-binding domains identical to b12, an HIV1 gp120-specific antibody, were used as negative controls in several experiments (Barbas et al., J Mol Biol. 1993 Apr. 5; 230(3):812-2). VH and V₁ domains of b12 (SEQ ID NO 59 and 60) were prepared by de novo gene synthesis (GeneArt Gene Synthesis; ThermoFisher Scientific, Germany) and cloned into expression vectors containing a human IgG1 heavy chain constant region (i.e. CH1, hinge, CH2 and CH3 region) of the human IgG1m(f) allotype (SEQ ID NO: 29) or a variant thereof (containing the L234F/L235E/G236R mutations and an additional, in the context of this study functionally irrelevant, K409R mutation in the Fc domain, abbreviated as the FERR mutations) (SEQ ID NO:62) or containing a human IgG4 heavy chain constant region (SEQ ID NO: 76); or the constant region of the human kappa light chain (LC) (SEQ ID NO: 27), as appropriate for the selected binding domains. Antibodies were obtained by transfection of heavy and light chain expression vectors in production cell lines and purified for functional characterization.

### Example 7: Binding of IgG1-PD1 to PD-1 from Various Species

Binding of IgG1-PD1 to PD-1 of species commonly used for nonclinical toxicology studies was assessed by flow cytometry using CHO-S cells transiently expressing PD-1 from different animal species.

CHO-S cells (5×10⁴ cells/well) were seeded in round-bottom 96-well plates. Antibody dilutions (1.7×10⁻⁴-30 μg/mL or 5.6×10⁻⁵-10 μg/mL, 3 fold dilutions) of IgG1-PD1, IgG1-ctrl-FERR, and pembrolizumab were prepared in Genmab (GMB) fluorescence-activated cell sorting (FACS) buffer (phosphate-buffered saline [PBS; Lonza, cat. no. BE17-517Q, diluted to 1×PBS in distilled water] supplemented with 0.1% [w/v] bovine serum albumin [BSA; Roche, cat. no. 10735086001] and 0.02% [w/v] sodium azide [NaN₃; bioWORLD, cat. no. 41920044-3]). An IgG4 isotype control (BioLegend, cat. no. 403702) for pembrolizumab was included only at the highest concentration tested (30 μg/mL or 10 μg/mL). Cells were centrifuged, supernatant was removed, and cells were incubated in 50 μL of the antibody dilutions for 30 min at 4° C. Cells were washed twice with GMB FACS buffer and incubated with 50 μL secondary antibody R-phycoerythrin (PE)-conjugated goat-anti-human IgG F(ab′)₂ (Jackson ImmunoResearch, cat. no. 109-116-098; diluted 1:500 in GMB FACS buffer) for 30 min at 4° C., protected from light. Cells were washed twice with GMB FACS buffer, resuspended in GMB FACS buffer supplemented with 2 mM ethylenediaminetetraacetic acid (EDTA; Sigma-Aldrich, cat. no. 03690) and 4′,6-diamidino-2-phenylindole (DAPI) viability marker (1:5,000; BD Pharmingen, cat. no. 564907). Antibody binding to viable cells (as identified by DAPI exclusion) was analyzed by flow cytometry on an Intellicyt® iQue PLUS Screener (Intellicyt Corporation) using FlowJo software. Binding curves were analyzed using non-linear regression analysis (four-parameter dose-response curve fits) in GraphPad Prism.

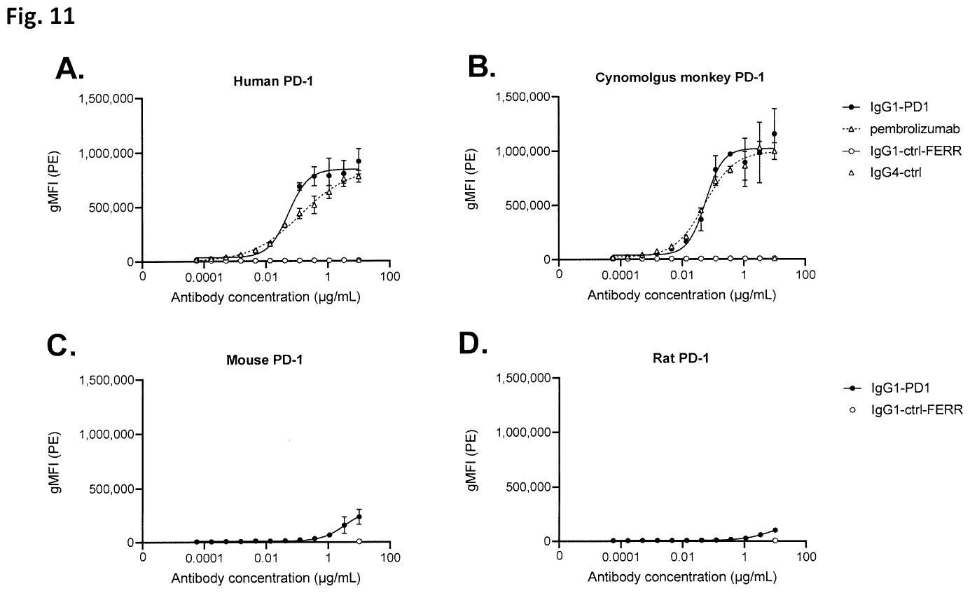

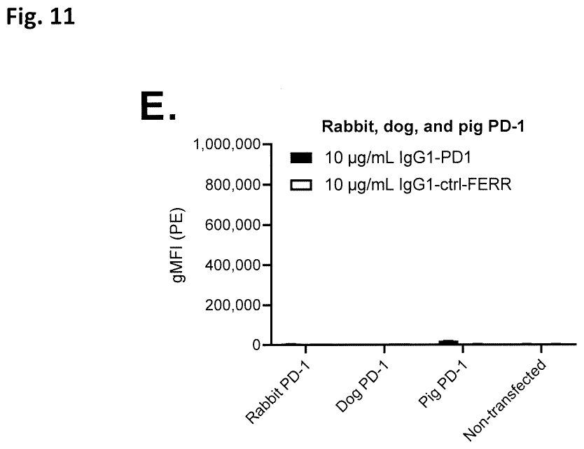

Binding of IgG1-PD1 to PD-1 of different species was evaluated by flow cytometry using CHO-S cells transiently transfected to express human, cynomolgus monkey, dog, rabbit, pig, rat, or mouse PD-1 protein on the cell surface. Dose-dependent binding of IgG1-PD1 was observed for human and cynomolgus monkey PD-1 (FIG. 11A-B). Pembrolizumab demonstrated comparable binding. Substantially reduced cross-reactivity of IgG1-PD1, and only at the highest concentrations, was observed to rodent PD-1 (mouse, rat; FIGS. 11C-D) and no binding was observed to PD-1 of other species frequently used in toxicology studies (rabbit, dog, pig; FIG. 11E). No IgG1-PD1 binding was observed to non-transfected control cells (FIG. 11E), nor was binding of IgG1-ctrl-FERR, included as a negative control, observed to PD-1 of any of the tested species (FIG. 11).

In conclusion, IgG1-PD1 showed comparable binding to membrane-expressed human and cynomolgus monkey PD-1 and significantly lower or no binding to mouse, rat, rabbit, dog, and pig PD-1.

### Example 8: Binding to Human and Cynomolgus Monkey PD-1 Determined by Surface Plasmon Resonance

Binding of immobilized IgG1-PD1, pembrolizumab, and nivolumab to human and cynomolgus monkey PD-1 was analyzed by surface plasmon resonance (SPR) using a Biacore 8K SPR system. Recombinant human and cynomolgus monkey PD-1 extracellular domain (ECD) with a C-terminal His-tag were obtained from Sino Biological (cat. no. HPLC-10377-H08H and 90311-C081H, respectively).

Biacore Series S Sensor Chips CM5 (Cytiva, cat. no. 29149603) were covalently coated with anti-Fc antibody using amine coupling and the Human Antibody Capture Kit, Type 2 (Cytiva, cat. no. BR100050 and BR100839) according to the manufacturer's instructions.

Subsequently, IgG1-PD1 (2 nM), nivolumab (Bristol-Myers Squibb, lot no. ABP6534; 1.25 nM), and pembrolizumab (Merck Sharp & Dohme, lot. no. T019263; 1.25 nM), diluted in HBS-EP+buffer (Cytiva, cat. no. BR100669; diluted to 1× in distilled water [B Braun, cat. no. 00182479E]), were captured onto the surface at 25° C., with a flow rate of 10 μL/min and a contact time of 60 seconds. This resulted in captured levels of approximately 50 resonance units (RU).

After three start-up cycles of HBS-EP+buffer, human or cynomolgus monkey PD-1 ECD samples (0.19-200 nM; 2-fold dilution in HBS-EP+buffer; 12 cycles) were injected to generate binding curves. Each sample that was analyzed on an antibody coated surface (active surface) was also analyzed on a parallel flow cell without antibody (reference surface), which was used for background correction.

At the end of each cycle, the surface was regenerated using 10 mM Glycine-HCl pH 1.5 (Cytiva, cat. no. BR100354). The data were analyzed using the predefined “Multi-cycle kinetics using capture” evaluation method in the Biacore Insight Evaluation software (Cytiva). The sample with the highest concentration of human or cynomolgus monkey PD-1 (200 nM) was omitted from analysis to allow better curve fits of the data.

Immobilized IgG1-PD1 bound to human PD-1 ECD with a binding affinity (K_{D}) of 1.45±0.05 nM (Table 19). Nivolumab and pembrolizumab bound human PD-1 ECD with a binding affinity comparable to the K_{D} of IgG1-PD1, i.e., with K_{D} values in the low nanomolar range (4.43±0.08 nM and 3.59±0.10 nM, respectively) (Table 19).

Immobilized IgG1-PD1 bound to cynomolgus monkey PD-1 ECD with a K_{D} of 2.74 t 0.58 nM (Table 20), comparable to the affinity of IgG1-PD1 for human PD-1. Nivolumab and pembrolizumab bound cynomolgus monkey PD-1 ECD with a binding affinity comparable to the K_{D} of IgG1-PD1 for cynomolgus monkey PD-1 ECD and comparable to the K_{D} of nivolumab and pembrolizumab for human PD-1 ECD, i.e., with K_{D} values in the low nanomolar range (2.93±0.58 nM and 0.90±0.06 nM, respectively) (Table 20).

TABLE 19
Binding affinities of PD-1 antibodies to the extracellular domain of human PD-1 as determined
by surface plasmon resonance. The association rate constant kₐ (1/Ms), dissociation rate constant
ka (1/s) and equilibrium dissociation constant K_{D} (M) of IgG1-PD1, nivolumab, and pembrolizumab
for the ECD of human PD-1 were determined by SPR.

| | K_{D} (M) | | ka (1/M × s) | | k_{d} (1/s) | |
|---|---|---|---|---|---|---|
| Antibody | Average | SD | Average | SD | Average | SD |
| IgG1-PD1^{a} | 1.45 × 10⁻⁹ | 4.51 × 10⁻¹¹ | 5.17 × 10⁵ | 2.03 × 10⁴ | 7.51 × 10⁻⁴ | 9.61 × 10⁻⁶ |
| Nivolumab^{b} | 4.43 × 10⁻⁹ | 8.49 × 10⁻¹¹ | 4.06 × 10⁵ | 3.54 × 10³ | 1.80 × 10⁻³ | 2.12 × 10⁻⁵ |
| Pembrolizumab^{b} | 3.59 × 10⁻⁹ | 9.90 × 10⁻¹¹ | 2.12 × 10⁶ | 1.36 × 10⁶ | 7.57 × 10⁻³ | 4.71 × 10⁻³ |

^{a}Average and SD from three independent experiments.
^{b}Average and SD from two independent experiments.
Abbreviations: K_{D} = equilibrium dissociation constant; kₐ = association rate constant; k_{d} = dissociation rate constant or off-rate; SD = standard deviation.

TABLE 20
Binding affinities of PD-1 antibodies to the extracellular domain of cynomolgus monkey PD-1
as determined by surface plasmon resonance. The association rate constant kₐ (1/Ms), dissociation rate
constant k_{d} (1/s) and equilibrium dissociation constant K_{D} (M) of IgG1-PD1, nivolumab, and
pembrolizumab for the ECD of cynomolgus monkey PD-1 were determined by SPR.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1^{a} | 2.74 × 10⁻⁹ | 5.79 × 10⁻¹⁰ | 5.34 × 10⁵ | 9.39 × 10⁴ | 1.43 × 10⁻³ | 7.23 × 10⁻⁵ |
| Nivolumab^{a} | 2.93 × 10⁻⁹ | 5.81 × 10⁻¹⁰ | 3.28 × 10⁵ | 5.07 × 10⁴ | 9.43 × 10⁻⁴ | 9.07 × 10⁻⁵ |
| Pembrolizumabb | 8.99 × 10⁻¹⁰ | 5.73 × 10⁻¹¹ | 7.14 × 10⁵ | 3.04 × 10⁴ | 6.42 × 10⁻⁴ | 6.86 × 10⁻⁵ |

^{a}Average and SD from three independent experiments.
^{b}Average and SD from two independent experiments.
Abbreviations: K_{D} = equilibrium dissociation constant; kₐ = association rate constant; k_{d} = dissociation rate constantor off-rate; SD = standard deviation.

### Example 9: Effect of IgG1-PD1 on PD-1 Ligand Binding and PD-1/PD-L1 Signaling

To confirm that IgG1-PD1 functions as a classical immune checkpoint inhibitor, the capacity of IgG1-PD1 to disrupt PD-1 ligand binding and PD-1 checkpoint function was assessed in vitro.

Competitive binding of IgG1-PD1 with recombinant human PD-L1 and PD-L2 to membrane-expressed human PD-1 was assessed by flow cytometry. CHO-S cells transiently transfected with human PD-1 (see Example 6; 5×10⁴ cells/well) were added to the wells of a round-bottom 96-well plate (Greiner, cat. no. 650180), pelleted, and placed on ice. Biotinylated recombinant human PD-L1 (R&D Systems, cat. no. AVI156) or PD-L2 (R&D Systems, cat. no. AVI1224), diluted in PBS (Cytiva, cat. no. SH3A3830.03), was added to the cells (final concentration: 1 μg/mL), immediately after which a concentration range of IgG1-PD1, pembrolizumab (MSD, lot no. T019263 and T036998), or IgG1-ctrl-FERR, diluted in PBS, was added (final concentrations: 30 μg/mL-0.5 ng/mL in three-fold dilution steps). Cells were then incubated for 45 min at RT. Cells were washed twice with PBS and incubated with 50 μL streptavidin-allophycocyanin (R&D Systems, cat. no. F0050; diluted 1:20 in PBS) for 30 min at 4° C., protected from light. Cells were washed twice with PBS and resuspended in 20 μL GMB FACS buffer. Streptavidin-allophycocyanin binding was analyzed by flow cytometry on an Intellicyt® iQue Screener PLUS (Sartorius) using FlowJo software.

The effect of IgG1-PD1 on the functional interaction of PD-1 and PD-L1 was determined using a bioluminescent cell-based PD-1/PD-L1 blockade reporter assay (Promega, cat. no. J1255), essentially as described by the manufacturer. Briefly, cocultures of PD-L1 aAPC/CHO-K1 Cells and PD-1 Effector Cells were incubated with serially diluted IgG1-PD1, pembrolizumab (MSD, lot no. 10749880 or T019263), nivolumab (Bristol-Myers Squibb, lot no. 11024601), or IgG1-ctrl-FERR (final assay concentrations: 15-0.0008 μg/mL in 3-fold dilutions or 10-0.0032 μg/mL in 5-fold dilutions) for 6 h at 37° C., 5% CO₂. Cells were then incubated at RT with reconstituted Bio-Glo™ for 5-30 min, after which luminescence (in relative light units [RLU]) was measured using an Infinite® F200 PRO Reader (Tecan) or an EnVision Multilabel Plate Reader (PerkinElmer).

Dose-response curves were analyzed by non-linear regression analysis (four-parameter dose-response curve fits) using GraphPad Prism software, and the concentrations at which 50% of the maximal (inhibitory) effect was observed (EC₅₀/IC₅₀) were derived from the fitted curves.

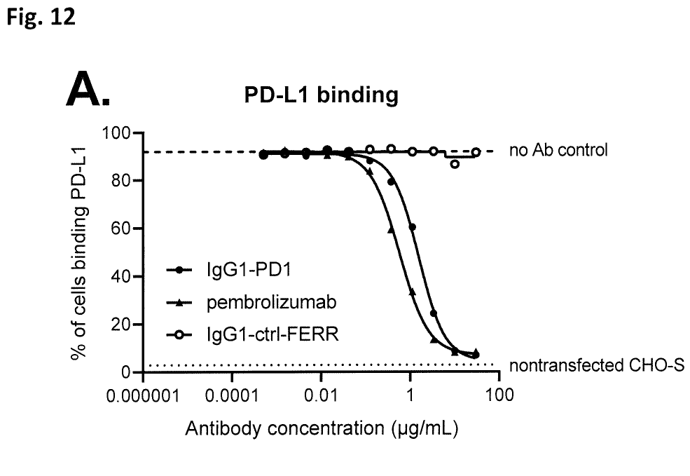

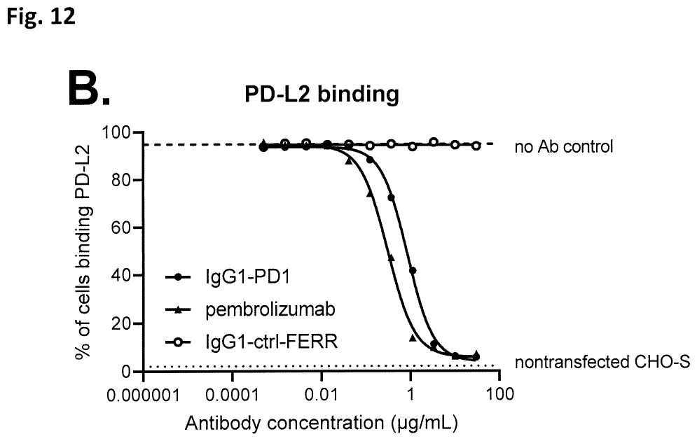

IgG1-PD1 disrupted binding of human PD-L1 and PD-L2 to membrane-expressed human PD-1 in a dose-dependent manner (FIG. 12), with IC₅₀ values of 2.059±0.653 μg/mL (13.9±4.4 nM) for PD-L1 binding inhibition and 1.659±0.721 μg/mL (11.2±4.9 nM) for PD-L2 binding inhibition, ie, in the nanomolar range (Table 21). Pembrolizumab showed PD-L1 and PD-L2 binding inhibition with comparable potency, i.e., with IC₅₀ values in the nanomolar range.

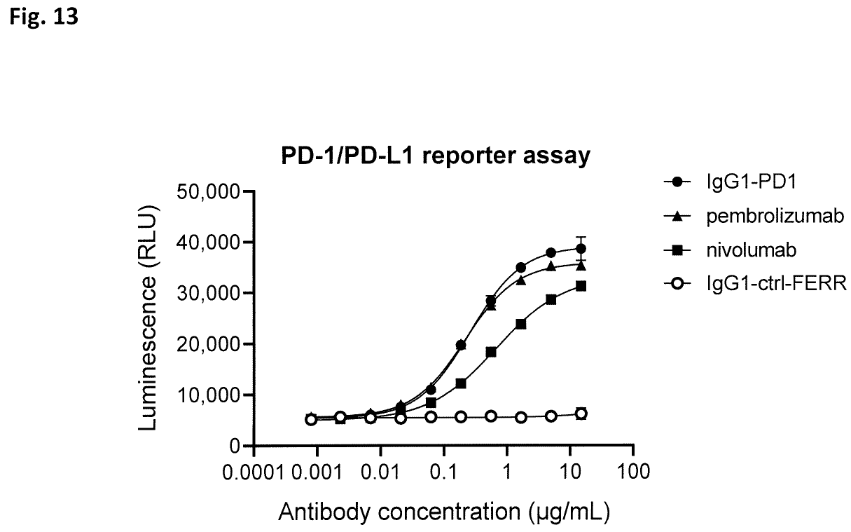

Functional blockade of the PD-1/PD-L1 axis was tested using a cell-based bioluminescent PD-1/PD-L1 blockade reporter assay. Cocultures of reporter Jurkat T cells expressing human PD-1 and harboring an NFAT-RE-driven luciferase, and PD-L1 aAPC/CHOK1 cells expressing human PD-L1 and an antigen-independent TCR activator, were incubated in absence and presence of concentration dilution series of IgG1-PD1, pembrolizumab, or nivolumab. IgG1-ctrl-FERR was included as a negative control. Blockade of the PD-1/PD-L1 interaction results in the release of the PD1/PDL1 mediated inhibitory signal, leading to TCR activation and NFAT-RE-mediated luciferase expression (luminescence measured). IgG1-PD1 induced a dose-dependent increase of TCR signaling in PD-1⁺ reporter T cells (FIG. 13). The EC₅₀ was 0.165 t 0.056 μg/mL (1.12±0.38 nM; Table 22). Pembrolizumab similarly alleviated PD-1 mediated inhibition of TCR signaling, with an EC₅₀ of 0.129±0.051 μg/mL (0.86±0.34 nM), i.e., with comparable potency. Nivolumab alleviated the inhibition of TCR signaling with an EC₅₀ of 0.479±0.198 μg/mL (3.28±1.36 nM), i.e., with slightly lower potency.

In summary, IgG1-PD1 acts as a classical immune checkpoint inhibitor in vitro, by blocking PD-1 ligand binding and disrupting PD-1 immune checkpoint function.

TABLE 21
IC₅₀ values of IgGI-PDI-mediated inhibition of PD-1 ligand binding.
IC₅₀ values were calculated from the competition binding curves.

| Competitive binding with human PD-L1 | | | | Competitive binding with human PD-L2 | | | |
|---|---|---|---|---|---|---|---|
| (average IC₅₀ [±SD]) | | | | (average IC₅₀ [±SD]) | | | |
| IgG1-PD1 | | pembrolizumab | | IgG1-PD1 | | pembrolizumab | |
| μg/mL | nM | μg/mL | nM | μg/mL | nM | μg/mL | nM |
| 2.059 | 13.9 | 1.134 | 7.6 | 1.659 | 11.2 | 1.186 | 8.0 |
| [±0.653] | [±4.4] | [±0.493] | [±3.3] | [±0.721] | [±4.9] | [±0.770] | [±5.2] |

Abbreviations: IC₅₀ = concentration at which 50% of the inhibitory effect was observed; PD-1 = programmed cell death protein 1; PD-L1 = programmed cell death 1 ligand 1; PD-L2 = programmed cell death 1 ligand 2; SD = standard deviation.

TABLE 22
EC₅₀ of PD-1/PD-L1 checkpoint blockade. Cocultures of PD-1⁺ reporter T cells
and PD-L1 aAPC/CHO-K cells were incubated with concentration series of IgG1-PD1,
pembrolizumab, or nivolumab in PD-1/PD-L1 blockade reporter assays. Inhibition of
PD-1/PD-L1 checkpoint function, resulting in downstream TCR signaling and
luciferase expression in the reporter T cells, was determined by measuring luminescence.
From the resulting dose-response curves, EC₅₀ values were calculated.
Average EC₅₀ [±SD]

| IgG1-PD1 | | Pembrolizumab | | Nivolumab | |
|---|---|---|---|---|---|
| μg/mL | nM | μg/mL | nM | μg/mL | nM |
| 0.165 [±0.056] | 1.12 [±0.38] | 0.129 [±0.051] | 0.86 [±0.34] | 0.479 [±0.198] | 3.28 [±1.36] |

Abbreviations: aAPC = artificial antigen-presenting cell; CHO = Chinese hamster ovary; EC₅₀ = concentration at which 50% of the maximal effect is observed; PD-1 = programmed cell death protein 1; PD-L1 = programmed cell death 1 ligand 1; SD = standard deviation; TCR = T-cell receptor.

### Example 10: Antigen-Specific Proliferation Assay to Determine the Capacity of IgG1-PD1 to Enhance Proliferation of Activated T Cells

To determine the capacity of IgG1-PD1 to enhance T-cell proliferation, an antigen-specific proliferation assay was conducted using PD-1-overexpressing human CD8 T cells.

HLA-A*02⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were cryopreserved for T-cell isolation. For differentiation into immature DCs (iDCs), 1×10⁶ monocytes/mL were cultured for five days in RPMI 1640 (Life Technologies GmbH, cat. no. 61870-010) containing 5% pooled human serum (One Lambda Inc., cat. no. A25761), 1 mM sodium pyruvate (Life technologies GmbH, cat. no. 11360-039), 1× non-essential amino acids (Life Technologies GmbH, cat. no. 11140-035), 100 IU/mL penicillin-streptomycin (Life Technologies GmbH, cat. no. 15140-122), 100 ng/mL granulocyte-macrophage colony-stimulating factor (GM-CSF; Miltenyi, cat. no. 130-093-868) and 50 ng/mL interleukin-4 (IL-4; Miltenyi, cat. no. 130-093-924). Once during these five days, half of the medium was replaced with fresh medium. iDCs were harvested by collecting non-adherent cells and adherent cells were detached by incubation with Dulbecco's phosphate-buffered saline (DPBS) containing 2 mM EDTA for 10 min at 37°. After washing with DPBS iDCs were cryopreserved in RPMI 1640 containing 10% DMSO (AppliChem GmbH, cat. no A3672,0050) and 10% human albumin (CSL Behring, PZN 00504775) for future use in antigen-specific T cell assays.

One day prior to the start of an antigen-specific CD8⁺ T cell proliferation assay, frozen PBLs and iDCs from the same donor were thawed. CD8 T cells were isolated from PBLs by MACS technology using anti-CD8 MicroBeads (Miltenyi, cat. no. 130-045-201), according to the manufacturer's instructions. About 10×10⁶ to 15×10⁶ CD8⁺ T cells were electroporated with each 10 μg of in vitro translated (IVT)-RNA encoding the alpha and beta chains of a murine TCR specific for human claudin-6 (CLDN6; HLA-A*02-restricted; described in WO 2015/150327 A1) plus 10 μg IVT-RNA encoding PD-1 (UniProt Q15116) in 250 μL X-Vivol5 medium (Lonza, cat. no. BE02-060Q). The cells were transferred to a 4-mm electroporation cuvette (VWR International GmbH, cat. no. 732-0023) and electroporated using the BTX ECM® 830 Electroporation System (BTX; 500 V, 1×3 ms pulse). Immediately after electroporation, cells were transferred into fresh IMDM GlutaMAX medium (Life Technologies GmbH, cat. no. 319800-030) containing 5% pooled human serum and rested at 37° C., 5% CO₂ for at least 1 hour. T cells were labeled using 1.6 μM carboxyfluorescein succinimidyl ester (CFSE; Life Technologies GmbH, cat. No V12883) in PBS according to the manufacturer's instructions and incubated in IMDM medium supplemented with 5% human AB serum overnight.

Up to 5×10⁶ thawed iDCs were electroporated with 2 μg IVT-RNA encoding full-length human CLDN6 (WO 2015/150327 A1), in 250 μL X-Vivol5 medium, using the electroporation system as described above (300 V, 1×12 ms pulse) and incubated in IMDM medium supplemented with 5% pooled human serum overnight.

The next day, cells were harvested. Cell-surface expression of CLDN6 on iDCs, as well as cell-surface expression of the CLDN6-specific TCR and PD-1 on T cells was confirmed by flow cytometry. To this end, iDCs were stained with a DyLight650-conjugated CLDN6-specific antibody (non-commercially available; in-house production). T cells were stained with a brilliant violet (BV)421-conjugated anti-mouse TCR-β chain antibody (Becton Dickinson GmbH, cat. no. 562839) and an allophycocyanin (APC)-conjugated anti-human PD-1 antibody (Thermo Fisher Scientific, cat. no. 17-2799-42).

Electroporated iDCs were incubated with electroporated, CFSE-labeled T cells at a ratio of 1:10 in the presence of IgG1-PD1, pembrolizumab (Keytruda®, MSD Sharp & Dohme GmbH, PZN 10749897), or nivolumab (Opdivo®, Bristol-Myers Squibb, PZN 11024601) at 4-fold serial dilutions (range 0.00005 to 0.8 μg/mL) in IMDM medium containing 5% pooled human serum in a 96-well round-bottom plate.

The negative control antibody IgG1-ctrl-FERR was used at a single concentration of 0.8 μg/mL. After 4 d of culture, the cells were stained with an APC-conjugated anti-human CD8 antibody. T-cell proliferation was evaluated by flow cytometry analysis of CFSE dilution in CD8⁺ T cells using a BD FACSCelesta™ flow cytometer (Becton Dickinson GmbH).

Flow cytometry data was analyzed using FlowJo software version 10.7.1. CFSE label dilution of CD8⁺ T cells was assessed using the proliferation modeling tool in FlowJo, and expansion indices calculated using the integrated formula. Dose-response curves were generated in GraphPad Prism version 9 (GraphPad Software, Inc.) using a 4-parameter logarithmic fit. Statistical significance was determined by Friedman's test and Dunn's multiple comparisons test using GraphPad Prism version 9.

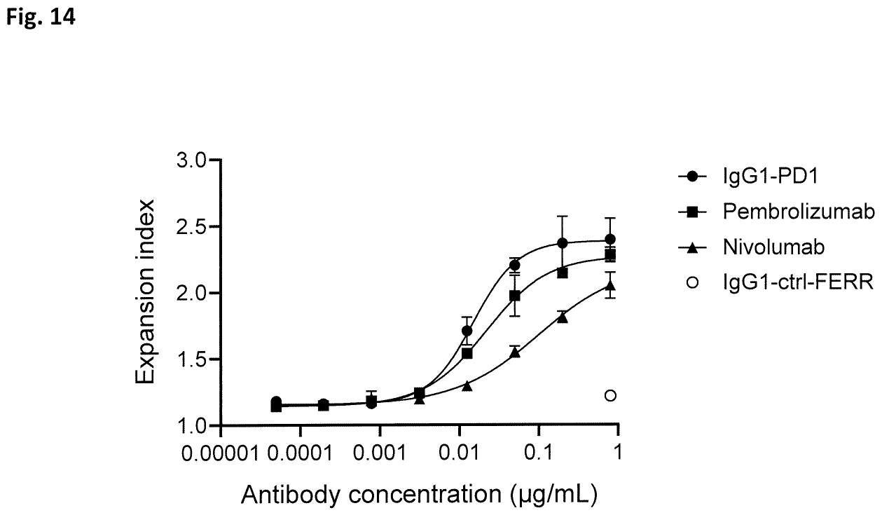

Antigen-specific proliferation of CD8⁻ T cells was enhanced by IgG1-PD1 in a dose-dependent manner (FIG. 14), with EC₅₀ values in the picomolar range (Table 23). Treatment with pembrolizumab or nivolumab also enhanced T-cell proliferation in a dose-dependent manner. The average EC₅₀ of pembrolizumab was comparable to IgG1-PD1, whereas the EC₅₀ of nivolumab was significantly (P=0.0267) higher than that of IgG1-PD1.

TABLE 23
EC₅₀ values in the antigen-specific proliferation assay. EC₅₀ values
of IgG1-PD1, pembrolizumab, and nivolumab were determined using the
CD8⁺ T-cell expansion indices as measured by an antigen-specific
T-cell proliferation assay. Data shown are the values calculated
based on the 4-parameter logarithmic fit.
Average EC₅₀ [±SD]

| IgG1-PD1 | | Pembrolizumab | | Nivolumab | |
|---|---|---|---|---|---|
| μg/mL | nM | μg/mL | nM | μg/mL | nM |
| 0.0124 | 0.0837 | 0.0152 | 0.1018 | 0.0701 | 0.4802 |
| [±0.0018] | [±0.0123] | [±0.0049] | [±0.0333] | [±0.0238] | [±0.1632] |

Abbreviations: EC₅₀ = half-maximal effective concentration; FERR = L234F/L235E/G236R-K409R; PD1 = programmed cell death protein 1; SD = standard deviation.

### Example 11: Effect of IgG1-PD1 on Cytokine Secretion in an Allogeneic MLR Assay

To investigate the capacity of IgG1-PD1 to enhance cytokine secretion in a mixed lymphocyte reaction (MLR) assay, three unique, allogeneic pairs of human mature dendritic cells (mDCs) and CD8⁺ T cells were cocultured in the presence of IgG1-PD1. The levels of IFNγ were measured using an IFNγ-specific immunoassay, while the levels of monocyte chemoattractant protein-1 (MCP-1), GM-CSF, interleukin (IL)-1β, IL-2, IL-4, IL-5, IL-6, IL-8, IL-10, IL12-p40, IL-15, IL-17α, and tumor necrosis factor (TNFα) were determined using a customized Luminex multiplex immunoassay.

Human CD4⁺ monocytes were obtained from healthy donors (BiolVT). For differentiation into immature dendritic cells (iDCs), monocytes were cultured for 6 d in RPMI-1640 complete medium (ATCC modification formula; Thermo Fisher, cat. no. A1049101) supplemented with 10% heat-inactivated fetal bovine serum (FBS; Gibco, cat. no. 16140071), 100 ng/mL GM-CSF and 300 ng/mL IL-4 (BioLegend, cat. no. 766206) at 37° C. On day 4, the medium was replaced with fresh medium with supplements. To mature the iDCs, the cells were incubated in RPMI-1640 complete medium supplemented with 10% FBS, 100 ng/mL, GM-CSF, 300 ng/mL IL-4, and 5 μg/mL lipopolysaccharide (LPS; Thermo Fisher Scientific, cat. no. 00 4976 93) at 37° C. for 24 h prior to start of the MLR assay. In parallel, purified CD8⁻ T cells obtained from allogeneic healthy donors (BioIVT) were thawed and incubated in RPMI-1640 complete medium supplemented with 10% FBS and 10 ng/mL IL-2 (BioLegend, cat. no. 589106) at 37° C. O/N.

The next day, the LPS-matured dendritic cells (mDCs) and allogeneic CD8⁺ T cells were harvested and resuspended in prewarmed AIM-V medium (Thermo Fisher Scientific, cat. no. 12055091) at 4×10⁵ cells/mL and 4×10⁶ cells/mL, respectively. The mDCs (20,000 cells/well) were incubated with allogeneic naive CD8⁺ T cells (200,000 cells/well) in the presence of an antibody concentration range (0.001-30 μg/mL) of IgG1-PD1, IgG1-ctrl-FERR, or pembrolizumab (MSD, cat. no. T019263) or in the presence of 30 μg/mL IgG4 isotype control (BioLegend, cat. no. 403702) in AIM-V medium in a 96-well round-bottom plate at 37° C.

After 5 d, cell-free supernatant was transferred from each well to a new 96-well plate and stored at −80° C. until further analysis of cytokine concentrations.

The IFNγ levels were determined using an IFNγ-specific immunoassay (Alpha Lisa IFNγ kit; Perkin Elmer, cat. no. AL217) on an Envision instrument, according to the manufacturer's instructions.

The levels of MCP-1, GM-CSF, IL-1β, IL-2, IL-4, IL-5, IL-6, IL-8, IL-10, IL12-p40, IL-15, IL-17α and TNFα were determined using a customized Luminex® multiplex immunoassay (Millipore, order no. SPR1526) based on the Human TH17 Magnetic Bead Panel (MILLIPLEX®). Briefly, cell-free supernatants were thawed and 10 μL of each sample was added to 10 μL Assay Buffer in wells of a 384-well plate (Greiner Bio-One, cat. no. 781096) prewashed with 1× Wash Buffer. In parallel, 10 μL of Standard or Control in Assay Buffer was added to the wells, after which 10 μL of assay medium was added. Magnetic beads against the different cytokines were mixed and diluted to 1× concentrations in Bead Diluent, after which 10 μL of the mixed beads was added to each well. The plate was sealed and incubated at 4° C., shaking, O/N. Wells were washed three times with 60 μL×Wash Buffer. Subsequently, 10 μL of Custom Detection Antibodies was added to each well, and the plate was sealed and incubated at RT, shaking, for 1 h. Next, 10 μL of streptavidin-PE was added to each well, and the plate was sealed and incubated at RT, shaking, for 30 min. Wells were washed three times with 60 μL 1× Wash Buffer as described above, after which beads were resuspended in 75 μL Luminex Sheath Fluid by shaking at RT for 5 min. Samples were run on a Luminex FlexMap 3D system.

At the start and at the end of the MLR assay, expression of PD-1 on the CD8⁺ T cells and expression of PD-L1 on the mDCs was confirmed by flow cytometry using PE-Cy7-conjugated anti-PD-1 (BioLegend, cat. no. 329918; 1:20), allophycocyanin-conjugated anti-PD-L1 (BioLegend, cat. no. 329708; 1:80), BUV496-conjugated anti-CD3 (BD Biosciences, cat. no. 612940; 1:20), and BUV395-conjugated anti-CD8 (BD Biosciences, cat. no. 563795; 1:20).

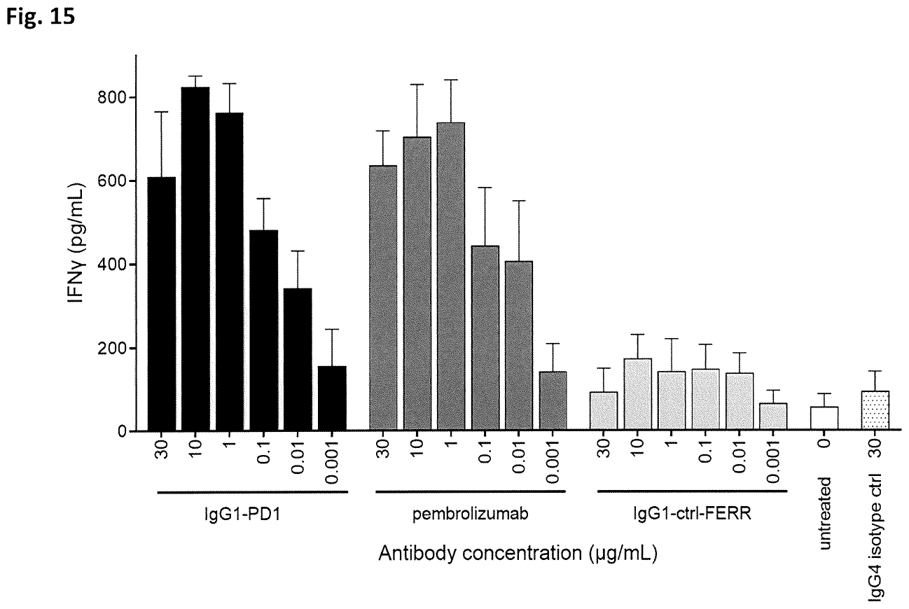

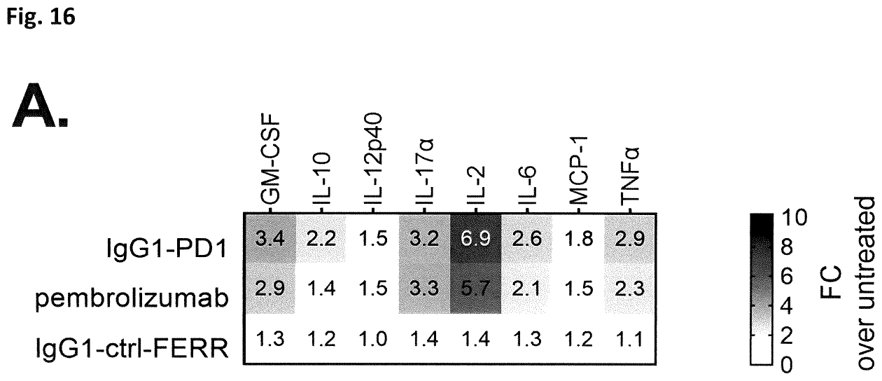

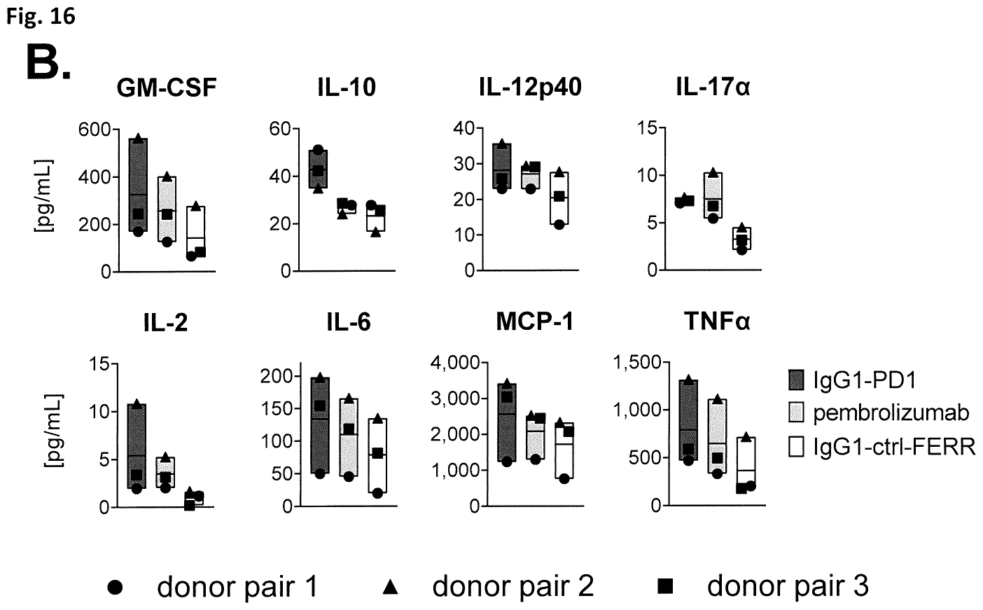

IgG1-PD1 consistently enhanced secretion of IFNγ (FIG. 15) in a dose-dependent manner. IgG1-PD1 also enhanced secretion of MCP-1, GM-CSF, IL-2, IL-6, IL-12p40, IL-17α, IL-10, and TNFα (FIG. 16). Pembrolizumab had a comparable effect on cytokine secretion.

### Example 12: Evaluation of C1q Binding to IgG1-PD1

Binding of complement protein C1q to IgG1-PD1 harboring the FER Fe-silencing mutations in the constant heavy chain region was assessed using activated human CD8⁻ T cells. As a positive control, IgG1-CD52-E430G was included, which has V_{H} and V_{L} domains based on the CD52 antibody CAMPATH-1H and which has an Fc-enhanced backbone that is known to efficiently bind C1q when bound to the cell surface. As non-binding negative control antibodies, IgG1-ctrl-FERR and IgG1-ctrl were included.

Human CD8⁺ T cells were purified (enriched) from buffy coats obtained from healthy volunteers (Sanquin) by negative selection using the RosetteSep™ Human CD8⁻ T Cell Enrichment Cocktail (Stemcell Technologies, cat. no. 15023C.2) or by positive selection via magnetic activated cell sorting (MACS), using CD8 MicroBeads (Miltenyi Biotec, cat. no. 130-045-201) and LS columns (Miltenyi Biotec, cat. no. 130-042-401), all according to the manufacturer's instructions. Purified T cells were resuspended in T-cell medium (Roswell Park Memorial Institute [RPMI]-1640 medium with 25 mM HEPES and L-glutamine [Lonza, cat. no. BE12-115F], supplemented with 10% heat-inactivated donor bovine serum with iron [DBSI; Gibco, cat. no. 20731-030] and penicillin/streptomycin [pen/strep; Lonza, cat. no. DE17-603E]).

Anti-CD3/CD28 beads (Dynabeads™ Human T-Activator CD3/CD28; ThermoFisher Scientific, cat. no. 11132D) were washed with PBS and resuspended in T-cell medium. The beads were added to the enriched human CD8⁺ T cells at a 1:1 ratio and incubated at 37° C., 5% CO₂ for 48 h. Next, the beads were removed using a magnet, and the cells were washed twice in PBS and counted again.

PD-1 expression on the activated CD8⁺ T cells was confirmed by flow cytometry, using IgG1-PD1 (30 μg/mL) and R-phycoerythrin (PE)-conjugated goat-anti-human IgG F(ab′)₂ (diluted 1:200 in GMB FACS buffer; Jackson ImmunoResearch, cat. no. 109-116-098), or a commercial PE-conjugated PD-1 antibody (BioLegend, cat. no. 329906; diluted 1:50).

Activated CD8⁺ T cells were seeded in a round-bottom 96-well plate (30,000 or 50,000 cells/well), pelleted, and resuspended in 30 μL assay medium (RPMI-1640 with 25 mM HEPES and L-glutamine, supplemented with 0.1% [w/v] bovine serum albumin fraction V [BSA; Roche, cat. no. 10735086001] and penicillin/streptomycin). Subsequently, 50 μL of IgG1-PD1, IgG1-ctrl-FERR, IgG1-CD52-E430G, or IgG1-ctrl (final concentrations of 1.7×10⁴-30 μg/mL in 3-fold dilution steps in assay medium) was added to each of the wells and incubated at 37° C. for 15 min to allow the antibodies to bind to the cells.

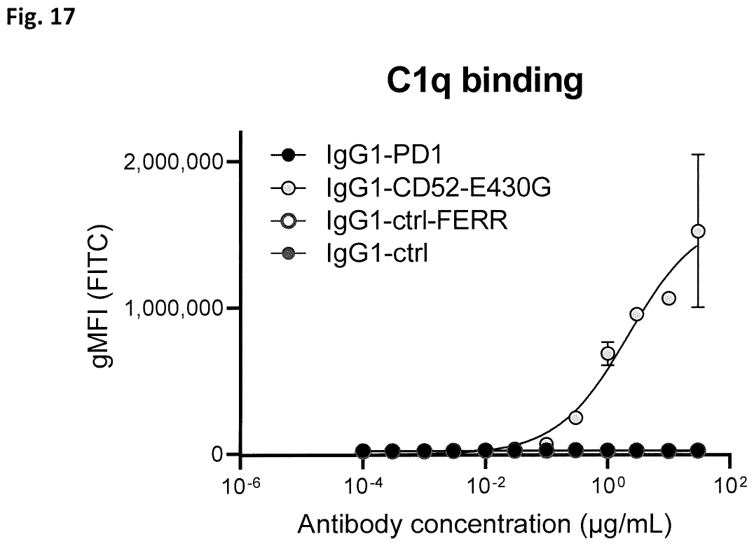

Human serum (20 μL/well; Sanquin, lot 20L15-02), as a source of C1q, was added to a final concentration of 20%. Cells were incubated on ice for 45 min, followed by two washes with cold GMB FACS buffer and incubation with 50 μL fluorescein isothiocyanate (FITC)-conjugated rabbit anti-human C1q (final concentration of 20 μg/mL [DAKO, cat no. F0254]; diluted 1:75 in GMB FACS buffer) in the presence or absence of allophycocyanin-conjugated mouse-anti-CD8 (BD Biosciences, cat. no. 555369; diluted 1:50 in GMB FACS buffer) in the dark at 4° C. for 30 min. Cells were washed twice with cold GMB FACS buffer, resuspended in 20 μL of GMB FACS buffer supplemented with 2 mM ethylenediaminetetraacetic acid (EDTA; Sigma-Aldrich, cat. no. 03690) and 4′,6-diamidino-2-phenylindole (DAPI) viability dye (1:5,000; BD Pharmingen, cat. no. 564907). C1q binding to viable cells (as identified by DAPI exclusion) was analyzed by flow cytometry on an IntelliCyt® iQue Screener PLUS (Sartorius) or iQue3 (Sartorius). Binding curves were analyzed using non-linear regression analysis (sigmoidal dose-response with variable slope) using GraphPad Prism software.

Whereas dose-dependent C1q binding was observed to membrane-bound IgG1-CD52-E430G, no C1q binding was observed to membrane-bound IgG1-PD1 or to the non-binding control antibodies (FIG. 17).

These results indicate that the functionally inert backbone of IgG1-PD1 does not bind C1q.

### Example 13: Binding of IgG1-PD1 to Fey Receptors as Determined by SPR

The binding of IgG1-PD1 to immobilized FcγRs (FcγRIa, FcγRIIa, FcγRIIb and FcγRIIIa) was assessed in vitro by SPR. Both polymorphic variants were included for FcγRIIa (H131 and R131) and FcγRIIIa (V158 and F158). As a positive control for FcγR binding, IgG1-ctrl with a wild-type Fc region was included.

In a first experiment, binding of IgG1-PD1, or IgG1-ctrl to immobilized human recombinant FcγR variants (FcγRIa, FcγRIIa, FcγRIIb, and FcγRIIIa) was analyzed using a Biacore 8K SPR system. In a second set of experiments, using the same method, binding of IgG1-PD1, nivolumab (Bristol-Meyers Squibb, lot no. ABP6534), pembrolizumab (Merck Sharp & Dohme, lot no. U013442), dostarlimab (GlaxoSmithKline, lot. no. 1822049), cemiplimab (Regeneron, lot no. 1F006A), IgG1-ctrl, or IgG4-ctrl was analyzed.

Biacore Series S Sensor Chips CM5 (Cytiva, cat. no. 29104988) were covalently coated with anti-Histidine (His) antibody using amine-coupling and His capture kits (Cytiva, cat. no. BR100050 and cat. no. 29234602) according to the manufacturer's instructions. FcγRIa, FcγRIIa (H131 and R131), FcγRIIb and FcγRIIIa (V158 and F158) (SinoBiological, cat. no. 10256-H08S-B, 10374-1108H1, 10374-H27H, 10259-112711, 10389-H27H1, and 10389-H27H, respectively) diluted in HBS-EP+(Cytiva, cat. no. BR 100669) were captured onto the surface of the anti-His coated sensor chip with a flow rate of 10 μL/min and a contact time of 60 seconds to result in captured levels of approximately 350-600 resonance units (RU).

After three start-up cycles of HBS-EP+buffer, test antibodies (IgG1-PD1, nivolumab, pembrolizumab, dostarlimab, cemiplimab, IgG1-ctrl, or IgG4-ctrl) were injected to generate binding curves, using antibody ranges as indicated in Table 24. Each sample that was analyzed on a surface with captured FcγRs (active surface) was also analyzed on a parallel flow cell without captured FcγRs (reference surface), which was used for background correction. The third start-up cycle containing HBS-EP+ as a (mock) analyte was subtracted from other sensorgrams to yield double-referenced data.

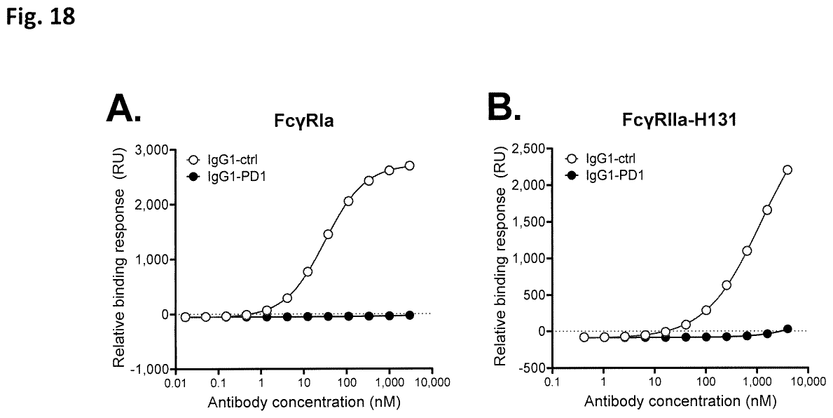

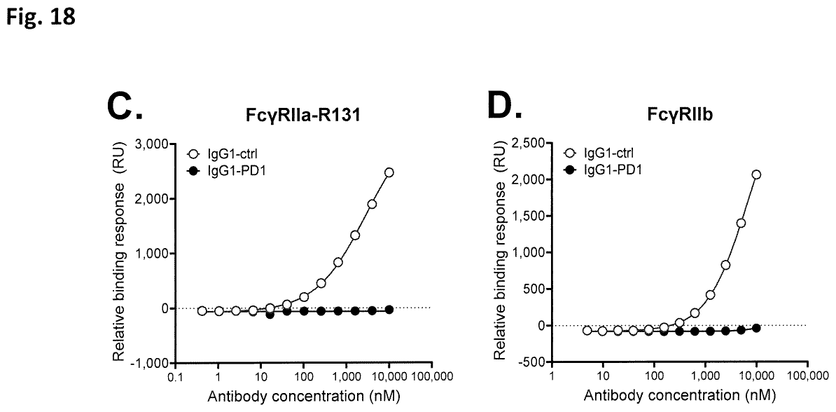

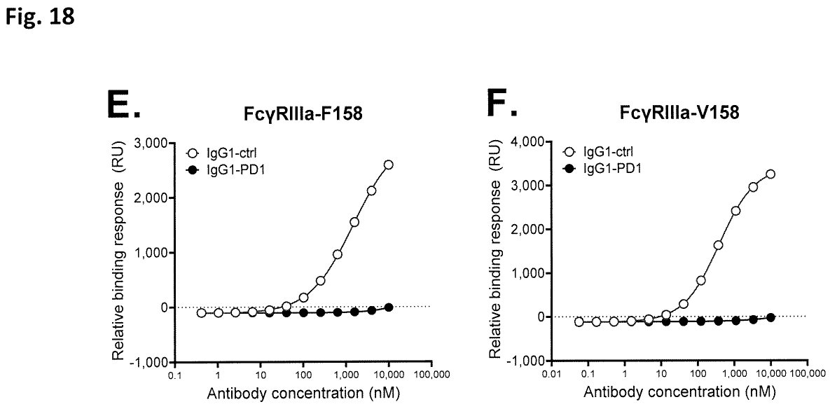

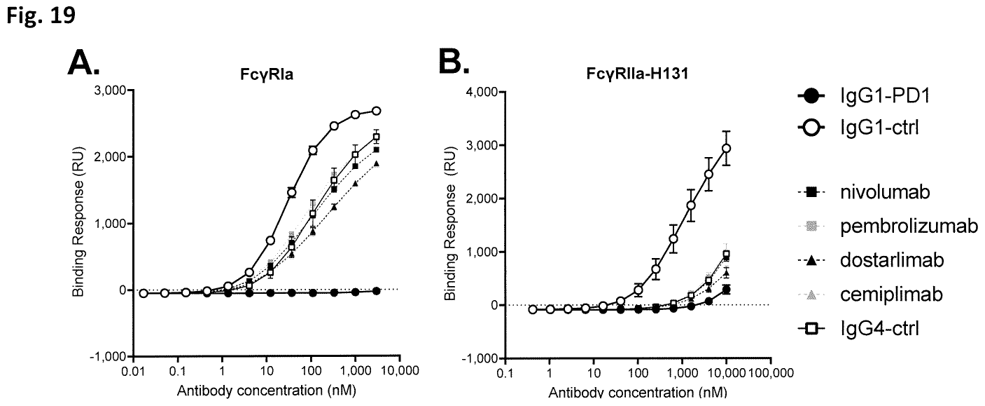

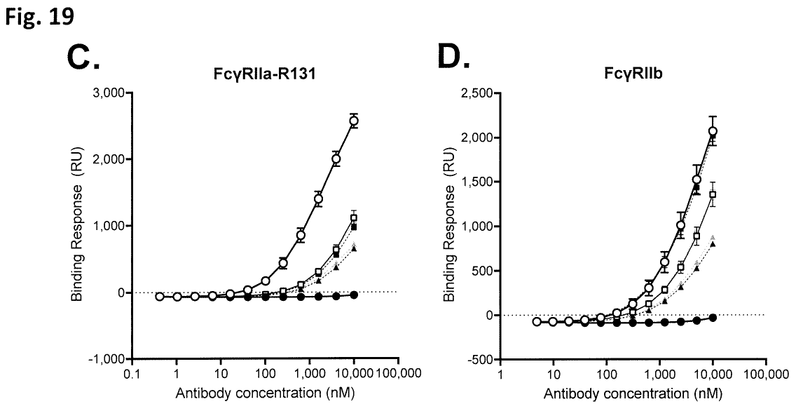

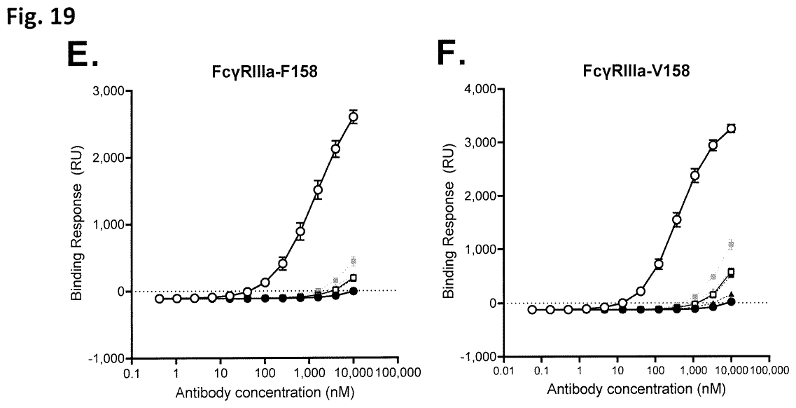

At the end of each cycle, the surface was regenerated using 10 mM Glycine-HCl pH 1.5 (Cytiva, cat. no. BR100354). Sensorgrams were generated using Biacore Insight Evaluation software (Cytiva) and a four-parameter logistic fit was applied on end-point measurements (binding plateau versus post-capture baseline). Data of the first experiment (n=1; qualified SPR assay) is shown in FIG. 18; data of the second set of experiments (n=3) is shown in FIG. 19.

TABLE 24
Test conditions for binding to individual FcγRs

| | Anti-PD-1 antibody concentration range tested | | |
|---|---|---|---|
| | Start | Fold | Lowest |
| FcγR | concentration (nM) | dilution | concentration (nM) |
| FcγRIa | 3,000 | 1:3 | 0.02 |
| FcγRIIa-H131 | 10,000 | 1:2.5 | 0.42 |
| FcγRIIa-R131 | 10,000 | 1:2.5 | 0.42 |
| FcγRIIb | 10,000 | 1:2 | 4.9 |
| FcγRIIIa-V158 | 10,000 | 1:3 | 0.06 |
| FcγRIIIa-F158 | 10,000 | 1:2.5 | 0.42 |

Results from the first experiment showed binding of IgG1-ctrl to all FcγRs, while no binding was observed for IgG1-PD1 to FcγRIa, FcγRIIa (H131 and R131), FcγRIIb, and FcγRIIIa (V158 and F158) (FIG. 18).

Results from the second set of experiments confirmed lack of FcγR binding for IgG1-PD1 (FIG. 19). IgG4-ctrl and the other anti-PD-1 antibodies tested (nivolumab, pembrolizumab, dostarlimab, and cemiplimab; all of the IgG4 subclass) demonstrated clear binding to FcγRIa, FcγRIIa-H131, FcγRIIa-R131, and FcγRIIb, and minimal to very minimal binding to FcγRIIIa-F158 and FcγRIIIa-V158.

These data confirm lack of FcγR binding for the Fc domain of IgG1-PD1 and demonstrate FcγR binding to nivolumab, pembrolizumab, dostarlimab, and cemiplimab. Taken together, these data suggest that the Fc domain of IgG1-PD1 is unable to induce FcγR-mediated effector functions (ADCC, ADCP).

### Example 14: Binding of IgG1-PD1 to Cell Surface Expressed FcγRIa as Determined by Flow Cytometry

Binding of IgG1-PD1, nivolumab, pembrolizumab, dostarlimab, and cemiplimab to human cell surface expressed FcγRIa was analyzed using flow cytometry.

FcγRIa was expressed on transiently transfected CHO-S cells, and cell surface expression was confirmed by flow cytometry using FITC-conjugated anti-FcγRI antibody (BioLegend, cat. no. 305006; 1:25). Binding of anti-PD-1 antibodies to transfected_CHO-S cells was assessed as described in Example 7. Briefly, antibody dilutions (final concentrations: 1.69×10⁴-10 μg/mL, 3-fold dilutions) of IgG1-PD1, nivolumab (Bristol-Meyers Squibb, lot no. ABP6534), pembrolizumab (Merck Sharp & Dohme, lot no. U013442), dostarlimab (GlaxoSmithKline, lot. no. 1822049), cemiplimab (Regeneron, lot no. 1F006A), IgG1-ctrl, and IgG1-ctrl-FERR were prepared in GMB FACS buffer. Cells were centrifuged, supernatant was removed, and cells (30,000 cells in 50 μL) were incubated with 50 μL of the antibody dilutions for 30 min at 4° C. Cells were washed twice with GMB FACS buffer and incubated with 50 μL secondary antibody (PE-conjugated goat-anti-human IgG F(ab′)₂; 1:500) for 30 min at 4° C., protected from light. Cells were washed twice with GMB FACS buffer and resuspended in GMB FACS buffer supplemented with 2 mM EDTA and DAPI viability marker (1:5,000).

Antibody binding to viable cells was analyzed by flow cytometry on an Intellicyt iQue PLUS Screener (Intellicyt Corporation) using FlowJo software by gating on PE-positive, DAPI-negative cells. Binding curves were analyzed using non-linear regression analysis (four-parameter dose-response curve fits) in GraphPad Prism.

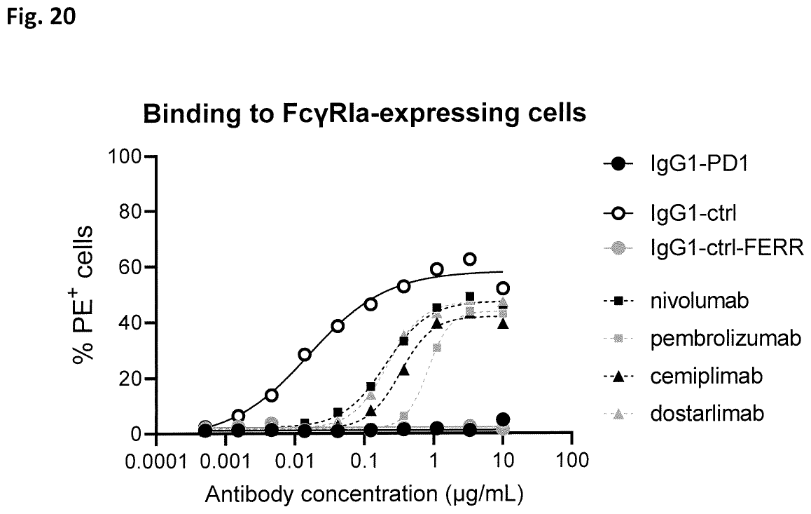

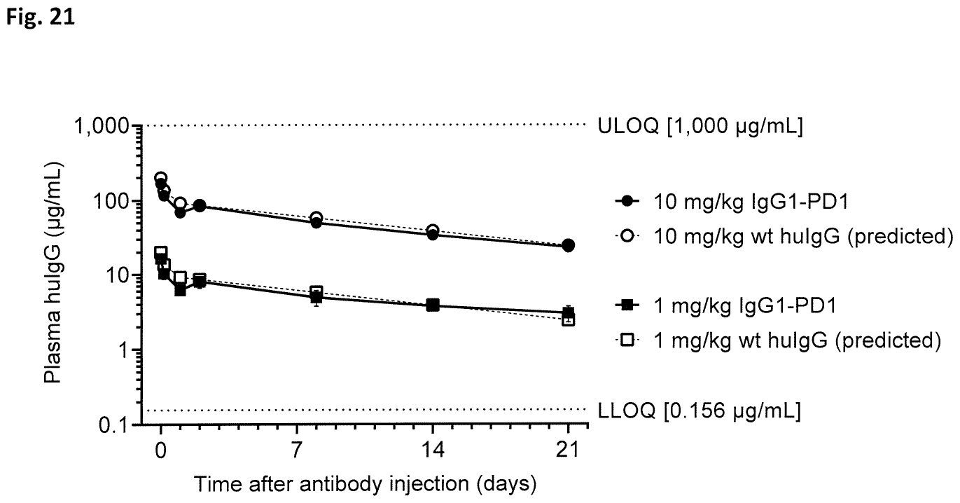

In the flow cytometry binding assays, the positive control antibody IgG1-ctrl (with a wild-type Fe region) showed binding to cells transiently expressing FcγRIa, while no binding was observed for the negative control antibody IgG1-ctrl-FERR (with an Fc region containing the FER inertness mutations and an additional, in the context of this study functionally irrelevant, K409R mutation) (FIG. 20). No binding was observed for IgG1-PD1, while concentration-dependent binding was observed for pembrolizumab, nivolumab, cemiplimab, and dostarlimab.

These data confirm lack of FcγRIa binding for the Fc domain of IgG1-PD1 and demonstrate FcγRIa binding to nivolumab, pembrolizumab, dostarlimab, and cemiplimab. Taken together, these data suggest that the Fc domain of IgG1-PD1 is unable to induce FcγRIa-mediated effector functions.

### Example 15: Binding to Neonatal Fc Receptor by IgG1-PD1

The neonatal Fc receptor (FcRn) is responsible for the long plasma half-life of IgG by protecting IgG from degradation. IgG binds to FcRn in an acidic (pH 6.0) endosomal environment but dissociates from FcRn at neutral pH (pH 7.4). This pH-dependent binding of antibodies to FcRn causes recycling of the antibody together with FcRn, preventing intracellular antibody degradation, and therefore is an indicator for the in vivo pharmacokinetics of that antibody. The binding of IgG1-PD1 to immobilized FcRn was assessed in vitro at pH 6.0 and pH 7.4 by means of surface plasmon resonance (SPR).

Binding of IgG1-PD1 to immobilized human FcRn was analyzed using a Biacore 8K SPR system. Biacore Series S Sensor Chips CM5 (Cytiva, cat. no. 29104988) were covalently coated with anti-histidine (His) antibody using amine coupling and His capture kits (Cytiva, cat. no. BR100050 and cat. no. 29234602) according to the manufacturer's instructions. FcRn (SinoBiological, cat. no. CT071-H27H-B) diluted to a 5 nM coating concentration in PBS-P+ buffer pH 7.4 (Cytiva, cat. no. 28995084) or in PBS-P+buffer with the pH adjusted to 6.0 (by addition of hydrochloric acid [Sigma-Aldrich, cat. no. 07102]) was captured onto the surface of the anti-His coated sensor chip with a flow rate of 10 μL/min and a contact time of 60 seconds. This resulted in captured levels of approximately 50 RU. After three start-up cycles of pH 6.0 or pH 7.4 PBS-P+buffer, test antibodies (6.25-100 nM two-fold dilution series of IgG1-PD1, pembrolizumab (MSD, lot. no. T019263), or nivolumab (Bristol-Myers Squibb, lot. no. ABP6534) in pH 6.0 or pH 7.4 PBS-P+buffer) were injected to generate binding curves. Each sample that was analyzed on a surface with captured FcRn (active surface) was also analyzed on a parallel flow cell without captured FcRn (reference surface), which was used for background correction. The third start-up cycle containing HBS-EP+ as a (mock) analyte was subtracted from other sensorgrams to yield double-referenced data. At the end of each cycle, the surface was regenerated using 10 mM Glycine HCL pH 1.5 (Cytiva, cat. no. BR100354). The data were analyzed using the predefined “Multi-cycle kinetics using capture” evaluation method in the Biacore Insight Evaluation software (Cytiva). Data is based on three separate experiments with technical duplicates.

At pH 6.0, IgG1-PD1 bound FcRn with an average affinity (K_{D}) of 50 nM (Table 25), which is comparable to an IgG1-ctrl antibody with a wild-type Fc region (a broad range of affinities is reported for wild-type IgG1 molecules in literature; in previous in-house experiments with the same assay set-up, an average K) of 34 nM was measured for IgG1-ctrl across 12 data points). The affinity of pembrolizumab and nivolumab was approximately two-fold lower (K_{D} of 116 nM and 133 nM, respectively). No FcRn binding was observed at pH 7.4 (not shown). Taken together, these results demonstrate that the FER inertness mutations in the IgG1-PD1 Fc region do not affect FcRn binding and suggest that IgG1-PD1 will retain typical IgG pharmacokinetic properties in vivo.

TABLE 25
Affinity for FcRn as determined by SPR. Binding of IgG1-PD1, pembrolizumab, and nivolumab
to sensor chips coated with human FcRn was analyzed by SPR. The average affinity and
SD are based on three independent measurements with technical duplicates.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1 | 4.99 × 10⁻⁸ | 6.85 × 10⁻⁹ | 8.65 × 10⁵ | 6.79 × 10⁴ | 4.29 × 10⁻² | 4.88 × 10⁻³ |
| Nivolumab | 1.33 × 10⁻⁷ | 2.01 × 10⁻⁸ | 5.18 × 10⁵ | 4.70 × 10⁴ | 6.89 × 10⁻² | 1.19 × 10⁻² |
| Pembrolizumab | 1.16 × 10⁻⁷ | 1.69 × 10⁻⁸ | 5.46 × 10⁵ | 4.42 × 10⁴ | 6.36 × 10⁻² | 1.15 × 10⁻² |

Abbreviations: K_{D} = equilibrium dissociation constant; kₐ = association rate constant; k_{d} = dissociation rate constant or off-rate; SD = standard deviation.

### Example 16: Pharmacokinetic Analysis of IgG1-PD1 in Absence of Target Binding

The pharmacokinetic properties of IgG1-PD1 were analyzed in mice. PD-1 is expressed mainly on activated B and T cells, and as such, its expression is expected to be limited in non-tumor bearing SCID mice, which lack mature B and T cells. Furthermore, IgG1-PD1 shows substantially reduced cross-reactivity to cells transiently overexpressing mouse PD-1 (Example 7). Therefore, the pharmacokinetic (PK) properties of IgG1-PD1 in non-tumor bearing SCID mice are expected to reflect the PK properties of IgG1-PD1 in absence of target binding.

The mice in this study were housed in the Central Laboratory Animal Facility (Utrecht, the Netherlands). All mice were kept in individually ventilated cages with food and water provided ad libitum. All experiments were in compliance with the Dutch animal protection law (WoD) translated from the directives (2010/63/EU) and were approved by the Dutch Central Commission for animal experiments and by the local Ethical committee). SCID mice (C.B-17/IcrHan® Hsd-Prkde^{scid}, Envigo) were injected intravenously with 1 or 10 mg/kg IgG1-PD1, using 3 mice per group. Blood samples (40 μL) were collected from the saphenous vein or the cheek veins at 10 min, 4 h, 1 day, 2 days, 8 days, 14 days, and 21 days after antibody administration. Blood was collected into vials containing K₂-ethylenediaminetetraacetic acid and stored at −65° C. until determination of antibody concentrations.

By a total human IgG (hIgG) electrochemiluminescence immunoassay (ECLIA), specific hIgG concentrations were determined. Meso Scale Discovery (MSD) standard plates (96-well MULTI-ARRAY plate, cat. no. L15XA-3) were coated with mouse anti-hIgG capture antibody (IgG2amm-1015-6A05) diluted in PBS (Lonza, cat. no. BE17-156Q) for 16-24 h at 2-8° C. After washing the plate with PBS-Tween (PBS-T; PBS supplemented with 0.05% (w/v) Tween-20 [Sigma, cat. no. P1379]) to remove non-bound antibody, the unoccupied surfaces were blocked for 60±5 min at RT (PBS-T supplemented with 3% (w/v) Blocker-A [MSD, cat. no. R93AA-1]) followed by washing with PBS-T. Mouse plasma samples were initially diluted 50-fold (2% mouse plasma) in assay buffer (PBS-T supplemented with 1% (w/v) Blocker-A). To create a reference curve, IgG1-PD1 (same batch as the material used for injection) was diluted (measuring range: 0.156-20.0 μg/mL; anchor points: 0.0781 and 40.0 μg/mL) in Calibrator Diluent (2% mouse plasma [K₂EDTA, pooled plasma, BIOIVT, cat. no. MSE00PLK2PNN] in assay buffer). To accommodate for the expected wide range of antibody concentrations present in the samples, samples were additionally diluted 1:10 or 1:50 in Sample Diluent (2% mouse plasma in assay buffer). The coated and blocked plates were incubated with 50 μL diluted mouse samples, the reference curve, and appropriate quality control samples (pooled mouse plasma spiked with IgG1-PD1, covering the range of the reference curve) at RT for 90±5 min. After washing with PBS-T, the plates were incubated with SULFO-TAG-conjugated mouse anti-hIgG detection antibody IgG2amm-1015-4A01 at RT for 90±5 min. After washing with PBS-T, immobilized antibodies were visualized by adding Read Buffer (MSD GOLD Read Buffer, cat. no. R92TG-2) and measuring light emission at ˜620 nm using an MSD Sector S600 plate reader. Processing of analytical data was performed using SoftMax Pro GxP Software v7.1. Extrapolation below the run lower limit of quantitation (LLOQ) or above the upper limit of quantitation (ULOQ) was not allowed.

The plasma clearance profile of IgG1-PD1 in absence of target binding was comparable to the clearance profile of a wild-type human IgG1 antibody in SCID mice predicted by a two-compartment model based on IgG1 clearance in humans (Bleeker et al., 2001, Blood. 98(10):3136-42) (FIG. 21). No clinical observations were noted, and no body weight loss was observed.

In conclusion, these data indicate that the PK properties of IgG1-PD1 are comparable to those of normal human IgG antibodies in absence of target binding.

### Example 17: Antitumor Activity of IgG1-PD1 in Human PD-1 Knock-In Mice

IgG1-PD1 shows only limited binding to cells transiently overexpressing mouse PD-1 (Example 7). Therefore, to assess antitumor activity of IgG1-PD1 in vivo, C57BL/6 mice engineered to express the human PD-1 extracellular domain (ECD) in the mouse PD-1 gene locus (hPD-1 knock-in [KI] mice) were used.

All animal experiments were performed at Crown Bioscience Inc. and approved by their Institutional Animal Care and Use Committee (IACUC) prior to execution. Animals were housed and handled in accordance with good animal practice as defined by the regulations of the Association for Assessment and Accreditation of Laboratory Animal Care (AAALAC). Female homozygous human PD-1 knock-in mice on a C57BL/6 background (hPD-1 KI mice; Beijing Biocytogen Co., Ltd; C57BL/6-Pdcd1^{tm1(PDCD1)}/Bcgen, stock no. 110003), 7-9 weeks old, were injected subcutaneously (SC) with syngeneic MC38 colon cancer cells (1×10⁶ cells) in the right lower flank. Tumor growth was evaluated using a caliper (three times per week after randomization), and tumor volumes (mm³) were calculated from caliper measurements as: tumor volume=0.5×(length×width²), where the length is the longest tumor dimension, and the width is the longest tumor dimension perpendicular to the length. Mice were randomized (9 mice per group) based on tumor volume and body weight when tumors had reached an average volume of approximately 60 mm³ (denoted as Day 0). At the start of treatment, mice were injected intravenously (IV; dosing volume 10 mL/kg in PBS) with 0.5, 2, or 10 mg/kg IgG1-PD1 or pembrolizumab (obtained from Merck by Crown Bioscience Inc., lot no. T042260), or with 10 mg/kg isotype control antibody IgG1-ctrl-FERR. Subsequent doses were administered intraperitoneally (IP). A dosing regimen of two doses weekly for three weeks (2QW×3) was used. Animals were monitored daily for morbidity and mortality and monitored routinely for other clinical observations. The experiment ended for individual mice when the tumor volume exceeded 1,500 mm³ or when the animals reached other humane endpoints.

To compare progression-free survival between the groups, curve fits were applied to the individual tumor growth graphs to establish the day of progression beyond a tumor volume of 500 mm³ for each mouse. These day values were plotted in a Kaplan-Meier survival curve and used to perform a Mantel-Cox analysis between individual curves using SPSS software. The difference in tumor volumes between the groups was compared using a nonparametric Mann-Whitney analysis (in GraphPad Prism) on the last day that all groups were still intact (ie, until the first tumor-related death in the study, ie, Day 11). P-values are presented accompanied by median values (per group) including the 95% confidence interval of the difference in median (Hodges Lehmann).

The mice showed no signs of illness, but two mice were found dead (one in the 2 mg/kg IgG1-PD1 group and one in the 2 mg/kg pembrolizumab treatment group). The cause of these deaths was undetermined.

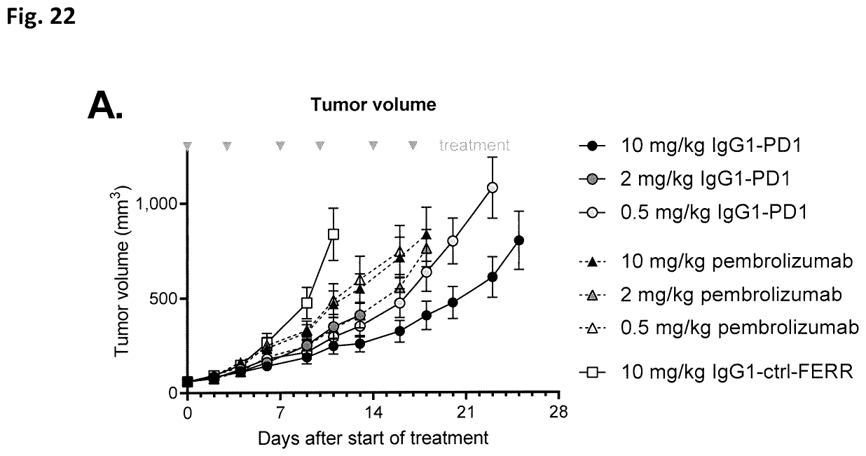

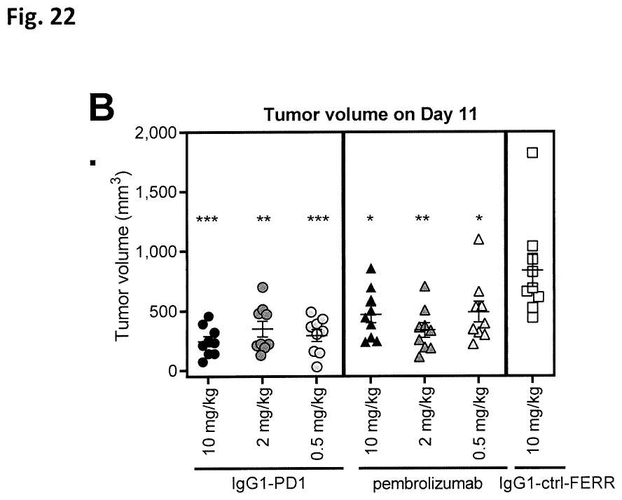

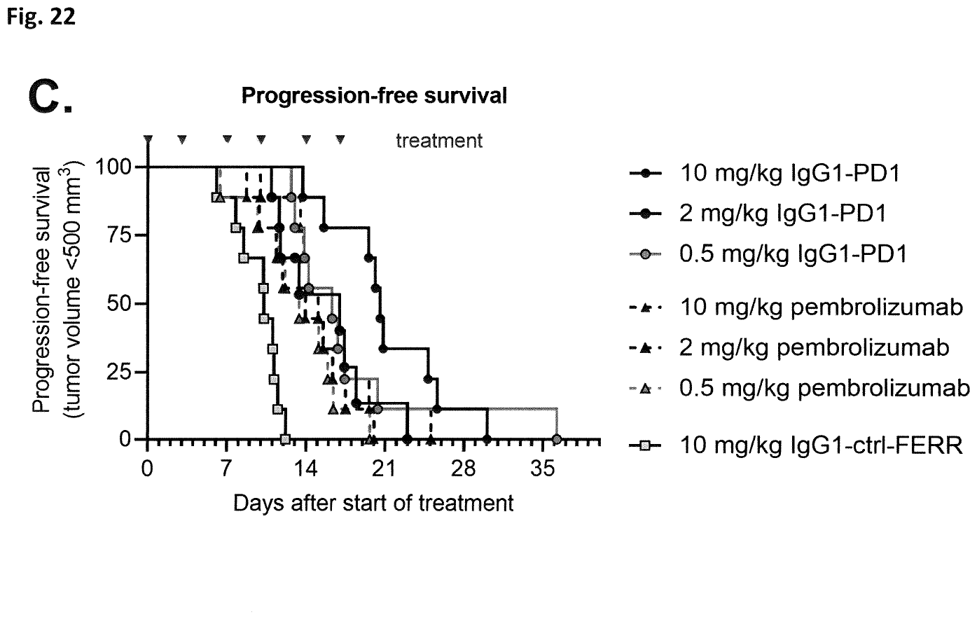

Treatment with IgG1-PD1 and pembrolizumab inhibited tumor growth at all doses tested (FIG. 22A). On Day 11, the last day that all treatment groups were complete, tumors in mice treated with IgG1-PD1 or pembrolizumab were significantly smaller at all doses tested than tumors in mice treated with 10 mg/kg IgG1-ctrl-FERR (FIG. 22B). In addition, at 10 mg/kg, tumor volumes in mice treated with IgG1-PD1 were significantly smaller than in mice treated with an equivalent dose of pembrolizumab (Mann-Whitney test, p=0.0188).

Treatment with IgG1-PD1 or pembrolizumab significantly increased progression-free survival (PFS) at all doses tested compared to mice treated with 10 mg/kg IgG1-ctrl-FERR (FIG. 22C). At 10 mg/kg, progression-free survival in mice treated with IgG1-PD1 was significantly extended as compared to mice treated with pembrolizumab (median PFS 10 mg/kg IgG1-PD1: 20.56 days, median PFS 10 mg/kg pembrolizumab: 13.94 days; P-value=0.0021).

In conclusion, IgG1-PD1 exhibited potent antitumor activity in MC38 tumor-bearing hPD-1 KI mice.

### Example 18: Effect of DuoBody-CD40×4-1BB in Combination with Anti-PD-(L)1 Antibodies on Cytokine Secretion in an Allogeneic MLR Assay

To analyze if the combination of DuoBody-CD40×4-1BB with atezolizumab, nivolumab or pembrolizumab could result in potentiation of cytokine production compared to single agent activity in a mixed lymphocyte reaction (MLR) assay, four unique, allogeneic pairs of human mature dendritic cells (mDCs) and CD8+ T cells were co-cultured in the presence of DuoBody-CD40×4-1BB alone, atezolizumab alone, nivolumab alone, pembrolizumab alone, or a combination of DuoBody-CD40×4-1BB with either atezolizumab, nivolumab or pembrolizumab. Cytokine secretion was assessed in the supernatants of the co-cultures using an IFNγ-specific immunoassay and a Luminex cytokine panel.

### Methods

Monocytes and T Cells from Healthy Donors

CD14+ monocytes and purified CD8+ T cells were obtained from BioIVT. Four unique allogeneic donor pairs were used for the MLR assay.

### Differentiation of Monocytes to Immature Dendritic Cells

Human CD14+ monocytes were obtained from healthy donors. For differentiation into immature dendritic cells (iDCs), 1-1.5×10⁶ monocytes/mL were cultured for six days in Roswell Park Memorial Institute (RPMI) 1640 complete medium (ATCC modification formula; ThermoFisher, cat. no. A1049101) supplemented with 10% heat-inactivated Fetal Bovine Serum (FBS; Gibco, cat. no. 16140071), 100 ng/mL granulocyte-macrophage colony-stimulating factor (GM-CSF; BioLegend, cat. no. 766106) and 300 ng/mL interleukin-4 (IL-4; BioLegend, cat. no. 766206) in T25 culture flasks (Falcon, cat. no. 353108) at 37° C. After four days, the medium was replaced with fresh medium and supplements.

Differentiation of iDCs to mDCs

Prior to start of the MLR assay, iDCs were harvested by collecting non-adherent cells and differentiated to mature DCs (mDCs) by incubating 1-1.5×10⁶ cells/mL in RPMI 1640 complete medium supplemented with 10% FBS, 100 ng/mL GM-CSF, 300 ng/mL IL-4 and 5 μg/mL lipopolysaccharide (LPS; ThermoFisher, cat. no. 00-4976-93) for 24 h at 37° C.

### Mixed Lymphocyte Reaction (MLR)

One day prior to the start of an MLR assay, purified CD8+ T cells obtained from allogeneic healthy donors were thawed, resuspended at 1×10⁶ cells/mL in RPMI 1640 complete medium supplemented with 10% FBS and 10 ng/mL IL-2 (BioLegend, cat. no. 589106) and incubated O/N at 37° C.

The next day, the LPS-matured dendritic cells (mDCs, see Maturation of iDCs to mDCs) and allogeneic purified CD8+ T cells were harvested and resuspended in AIM-V medium (ThermoFisher, cat. no. 12055091) at 4×10⁵ cells/mL and 4×10⁶ cells/mL, respectively. Co-cultures were seeded at a DC:T cell ratio of 1:10, corresponding to 20,000 mDCs incubated with 200,000 allogeneic purified CD8+ T cells, and cultured in the presence of atezolizumab (1 μg/mL; non-clinical/research-grade version of the clinical product atezolizumab; Selleckchem, cat. no. A2004), nivolumab (1 μg/mL; non-clinical/research-grade version of the clinical product nivolumab; Selleckchem, cat. no. A2002), pembrolizumab (1 μg/mL; non-clinical/research-grade version of the clinical product pembrolizumab; Selleckchem, cat. no. A2005) or DuoBody-CD40×4-1BB (0.001-30 μg/mL) as single agent, or DuoBody-CD40×4-1BB combined with either atezolizumab, nivolumab or pembrolizumab in AIM-V medium in a 96-well round-bottom plate (Falcon, cat. no. 353227) at 37° C. for 5 days. Co-cultures treated with bsIgG1-CD40×ctrl (30 μg/mL), bsIgG1-ctrl×4-1BB (30 μg/mL) or IgG1-ctrl-FEAL (30 μg/mL) were included as controls. After 5 days, the plates were centrifuged at 500×g for 5 min and the supernatant was carefully transferred from each well to a new 96-well round bottom plate.

The collected supernatants from the MLR assay were analyzed for IFNγ levels by enzyme-linked immunosorbent assay (ELISA) using an Alpha Lisa IFNγ kit (Perkin Elmer, cat. no. AL217) on an Envision instrument, according to the manufacturer's instructions.

The collected supernatants from the MLR assay were analyzed for interleukin (IL)-10, IL-12p40, IL-15, IL-17a, IL-1β, IL-2, IL-4, IL-23, IL-5, IL-6, IL-8, tumor necrosis factor (TNF)α, granulocyte-macrophage colony-stimulating factor (GM-CSF), monocyte chemoattractant protein-1 (MCP-1) and granzyme B on a Luminex FLEXMAP 3D® System using a custom Milliplex Chemokine Magnetic Bead Panel (Millipore Sigma, cat. no. HCYTOMAG-60K-08, Lot 3730985) according to manufacturer's instructions.

TABLE 26
Antibodies

| Test compound | Supplier, cat. no. | Comprising SEQ ID NOs |
|---|---|---|
| DuoBody-CD40 × 4-1BB | N/A | SEQ ID NO: 9, 10, 19, 20, 25, 26, 27 |
| bsIgG1-CD40 × ctrl¹ | N/A | SEQ ID NO: 9, 10, 25, 26, 27 |
| bslgG1-ctrl × 4-1BB¹ | N/A | SEQ ID NO: 19, 20, 25, 26, 27 |
| Atezolizumab | Selleckchem (non-clinical/research-grade | N/A |
| | version of the clinical product | |
| | atezolizumab; cat. no. A2004) | |
| Nivolumab | Selleckchem (non-clinical/research-grade | N/A |
| | version of the clinical product | |
| | nivolumab; cat. no. A2002, Lot no. #05) | |
| Pembrolizumab | Selleckchem (non-clinical/research-grade | N/A |
| | version of the clinical product | |
| | pembrolizumab; cat. no. A2005, Lot no. | |
| | A200504) | |
| IgG1-ctrl-FEAL¹ | N/A | SEQ ID NO: 25, 27 |

¹Control binding moiety based on anti-HIV gp120 antibody IgG1-b12 (Barbas et al., J Mol Biol 1993, 230: 812-823)

### Results & Conclusion

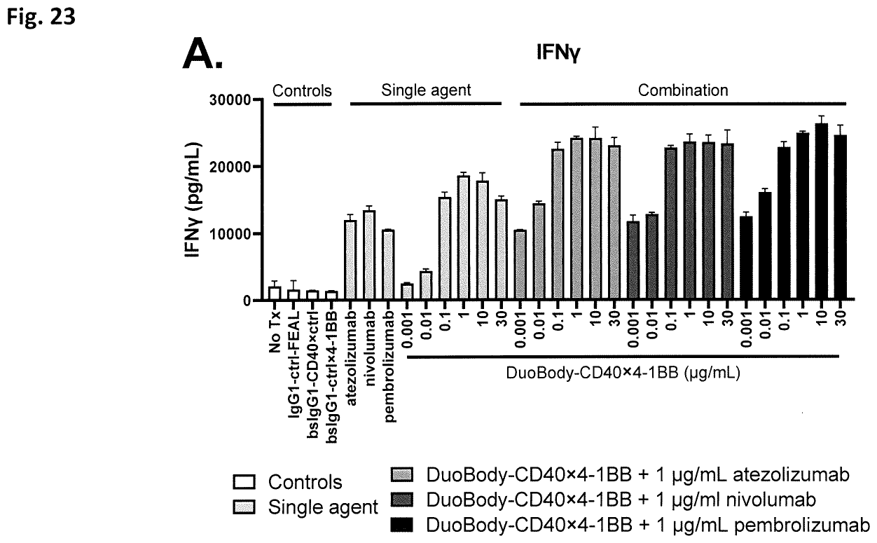

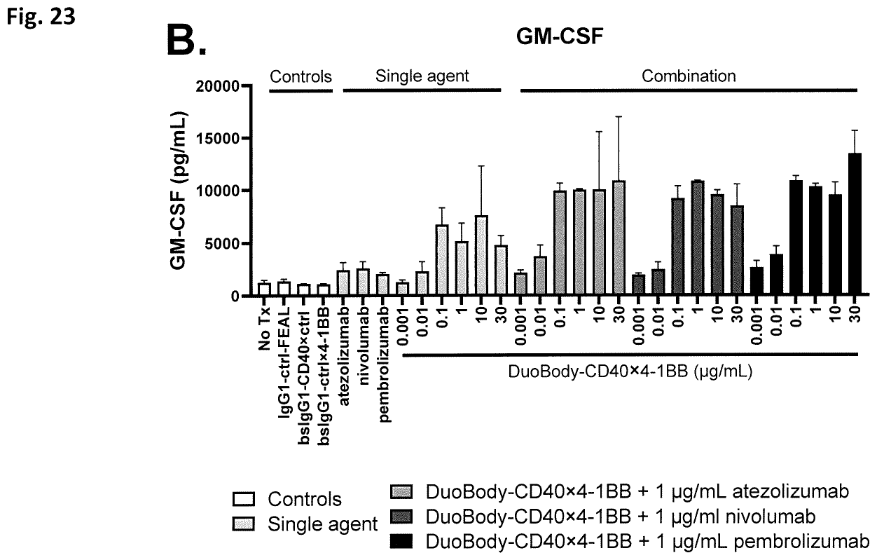

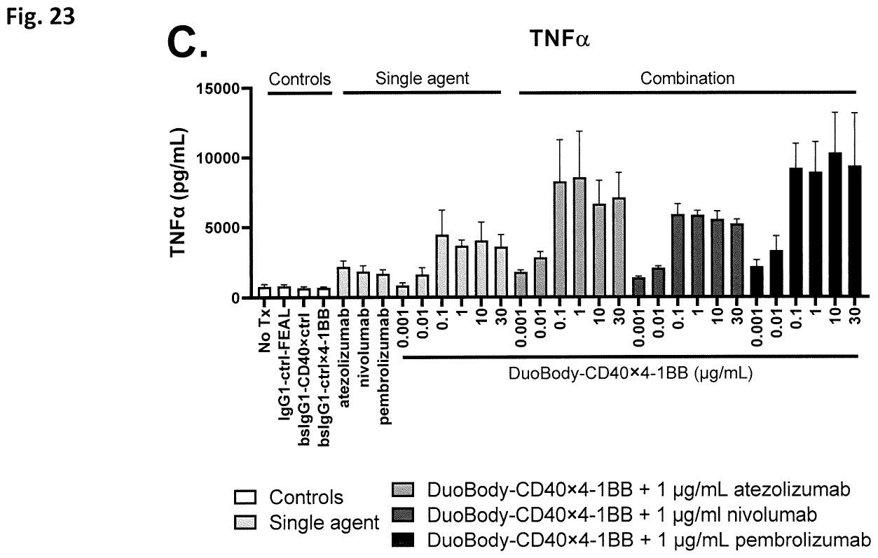

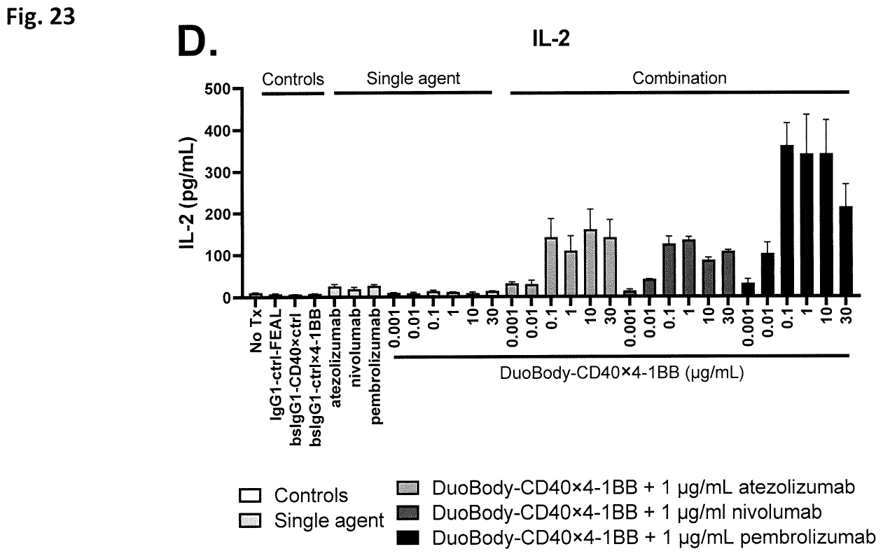

Treatment with DuoBody-CD40×4-1BB, atezolizumab, nivolumab or pembrolizumab alone enhanced the secretion of IFNγ, GM-CSF, TNFα, IL-2 and IL-6 in the MLR assay. Combination of ≥0.1 μg/mL DuoBody-CD40×4-1BB with 1 μg/mL atezolizumab, 1 μg/mL nivolumab or 1 μg/mL pembrolizumab further potentiated secretion of IFNγ, GM-CSF, TNFα, IL-2 and IL-6 compared to single-agent activity (FIG. 23). Combining DuoBody-CD40×4-1BB with atezolizumab, nivolumab or pembrolizumab increased the levels of IFNγ, GM-CSF and IL-6 to the same extent. TNFα and IL-2 secretion was highest after combining DuoBody-CD40×4-1BB with pembrolizumab. These data suggest that the magnitude of immune response can be amplified through targeting of the PD-1/PD-L1 axis in combination with CD40 and 4-1BB co-stimulation.

### Example 19: Antigen-Specific Stimulation Assay to Determine the Capacity of DuoBody-CD40×4-1BB in Combination with Anti-PD-(L)1 Antibodies to Enhance T-Cell Proliferation and Cytokine Secretion

To determine the combinatorial effect of DuoBody-CD40×4-1BB and anti-PD-(L)1 antibodies on T-cell proliferation and cytokine production compared to single-agent activity, an antigen-specific stimulation assay was conducted using co-cultures of PD-1-overexpressing human CD8+ T cells and cognate antigen-expressing immature dendritic cells (iDCs).

### Methods

Isolation of Cells and Differentiation of Monocytes to iDCs

HLA-A*02⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were cryopreserved for T-cell isolation. For differentiation into iDCs, 1×10⁶ monocytes/mL were cultured for five days in RPMI 1640 (Life Technologies GmbH, cat. no. 61870-010) containing 5% pooled human serum (One Lambda Inc., cat. no. A25761), 1 mM sodium pyruvate (Life technologies GmbH, cat. no. 11360-039), 1× non-essential amino acids (Life Technologies GmbH, cat. no. 11140-035), 200 ng/mL granulocyte-macrophage colony-stimulating factor (GM-CSF; Miltenyi, cat. no. 130-093-868) and 200 ng/mL interleukin-4 (IL-4; Miltenyi, cat. no. 130-093-924). On day 3, half of the medium was replaced with fresh medium containing supplements. iDCs were harvested by collecting non-adherent cells and adherent cells were detached by incubation with Dulbecco's phosphate-buffered saline (DPBS) containing 2 mM EDTA for 10 min at 37° C. After washing with DPBS iDCs were cryopreserved in FBS (Sigma-Aldrich, cat. no. F7524) containing 10% DMSO (AppliChem GmbH, cat. no A3672,0050) for future use in antigen-specific T cell assays.

Electroporation of iDCs and CD8+ T Cells and CFSE-Labeling

One day prior to the start of an antigen-specific CD8+ T cell stimulation assay, frozen PBLs and iDCs from the same donor were thawed. CD8+ T cells were isolated from PBLs by MACS technology using anti-CD8 MicroBeads (Miltenyi, cat. no. 130-045-201), according to the manufacturer's instructions. About 10×10⁶ to 15×10⁶ CD8+ T cells were electroporated with each 10 μg of in vitro transcribed (IVT)-RNA encoding the alpha and beta chains of a murine TCR specific for human claudin-6 (CLDN6; HLA-A*02-restricted; described in WO 2015/150327 A1) plus 10 μg IVT-RNA encoding human PD-1 (UniProt Q15116) in 250 μL X-Vivol5 medium (Lonza, cat. no. BE02-060Q). The cells were transferred to a 4-mm electroporation cuvette (VWR International GmbH, cat. no. 732-0023) and electroporated using the BTX ECM® 830 Electroporation System (BTX; 500 V, 3 ms pulse). Immediately after electroporation, cells were transferred into fresh IMDM GlutaMAX medium (Life Technologies GmbH, cat. no. 319800-030) containing 5% pooled human serum and rested at 37° C., 5% CO₂ for at least 1 hour. T cells were labeled using 0.8 μM carboxyfluorescein succinimidyl ester (CFSE; Life Technologies GmbH, cat. No V12883) in PBS according to the manufacturer's instructions and incubated in IMDM medium supplemented with 5% pooled human serum overnight.

Up to 5×10⁶ thawed iDCs were electroporated with 2 μg IVT-RNA encoding full-length human CLDN6 (WO 2015/150327 A1), in 250 μL X-Vivol5 medium, using the electroporation system as described above (300 V, 12 ms pulse) and incubated in IMDM medium supplemented with 5% pooled human serum overnight.

The next day, cells were harvested. Cell-surface expression of CLDN6 on iDCs, as well as cell-surface expression of the CLDN6-specific TCR and PD-1 on T cells was confirmed by flow cytometry. To this end, iDCs were stained with a fluorescently labeled CLDN6-specific antibody (non-commercially available; in-house production). T cells were stained with a brilliant violet (BV)421-conjugated anti-mouse TCR-β chain antibody (Becton Dickinson GmbH, cat. no. 562839) and an allophycocyanin (APC)-conjugated anti-human PD-1 antibody (Thermo Fisher Scientific, cat. no. 17-2799-42).

### Antigen-Specific In Vitro T-Cell Stimulation Assay

Electroporated iDCs were incubated with electroporated, CFSE-labeled T cells at a ratio of 1:10 in the presence of DuoBody-CD40×4-1BB (0.0022, 0.0067, or 0.2 μg/mL), either alone or in combination with the anti-PD-1 antibodies pembrolizumab (Keytruda®, Merck Sharp & Dohme GmbH, PZN 10749897) (0.8 μg/mL), nivolumab (Opdivo®, Bristol-Myers-Squib GmbH, PZN 11024601) (1.6 μg/mL), or IgG1-PD1 (0.8 μg/mL), the anti-PD-L1 antibody atezolizumab (Tecentriq®, Roche Pharma AG, PZN 11306050) (0.4 μg/mL), or the negative control antibody IgG1-ctrl-FERR (0.8 μg/mL), in IMDM medium (Life Technologies GmbH, cat. No 31980030) containing 5% pooled human serum (One Lambda Inc., cat. No A25761) in a 96-well round-bottom plate (VWR International GmbH, cat. No 734-1797). After 4 days of culture, the cells were stained with an APC-conjugated anti-human CD8 antibody. T-cell proliferation was evaluated by flow cytometry analysis of CFSE dilution in CD8+ T cells using a BD FACSCelesta™ flow cytometer (Becton Dickinson GmbH).

Flow cytometry data was analyzed using FlowJo software version 10.7.1. CFSE label dilution of CD8+ T cells was assessed using the proliferation modeling tool in FlowJo, and expansion indices calculated using the integrated formula.

### Determination of Cytokine Concentrations

Cytokine concentrations in supernatants that had been collected from T cell/iDC co-cultures after 4 days were determined by multiplexed electrochemiluminescence immunoassay using a custom-made U-Plex biomarker group 1 (human) assay for the detection of panel of 10 human cytokines (GM-CSF, IL-2, IL-8, IL-10, IL-12p70, IL-13, IFNγ, IFNγ-inducible protein [IP]-10 [also known as C-X-C motif chemokine ligand 10], macrophage chemoattractant protein [MCP] 1, and TNFα; Meso Scale Discovery, cat. No. K15067L-2) following the manufacturer's protocol.

TABLE 27
Antibodies used in the antigen-specific T cell stimulation assay

| Test compound | Supplier, cat. no. | Comprising SEQ ID NOs |
|---|---|---|
| DuoBody-CD40 × 4-1BB | N/A | SEQ ID NO: 9, 10, 19, 20, 25, 26, 27 |
| IgG1-PD1 | N/A | SEQ ID NO: 27, 43, 44, 61 |
| IgG1-ctrl-FERR¹ | N/A | SEQ ID NO: 27, 59, 60, 62 |
| Pembrolizumab | Keytruda ®, Merck Sharp & | N/A |
| | Dohme GmbH, PZN 10749897 | |
| Nivolumab | Opdivo ®, Bristol-Myers-Squibb | N/A |
| | GmbH, PZN 11024601 | |
| Atezolizumab | Tecentriq ®, Roche Pharma AG, | N/A |
| | PZN 11306050 | |

¹Control binding moiety based on anti-HIV gp120 antibody IgG1-b12 (Barbas et al., J Mol Biol 1993, 230: 812-823)

### Results & Conclusion

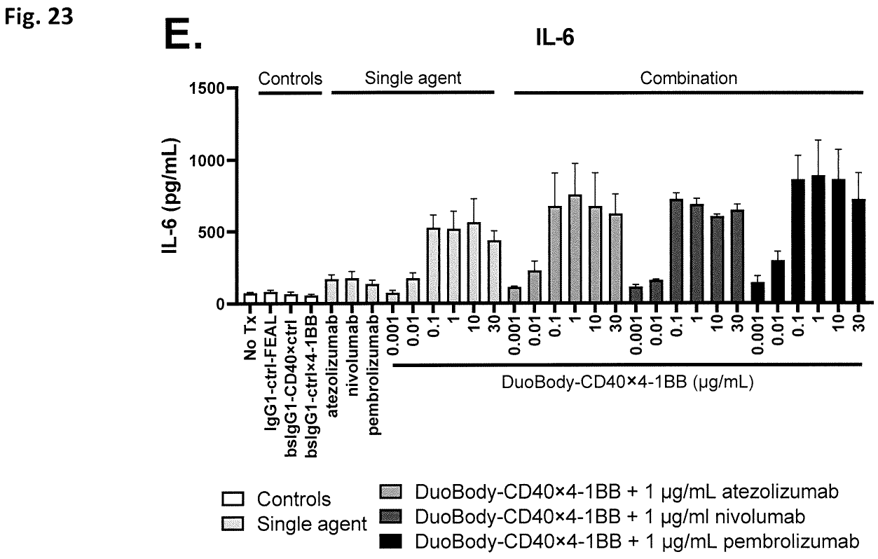

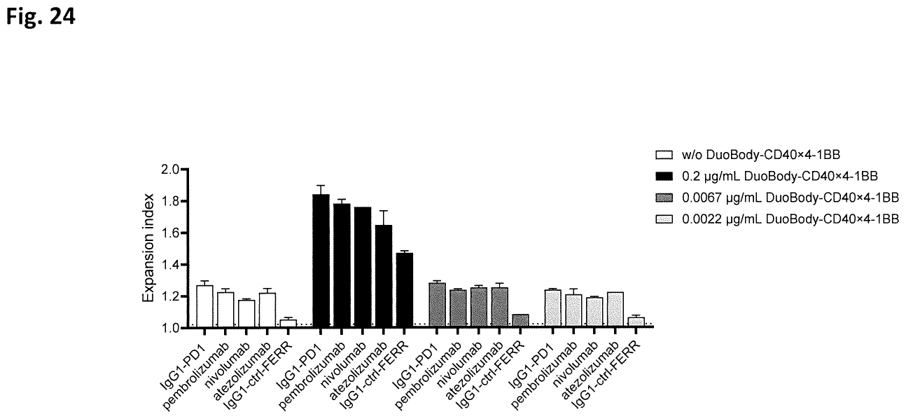

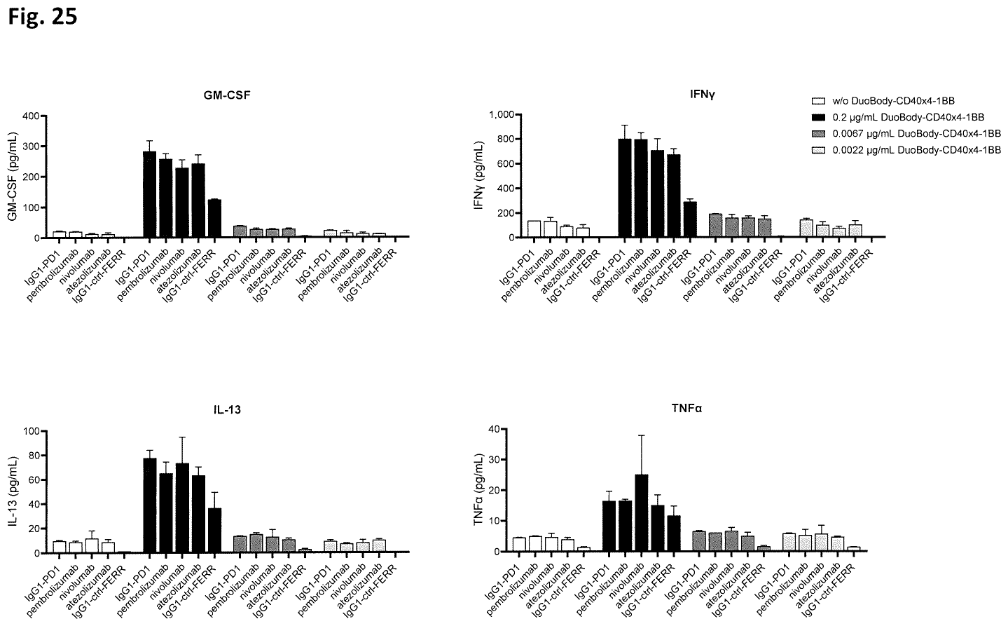

Combination treatment with DuoBody-CD40×4-1BB and anti-PD-(L)1 antibodies potentiated CD8+ T-cell proliferation, compared to DuoBody-CD40×4-1BB combined with non-binding control antibody IgG1-ctrl-FERR and compared to anti-PD-(L)1 antibodies as single treatment (FIG. 24). An increase in proliferation was seen when a high concentration of DuoBody-CD40×4-1BB (0.2 μg/mL) was used in combination treatment, compared to both single-agent treatments. Combination of anti-PD-(L)1 antibodies with low (0.0022 μg/mL) or intermediate (0.0067 μg/mL) concentrations of DuoBody-CD40×4-1BB had minimal or no effect on proliferation, compared to anti-PD-(L)1 antibodies alone. Combination treatment with DuoBody-CD40×4-1BB and anti-PD-(L)1 antibodies enhanced the secretion of the proinflammatory cytokines GM-CSF, IFNγ, IL-13, and TNFα, compared to DuoBody-CD40×4-1BB combined with IgG1-ctrl-FERR and compared to anti-PD-(L)1 antibodies as single treatment (FIG. 25). An increase in cytokine secretion was seen when a high concentration of DuoBody-CD40×4-1BB (0.2 μg/mL) was used in combination treatment, compared to both single-agent treatments. Combination of anti-PD-(L)1 antibodies with low (0.0022 μg/mL) or intermediate (0.0067 μg/mL) concentrations of DuoBody-CD40×4-1BB had minimal or no effect on cytokine secretion, compared to anti-PD-(L)1 antibodies alone. Secretion of other cytokines tested were not consistently enhanced, or not enhanced, compared to single-agent treatments.

### Example 20: Polyclonal Stimulation Assay to Determine the Capacity of DuoBody-CD40×4-1BB in Combination with Anti-PD-(L)1 Antibodies to Enhance T-Cell Proliferation

To determine the combinatorial effect of DuoBody-CD40×4-1BB and anti-PD-(L)1 antibodies on T-cell proliferation compared to single-agent activity, a polyclonal stimulation assay was conducted using PBMC cultures stimulated with an anti-CD3 antibody.

### Methods

### Polyclonal In Vitro T-Cell Stimulation Assay

PBMCs were obtained from healthy donors (Transfusionszentrale, University Hospital, Mainz, Germany). PBMCs were labeled using 2.5 μM CellTrace™ Violet (Thermo Fisher Scientific, cat. No C34557) in PBS according to the manufacturer's instructions.

The PBMCs were cultured with 0.09 μg/mL of an anti-CD3 antibody (clone UCHT1, R&D systems, cat. No MAB100-500) in the presence of DuoBody-CD40×4-1BB (0.2 μg/mL), either alone or in combination with the anti-PD-1 antibodies pembrolizumab (Keytruda®, Merck Sharp & Dohme GmbH, PZN 10749897) or nivolumab (Opdivo®, Bristol-Myers-Squib GmbH, PZN 11024601), or the anti-PD-L 1 antibody atezolizumab (Tecentriq®, Roche Pharma AG, PZN 11306050) (all used at 0.0, 0.5, and 5 μg/mL), in IMDM medium (Life Technologies GmbH, cat. No 31980030) containing 5% pooled human serum (One Lambda Inc., cat. No A25761) in a 96-well round-bottom plate (VWR International GmbH, cat. No 734-1797). The nonbinding antibody IgG1-ctrl-FEAL (0.2 μg/mL) was included as a negative control. After 4 days of culture, the cells were stained with APC-conjugated anti-human CD8 (Becton Dickinson GmbH, cat. No 640584) and PE-conjugated anti-human CD4 (TONBO Biosciences, cat. No 50-0049) antibodies. T-cell proliferation was evaluated by flow cytometry analysis of CellTrace™ Violet dilution in CD8+ and CD4+ T cells using a BD FACSCelesta™ flow cytometer (Becton Dickinson GmbH).

TABLE 28
Antibodies used in polyclonal stimulation assay

| Test compound | Supplier, cat. no. | Comprising SEQ ID NOs |
|---|---|---|
| DuoBody-CD40 × 4-1BB | N/A | SEQ ID NO: 9, 10, 19, 20, 25, 26, 27 |
| IgG1-ctrl-FEAL¹ | N/A | SEQ ID NO: 25, 27 |
| Pembrolizumab | Keytruda ®, Merck Sharp & | N/A |
| | Dohme GmbH, PZN 10749897 | |
| Nivolumab | Opdivo ®, Bristol-Myers-Squibb | N/A |
| | GmbH, PZN 11024601 | |
| Atezolizumab | Tecentriq ®, Roche Pharma AG, | N/A |
| | PZN 11306050 | |

¹Control binding moiety based on anti-HIV gp120 antibody IgG1-b12 (Barbas et al., J Mol Biol 1993, 230: 812-823)

### Results

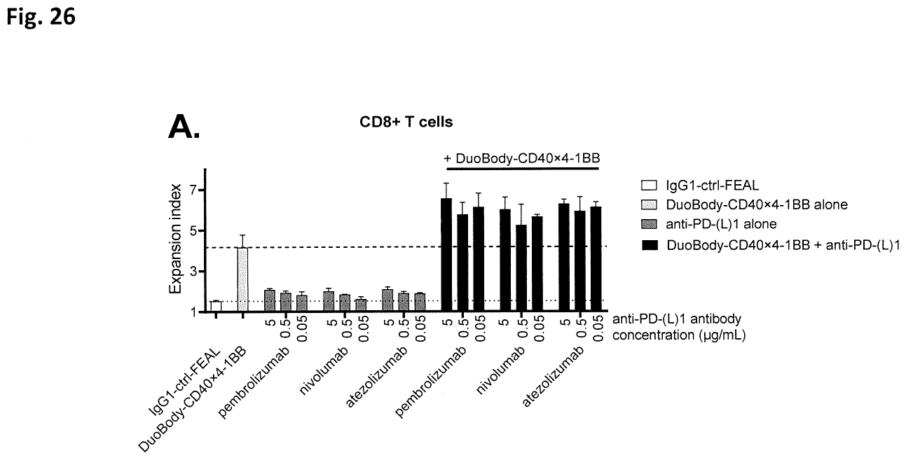

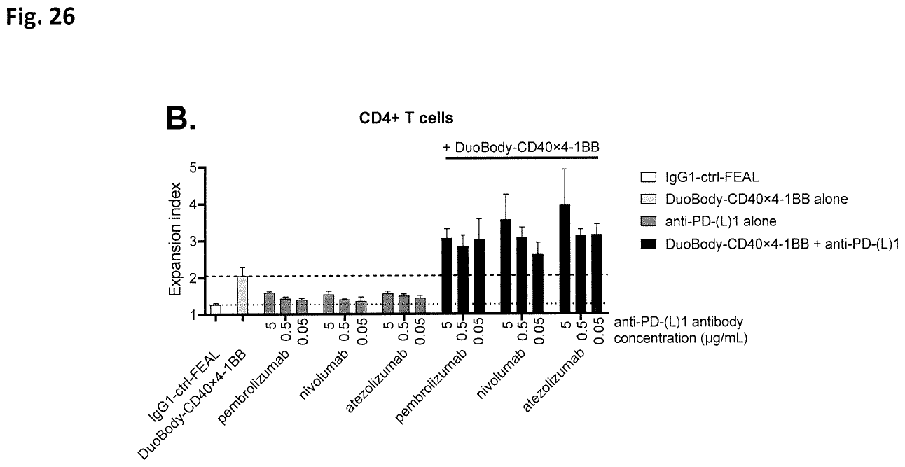

Combination treatment with DuoBody-CD40×4-1BB and anti-PD-(L)1 antibodies potentiated CD8+ and CD4+ T-cell proliferation, compared to DuoBody-CD40×4-1BB and compared to anti-PD-(L)1 antibodies as single treatment (FIG. 26). Potentiation of single-agent activity was observed at all tested concentrations of anti-PD-(L)1 antibodies.

### Example 21: Effect of DuoBody-CD40×4-1BB in Combination with Pembrolizumab on Cytokine Secretion in an Allogeneic MLR Assay of LPS-Matured Dendritic Cells and In Vitro Exhausted T Cells

To analyze if the combination of DuoBody-CD40×4-1BB with pembrolizumab could reverse T-cell exhaustion in a mixed lymphocyte reaction (MLR) assay, two unique, allogeneic pairs of human mature dendritic cells (mDCs) and in vitro exhausted T cells (Tex) were co-cultured in the presence of DuoBody-CD40×4-1BB alone, pembrolizumab alone, or a combination of both antibodies. Expression of inhibitory receptors on Tex was determined by flow cytometry and secretion of interferon (IFN)γ and interleukin (IL)-2 was assessed in the supernatants of the co-cultures.

### Methods

Monocytes and T Cells from Healthy Donors

CD14+ monocytes and purified CD3+ T cells were obtained from BioIVT. Two unique allogeneic donor pairs were used for the MLR assay.

### Differentiation of Monocytes to Immature Dendritic Cells

Human CD14+ monocytes were obtained from healthy donors. For differentiation into immature dendritic cells (iDCs), 1-1.5×10⁶ monocytes/mL were cultured for six days in Roswell Park Memorial Institute (RPMI) 1640 complete medium (ATCC modification formula; ThermoFisher, cat. no. A1049101) supplemented with 10% heat-inactivated fetal bovine serum (FBS; Gibco, cat. no. 16140071), 100 ng/mL granulocyte-macrophage colony-stimulating factor (GM-CSF; BioLegend, cat. no. 766106) and 300 ng/mL IL-4 (BioLegend, cat. no. 766206) in T25 culture flasks (Falcon, cat. no. 353108) at 37° C. After four days, the medium was replaced with fresh medium and supplements.

Differentiation of iDCs to mDCs

Prior to start of the MLR assay, iDCs were harvested by collecting non-adherent cells and differentiated to mDCs by incubating 1-1.5×10⁶ cells/mL in RPMI 1640 complete medium supplemented with 10% FBS, 100 ng/mL GM-CSF, 300 ng/mL IL-4 and 5 μg/mL lipopolysaccharide (LPS; ThermoFisher, cat. no. 00-4976-93) for 24 h at 37° C.

### Exhaustion of T Cells

Purified CD3+ T cells obtained from healthy donors were thawed and resuspended at 1×10⁶ cells/mL in AIM-V medium (ThermoFisher, cat. no. 12055091) supplemented with 5% FBS and 10 ng/mL IL-2 (BioLegend, cat. no. 589106). To induce T cells with an exhausted-like phenotype the cells were stimulated for two rounds with of Dynabeads™ Human T Activator CD3/CD28 (Gibco, cat. no. 11161D) at a bead:cell ratio of 1:1 for 48 h at 37° C. and 5% CO₂. After two rounds of stimulation the exhausted CD3+ T cells (Tex) were rested for 24 h.

As an unstimulated control, purified CD3+ T cells obtained from healthy donors were thawed one day prior to the start of the MLR assay, resuspended at 1×10⁶ cells/mL in RPMI 1640 complete medium supplemented with 10% FBS and 10 ng/mL IL-2 and incubated O/N at 37° C. Prior to the MLR assay, aliquots of unstimulated T cells and Tex were collected for flow cytometry.

### Flow Cytometry

For flow cytometry analysis of inhibitory receptors on Tex, cells were pelleted at 400×g for 5 min, washed in phosphate-buffered saline (PBS), pelleted again, resuspended in 1 mL PBS supplemented with LIVE/DEAD™ Fixable Near-IR Dead Cell Stain (ThermoFisher Scientific, cat. no. L10119, diluted 1:500) and incubated for 20 min at 4° C. in the dark. Next, cells were washed, pelleted, resuspended to 8×10⁶ cells/mL in FACS buffer (Dulbecco's phosphate-buffered saline [DPBS, Gibco, cat. no. 14190136] supplemented with 0.5% bovine serum albumin [BSA, Sigma, cat. no. A9576] and 2 mM ethylenediaminetetraacetic acid [EDTA, Invitrogen, cat. no. 15575-038]) containing 5% human serum (Sigma, cat. no. H4522), and incubated 15 min at 4° C. Then 25 μL containing 200,000 cells was transferred to a new 96-well plate containing 150 μL staining mix with PE-labeled anti-human LAG3 (Biolegend, cat. no. 369306, diluted 1:60) in FACS buffer supplemented with Brilliant Stain Buffer Plus (BD Horizon, cat. no. 566385) and incubated for 20 min at RT in the dark. Cells were pelleted, washed using FACS buffer, resuspended in 100 μL Fixation Buffer (Biolegend, cat. no. 420801) and incubated for 15 min at 4° C. in the dark. Cells were pelleted again, washed and resuspended in 100 μL FACS buffer. Samples were analyzed on a Cytek® Aurora flow cytometer (Cytek Biosciences).

### MLR Assay

The mDCs (see Maturation of iDCs to mDCs) were harvested and resuspended in AIM-V medium at 4×10⁵ cells/mL. Tex and unstimulated CD3+ T cells (see Exhaustion of T cells) were harvested and resuspended in AIM-V medium at 4×10⁶ cells/mL. Co-cultures of mDC and Tex were seeded at a DC:T cell ratio of 1:4 or 1:10, corresponding to 20,000 mDCs incubated with 80,000 or 200,000 Tex, and cultured in the presence of pembrolizumab (1 μg/mL; non-clinical/research-grade version of the clinical product pembrolizumab; Selleckchem, cat. no. A2005) or DuoBody-CD40×4-1BB (0.001-30 μg/mL) as single agent, or both agents combined in AIM-V medium in a 96-well round-bottom plate (Falcon, cat. no. 353227) at 37° C. for 5 days. Co-cultures treated with bsIgG1-CD40×ctrl (30 μg/mL), bsIgG1-ctrl×4-1BB (30 μg/mL) or IgG1-ctrl-FEAL (30 μg/mL) were included as controls. In parallel, co-cultures of mDC and unstimulated CD3+ T cells at a DC:T cell ratio of 1:10, corresponding to 20,000 mDCs incubated with 200,000 T cells, were cultured with and without 1 μg/mL pembrolizumab. After 5 days, the plates were centrifuged at 500×g for 5 min and the supernatant was carefully transferred from each well to a new 96-well round bottom plate.

The collected supernatants were analyzed for IFNγ levels by enzyme-linked immunosorbent assay (ELISA) using an AlphaLISA IFNγ kit (Perkin Elmer, cat. no. AL217) on an Envision instrument, according to the manufacturer's instructions. The collected supernatants were analyzed for IL-2 on a Meso Sector S 600 (Meso Scale Discovery [MSD], cat. no. R31QQ-3) using the V-Plex Proinflammatory Panel 1 Human Kit (MSD, cat. no. K15049D) according to manufacturer's instructions.

TABLE 29
Antibodies

| Test compound | Supplier, cat. no. | Comprising SEQ ID NOs |
|---|---|---|
| DuoBody-CD40 × 4-1BB | N/A | SEQ ID NO: 9, 10, 19, 20, 25, 26, 27 |
| bsIgG1-CD40 × ctrl¹ | N/A | SEQ ID NO: 9, 10, 25, 26, 27 |
| bsIgG1-ctrl × 4-1BB¹ | N/A | SEQ ID NO: 19, 20, 25, 26, 27 |
| Pembrolizumab | Selleckchem (non-clinical/research-grade | N/A |
| | version of the clinical product | |
| | pembrolizumab; cat. no. A2005, Lot no. | |
| | A200504) | |
| IgG1-ctrl-FEAL¹ | N/A | SEQ ID NO: 25, 27 |

¹Control binding moiety based on anti-HIV gp120 antibody IgG1-b12 (Barbas et al., J Mol Biol 1993, 230: 812-823)

### Results & Conclusion

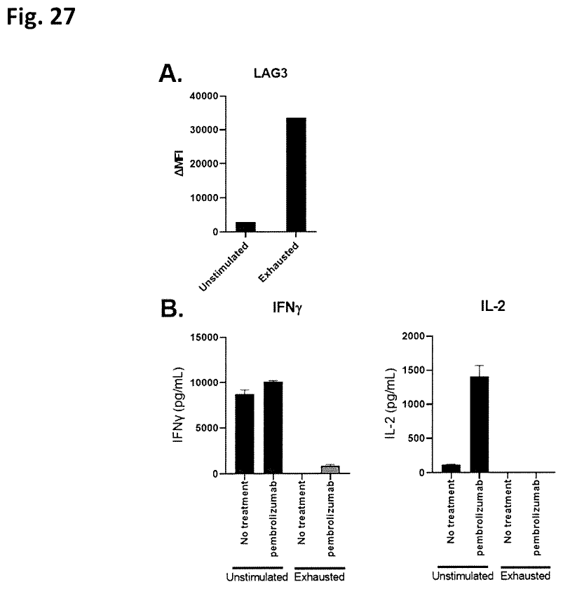

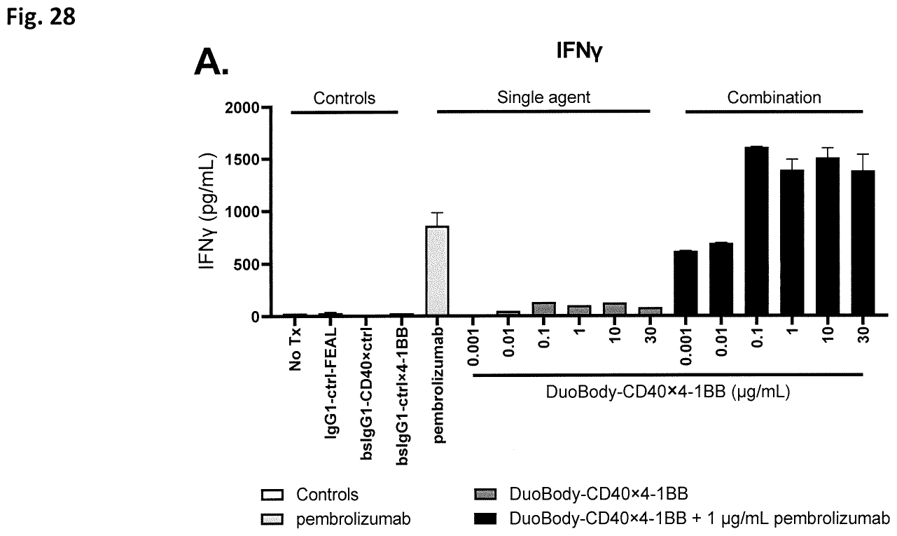

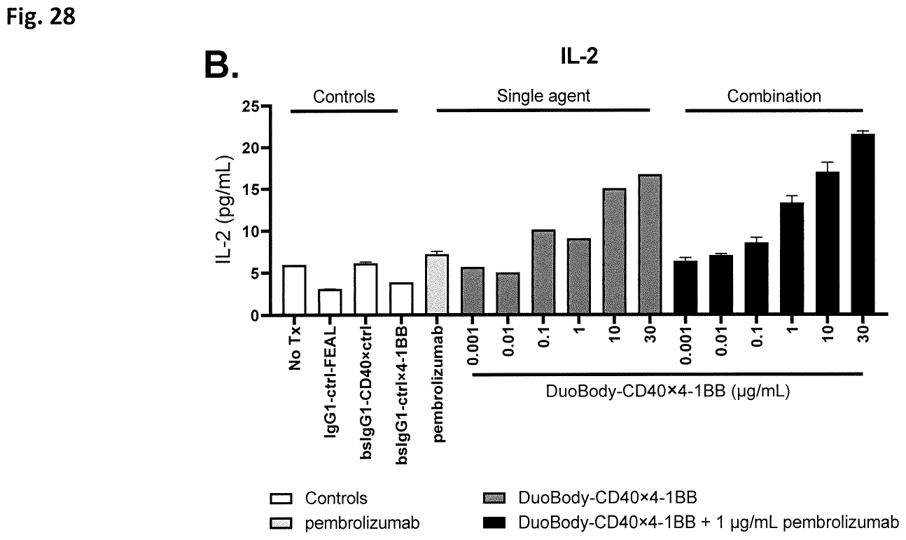

After two rounds of stimulation with CD3/CD28 beads, the T cells expressed inhibitory receptor LAG3 and became hyporesponsive to dual anti-CD3 and anti-CD28 stimulation, as demonstrated by reduced secretion of IFNγ and IL-2 in MLR assays of mDCs and Tex as compared to unstimulated CD3+ T cells (FIG. 27). Treatment with pembrolizumab or DuoBody-CD40×4-1BB as single agents partially rescued IFNγ or IL-2 secretion, respectively. Combination of ≥0.1 μg/mL DuoBody-CD40×4-1BB with 1 μg/mL pembrolizumab further potentiated secretion of IFNγ compared to single-agent activity in the mDC:Tex MLR assays (FIG. 28). These data suggest that loss of cytokine secretion by exhausted T cells can be partially reversed through targeting of the PD-1/PD-11 axis in combination with CD40 and 4-1BB co-stimulation.

1. A binding agent for use in a method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137.

2. The binding agent for use of claim 1, wherein CD40 is human CD40, in particular human CD40 comprising the sequence set forth in SEQ ID NO: 36, and/or CD137 is human CD137, in particular human CD137 comprising the sequence set forth in SEQ ID NO: 38.

3. The binding agent for use of claim 1 or 2, wherein the checkpoint inhibitor is at least one selected from the group consisting of PD-1 inhibitors, PD-L1 inhibitors, PD-L2 inhibitors, CTLA-4 inhibitors, TIM-3 inhibitors, KIR inhibitors, LAG-3 inhibitors, TIGIT inhibitors, VISTA inhibitors, and GARP inhibitors.

4. The binding agent for use of any one of claims 1 to 3, wherein the checkpoint inhibitor is an antibody, such as a PD-1 blocking antibody, in particular pembrolizumab.

5. The binding agent for use of any one of the preceding claims, wherein one or both of the binding agent and the checkpoint inhibitor is/are administered systemically, preferably intravenously.

6. The binding agent for use of any one of the preceding claims, wherein
a) the first binding region comprises a heavy chain variable region (VH) comprising the CDR1, CDR2, and CDR3 sequences of SEQ ID NO: 7 or 9, and a light chain variable region (VL) comprising the CDR1, CDR2, and CDR3 sequences of SEQ ID NO: 8 or 10;
and
b) the second antigen-binding region comprises a heavy chain variable region (VII) comprising the CDR1, CDR2, and CDR3 sequences of SEQ ID NO: 17 or 19, and a light chain variable region (VL) comprising the CDR1, CDR2, and CDR3 sequences of SEQ ID NO: 18 or 20.

7. The binding agent for use of any one of the preceding claims, wherein
a) the first binding region comprises a heavy chain variable region (VH) comprising the CDR1, CDR2, and CDR3 sequences set forth in: SEQ ID NO: 1, 2, and 3, respectively, and a light chain variable region (VL) comprising the CDR1, CDR2, and CDR3 sequences set forth in: SEQ ID NO: 4, 5, and 6, respectively;
and
b) the second antigen-binding region comprises a heavy chain variable region (VH) comprising the CDR1, CDR2, and CDR3 sequences set forth in: SEQ ID NO: 11, 12, and 13, respectively, and a light chain variable region (VL) comprising the CDR1, CDR2, and CDR3 sequences set forth in: SEQ ID NO: 14, 15, and 16, respectively.

8. The binding agent for use of any one of the preceding claims, wherein
a) the first binding region comprises a heavy chain variable region (VH) comprising an amino acid sequence having at least 90%, at least 95%, at least 97%, at least 99%, or 100% sequence identity to SEQ ID NO: 7 or 9 and a light chain variable region (VL) region and comprising an amino acid sequence having at least 90%, at least 95%, at least 97%, at least 99%, or 100% sequence identity to SEQ ID NO: 8 or 10;
b) the second binding region comprises a heavy chain variable region (VH) comprising an amino acid sequence having at least 90%, at least 95%, at least 97%, at least 99%, or 25 100% sequence identity to SEQ ID NO: 17 or 19 and a light chain variable region (VL) region comprising an amino acid sequence having at least 90%, at least 95%, at least 97%, at least 99%, or 100% sequence identity to SEQ ID NO: 18 or 20.

9. The binding agent for use of any one of the preceding claims, wherein
a) the first binding region comprises a heavy chain variable region (VH) comprising the amino acid sequence set forth in SEQ ID NO: 7 or 9 and a light chain variable region (VL) region comprising the amino acid sequence set forth in SEQ ID NO: 8 or 10;
and
b) the second binding region comprises a heavy chain variable region (VH) comprising the amino acid sequence set forth in SEQ ID NO: 17 or 19 and a light chain variable region (VL) region comprising the amino acid sequence set forth in SEQ ID NO: 18 or 20.

10. The binding agent for use of any one of the preceding claims, wherein
a) the first binding region comprises a heavy chain variable region (VH) comprising the amino acid sequence set forth in SEQ ID NO: 9 and a light chain variable region (VL) region comprising the amino acid sequence set forth in SEQ ID NO: 10;
and
b) the second binding region comprises a heavy chain variable region (VH) comprising the amino acid sequence set forth in SEQ ID NO: 19 and a light chain variable region (VL) region comprising the amino acid sequence set forth in SEQ ID NO: 20.

11. The binding agent for use of any one of the preceding claims, wherein the binding agent is a multispecific antibody, such as a bispecific antibody.

12. The binding agent for use of any one of the preceding claims, wherein the binding agent is in the format of a full-length antibody or an antibody fragment.

13. The binding agent for use of any one of claims 6-12, wherein each variable region comprises three complementarity determining regions (CDR1, CDR2, and CDR3) and four framework regions (FR1, FR2, FR3, and FR4).

14. The binding agent for use of claim 13, wherein said complementarity determining regions and said framework regions are arranged from amino-terminus to carboxy-terminus in the following order: FR1, CDR1, FR2, CDR2, FR3, CDR3, FR4.

15. The binding agent for use of any one of claims 6-14, which comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

16. The binding agent for use of any one of claims 6-15, which comprises
i) a polypeptide comprising said first light chain variable region (VL) and further comprising a first light chain constant region (CL), and
ii) a polypeptide comprising said second light chain variable region (VL) and further comprising a second light chain constant region (CL).

17. The binding agent for use of any one of claims 6-16, wherein the binding agent is an antibody comprising a first binding arm and a second binding arm, wherein
the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).

18. The binding agent for use of any one of the preceding claims, which comprises
i) a first heavy chain and light chain comprising said antigen-binding region capable of binding to CD40, and
ii) a second heavy chain and light chain comprising said antigen-binding region capable of binding CD137.

19. The binding agent for use of any one of the preceding claims, wherein said binding agent comprises
i) a first heavy chain and light chain comprising said antigen-binding region capable of binding to CD40, the first heavy chain comprising a first heavy chain constant region and the first light chain comprising a first light chain constant region; and
ii) a second heavy chain and light chain comprising said antigen-binding region capable of binding CD137, the second heavy chain comprising a second heavy chain constant region and the second light chain comprising a second light chain constant region.

20. The binding agent for use of any one of claims 15-19, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

21. The binding agent for use of any one of claims 15-20, wherein each of the first and second heavy chain constant regions (CHs) comprises a CH3 region and wherein the two CH3 regions comprise asymmetrical mutations.

22. The binding agent for use of any one of claims 15-21, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

23. The binding agent for use of claim 22, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

24. The binding agent for use of any of the preceding claims, wherein said binding agent induces Fe-mediated effector function to a lesser extent compared to another antibody comprising the same first and second antigen binding regions and two heavy chain constant regions (CHs) comprising human IgG1 hinge, CH2 and CH3 regions.

25. The binding agent for use of claim 24, wherein said first and second heavy chain constant regions (CHs) are modified so that the antibody induces Fc-mediated effector function to a lesser extent compared to an antibody which is identical except for comprising non-modified first and second heavy chain constant regions (CHs).

26. The binding agent for use of claim 25, wherein each of said non-modified first and second heavy chain constant regions (CHs) comprises the amino acid sequence set forth in SEQ ID NO: 21 or 29.

27. The binding agent for use of claim 25 or 26, wherein said Fe-mediated effector function is measured by binding to Fey receptors, binding to C1q, or induction of Fe-mediated crosslinking of Fey receptors.

28. The binding agent for use of claim 27, wherein said Fe-mediated effector function is measured by binding to C1q.

29. The binding agent for use of any one of claims 24-28, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

30. The binding agent for use of any one of claims 15-29, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

31. The binding agent for use of claim 30, wherein the positions corresponding to positions L234 and L235 in a human IgG1 heavy chain according to EU numbering are F and E, respectively, in said first and second heavy chains.

32. The binding agent for use of claim 30 or 31, wherein the positions corresponding to positions L234, L235, and D265 in a human IgG1 heavy chain according to EU numbering are F, E, and A, respectively, in said first and second heavy chain constant regions (HCs).

33. The binding agent for use of any one of claims 30-32, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

34. The binding agent for use of any one of claims 30-33, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

35. The binding agent for use of any one of claims 15-34, wherein the constant region of said first and/or second heavy chain comprises or consists essentially of or consists of an amino acid sequence selected from the group consisting of
a) the sequence set forth in SEQ ID NO: 21 or 29 [IgG1-FC];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4, at most 3, at most 2 substitutions or at most 1 substitution, compared to the amino acid sequence defined in a) or b).

36. The binding agent for use of any one of claims 15-34, wherein the constant region of said first or second heavy chain, such as the second heavy chain, comprises or consists essentially of or consists of an amino acid sequence selected from the group consisting of
a) the sequence set forth in SEQ ID NO: 22 or 30 [IgG1-F405L];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 9 substitutions, such as at most 8, at most 7, at most 6, at most 5, at most 4, at most 3, at most 2 substitutions or at most 1 substitution, compared to the amino acid sequence defined in a) or b).

37. The binding agent for use of any one of claims 15-34, wherein the constant region of said first or second heavy chain, such as the first heavy chain comprises or consists essentially of or consists of an amino acid sequence selected from the group consisting of
a) the sequence set forth in SEQ ID NO: 23 or 31 [IgG1-F409R];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

38. The binding agent for use of any one of claims 15-34, wherein the constant region of said first and/or second heavy chain comprises or consists essentially of or consists of an amino acid sequence selected from the group consisting of
a) the sequence set forth in SEQ ID NO: 24 or 32 [IgG1-Fc_FEA];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 7 substitutions, such as at most 6 substitutions, at most 5, at most 4, at most 3, at most 2 substitutions or at most 1 substitution, compared to the amino acid sequence defined in a) or b).

39. The binding agent for use of any one of claims 15-38, wherein the constant region of said first and/or second heavy chain, such as the second heavy chain, comprises or consists essentially of or consists of an amino acid sequence selected from the group consisting of
a) the sequence set forth in SEQ ID NO: 25 or 33 [IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).

40. The binding agent for use of any one of claims 15-39, wherein the constant region of said first and/or second heavy chain, such as the first heavy chain, comprises or consists essentially of or consists of an amino acid sequence selected from the group consisting of
a) the sequence set forth in SEQ ID NO: 26 or 34 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).

41. The binding agent for use of any one of the preceding claims, wherein said binding agent comprises a kappa (κ) light chain constant region.

42. The binding agent for use of any one of the preceding claims, wherein said binding agent comprises a lambda (λ) light chain constant region.

43. The binding agent for use of any one of the preceding claims, wherein said first light chain constant region is a kappa (κ) light chain constant region or a lambda (λ) light chain constant region.

44. The binding agent for use of any one of the preceding claims, wherein said second light chain constant region is a lambda (λ) light chain constant region or a kappa (κ) light chain constant region.

45. The binding agent for use of any one of the preceding claims, wherein said first light chain constant region is a kappa (κ) light chain constant region and said second light chain constant region is a lambda (λ) light chain constant region or said first light chain constant region is a lambda (λ) light chain constant region and said second light chain constant region is a kappa (κ) light chain constant region.

46. The binding agent for use of any one of claims 41-45, wherein the kappa (κ) light chain comprises an amino acid sequence selected from the group consisting of
a) the sequence set forth in SEQ ID NO: 27,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

47. The binding agent for use of any one of claims 42-46, wherein the lambda (λ) light chain comprises an amino acid sequence selected from the group consisting of
a) the sequence set forth in SEQ ID NO: 28,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

48. The binding agent for use of any one of the preceding claims, wherein the binding agent is of an isotype selected from the group consisting of IgG1, IgG2, IgG3, and IgG4.

49. The binding agent for use of any one of the preceding claims, wherein the binding agent is a full-length IgG1 antibody.

50. The binding agent for use of any one of the preceding claims, wherein the binding agent is an antibody of the IgG1m(f) allotype.

51. The binding agent for use of any one of the preceding claims, wherein the subject is a human subject.

52. The binding agent for use of any one of the preceding claims, wherein the tumor or cancer is a solid tumor or cancer.

53. The binding agent for use of any one of the preceding claims, wherein the tumor or cancer is selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, endometrial cancer, prostate cancer, penile cancer, cervical cancer, Hodgkin's lymphoma, non-Hodgkin's lymphoma, Merkel cell carcinoma and mesothelioma.

54. The binding agent for use of any one of the preceding claims, wherein the tumor or cancer is selected from the group consisting of melanoma, lung cancer, colorectal cancer, pancreatic cancer, and head and neck cancer.

55. The binding agent for use of claim 53 or 54, wherein the tumor or cancer is melanoma, such as cutaneous or acral melanoma.

56. The binding agent for use of claim 55, wherein the melanoma is unresectable melanoma, in particular unresectable Stage III or Stage IV melanoma.

57. The binding agent for use of claim 55 or 56, wherein the subject has not received prior treatment with a checkpoint inhibitor.

58. The binding agent for use of any one of claims 55-57, wherein the subject has not received prior systemic anticancer treatment for unresectable or metastatic melanoma.

59. The binding agent for use of claim 53 or 54, wherein the tumor or cancer is lung cancer, in particular a non-small cell lung cancer (NSCLC), such as a squamous or non-squamous NSCLC.

60. The binding agent for use of claim 59, wherein the lung cancer, in particular NSCLC, does not have an epidermal growth factor (EGFR)-sensitizing mutation and/or anaplastic lymphoma (ALK) translocation/ROS1 rearrangement.

61. The binding agent for use of claim 59 or 60, wherein the lung cancer, in particular NSCLC, comprises cancer cells and PD-L1 is expressed in 1% of the cancer cells.

62. The binding agent for use of any one of claims 59-61, wherein the subject has not received prior treatment with a checkpoint inhibitor.

63. The binding agent for use of claim 53 or 54, wherein the tumor or cancer is head and neck cancer, in particular head and neck squamous cell carcinoma (HNSCC).

64. The binding agent for use of claim 63, wherein the subject has not received prior treatment with a checkpoint inhibitor.

65. The binding agent for use of claim 53 or 54, wherein the tumor or cancer is pancreatic cancer, in particular pancreatic ductal adenocarcinoma (PDAC).

66. The binding agent for use of claim 65, wherein the subject has not received prior treatment of metastatic disease by radiotherapy, surgery, chemotherapy, or investigational therapy.

67. The binding agent for use of claim 65 or 66, wherein the subject has not received prior treatment with a checkpoint inhibitor.

68. The binding agent for use of claim 53 or 54, wherein the tumor or cancer is colorectal cancer.

69. The binding agent for use of claim 68, wherein the subject has not received prior treatment with a checkpoint inhibitor.

70. The binding agent for use of any one of the preceding claims, wherein the binding agent and the checkpoint inhibitor are administered in at least one treatment cycle, each treatment cycle being three weeks (21 days).

71. The binding agent for use of any one of the preceding claims, wherein one dose of the binding agent and one dose of the checkpoint inhibitor are administered every third week (1Q3W).

72. The binding agent for use of any one of the preceding claims, wherein one dose of the binding agent and one dose of the checkpoint inhibitor are administered on day 1 of each treatment cycle.

73. The binding agent for use of any one of the preceding claims, wherein the method further comprises administering to said subject one or more additional therapeutic agents.

74. The binding agent for use of claim 73, wherein the one or more additional therapeutic agents comprise one or more chemotherapeutic agents, such as platinum-based compounds (e.g., cisplatin, oxaliplatin, and carboplatin), taxane-based compounds (e.g., paclitaxel and nab-paclitaxel), nucleoside analogs (e.g., 5-fluorouracil and gemcitabine), and combinations thereof (e.g., cisplatin/carboplatin+5-fluorouracil or nab-paclitaxel+gemcitabine).

75. The binding agent for use of claim 73 or 74, wherein the one or more additional therapeutic agents are administered in at least one treatment cycle, each treatment cycle being three weeks (21 days).

76. The binding agent for use of any one of claims 73-75, wherein one dose of the one or more additional therapeutic agents is administered at least every third week (1Q3W) for at least the first treatment cycle, such as twice every third week (2Q3W) for at least the first treatment cycle.

77. The binding agent for use of any one of claims 73-76, wherein one dose of the one or more additional therapeutic agents is administered at least on day 1 of at least the first treatment cycle, such as on days 1 and 8 of at least the first treatment cycle.

78. A kit comprising (i) a binding agent comprising a first binding region binding to CD40 and a second binding region binding to CD137, (ii) a checkpoint inhibitor, and optionally (iii) one or more additional therapeutic agents.

79. The kit according to claim 78, wherein the binding agent and/or the checkpoint inhibitor and/or the one or more additional therapeutic agents is/are as defined in any one of claims 1-50 and 74.

80. The kit according to claim 78 or 79, wherein the binding agent, the checkpoint inhibitor, and, if present, the one or more additional therapeutic agents are for systemic administration, in particular for injection or infusion, such as intravenous injection or infusion.

81. The kit according to any one of claims 78-80 for use in a method for reducing or preventing progression of a tumor or treating cancer in a subject.

82. The kit for use according to claim 81, wherein the tumor or cancer and/or the subject and/or the method is/are as defined in any one of claims 51-77.

83. A method for reducing or preventing progression of a tumor or treating cancer in a subject, said method comprising administering to said subject the binding agent prior to, simultaneously with, or after administration of a checkpoint inhibitor, wherein the binding agent comprises a first binding region binding to CD40 and a second binding region binding to CD137.

84. The method of claim 83, wherein the tumor or cancer and/or the subject and/or the method and/or the binding agent and/or the checkpoint inhibitor is/are as defined in any one of claims 1-77.


SEQUENCE LISTING (76 sequences)

SEQ ID NO: 1 - protein, 8 aa, synthetic construct
  REGION 1..8: note = VH_CD40-001_CDR1
GYTFTEYI

SEQ ID NO: 2 - protein, 8 aa, synthetic construct
  REGION 1..8: note = VH_CD40-001_CDR2
IIPNNGGT

SEQ ID NO: 3 - protein, 15 aa, synthetic construct
  REGION 1..15: note = VH_CD40-001_CDR3
TRREVYGRNYYALDY

SEQ ID NO: 4 - protein, 6 aa, synthetic construct
  REGION 1..6: note = VL_CD40-001_CDR1
QGINNY

SEQ ID NO: 5 - skipped

SEQ ID NO: 6 - protein, 9 aa, synthetic construct
  REGION 1..9: note = VL_CD40-001_CDR3
QQYSNLPYT

SEQ ID NO: 7 - protein, 122 aa, synthetic construct
  REGION 1..122: note = VH_CD40-001
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSS

SEQ ID NO: 8 - protein, 107 aa, synthetic construct
  REGION 1..107: note = VL_CD40-001
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIK

SEQ ID NO: 9 - protein, 122 aa, synthetic construct
  REGION 1..122: note = VH_CD40-001 humanized
EVQLVQSGAEVKKPGASVKVSCKTSGYTFTEYIMHWVRQAPGQGLEWMGGIIPNNGGTSYNQKFQGRVTMTVDKSTSTGYMELSSLRSEDTAVYYCTRREVYGRNYYALDYWGQGTLVTVSS

SEQ ID NO: 10 - protein, 107 aa, synthetic construct
  REGION 1..107: note = VL_CD40-001 humanized
DIQMTQSPSSLSASVGDRVTITCSASQGINNYLNWYQQKPGKAVKLLIYYTSSLHSGVPSRFSGSGSGTDYTFTISSLQPEDIATYYCQQYSNLPYTFGGGTKVEIK

SEQ ID NO: 11 - protein, 8 aa, synthetic construct
  REGION 1..8: note = VH_CD137-009_CDR1
GFSLNDYW

SEQ ID NO: 12 - protein, 7 aa, synthetic construct
  REGION 1..7: note = VH_CD137-009_CDR2
IDVGGSL

SEQ ID NO: 13 - protein, 11 aa, synthetic construct
  REGION 1..11: note = VH_CD137-009_CDR3
ARGGLTYGFDL

SEQ ID NO: 14 - protein, 6 aa, synthetic construct
  REGION 1..6: note = VL_CD137-009_CDR1
EDISSY

SEQ ID NO: 15 - skipped

SEQ ID NO: 16 - protein, 12 aa, synthetic construct
  REGION 1..12: note = VL_CD137-009_CDR3
HYYATISGLGVA

SEQ ID NO: 17 - protein, 114 aa, synthetic construct
  REGION 1..114: note = VH_CD137-009
QSLEESGGRLVTPGTPLTLTCTVSGFSLNDYWMSWVRQAPGKGLEWIGYIDVGGSLYYASWAKGRFTISRTSTTVDLKMTSLTTEDTATYFCARGGLTYGFDLWGPGTLVTVSS

SEQ ID NO: 18 - protein, 110 aa, synthetic construct
  REGION 1..110: note = VL_CD137-009
DIVMTQTPASVSEPVGGTVTINCQASEDISSYLAWYQQKPGQRPKRLIYGASDLASGVPSRFSASGSGTEYALTISDLESADAATYYCHYYATISGLGVAFGGGTEVVVK

SEQ ID NO: 19 - protein, 117 aa, synthetic construct
  REGION 1..117: note = VH_CD137-009 humanized
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 20 - protein, 110 aa, synthetic construct
  REGION 1..110: note = VL_CD137-009 humanized
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 21 - protein, 330 aa, synthetic construct
  REGION 1..330: note = IgG1-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, synthetic construct
  REGION 1..330: note = IgG1-Fc_F405L
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 23 - protein, 330 aa, synthetic construct
  REGION 1..330: note = IgG1-Fc_K409R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 24 - protein, 330 aa, synthetic construct
  REGION 1..330: note = IgG1-Fc_FEA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 330 aa, synthetic construct
  REGION 1..330: note = IgG1-FEAL-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 26 - protein, 330 aa, synthetic construct
  REGION 1..330: note = IgG1-FEAR-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 27 - protein, 107 aa, synthetic construct [sequence omitted]

SEQ ID NO: 28 - protein, 106 aa, synthetic construct
  REGION 1..106: note = Lambda-C
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 329 aa, synthetic construct
  REGION 1..329: note = IgG1-Fc without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 329 aa, synthetic construct
  REGION 1..329: note = IgG1-Fc_F405L without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 31 - protein, 329 aa, synthetic construct
  REGION 1..329: note = IgG1-Fc_K409R without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 32 - protein, 329 aa, synthetic construct
  REGION 1..329: note = IgG1-Fc_FEA without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 329 aa, synthetic construct
  REGION 1..329: note = IgG1-FEAL-Fc without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 34 - protein, 329 aa, synthetic construct
  REGION 1..329: note = IgG1-FEAR-Fc without C-terminal Lys
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 35 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 36 - protein, 257 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 37 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 38 - protein, 232 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 39 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 40 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 41 - protein, 290 aa, Macaca fascicularis
MRIFAVFIFTIYWHLLNAFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTHLVILGAIFLLLGVALTFIFYLRKGRMMDMKKCGIRVTNSKKQRDTQLEET

SEQ ID NO: 42 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 43 - protein, 118 aa, synthetic construct
  REGION 1..118: note = VH_MAB-19-0618
QVQLVESGGGLVQPGTSLRLSCSVSGFSLYSYNMGWVRQAPGKGLEYIGIISGGTIGHYASWAKGRFTISRDTSKTTLYLQMNSLTTEDTATYFCARAFYDDYDYNVWGPGTLVTVSS

SEQ ID NO: 44 - protein, 111 aa, synthetic construct
  REGION 1..111: note = VL_MAB-19-0618
AIQLTQSPSSLSASVGGTVTITCQSSQSVYGNNQLSWYQQKPGQPPKLLIYQASKLETGVPSRFRGSGSGTQFTLTISSLQSEDFATYYCAGGYSSSSDTTFGGGTEVVVK

SEQ ID NO: 45 - protein, 8 aa, synthetic construct
  REGION 1..8: note = VH_MAB-19-0618_CDR1
GFSLYSYN

SEQ ID NO: 46 - protein, 8 aa, synthetic construct
  REGION 1..8: note = VH_MAB-19-0618_CDR2
IISGGTIG

SEQ ID NO: 47 - protein, 10 aa, synthetic construct
  REGION 1..10: note = VH_MAB-19-0618_CDR3
AFYDDYDYNV

SEQ ID NO: 48 - protein, 8 aa, synthetic construct
  REGION 1..8: note = VL_MAB-19-0618_CDR1
QSVYGNNQ

SEQ ID NO: 49 - skipped

SEQ ID NO: 50 - protein, 11 aa, synthetic construct
  REGION 1..11: note = VL_MAB-19-0618_CDR3
AGGYSSSSDTT

SEQ ID NO: 51 - protein, 8 aa, synthetic construct
  REGION 1..8: note = IgG1-PD1-MDX1106-FEAL VH CDR1
GITFSNSG

SEQ ID NO: 52 - protein, 8 aa, synthetic construct
  REGION 1..8: note = IgG1-PD1-MDX1106-FEAL VH CDR2
IWYDGSKR

SEQ ID NO: 53 - protein, 6 aa, synthetic construct
  REGION 1..6: note = IgG1-PD1-MDX1106-FEAL VH CDR3
ATNDDY

SEQ ID NO: 54 - protein, 6 aa, synthetic construct
  REGION 1..6: note = IgG1-PD1-MDX1106-FEAL VL CDR1
QSVSSY

SEQ ID NO: 55 - skipped

SEQ ID NO: 56 - protein, 9 aa, synthetic construct
  REGION 1..9: note = IgG1-PD1-MDX1106-FEAL VL CDR3
QQSSNWPRT

SEQ ID NO: 57 - protein, 113 aa, synthetic construct [sequence omitted]

SEQ ID NO: 58 - protein, 107 aa, synthetic construct [sequence omitted]

SEQ ID NO: 59 - protein, 127 aa, synthetic construct
  REGION 1..127: note = IgG1-b12 VH
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 60 - protein, 108 aa, synthetic construct
  REGION 1..108: note = IgG1-b12 VL
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 61 - protein, 329 aa, synthetic construct
  REGION 1..329: note = IgG1m(f)-L234F-L235E-G236R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 329 aa, synthetic construct
  REGION 1..329: note = IgG1m(f)-L234F-L235E-G236R-K409R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 63 - skipped

SEQ ID NO: 64 - protein, 5 aa, synthetic construct
  REGION 1..5: note = VH_MAB-19-0618_CDR1 (Kabat)
SYNMG

SEQ ID NO: 65 - protein, 7 aa, synthetic construct
  REGION 1..7: note = VH_MAB-19-0618_CDR2 (intersection of Kabat and IMGT)
ISGGTIG

SEQ ID NO: 66 - protein, 16 aa, synthetic construct
  REGION 1..16: note = VH_MAB-19-0618_CDR2 (Kabat)
IISGGTIGHYASWAKG

SEQ ID NO: 67 - protein, 12 aa, synthetic construct
  REGION 1..12: note = VH_MAB-19-0618_CDR3 (IMGT)
ARAFYDDYDYNV

SEQ ID NO: 68 - protein, 13 aa, synthetic construct
  REGION 1..13: note = VL_MAB-19-0618_CDR1 (Kabat)
QSSQSVYGNNQLS

SEQ ID NO: 69 - protein, 7 aa, synthetic construct
  REGION 1..7: note = VL_MAB-19-0618_CDR2 (Kabat)
QASKLET

SEQ ID NO: 70 - protein, 329 aa, synthetic construct
  REGION 1..329: note = Heavy chain human IgG1-LALA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 116 aa, synthetic construct
  REGION 1..116: note = VH_MAB-19-0202
QSVEESGGRLVTPGTPLTLTCTVSGFSLYSYNMGWVRQAPGKGLEYIGIISGGTIGHYASWAKGRFTISKTSSTTVDLKMTSLTTEDTATYFCARAFYDDYDYNVWGPGTLVTVSS

SEQ ID NO: 72 - protein, 111 aa, synthetic construct
  REGION 1..111: note = VL_MAB-19-0202
AAVLTQTPSPVSAAVGGTVTISCQSSQSVYGNNQLSWYQQKPGQPPKLLIYQASKLETGVPSRFKGSGSGTQFTLTISDLESDDAATYYCAGGYSSSSDTTFGGGTEVVVK

SEQ ID NO: 73 - protein, 329 aa, synthetic construct
  REGION 1..329: note = constant region human HC IgG1m(f)-E430G
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 74 - protein, 121 aa, synthetic construct
  REGION 1..121: note = VH_CD52-CAMPATH-1H
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 75 - protein, 107 aa, synthetic construct
  REGION 1..107: note = VL_CD52-CAMPATH-1H
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIK

SEQ ID NO: 76 - protein, 327 aa, synthetic construct
  REGION 1..327: note = constant region human HC IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK